(19) United States
(12) Patent Application Publication
(10) Pub. No.: US 2024/0254140 A1
(43) Pub. Date: Aug. 1, 2024

(54) COMPOUNDS AND THEIR USE IN THE TREATMENT OF BACTERIAL INFECTION

(71) Applicant: Hoffmann-La Roche Inc., Little Falls, NJ (US)

(72) Inventors: Daniele ANDREOTTI, Verona (IT); John G. CUMMING, Therwil (CH); Giacomo FOSSATI, Verona (IT); Susanna SAMPAOLESI, Verona (IT); Elena SERRA, Verona (IT)

(73) Assignee: Hoffmann-La Roche Inc., Little Falls, NJ (US)

(21) Appl. No.: 18/609,825

(22) Filed: Mar. 19, 2024

Related U.S. Application Data
(63) Continuation of application No. PCT/EP2022/075878, filed on Sep. 19, 2022.

(30) Foreign Application Priority Data
Sep. 20, 2021 (EP) 21197649.3

(51) Int. Cl. C07D 498/04; A61K 9/08; A61K 9/20; A61K 9/48; A61K 31/5383; A61K 47/10; A61K 47/12; A61P 31/04; C07D 519/00

(52) U.S. Cl. CPC C07D 498/04; A61K 9/08; A61K 9/2013; A61K 9/2018; A61K 9/2054; A61K 9/4858; A61K 9/4866; A61K 31/5383; A61K 47/10; A61K 47/12; A61P 31/04; C07D 519/00

(57) ABSTRACT

The invention provides novel compounds having the general formula (I)

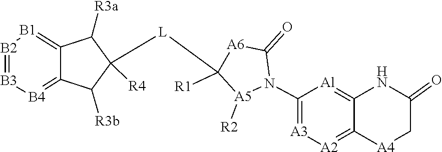

wherein R¹, R², R^{3a}, R^{3b}, R⁴, A¹, A², A³, A⁴, A⁵, A⁶, B¹, B², B³, B⁴, and L are as described herein, compositions including the compounds and methods of using the compounds.

### CROSS REFERENCE TO RELATED APPLICATIONS

This application is a continuation of International Application No. PCT/EP2022/075878, filed Sep. 19, 2022, which claims priority to European Patent Application No. 21197649.3 filed Sep. 20, 2021 both of which are incorporated herein by reference in their entirety.

### FIELD OF INVENTION

The present invention provides compounds which exhibit broad spectrum antibacterial activity, their manufacture, pharmaceutical compositions comprising them and their use as medicaments for the treatment of microbial infections, in particular for the treatment of diseases and infections caused by Pseudomonas aeruginosa, Klebsiella pneumoniae, Escherichia coli, Acinetobacter baumannii and Staphylococcus aureus, more particularly caused by Gram negative bacterias, furthermore particularly to Escherichia coli.

### BACKGROUND OF INVENTION

Bacterial infections pose a continuing medical problem because anti-bacterial drugs eventually engender resistance in the bacteria on which they are used. Bacterial resistance against virtually all current antibiotic drugs is increasing. Many forms of antibiotic resistance can even cross international boundaries and spread with remarkable speed. Thus novel classes of antibacterial compounds are urgently needed.

### SUMMARY OF THE INVENTION

The invention provides novel compounds of formula (I)

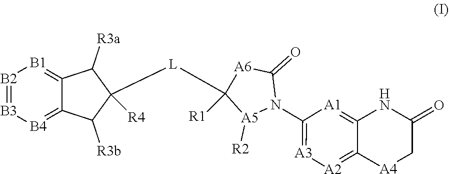

wherein
A¹ is —N—;
A² is selected from
   i) —N—, and
   ii) —CH—;
A³ is —CH—;
A⁴ is selected from
   i) —O—, and
   ii) —S—;
A⁵ is —CH—;
A⁶ is —O—;
B¹ is selected from
   i) —N—, and
   ii) —CR⁵—;
B² is selected from
   i) —N—, and
   ii) —CR⁶—;
B³ is —CNH—C(═O)R⁷—;
B⁴ is selected from
   i) —N—, and
   ii) —CR⁸—;
R² is H;
R³ is selected from
   i) H,
   ii) halogen, and
   iii) OH;
R^{3b} is selected from
   i) H,
   ii) halogen, and
   iii) OH;
   wherein at least one of R^{3a} and R^{3b} is H;
R⁴ is selected from
   i) H,
   ii) halogen,
   iii) C₁₋₆-alkoxy, and
   iii) OH;
R⁵ is selected from
   i) H,
   ii) halogen,
   iii) cyano, and
   iv) C₁₋₆-alkyl;
R⁶ is selected from
   i) H, and
   ii) C₁₋₆-alkyl;
R⁷ is selected from
   i) CR^{a}R^{b}NR^{c}R^{d},
   ii) CR^{a}R^{b}OH,
   iii) CHR^{e}R^{f},
   iv) heterocycloalkyl,
   v) heterocycloalkylalkyl,
   vi) substituted heterocycloalkyl,
   vii) substituted heterocycloalkylalkyl, and
   vii) substituted heteroaryl,
   wherein substituted heterocycloalkyl, substituted heterocycloalkylalkyl or substituted heteroaryl is substituted by 1 to 2 substituents independently selected from C₁₋₆-alkyl, C₁₋₆-alkoxy, OH, hydroxyalkyl, amino, and dialkylamino, or two substituents together form a 3-to-6-membered cycloalkyl ring;
R⁸ is selected from
   i) H,
   ii) halogen, and
   iii) cyano;
L is -L₁-NR⁹-L₂-;
L₁ is (CH₂)ₓ, wherein x represents an integer of 1 to 6;
L₂ is (CH₂)_{y}, wherein y represents an integer of 1 to 6,
R¹ is H and R⁹ is selected from
   i) H, and
   ii) C₁₋₆-alkyl;
or R¹, R⁹ and L₂ and the atoms to which they are bonded form a 4-6-membered heterocycle ring comprising a single N heteroatom;
or R¹ and L₂ and the atoms to which they are bonded form a 3-6-membered cycloalkyl ring;
R^{a} and R^{b} are independently selected from
   i) H,
   ii) C₁₋₆-alkyl, and
   iii) halogen,
   or R^{a} and R^{b} form a 3-membered cycloalkyl ring;
R^{c} and R^{d} are independently selected from
   i) H,
   ii) C₁₋₆-alkyl,
   iii) C₃-C₆-cycloalkyl, and
   iv) 4-6-membered heterocycloalkyl comprising a single oxygen heteroatom,
   or R^{c} and R^{d} form a 4 membered ring comprising a single N heteroatom;
R^{e} and R^{f} are independently selected from
   i) H,
   ii) NH₂,
   iii) hydroxyalkyl, and
   iv) alkyl-COOH;
or pharmaceutically acceptable salts thereof.

Objects of the present invention are novel compounds of formula (I), their manufacture, medicaments based on a compound in accordance with the invention and their production as well as the use of compounds of formula (I), for the treatment or prophylaxis of bacterial infection, in particular for the treatment of diseases and infections caused by Escherichia coli.

### DETAILED DESCRIPTION OF THE INVENTION

The term “C₁₋₆-alkyl” denotes a monovalent linear or branched saturated hydrocarbon group of 1 to 6 carbon atoms. Examples of C₁₋₆-alkyl include methyl, ethyl, propyl, isopropyl, n-butyl, iso-butyl, sec-butyl, tert-butyl and pentyl. Particular C₁₋₆-alkyl groups are methyl, ethyl and n-butyl. More particular example is methyl.

The term “cyano” denotes a —C≡N group.

The term “C₁₋₆-alkoxy” denotes a group of the formula —O—R′, wherein R′ is a C₁₋₆-alkyl group. Examples of C₁₋₆-alkoxy groups include methoxy, ethoxy, n-propoxy, isopropoxy, n-butoxy, isobutoxy and tert-butoxy. Particular example is methoxy.

The term “C₃₋₈-cycloalkyl” denotes a monovalent saturated monocyclic or bicyclic hydrocarbon group of 3 to 8 ring carbon atoms. Bicyclic means a ring system consisting of two saturated carbocycles having one or two carbon atoms in common. Examples of monocyclic C₃₋₈-cycloalkyl are cyclopropyl, cyclobutanyl, cyclopentyl, cyclohexyl or cycloheptyl. Particular monocyclic cycloalkyl groups are C₃₋₆-cycloalkyl, such as cyclopropyl, cyclobutanyl, cyclopentyl and cyclohexyl. More particular monocyclic cycloalkyl group is cyclopropyl.

The term “halogen” denotes fluoro, chloro, bromo or iodo. Particular halogens are chloro and fluoro.

The term “heterocycloalkyl” denotes a monovalent saturated or partly unsaturated mono- or bicyclic ring system of 4 to 9 ring atoms, comprising 1, 2, or 3 ring heteroatoms selected from N, O and S, the remaining ring atoms being carbon which is optionally substituted with oxo. Bicyclic means consisting of two cycles having one or two ring atoms in common. The heterocycloalkyl is preferably a monovalent saturated or partly unsaturated monocyclic ring system of 4 to 6 ring atoms, comprising 1 or 2 ring heteroatoms selected from N, O and S (4- to 6-membered heterocycloalkyl). Examples for monocyclic saturated heterocycloalkyl are 4,5-dihydro-oxazolyl, oxetanyl, azetidinyl, pyrrolidinyl, 2-oxo-pyrrolidin-4-yl, 3-oxo-morpholin-6-yl, tetrahydrofuranyl, tetrahydrothiophenyl, pyrazolidinyl, imidazolidinyl, oxazolidinyl, isoxazolidinyl, thiazolidinyl, piperidinyl, tetrahydropyranyl, tetrahydrothiopyranyl, piperazinyl, morpholinyl, thiomorpholinyl, 1,1-dioxo-thiomorpholin-4-yl, azepanyl, diazepanyl, homopiperazinyl, or oxazepanyl. Examples for bicyclic saturated heterocycloalkyl are oxabicyclo[2.2.1]heptanyl, oxaspiro[3.3]heptanyl, 8-aza-bicyclo[3.2.1]octyl, quinuclidinyl, 8-oxa-3-aza-bicyclo[3.2.1]octyl, 9-aza-bicyclo[3.3.1]nonyl, 3-oxa-9-aza-bicyclo[3.3.1]nonyl, or 3-thia-9-aza-bicyclo[3.3.1]nonyl. Examples for partly unsaturated heterocycloalkyl are dihydrofuryl, imidazolinyl, dihydro-oxazolyl, tetrahydro-pyridinyl or dihydropyranyl. Heterocyclyl is preferably azetidinyl, piperidyl, pyrrolidinyl, tetrahydrofuranyl, or oxetanyl.

The term “heterocycloalkylalkyl” denotes an alkyl group wherein at least one of the hydrogen atoms of the alkyl group has been replaced a heterocycloalkyl group. Examples are tetrahydrofuranylalkyl, pyrrolidinylalkyl, piperidylalkyl and azetidinylalkyl. More particularly, pyrrolidinylmethyl, piperidylmethyl, and azetidinylmethyl

The term “heteroaryl” denotes a monovalent aromatic mono- or bicyclic ring system of 4 to 9 ring atoms, comprising 1, 2, 3, or 4 ring heteroatoms selected from N, O and S, the remaining ring atoms being carbon which is optionally substituted with oxo. Bicyclic means consisting of two cycles having one or two ring atoms in common. The heteroaryl is preferably a monovalent aromatic ring system of 5 to 6 ring atoms, comprising 1 or 2 ring heteroatoms selected from N, O and S (5- to 6-membered heteroaryl). Example for heteroaryl are imidazole, pyrazole, oxazole, isoxazole, triazole, thiazole, tetrazole, pyridine, pyrimidine, pyrazine, or pyridazine. Particular heteroaryl is oxazole.

The term “hydroxy” denotes an —OH group.

The term “hydroxyalkyl” denotes an alkyl group wherein at least one of the hydrogen atoms of the alkyl group has been replaced by a hydroxy group. Examples of hydroxyalkyl include hydroxymethyl, hydroxyethyl, hydroxy-1-methyl-ethyl, hydroxypropyl, hydroxymethylpropyl and dihydroxypropyl.

Particular example is hydroxymethyl.

The term “amino” denotes an —NH₂ group.

The term “alkylamino” denotes an amino group wherein one of the hydrogen atoms of the amino group have been replaced by an alkyl group. Example is methylamino.

The term “dialkylamino” denotes an amino group wherein two of the hydrogen atoms of the amino group have been replaced by two alkyl groups. Example is dimethylamino.

The term “4- to 6-membered ring” denotes a monocyclic saturated or unsaturated ring system of 4 to 6 ring atoms. “4- to 6-membered ring” can comprise 1, 2, or 3 ring heteroatoms selected from N, O and S, the remaining ring atoms being carbon which is optionally substituted with oxo. Example for 4- to 6-membered ring are cyclohexane, cyclopentane, cyclobutane, azetidine, pyrrolidine, and piperidine. Particular 4- to 6-membered ring is cyclohexane.

The term “pharmaceutically acceptable salts” refers to those salts which retain the biological effectiveness and properties of the free bases or free acids, which are not biologically or otherwise undesirable. The salts are formed with inorganic acids such as hydrochloric acid, hydrobromic acid, sulfuric acid, nitric acid, phosphoric acid and the like, in particular hydrochloric acid, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, N-acetylcysteine, lactic acid and the like. In addition, these salts may be prepared by addition of an inorganic base or an organic base to the free acid. Salts derived from an inorganic base include, but are not limited to, the sodium, potassium, lithium, ammonium, calcium, magnesium salts and the like. Salts derived from organic bases include, but are not limited to salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, lysine, arginine, N-ethylpiperidine, piperidine, polyimine resins and the like. Particular pharmaceutically acceptable salts of compounds of formula (I) are the hydrochloride salts, methanesulfonic acid salts, lactic acid salts and citric acid salts.

The compounds of the present invention may absorb moisture, have absorbed water or form hydrates when left in the air. Such hydrates are also included in the salts of the present invention.

Further, the compounds of the present invention may absorb certain other solvents to form solvates. Such solvates are also encompassed in the present invention as salts of the compounds of the formula (I).

The term “protecting group” (PG) denotes a group which selectively blocks a reactive site in a multifunctional compound such that a chemical reaction can be carried out selectively at another unprotected reactive site in the meaning conventionally associated with it in synthetic chemistry.

Protecting groups can be removed at the appropriate point. Exemplary protecting groups are amino-protecting groups, carboxy-protecting groups or hydroxy-protecting groups. Particular protecting groups are the tert-butoxycarbonyl (Boc), benzyloxycarbonyl (Cbz), fluorenylmethoxycarbonyl (Fmoc) and benzyl (Bn) groups. Further particular protecting groups are the tert-butoxycarbonyl (Boc) and the fluorenylmethoxycarbonyl (Fmoc) groups. More particular protecting group is the tert-butoxycarbonyl (Boc) group.

The abbreviation uM means microMolar and is equivalent to the symbol μM.

The abbreviation uL means microliter and is equivalent to the symbol μL.

The abbreviation ug means microgram and is equivalent to the symbol μg.

The compounds of formula (I) can contain several asymmetric centers and can be present in the form of optically pure enantiomers, mixtures of enantiomers such as, for example, racemates, optically pure diastereoisomers, mixtures of diastereoisomers, diastereoisomeric racemates or mixtures of diastereoisomeric racemates.

According to the Cahn-Ingold-Prelog Convention the asymmetric carbon atom can be of the “R” or “S” configuration.

Also an embodiment of the present invention are compounds according to formula (I) as described herein and pharmaceutically acceptable salts thereof, in particular compounds according to formula (I) as described herein and pharmaceutically acceptable salts thereof, more particularly compounds according to formula (I) as described herein.

An embodiment of the present invention are compounds according to formula (I) as described herein, wherein
A¹ is —N—;
A² is selected from
   i) —N—, and
   ii) —CH—;
A³ is —CH—;
A⁴ is selected from
   i) —O—, and
   ii) —S—;
A⁵ is —CH—;
A⁶ is —O—;
B¹ is selected from
   i) —N—, and
   ii) —CR⁵—;
B² is selected from
   i) —N—, and
   ii) —CR⁶—;
B³ is —CNH—C(═O)R⁷—;
B⁴ is selected from
   i) —N—, and
   ii) —CR⁸—;
R² is H;
R^{3a} is selected from
   i) H,
   ii) halogen, and
   iii) OH;
R^{3b} is selected from
   i) H,
   ii) halogen, and
   iii) OH;
   wherein at least one of R^{3a} and R^{3b} is H;
R⁴ is selected from
   i) H,
   ii) halogen,
   iii) C₁₋₆-alkoxy, and
   iii) OH;
R⁵ is selected from
   i) H,
   ii) halogen,
   iii) cyano, and
   iv) C₁₋₆-alkyl;
R⁶ is selected from
   i) H, and
   ii) C₁₋₆-alkyl;
R⁷ is selected from
   i) CR^{a}R^{b}NR^{c}R^{d},
   ii) CR^{a}R^{b}OH,
   iii) CHR^{e}R^{f},
   iv) heterocycloalkyl,
   v) heterocycloalkylalkyl,
   vi) substituted heterocycloalkyl,
   vii) substituted heterocycloalkylalkyl, and
   vii) substituted heteroaryl,
   wherein substituted heterocycloalkyl, substituted heterocycloalkylalkyl or substituted heteroaryl is substituted by 1 to 2 substituents independently selected from C₁₋₆-alkyl, C₁₋₆-alkoxy, OH, hydroxyalkyl, amino, and dialkylamino, or two substituents together form a 3-to-6-membered cycloalkyl ring.
R⁸ is selected from
   i) H,
   ii) halogen, and
   iii) cyano;
L is -L₁-NR⁹-L₂-;
L₁ is (CH₂)ₓ, wherein x represents an integer of 1 to 6;
L₂ is (CH₂)_{y}, wherein y represents an integer of 1 to 6,
R¹ is H and R⁹ is selected from
   i) H, and
   ii) C₁₋₆-alkyl;
or R¹, R⁹ and L₂ and the atoms to which they are bonded form a 4-6-membered heterocycle ring comprising a single N heteroatom;
or R¹ and L₂ and the atoms to which they are bonded form a 3-6-membered cycloalkyl ring;
R^{a} and R^{b} are independently selected from
   i) H,
   ii) C₁₋₆-alkyl, and
   iii) halogen,
   or R^{a} and R^{b} form a 3-membered cycloalkyl ring;
R^{c} and R^{d} are independently selected from
   i) H,
   ii) C₁₋₆-alkyl,
   iii) C₃-C₆-cycloalkyl, and
   iv) 4-6-membered heterocycloalkyl comprising a single oxygen heteroatom,
   or R^{c} and R^{d} form a 4 membered ring comprising a single N heteroatom;
R^{e} and R^{f} are independently selected from
   i) H,
   ii) NH₂,
   iii) hydroxyalkyl, and
   iv) alkyl-COOH;
or pharmaceutically acceptable salts thereof.

Another embodiment of the present invention are compounds according to formula (I) as described herein, wherein
A¹ is —N—;
A² is —N—;
A³ is —CH—;
A⁴ is —O—;
A⁵ is —CH—;
A⁶ is —O—;
B¹ is —CR⁵—;
B² is —CR⁶—;
B³ is —CNH—C(═O)R⁷—;
B⁴ is —CR⁸—;
R² is H;
R^{3a} H;
R^{3b} is H;
R⁴ is H;
R⁵ is selected from
   i) halogen, and
   ii) cyano;
R⁶ is H;
R⁷ is selected from
   i) CR^{a}R^{b}NR^{c}R^{d},
   ii) CR^{a}R^{b}OH,
   iii) a 4-membered heterocycloalkylalkyl comprising a single N heteroatom,
   iv) a 5-membered substituted heterocycloalkyl comprising a single N heteroatom, wherein substituted heterocycloalkyl is substituted by 1 to 2 substituents independently selected from C₁₋₆-alkyl, OH, and hydroxyalkyl.
R⁸ is selected from
   i) H, and
   ii) halogen;
L is -L₁-NR⁹-L₂-;
L₁ is (CH₂)ₓ, wherein x is 1;
L₂ is (CH₂)_{y}, wherein y is 2;
R¹ and R⁹ are H,
or R¹, R⁹ and L₂ and the atoms to which they are bonded form the piperidine ring as below,

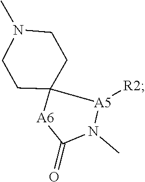

R^{a} and R^{b} are independently selected from
   i) H,
   ii) C₁₋₆-alkyl, and
   iii) halogen,
R^{c} and R^{d} are C₁₋₆-alkyl, or R^{e} and R^{d} form a 4 membered ring comprising a single N heteroatom;
or pharmaceutically acceptable salts thereof.

Also a particular embodiment of the present invention provides compounds according to formula (I) as described herein, wherein
A¹ is —N—;
A² is —N—;
A³ is —CH—;
A⁴ is —O—;
A⁵ is —CH—;
A⁶ is —O—;
B¹ is —CR⁵—;
B² is —CR⁶—;
B³ is —CNH—C(═O)R⁷—;
B⁴ is —CR⁸—;
R² is H;
R^{3a} H;
R^{3b} is H;
R⁴ is H;
R⁵ is halogen;
R⁶ is H;
R⁷ is CR^{a}R^{b}NR^{c}R^{d};
R⁸ is H;
L is -L₁-NR⁹-L₂-;
L₁ is (CH₂)ₓ, wherein x is 1;
R¹ and R⁹ are H;
L₂ is (CH₂)_{y}, wherein y is 2;
R^{a} and R^{b} are independently selected from
   i) H, and
   ii) C₁₋₆-alkyl;
R^{c} and R^{d} are C₁₋₆-alkyl;
or pharmaceutically acceptable salts thereof.

An embodiment of the present invention provides compounds according to formula (I) as described herein, wherein A⁴ is —O—.

An embodiment of the present invention provides compounds according to formula (I) as described herein, wherein R⁶ is H.

An embodiment of the present invention provides compounds according to formula (I) as described herein, wherein R⁸ is H or halogen.

A particular embodiment of the present invention provides compounds according to formula (I) as described herein, wherein R⁸ is H.

An embodiment of the present invention provides compounds according to formula (I) as described herein, wherein A² is —N—.

An embodiment of the present invention provides compounds according to formula (I) as described herein, wherein B¹ is —CR⁵—.

An embodiment of the present invention provides compounds according to formula (I) as described herein, wherein B² is —CR⁶—.

An embodiment of the present invention provides compounds according to formula (I) as described herein, wherein B⁴ is —CR⁸—.

An embodiment of the present invention provides compounds according to formula (I) as described herein, wherein R⁴ is H.

An embodiment of the present invention provides compounds according to formula (I) as described herein, wherein R⁵ is cyano or halogen.

A particular embodiment of the present invention provides compounds according to formula (I) as described herein, wherein R⁵ is halogen.

An embodiment of the present invention provides compounds according to formula (I) as described herein, wherein R⁷ is selected from
i) CR^{a}R^{b}NR^{c}R^{d},
ii) CR^{a}R^{b}OH,
iii) CHR^{e}R^{f},
iv) a 5-membered heterocycloalkyl comprising a single N heteroatom,
v) a 3-6-membered heterocycloalkylalkyl comprising a single N heteroatom,
vi) a 5-membered substituted heterocycloalkyl comprising a single N or O heteroatom,
vii) a 5-membered substituted heterocycloalkylalkyl comprising a single N heteroatom, and
viii) substituted oxazole,
wherein substituted heterocycloalkyl, substituted heterocycloalkylalkyl or substituted oxazole is substituted by 1 to 2 substituents independently selected from C₁₋₆-alkyl, C₁₋₆-alkoxy, OH, hydroxyalkyl, amino, and dialkylamino, or two substituents together form a 3-to-6-membered cycloalkyl ring.

An embodiment of the present invention provides compounds according to formula (I) as described herein, wherein R⁷ is selected from
i) CR^{a}R^{b}NR^{c}R^{d},
ii) CR^{a}R^{b}OH,
iii) a 4-membered heterocycloalkylalkyl comprising a single N heteroatom,
iv) a 5-membered substituted heterocycloalkyl comprising a single N heteroatom,
wherein substituted heterocycloalkyl is substituted by 1 to 2 substituents independently selected from C₁₋₆-alkyl, OH, or hydroxyalkyl.

A particular embodiment of the present invention provides compounds according to formula (I) as described herein, wherein R⁷ is CR^{a}R^{b}NR^{c}R^{d}.

An embodiment of the present invention provides compounds according to formula (I) as described herein, wherein R⁹ is H.

An embodiment of the present invention provides compounds according to formula (I) as described herein, wherein R^{a} and R^{b} are independently selected from H, C₁₋₆-alkyl and halogen.

A particular embodiment of the present invention provides compounds according to formula (I) as described herein, wherein R^{a} is H and R^{b} is C₁₋₆-alkyl.

An embodiment of the present invention provides compounds according to formula (I) as described herein, wherein R^{c} and R^{d} are independently selected from C₁₋₆-alkyl or together form a 4-membered ring comprising a single N heteroatom.

A particular embodiment of the present invention provides compounds according to formula (I) as described herein, wherein R^{c} and R^{d} are both C₁₋₆-alkyl.

An embodiment of the present invention provides compounds according to formula (I) as described herein, wherein x is 2.

An embodiment of the present invention provides compounds according to formula (I) as described herein, wherein y is 1.

An embodiment of the present invention provides compounds according to formula (I) as described herein, wherein R¹, R⁹ and L₂ and the atoms to which they are bonded form the piperidine ring as below,

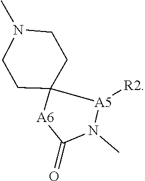

Processes for the manufacture of compounds of formula (I) as described herein are also an object of the invention.

The preparation of compounds of formula (I) of the present invention may be carried out in sequential or convergent synthetic routes. Syntheses of the invention are shown in the following general schemes. The skills required for carrying out the reactions and purifications of the resulting products are known to those skilled in the art. The substituents and indices used in the following description of the processes have the significance given herein before unless indicated to the contrary.

In more detail, the compounds of formula (I) can be manufactured by the methods given below, by the methods given in the examples or by analogous methods. Appropriate reaction conditions for the individual reaction steps are known to a person skilled in the art. The reaction sequence is not limited to the ones displayed in schemes 1-11, however, depending on the starting materials and their respective reactivity the sequence of reaction steps can be freely altered. Starting materials are either commercially available or can be prepared by methods analogous to the methods given below, by methods described in references cited in the description or in the examples, or by methods known in the art.

The compounds of formula (I), wherein L₁ is (CH₂)x and x is 1 to 6, and their pharmaceutically acceptable salts can be prepared by a process described below (Scheme 1).

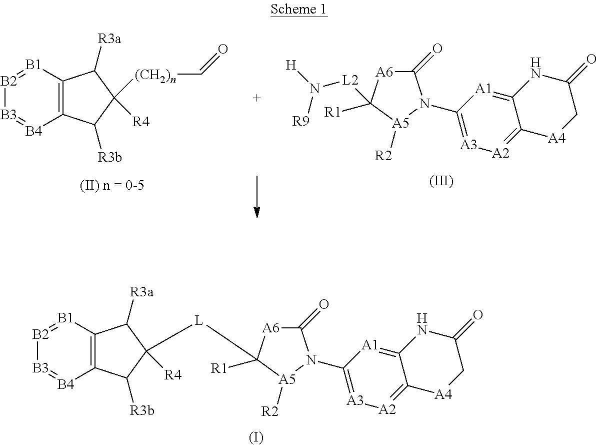

Reductive amination of aldehydes of formula (II) with amines of formula (III) affords compounds of formula (I). Typical conditions include sodium triacetoxyborohydride or sodium cyanoborohydride, optionally in the presence of additives such as N,N-diisopropylethylamine or triethylamine or acetic acid or powdered molecular sieves in a solvent such as methanol, THF or 1,2-dichloroethane at a temperature such as room temperature.

Alternatively, compounds of formula (I) may be prepared as illustrated in scheme 2.

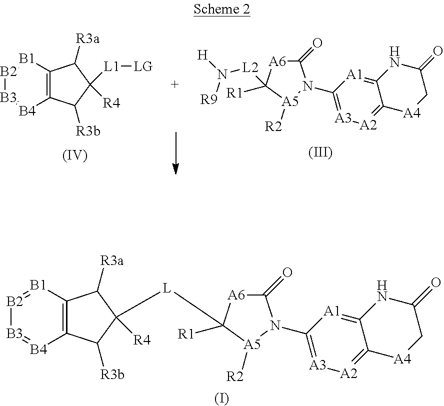

Reaction of intermediates of formula (IV) wherein LG is a leaving group such as iodo, bromo, mesyloxy or tosyloxy with amines of formula (III) affords compounds of formula (I). Typical conditions include a base such as potassium carbonate or caesium carbonate in a solvent such as MeCN, DMF, DMA or NMP at an elevated temperature such as 80° C.

Alternatively, compounds of formula (I) may be prepared as illustrated in scheme 3.

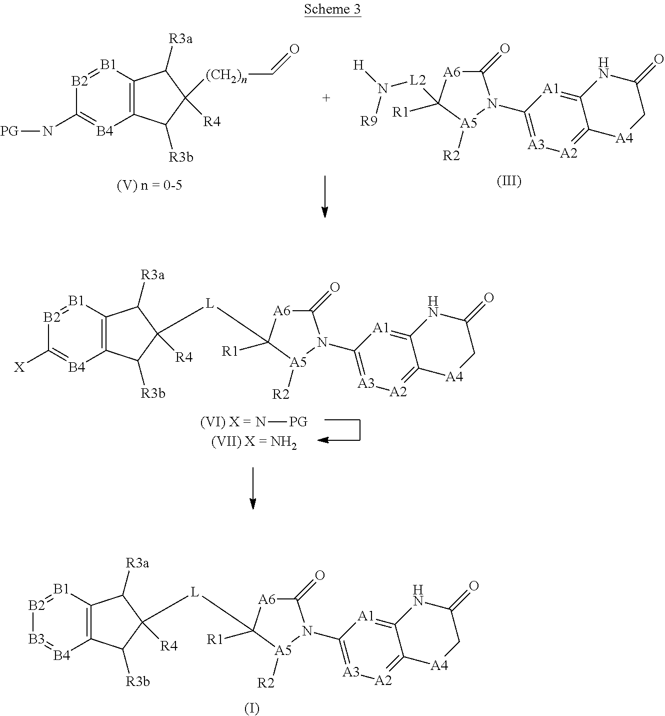

Reductive amination of aldehydes of formula (V), wherein X is a protected amine functionality, with amines of formula (III) affords intermediates of formula (VI). Typical conditions include sodium triacetoxyborohydride or sodium cyanoborohydride, optionally in the presence of additives such as N,N-diisopropylethylamine or triethylamine or acetic acid or powdered molecular sieves in a solvent such as methanol, THF or 1,2-dichloroethane at a temperature such as room temperature.

Deprotection of protected amines of formula (VI) affords amines of formula (VII). Typical protecting groups include Boc for which typical deprotection conditions include an acid such as trifluoroacetic acid or hydrochloric acid in a solvent such as DCM or EtOAc at a temperature such as room temperature. Alternatively, the protected amine functionality may be a nitro group which may be reduced to afford amines of formula (VII). Typical conditions include hydrogenation under an atmosphere of hydrogen in the presence of a catalyst such as palladium on carbon in a solvent such as ethanol at a temperature such as room temperature.

Reaction of amines of formula (VII) with carboxylic acids affords compounds of formula (I). Typical conditions include a coupling agent such as TCFH or HATU in the presence of a base such as DIPEA or 1-methylimidazole in a solvent such as DCM, DMF or MeCN at a temperature such as room temperature.

Intermediates of formula (II) may be prepared as illustrated in scheme 4.

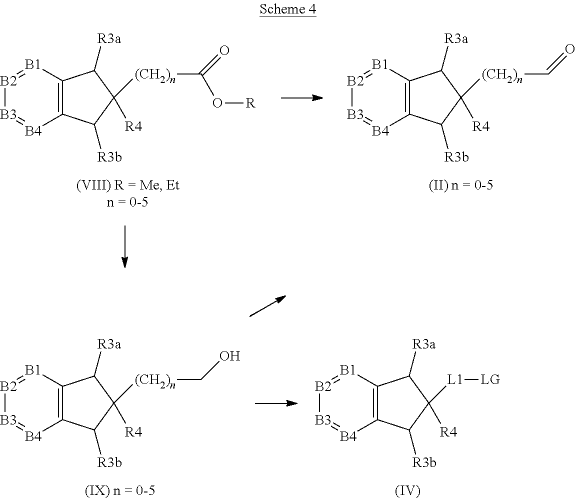

Reduction of esters of formula (VIII) affords alcohols of formula (IX). Typical conditions include a reducing agent such as lithium aluminium hydride in a solvent such as THF at a temperature such as between −78° C. and room temperature. Oxidation of alcohols of formula (IX) affords aldehydes of formula (II). Typical conditions include Dess-Martin periodinane in a solvent such as DCM at a temperature such as 0 to 25° C., or sulfur trioxide pyridine complex in the presence of triethylamine and dry dimethylsulfoxide in a solvent such as DCM at a temperature such as 0 to 25° C.

Alternatively, reduction of esters of formula (VIII) affords aldehydes of formula (II). Typical conditions include a reducing agent such as lithium aluminium hydride in a solvent such as THF at a reduced temperature such as between −78° C. and −20° C.

Intermediates of formula (IV), wherein LG is a leaving group such as iodo, bromo, mesyloxy or tosyloxy, may be prepared as illustrated in scheme 4. Activation of alcohols of formula (IX) affords intermediates of formula (IV). Typical conditions include p-toluenesulfonyl chloride or methanesulfonyl chloride, N,N-diisopropylethylamine or triethylamine and DMAP in a solvent such as DCM at a temperature such as between 0° C. and room temperature, optionally followed by reaction of the resulting mesyloxy or tosyloxy derivative with a halide salt such as sodium iodide or potassium bromide in a solvent such as MeCN at a temperature such as 60° C.

Intermediates of formula (VIII) and (IX) may be prepared as illustrated in scheme 5.

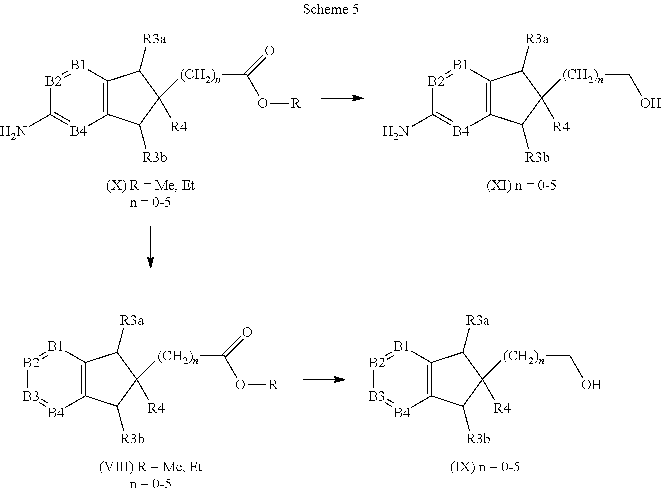

Reaction of amines of formula (X) or formula (XI) with carboxylic acids affords intermediates of formula (VIII) and formula (IX) respectively. Typical conditions include a coupling agent such as TCFH, T3P or HATU in the presence of a base such as DIPEA, DMAP or 1-methylimidazole in a solvent such as DCM, DMF or MeCN at a temperature such as room temperature. Alternatively, reaction of amines of formula (X) with acid chlorides affords intermediates of formula (VIII). Typical conditions include the presence of a base such as triethylamine or DIPEA in a solvent such as DCM, DMA or THF at a temperature such as between −10° C. and 25° C.

Intermediates of formula (XI) may be prepared as illustrated in scheme 5. Reduction of esters of formula (X) affords alcohols of formula (XI). Typical conditions include a reducing agent such as lithium aluminium hydride in a solvent such as THF at a temperature such as between −78° C. and room temperature.

Intermediates of formula (X) may be prepared as illustrated in scheme 6.

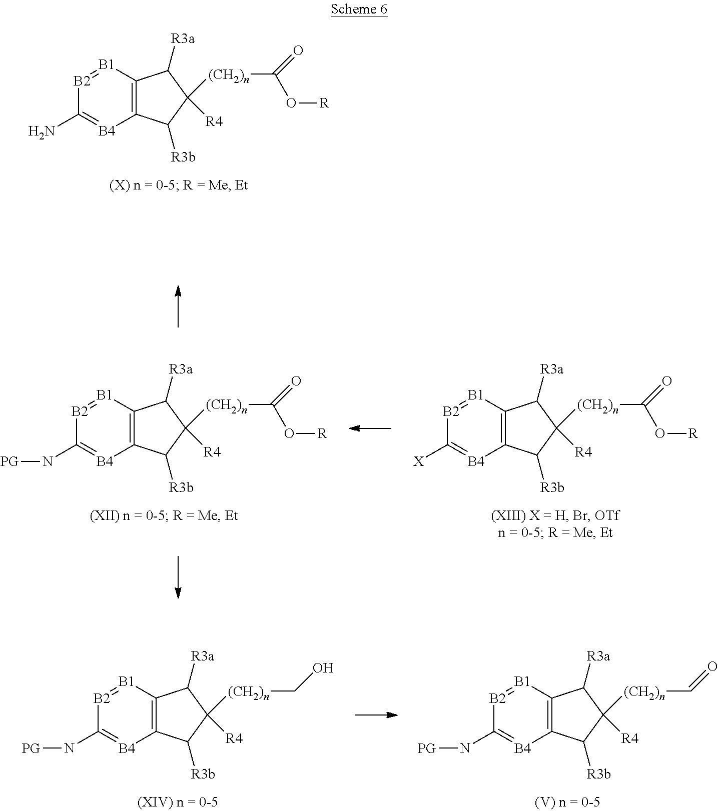

Nitration of intermediates of formula (XIII), wherein X is H, affords protected amines of formula (XII), wherein the amine protecting group is nitro. Typical conditions include a mixture of nitric acid and sulfuric acid at a reduced temperature such as between −10 and 0° C. Reduction of protected amines of formula (XII) wherein the amine protecting group is nitro affords amines of formula (X). Typical conditions include hydrogenation under an atmosphere of hydrogen in the presence of a catalyst such as palladium on carbon in a solvent such as ethanol at a temperature such as room temperature.

Amination of intermediates of formula (XIII), wherein X is Br or triflate, affords protected amines of formula (XII), wherein the amine protecting group is Boc or diphenylmethanimine. Typical conditions include reaction with tert-butyl carbamate or diphenylmethanimine in the presence of a palladium catalyst such as tris(dibenzylideneacetone)dipalladium (0) and a phosphine ligand such as xantphos or XPhos and a base such as cesium carbonate in a solvent such as 1,4-dioxane at a temperature such as between 90 and 100° C. Deprotection of protected amines of formula (XII) wherein the amine protecting group is Boc or diphenylmethanimine affords amines of formula (X). Typical conditions include an acid such as trifluoroacetic acid or hydrochloric acid in a solvent such as DCM, THF or EtOAc at a temperature such as room temperature.

Intermediates of formula (XIII), wherein X is H, may be converted into intermediates of formula (XIII), wherein X is triflate, using a three step procedure. Typical conditions for the first step include 4,4,5,5-tetramethyl-1,3,2-dioxaborolane and 3,4,7,8-tetramethyl-1,10-phenanthroline in the presence of a catalyst such as (1,5-cyclooctadiene)(methoxy)iridium(I) dimer at a temperature such as 65° C. Typical conditions for the second step include sodium perborate monohydrate in water at a temperature such as between 0 and 25° C. Typical conditions for the third step include trifluoromethanesulfonic anhydride in the presence of a base such as 2,6-lutidine in a solvent such as DCM at a temperature such as 0° C.

Intermediates of formula (V) may be prepared as illustrated in scheme 6. Reduction of esters of formula (XII) affords alcohols of formula (XIV). Typical conditions include a reducing agent such as lithium aluminium hydride in a solvent such as THF at a temperature such as between −78° C. and room temperature. Oxidation of alcohols of formula (XIV) affords aldehydes of formula (V). Typical conditions include Dess-Martin periodinane in a solvent such as DCM at a temperature such as between 0 and 25° C., or sulfur trioxide pyridine complex in the presence of triethylamine and dry dimethylsulfoxide in a solvent such as DCM at a temperature such as between 0 and 25° C.

Intermediates of formula (XII) and (XIII) may be prepared as illustrated in scheme 7.

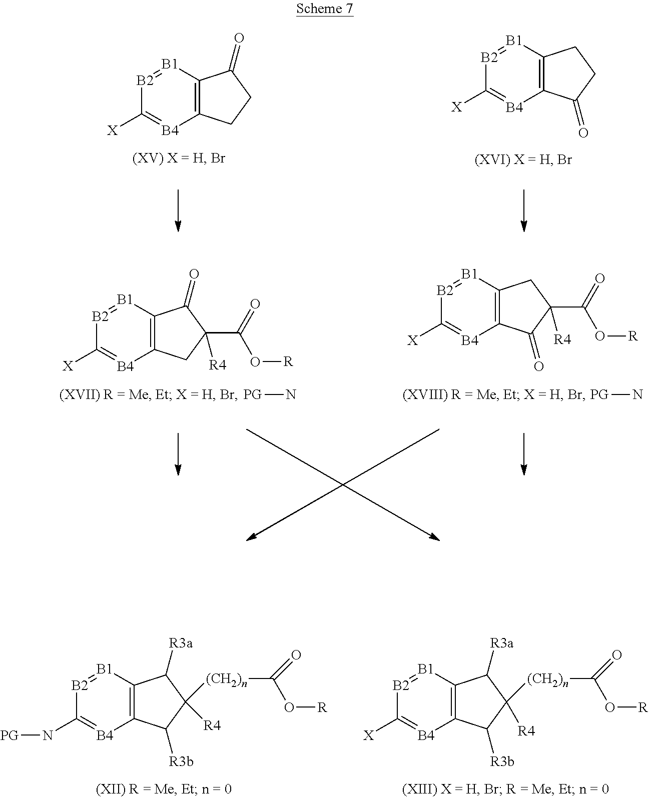

Reaction of ketones of formula (XV) or formula (XVI) with diethyl or dimethyl carbonate affords β-keto esters of formula (XVII) and formula (XVIII) respectively, wherein X is H or Br and R⁴ is H. Typical conditions include a base such as sodium hydride in a solvent such as toluene at a temperature such as 120° C.

Amination of β-keto esters of formula (XVII) or formula (XVIII), wherein X is Br, affords β-keto esters of formula (XVII) and formula (XVIII) respectively, wherein X is protected amino. Typical conditions include tert-butyl carbamate in the presence of a palladium catalyst such as tris(dibenzylideneacetone)dipalladium (0) and a phosphine ligand such as xantphos or XPhos and a base such as cesium carbonate in a solvent such as 1,4-dioxane at a temperature such as between 90 and 100° C.

β-Keto-esters of formula (XVII) or formula (XVIII), wherein R⁴ is H, may be reduced to esters of formula (XIII) or (XII) wherein R^{3a}, R^{3b} and R⁴ are H. Typical conditions include triethylsilane and trifluoroacetic acid at a temperature such as between 0° C. and 25° C.

Reduction of β-keto esters of formula (XVII) or formula (XVIII), affords alcohols of formula (XIII) or formula (XII), wherein R^{3a} or R^{3b} respectively is OH. Typical conditions include sodium borohydride in a solvent such as ethanol at a temperature such as 0° C., or hydrogenation in the presence of a catalyst such as palladium on carbon in a solvent such as EtOAc at a temperature such as room temperature.

Reaction of alcohols of formula (XIII) or formula (XII), wherein R^{3a} or R^{3b} or R⁴ is OH, with a reagent such as diethylaminosulfur trifluoride in a solvent such as DCM at a temperature such as −78° C. affords intermediates of formula (XIII) or formula (XII), wherein R^{3a} or R^{3b} or R⁴ respectively is F.

Alcohols of formula (XIII) or formula (XII), wherein R^{3a} or R^{3b} is OH, may be converted into intermediates of formula (XIII) or formula (XII), wherein R^{3a} and R^{3b} are H using a two step procedure. Typical conditions for the first step include thionyl chloride and a base such as triethylamine in a solvent such as DCM at a temperature such as 0° C. Typical conditions for the second step include hydrogenation in the presence of a catalyst such as palladium on carbon in a solvent such as EtOAc at a temperature such as room temperature.

Reaction of β-keto esters of formula (XVII) or formula (XVIII), wherein R⁴ is H, with a reagent such as 3-chloroperoxybenzoic acid in a solvent such as DCM at a temperature such as 0° C. affords intermediates of formula (XVII) or formula (XVIII), wherein R⁴ is OH.

Alternatively, Intermediates of formula (XIII), wherein R^{3a}, R^{3b} and R⁴ are H, may be prepared as illustrated in scheme 8.

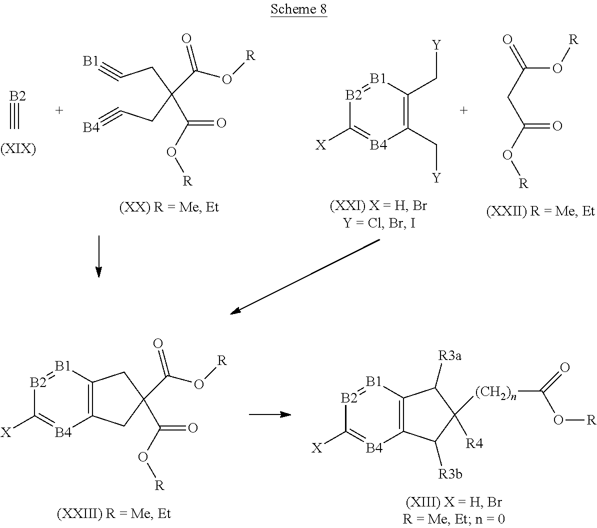

Cycloaddition of an alkyne or isocyanate of formula (XIX) with a diyne or alkyne-nitrile of formula (XX) affords intermediates of formula (XXIII). Typical conditions include use of a catalyst mixture such as chloro(1,5-cyclooctadiene)iridium(I) dimer and 1,1′-bis(diphenylphosphino)ferrocene in a solvent such as benzene at an elevated temperature or a catalyst such as chloro(1,5-cyclooctadiene(pentamethylcyclopentadienyl)ruthenium(II) in a solvent such as 1,2-dichloroethane at an elevated temperature such as 90° C.

Alternatively, reaction of a dihalomethyl compound of formula (XXI) with a malonate diester of formula (XXII) affords diesters of formula (XXIII). Typical conditions include the use of a base such as sodium hydride in a solvent such as THF at a temperature such as 0° C.

Diesters of formula (XXIII) may be converted to esters of formula (XIII) wherein R^{3a}, R^{3b} and R⁴ are H. Typical conditions include use of a salt such as lithium chloride in DMSO and water at an elevated temperature such as 160° C.

Alternatively, diesters of formula (XXIII) may be treated with an acid such as HCl in water at a temperature such as 100° C. followed by reaction with an acid such as sulfuric acid in a solvent such as methanol or ethanol at a temperature such as 80° C. to afford esters of formula (XIII) wherein R^{3a}, R^{3b} and R⁴ are H.

Intermediates of formula (III) may be prepared as illustrated in scheme 9.

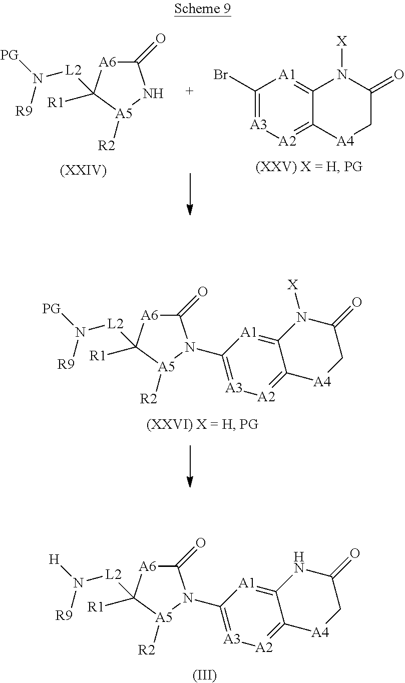

Coupling of compounds of formula (XXIV) with heteroaryl bromides of formula (XXV), optionally protected, affords intermediates of formula (XXVI) optionally protected. Typical conditions include the use of a catalytic quantity of copper (I) iodide and trans-N,N′-dimethylcyclohexane-1,2-diamine or of tris(dibenzylideneacetone)dipalladium (0) and 9,9-dimethyl-4,5-bis(diphenylphosphino)xanthene in the presence of a base such as potassium carbonate in a solvent such as 1,4-dioxane at a temperature such as 100° C. Typical optional protecting groups in heteroaryl bromides of formula (XXV) include 2-trimethylsilylethoxymethyl. Deprotection of intermediates of formula (XXVI) affords intermediates of formula (III). Typical conditions include an acid such as hydrochloric acid in a solvent such as methanol or ethyl acetate, or trifluroacetic acid in a solvent such as DCM at a temperature such as 25° C.

Particular examples of compounds of formula (I) as described herein are selected from
2-(dimethylamino)-N-[7-fluoro-2-[[2-[2-oxo-3-(3-oxo-4H-pyrazino[2,3-b][1,4]oxazin-6-yl)oxazolidin-5-yl]ethylamino]methyl]indan-5-yl]acetamide;
1-(dimethylamino)-N-[7-fluoro-2-[[2-[2-oxo-3-(3-oxo-4H-pyrazino[2,3-b][1,4]oxazin-6-yl)oxazolidin-5-yl]ethylamino]methyl]indan-5-yl]cyclopropanecarboxamide;
2-(dimethylamino)-N-[7-fluoro-2-[[2-[2-oxo-3-(3-oxo-4H-pyrazino[2,3-b][1,4]oxazin-6-yl)oxazolidin-5-yl]ethylamino]methyl]indan-5-yl]-2-methyl-propanamide;
(2R)-2-(dimethylamino)-N-[7-fluoro-2-[[2-[2-oxo-3-(3-oxo-4H-pyrazino[2,3-b][1,4]oxazin-6-yl)oxazolidin-5-yl]ethylamino]methyl]indan-5-yl]propanamide;
N-[7-fluoro-2-[[2-[2-oxo-3-(3-oxo-4H-pyrazino[2,3-b][1,4]oxazin-6-yl)oxazolidin-5-yl]ethylamino]methyl]indan-5-yl]-2-morpholino-acetamide;
2-(dimethylamino)-N-[7-fluoro-2-[[2-oxo-3-(3-oxo-4H-pyrazino[2,3-b][1,4]oxazin-6-yl)-1-oxa-3,8-diazaspiro[4.5]decan-8-yl]methyl]indan-5-yl]-2-methyl-propanamide;
(2R)-2-(dimethylamino)-N-[7-fluoro-2-[[2-oxo-3-(3-oxo-4H-pyrazino[2,3-b][1,4]oxazin-6-yl)-1-oxa-3,8-diazaspiro[4.5]decan-8-yl]methyl]indan-5-yl]propanamide;
2-(azetidin-1-yl)-N-[7-fluoro-2-[[2-[2-oxo-3-(3-oxo-4H-pyrazino[2,3-b][1,4]oxazin-6-yl)oxazolidin-5-yl]ethylamino]methyl]indan-5-yl]acetamide;
(2S)-2-(dimethylamino)-N-[7-fluoro-2-[[2-[2-oxo-3-(3-oxo-4H-pyrazino[2,3-b][1,4]oxazin-6-yl)oxazolidin-5-yl]ethylamino]methyl]indan-5-yl]propanamide;
(2S)—N-[7-fluoro-2-[[2-[2-oxo-3-(3-oxo-4H-pyrazino[2,3-b][1,4]oxazin-6-yl)oxazolidin-5-yl]ethylamino]methyl]indan-5-yl]-1-methyl-pyrrolidine-2-carboxamide;
N-[7-fluoro-2-[[2-[2-oxo-3-(3-oxo-4H-pyrazino[2,3-b][1,4]oxazin-6-yl)oxazolidin-5-yl]ethylamino]methyl]indan-5-yl]-2-[methyl(oxetan-3-yl)amino]acetamide;
2-(3-azabicyclo[3.1.0]hexan-3-yl)-N-[7-fluoro-2-[[2-[2-oxo-3-(3-oxo-4H-pyrazino[2,3-b][1,4]oxazin-6-yl)oxazolidin-5-yl]ethylamino]methyl]indan-5-yl]acetamide;
2-[cyclopropyl(methyl)amino]-N-[7-fluoro-2-[[2-[2-oxo-3-(3-oxo-4H-pyrazino[2,3-b][1,4]oxazin-6-yl)oxazolidin-5-yl]ethylamino]methyl]indan-5-yl]acetamide;
(2R)—N-[7-fluoro-2-[[2-[2-oxo-3-(3-oxo-4H-pyrazino[2,3-b][1,4]oxazin-6-yl)oxazolidin-5-yl]ethylamino]methyl]indan-5-yl]-1-methyl-pyrrolidine-2-carboxamide;
N-[7-fluoro-2-[[2-[2-oxo-3-(3-oxo-4H-pyrazino[2,3-b][1,4]oxazin-6-yl)oxazolidin-5-yl]ethylamino]methyl]indan-5-yl]-2-methyl-oxazole-5-carboxamide;
2-(azetidin-1-yl)-N-[7-fluoro-2-[[2-[(5R)-2-oxo-3-(3-oxo-4H-pyrazino[2,3-b][1,4]oxazin-6-yl)oxazolidin-5-yl]ethylamino]methyl]indan-5-yl]propanamide;
(2S,4R)—N-[7-fluoro-2-[[2-[2-oxo-3-(3-oxo-4H-pyrazino[2,3-b][1,4]oxazin-6-yl)oxazolidin-5-yl]ethylamino]methyl]indan-5-yl]-4-methoxy-1-methyl-pyrrolidine-2-carboxamide;
N-[7-fluoro-2-[[2-[2-oxo-3-(3-oxo-4H-pyrazino[2,3-b][1,4]oxazin-6-yl)oxazolidin-5-yl]ethylamino]methyl]indan-5-yl]-2-hydroxy-2-methyl-propanamide;
(3R)-3-(dimethylamino)-N-[7-fluoro-2-[[2-[2-oxo-3-(3-oxo-4H-pyrazino[2,3-b][1,4]oxazin-6-yl)oxazolidin-5-yl]ethylamino]methyl]indan-5-yl]tetrahydrofuran-3-carboxamide;
N-[7-cyano-2-[[2-[2-oxo-3-(3-oxo-4H-pyrazino[2,3-b][1,4]oxazin-6-yl)oxazolidin-5-yl]ethylamino]methyl]indan-5-yl]-2-hydroxy-2-methyl-propanamide;
N-[7-cyano-2-[[2-[2-oxo-3-(3-oxo-4H-pyrido[3,2-b][1,4]oxazin-6-yl)oxazolidin-5-yl]ethylamino]methyl]indan-5-yl]-2-hydroxy-2-methyl-propanamide;
(2R)—N-[7-cyano-2-[[2-[(5R)-2-oxo-3-(3-oxo-4H-pyrazino[2,3-b][1,4]oxazin-6-yl)oxazolidin-5-yl]ethylamino]methyl]indan-5-yl]-2-(dimethylamino)propanamide;
(2R)-2-(dimethylamino)-N-[4-fluoro-2-[[2-[2-oxo-3-(3-oxo-4H-pyrazino[2,3-b][1,4]oxazin-6-yl)oxazolidin-5-yl]ethylamino]methyl]indan-5-yl]propanamide;
2-(dimethylamino)-N-[4-methyl-6-[[2-[2-oxo-3-(3-oxo-4H-pyrazino[2,3-b][1,4]oxazin-6-yl)oxazolidin-5-yl]ethylamino]methyl]-6,7-dihydro-5H-cyclopenta[b]pyridin-2-yl]acetamide;
(2R)-2-(dimethylamino)-N-[4-methyl-6-[[2-[2-oxo-3-(3-oxo-4H-pyrazino[2,3-b][1,4]oxazin-6-yl)oxazolidin-5-yl]ethylamino]methyl]-6,7-dihydro-5H-cyclopenta[b]pyridin-2-yl]propanamide;
2-(azetidin-1-yl)-N-[4-methyl-6-[[2-[2-oxo-3-(3-oxo-4H-pyrazino[2,3-b][1,4]oxazin-6-yl)oxazolidin-5-yl]ethylamino]methyl]-6,7-dihydro-5H-cyclopenta[b]pyridin-2-yl]acetamide;
(2S)-1-methyl-N-[4-methyl-6-[[2-[2-oxo-3-(3-oxo-4H-pyrazino[2,3-b][1,4]oxazin-6-yl)oxazolidin-5-yl]ethylamino]methyl]-6,7-dihydro-5H-cyclopenta[b]pyridin-2-yl]pyrrolidine-2-carboxamide;
(2R)-2-(dimethylamino)-N-[1-methyl-6-[[2-[2-oxo-3-(3-oxo-4H-pyrazino[2,3-b][1,4]oxazin-6-yl)oxazolidin-5-yl]ethylamino]methyl]-6,7-dihydro-5H-cyclopenta[c]pyridin-3-yl]propanamide;
(2R)-2-(dimethylamino)-N-[1-methyl-6-[[2-oxo-3-(3-oxo-4H-pyrazino[2,3-b][1,4]oxazin-6-yl)-1-oxa-3,8-diazaspiro[4.5]decan-8-yl]methyl]-6,7-dihydro-5H-cyclopenta[c]pyridin-3-yl]propanamide;
(2R)—N-[4,7-difluoro-2-hydroxy-2-[[2-[2-oxo-3-(3-oxo-4H-pyrazino[2,3-b][1,4]oxazin-6-yl)oxazolidin-5-yl]ethylamino]methyl]indan-5-yl]-2-(dimethylamino)propanamide;
(2R)—N-[4,7-difluoro-2-methoxy-2-[[2-[2-oxo-3-(3-oxo-4H-pyrazino[2,3-b][1,4]oxazin-6-yl)oxazolidin-5-yl]ethylamino]methyl]indan-5-yl]-2-(dimethylamino)propanamide;
(2R)-2-(dimethylamino)-N-[2,4,7-trifluoro-2-[[2-[2-oxo-3-(3-oxo-4H-pyrazino[2,3-b][1,4]oxazin-6-yl)oxazolidin-5-yl]ethylamino]methyl]indan-5-yl]propanamide;
(2R)—N-[2,7-difluoro-2-[[2-[2-oxo-3-(3-oxo-4H-pyrazino[2,3-b][1,4]oxazin-6-yl)oxazolidin-5-yl]ethylamino]methyl]indan-5-yl]-2-(dimethylamino)propanamide;
(2R)—N-[7-cyano-2-hydroxy-2-[[2-[2-oxo-3-(3-oxo-4H-pyrazino[2,3-b][1,4]oxazin-6-yl)oxazolidin-5-yl]ethylamino]methyl]indan-5-yl]-2-(dimethylamino)propanamide;
(2R)—N-[7-cyano-2-hydroxy-2-[[2-oxo-3-(3-oxo-4H-pyrazino[2,3-b][1,4]oxazin-6-yl)-1-oxa-3,8-diazaspiro[4.5]decan-8-yl]methyl]indan-5-yl]-2-(dimethylamino)propanamide;
(2S)—N-[7-fluoro-2-[[2-[2-oxo-3-(3-oxo-4H-pyrazino[2,3-b][1,4]oxazin-6-yl)oxazolidin-5-yl]ethylamino]methyl]indan-5-yl]pyrrolidine-2-carboxamide;
(2S,4R)—N-[7-fluoro-2-[[2-[2-oxo-3-(3-oxo-4H-pyrazino[2,3-b][1,4]oxazin-6-yl)oxazolidin-5-yl]ethylamino]methyl]indan-5-yl]-4-hydroxy-pyrrolidine-2-carboxamide;
(2R)-2-amino-N-[7-fluoro-2-[[2-[2-oxo-3-(3-oxo-4H-pyrazino[2,3-b][1,4]oxazin-6-yl)oxazolidin-5-yl]ethylamino]methyl]indan-5-yl]-3-hydroxy-propanamide;
(2R)-2-amino-N-[7-fluoro-2-[[2-[2-oxo-3-(3-oxo-4H-pyrazino[2,3-b][1,4]oxazin-6-yl)oxazolidin-5-yl]ethylamino]methyl]indan-5-yl]-3-methoxy-propanamide;
(3R)-3-amino-N-[7-fluoro-2-[[2-[2-oxo-3-(3-oxo-4H-pyrazino[2,3-b][1,4]oxazin-6-yl)oxazolidin-5-yl]ethylamino]methyl]indan-5-yl]tetrahydrofuran-3-carboxamide;
(3R)-3-amino-N-[7-fluoro-2-[[2-[2-oxo-3-(3-oxo-4H-pyrazino[2,3-b][1,4]oxazin-6-yl)oxazolidin-5-yl]ethylamino]methyl]indan-5-yl]tetrahydrofuran-3-carboxamide; 2,2,2-trifluoroacetic acid;
(3S)-3-amino-N-[7-fluoro-2-[[2-[2-oxo-3-(3-oxo-4H-pyrazino[2,3-b][1,4]oxazin-6-yl)oxazolidin-5-yl]ethylamino]methyl]indan-5-yl]tetrahydrofuran-3-carboxamide;
(3S)-3-amino-N-[7-fluoro-2-[[2-[2-oxo-3-(3-oxo-4H-pyrazino[2,3-b][1,4]oxazin-6-yl)oxazolidin-5-yl]ethylamino]methyl]indan-5-yl]tetrahydrofuran-3-carboxamide; hydrochloride;
(2S,4R)—N-[7-fluoro-2-[[2-oxo-3-(3-oxo-4H-pyrazino[2,3-b][1,4]oxazin-6-yl)-1-oxa-3,8-diazaspiro[4.5]decan-8-yl]methyl]indan-5-yl]-4-hydroxy-pyrrolidine-2-carboxamide;
2-amino-N-[7-fluoro-2-[[2-[2-oxo-3-(3-oxo-4H-pyrazino[2,3-b][1,4]oxazin-6-yl)oxazolidin-5-yl]ethylamino]methyl]indan-5-yl]-2-methyl-propanamide;
(2S,4R)—N-[7-fluoro-2-[[2-[2-oxo-3-(3-oxo-4H-pyrazino[2,3-b][1,4]oxazin-6-yl)oxazolidin-5-yl]ethylamino]methyl]indan-5-yl]-4-methoxy-pyrrolidine-2-carboxamide;
(2R,4R)—N-[7-fluoro-2-[[2-[2-oxo-3-(3-oxo-4H-pyrazino[2,3-b][1,4]oxazin-6-yl)oxazolidin-5-yl]ethylamino]methyl]indan-5-yl]-4-hydroxy-pyrrolidine-2-carboxamide;
(2S,4S)—N-[7-fluoro-2-[[2-[2-oxo-3-(3-oxo-4H-pyrazino[2,3-b][1,4]oxazin-6-yl)oxazolidin-5-yl]ethylamino]methyl]indan-5-yl]-4-hydroxy-pyrrolidine-2-carboxamide;
(2R,4S)—N-[7-fluoro-2-[[2-[2-oxo-3-(3-oxo-4H-pyrazino[2,3-b][1,4]oxazin-6-yl)oxazolidin-5-yl]ethylamino]methyl]indan-5-yl]-4-hydroxy-pyrrolidine-2-carboxamide;
(3S)-3-amino-4-[[7-fluoro-2-[[2-[2-oxo-3-(3-oxo-4H-pyrazino[2,3-b][1,4]oxazin-6-yl)oxazolidin-5-yl]ethylamino]methyl]indan-5-yl]amino]-4-oxo-butanoic acid;
(3S)-3-amino-4-[[7-fluoro-2-[[2-[2-oxo-3-(3-oxo-4H-pyrazino[2,3-b][1,4]oxazin-6-yl)oxazolidin-5-yl]ethylamino]methyl]indan-5-yl]amino]-4-oxo-butanoic acid; 2,2,2-trifluoroacetic acid;
(2S,4R)—N-[7-fluoro-2-[[2-[2-oxo-3-(3-oxo-4H-pyrazino[2,3-b][1,4]oxazin-6-yl)oxazolidin-5-yl]ethylamino]methyl]indan-5-yl]-4-hydroxy-1-methyl-pyrrolidine-2-carboxamide;
N-[7-fluoro-2-[[2-[2-oxo-3-(3-oxo-4H-pyrazino[2,3-b][1,4]oxazin-6-yl)oxazolidin-5-yl]ethylamino]methyl]indan-5-yl]-2-[(3R)-3-hydroxypyrrolidin-1-yl]acetamide;
(2S)-2-(dimethylamino)-N-[7-fluoro-2-[[2-oxo-3-(3-oxo-4H-pyrazino[2,3-b][1,4]oxazin-6-yl)-1-oxa-3,8-diazaspiro[4.5]decan-8-yl]methyl]indan-5-yl]propanamide;
(2S,4R)—N-[4,7-difluoro-2-[[2-oxo-3-(3-oxo-4H-pyrazino[2,3-b][1,4]oxazin-6-yl)-1-oxa-3,8-diazaspiro[4.5]decan-8-yl]methyl]indan-5-yl]-4-hydroxy-pyrrolidine-2-carboxamide;
(2R)—N-[7-fluoro-2-[[2-oxo-3-(3-oxo-4H-pyrazino[2,3-b][1,4]oxazin-6-yl)-1-oxa-3,8-diazaspiro[4.5]decan-8-yl]methyl]indan-5-yl]-2-(hydroxymethyl)pyrrolidine-2-carboxamide;
(2R)—N-[7-fluoro-2-[[2-oxo-3-(3-oxo-4H-pyrazino[2,3-b][1,4]oxazin-6-yl)-1-oxa-3,8-diazaspiro[4.5]decan-8-yl]methyl]indan-5-yl]-2-(hydroxymethyl)pyrrolidine-2-carboxamide; 2,2,2-trifluoroacetic acid;
(2S,3S)—N-[7-fluoro-2-[[2-oxo-3-(3-oxo-4H-pyrazino[2,3-b][1,4]oxazin-6-yl)-1-oxa-3,8-diazaspiro[4.5]decan-8-yl]methyl]indan-5-yl]-3-hydroxy-pyrrolidine-2-carboxamide;
(2S,3S)—N-[7-fluoro-2-[[2-oxo-3-(3-oxo-4H-pyrazino[2,3-b][1,4]oxazin-6-yl)-1-oxa-3,8-diazaspiro[4.5]decan-8-yl]methyl]indan-5-yl]-3-hydroxy-pyrrolidine-2-carboxamide; 2,2,2-trifluoroacetic acid;
(2R,4R)—N-[7-fluoro-2-[[2-oxo-3-(3-oxo-4H-pyrazino[2,3-b][1,4]oxazin-6-yl)-1-oxa-3,8-diazaspiro[4.5]decan-8-yl]methyl]indan-5-yl]-4-hydroxy-pyrrolidine-2-carboxamide;
(2R,4S)—N-[7-fluoro-2-[[2-oxo-3-(3-oxo-4H-pyrazino[2,3-b][1,4]oxazin-6-yl)-1-oxa-3,8-diazaspiro[4.5]decan-8-yl]methyl]indan-5-yl]-4-hydroxy-pyrrolidine-2-carboxamide;
(2S,4S)—N-[7-fluoro-2-[[2-oxo-3-(3-oxo-4H-pyrazino[2,3-b][1,4]oxazin-6-yl)-1-oxa-3,8-diazaspiro[4.5]decan-8-yl]methyl]indan-5-yl]-4-hydroxy-pyrrolidine-2-carboxamide;
(2R)-2-(dimethylamino)-N-[7-fluoro-2-[[methyl-[2-[2-oxo-3-(3-oxo-4H-pyrazino[2,3-b][1,4]oxazin-6-yl)oxazolidin-5-yl]ethyl]amino]methyl]indan-5-yl]propanamide;
(2S,4R)—N-[7-cyano-2-[[2-oxo-3-(3-oxo-4H-pyrazino[2,3-b][1,4]oxazin-6-yl)-1-oxa-3,8-diazaspiro[4.5]decan-8-yl]methyl]indan-5-yl]-4-hydroxy-pyrrolidine-2-carboxamide;
(2S,4R)-4-hydroxy-N-[6-[[2-oxo-3-(3-oxo-4H-pyrazino[2,3-b][1,4]oxazin-6-yl)-1-oxa-3,8-diazaspiro[4.5]decan-8-yl]methyl]-6,7-dihydro-5H-cyclopenta[b]pyridin-3-yl]pyrrolidine-2-carboxamide;
(2S,4R)—N-[7-cyano-2-[[2-[2-oxo-3-(3-oxo-4H-pyrazino[2,3-b][1,4]oxazin-6-yl)oxazolidin-5-yl]ethylamino]methyl]indan-5-yl]-4-hydroxy-pyrrolidine-2-carboxamide;
(2R)—N-[7-cyano-2-[[2-[2-oxo-3-(3-oxo-4H-pyrazino[2,3-b][1,4]oxazin-6-yl)oxazolidin-5-yl]ethylamino]methyl]indan-5-yl]-1-methyl-pyrrolidine-2-carboxamide;
(2S,4R)—N-[7-fluoro-2-[[methyl-[2-[2-oxo-3-(3-oxo-4H-pyrazino[2,3-b][1,4]oxazin-6-yl)oxazolidin-5-yl]ethyl]amino]methyl]indan-5-yl]-4-hydroxy-pyrrolidine-2-carboxamide;
(2S,4R)—N-[7-fluoro-2-[[2-oxo-3-(3-oxo-4H-pyrazino[2,3-b][1,4]oxazin-6-yl)-1-oxa-3,8-diazaspiro[4.5]decan-8-yl]methyl]indan-5-yl]-4-hydroxy-1-methyl-pyrrolidine-2-carboxamide;
(2R)-2-(dimethylamino)-N-[7-fluoro-3-hydroxy-2-[[2-oxo-3-(3-oxo-4H-pyrazino[2,3-b][1,4]oxazin-6-yl)-1-oxa-3,8-diazaspiro[4.5]decan-8-yl]methyl]indan-5-yl]propanamide;
(2R)-2-(dimethylamino)-N-[7-fluoro-3-hydroxy-2-[[2-[(5R)-2-oxo-3-(3-oxo-4H-pyrazino[2,3-b][1,4]oxazin-6-yl)oxazolidin-5-yl]ethylamino]methyl]indan-5-yl]propanamide;
(2R)—N-[3,7-difluoro-2-[[2-[(5R)-2-oxo-3-(3-oxo-4H-pyrazino[2,3-b][1,4]oxazin-6-yl)oxazolidin-5-yl]ethylamino]methyl]indan-5-yl]-2-(dimethylamino)propanamide;
(2R)-2-(dimethylamino)-N-[7-fluoro-2-[[2-[(5S)-2-oxo-3-(3-oxo-4H-pyrazino[2,3-b][1,4]oxazin-6-yl)oxazolidin-5-yl]ethylamino]methyl]indan-5-yl]propanamide;
(2R)-2-(dimethylamino)-N-[7-fluoro-2-[[2-[(5R)-2-oxo-3-(3-oxo-4H-pyrazino[2,3-b][1,4]oxazin-6-yl)oxazolidin-5-yl]ethylamino]methyl]indan-5-yl]propanamide;

or pharmaceutically acceptable salts thereof.

Another particular example of compounds of formula (I) as described herein is (2S,4R)—N-[7-fluoro-2-[[2-oxo-3-(3-oxo-4H-pyrazino[2,3-b][1,4]oxazin-6-yl)-1-oxa-3,8-diazaspiro[4.5]decan-8-yl]methyl]indan-5-yl]-4-hydroxy-2-methyl-pyrrolidine-2-carboxamide;

Further particular examples of formula (I) as described herein are selected from
2-(dimethylamino)-N-[7-fluoro-2-[[2-[2-oxo-3-(3-oxo-4H-pyrazino[2,3-b][1,4]oxazin-6-yl)oxazolidin-5-yl]ethylamino]methyl]indan-5-yl]acetamide;
2-(dimethylamino)-N-[7-fluoro-2-[[2-[2-oxo-3-(3-oxo-4H-pyrazino[2,3-b][1,4]oxazin-6-yl)oxazolidin-5-yl]ethylamino]methyl]indan-5-yl]-2-methyl-propanamide;
(2R)-2-(dimethylamino)-N-[7-fluoro-2-[[2-[2-oxo-3-(3-oxo-4H-pyrazino[2,3-b][1,4]oxazin-6-yl)oxazolidin-5-yl]ethylamino]methyl]indan-5-yl]propanamide;
2-(dimethylamino)-N-[7-fluoro-2-[[2-oxo-3-(3-oxo-4H-pyrazino[2,3-b][1,4]oxazin-6-yl)-1-oxa-3,8-diazaspiro[4.5]decan-8-yl]methyl]indan-5-yl]-2-methyl-propanamide;
(2R)-2-(dimethylamino)-N-[7-fluoro-2-[[2-oxo-3-(3-oxo-4H-pyrazino[2,3-b][1,4]oxazin-6-yl)-1-oxa-3,8-diazaspiro[4.5]decan-8-yl]methyl]indan-5-yl]propanamide;
2-(azetidin-1-yl)-N-[7-fluoro-2-[[2-[2-oxo-3-(3-oxo-4H-pyrazino[2,3-b][1,4]oxazin-6-yl)oxazolidin-5-yl]ethylamino]methyl]indan-5-yl]acetamide;
(2S)-2-(dimethylamino)-N-[7-fluoro-2-[[2-[2-oxo-3-(3-oxo-4H-pyrazino[2,3-b][1,4]oxazin-6-yl)oxazolidin-5-yl]ethylamino]methyl]indan-5-yl]propanamide;
(2S)—N-[7-fluoro-2-[[2-[2-oxo-3-(3-oxo-4H-pyrazino[2,3-b][1,4]oxazin-6-yl)oxazolidin-5-yl]ethylamino]methyl]indan-5-yl]-1-methyl-pyrrolidine-2-carboxamide;
(2R)—N-[7-fluoro-2-[[2-[2-oxo-3-(3-oxo-4H-pyrazino[2,3-b][1,4]oxazin-6-yl)oxazolidin-5-yl]ethylamino]methyl]indan-5-yl]-1-methyl-pyrrolidine-2-carboxamide;
2-(azetidin-1-yl)-N-[7-fluoro-2-[[2-[(5R)-2-oxo-3-(3-oxo-4H-pyrazino[2,3-b][1,4]oxazin-6-yl)oxazolidin-5-yl]ethylamino]methyl]indan-5-yl]propanamide;
N-[7-fluoro-2-[[2-[2-oxo-3-(3-oxo-4H-pyrazino[2,3-b][1,4]oxazin-6-yl)oxazolidin-5-yl]ethylamino]methyl]indan-5-yl]-2-hydroxy-2-methyl-propanamide;
(2R)—N-[7-cyano-2-[[2-[(5R)-2-oxo-3-(3-oxo-4H-pyrazino[2,3-b][1,4]oxazin-6-yl)oxazolidin-5-yl]ethylamino]methyl]indan-5-yl]-2-(dimethylamino)propanamide;
(2S,4R)—N-[7-fluoro-2-[[2-oxo-3-(3-oxo-4H-pyrazino[2,3-b][1,4]oxazin-6-yl)-1-oxa-3,8-diazaspiro[4.5]decan-8-yl]methyl]indan-5-yl]-4-hydroxy-pyrrolidine-2-carboxamide;
(2R,4S)—N-[7-fluoro-2-[[2-[2-oxo-3-(3-oxo-4H-pyrazino[2,3-b][1,4]oxazin-6-yl)oxazolidin-5-yl]ethylamino]methyl]indan-5-yl]-4-hydroxy-pyrrolidine-2-carboxamide;
(2S,4R)—N-[7-fluoro-2-[[2-[2-oxo-3-(3-oxo-4H-pyrazino[2,3-b][1,4]oxazin-6-yl)oxazolidin-5-yl]ethylamino]methyl]indan-5-yl]-4-hydroxy-1-methyl-pyrrolidine-2-carboxamide;
(2S)-2-(dimethylamino)-N-[7-fluoro-2-[[2-oxo-3-(3-oxo-4H-pyrazino[2,3-b][1,4]oxazin-6-yl)-1-oxa-3,8-diazaspiro[4.5]decan-8-yl]methyl]indan-5-yl]propanamide;
(2S,4R)—N-[4,7-difluoro-2-[[2-oxo-3-(3-oxo-4H-pyrazino[2,3-b][1,4]oxazin-6-yl)-1-oxa-3,8-diazaspiro[4.5]decan-8-yl]methyl]indan-5-yl]-4-hydroxy-pyrrolidine-2-carboxamide;
(2R)—N-[7-fluoro-2-[[2-oxo-3-(3-oxo-4H-pyrazino[2,3-b][1,4]oxazin-6-yl)-1-oxa-3,8-diazaspiro[4.5]decan-8-yl]methyl]indan-5-yl]-2-(hydroxymethyl)pyrrolidine-2-carboxamide;
(2R)—N-[7-fluoro-2-[[2-oxo-3-(3-oxo-4H-pyrazino[2,3-b][1,4]oxazin-6-yl)-1-oxa-3,8-diazaspiro[4.5]decan-8-yl]methyl]indan-5-yl]-2-(hydroxymethyl)pyrrolidine-2-carboxamide; 2,2,2-trifluoroacetic acid;
(2S,3S)—N-[7-fluoro-2-[[2-oxo-3-(3-oxo-4H-pyrazino[2,3-b][1,4]oxazin-6-yl)-1-oxa-3,8-diazaspiro[4.5]decan-8-yl]methyl]indan-5-yl]-3-hydroxy-pyrrolidine-2-carboxamide;
(2S,3S)—N-[7-fluoro-2-[[2-oxo-3-(3-oxo-4H-pyrazino[2,3-b][1,4]oxazin-6-yl)-1-oxa-3,8-diazaspiro[4.5]decan-8-yl]methyl]indan-5-yl]-3-hydroxy-pyrrolidine-2-carboxamide; 2,2,2-trifluoroacetic acid;
(2R,4R)—N-[7-fluoro-2-[[2-oxo-3-(3-oxo-4H-pyrazino[2,3-b][1,4]oxazin-6-yl)-1-oxa-3,8-diazaspiro[4.5]decan-8-yl]methyl]indan-5-yl]-4-hydroxy-pyrrolidine-2-carboxamide;
(2R,4S)—N-[7-fluoro-2-[[2-oxo-3-(3-oxo-4H-pyrazino[2,3-b][1,4]oxazin-6-yl)-1-oxa-3,8-diazaspiro[4.5]decan-8-yl]methyl]indan-5-yl]-4-hydroxy-pyrrolidine-2-carboxamide;
(2S,4S)—N-[7-fluoro-2-[[2-oxo-3-(3-oxo-4H-pyrazino[2,3-b][1,4]oxazin-6-yl)-1-oxa-3,8-diazaspiro[4.5]decan-8-yl]methyl]indan-5-yl]-4-hydroxy-pyrrolidine-2-carboxamide;
(2S,4R)—N-[7-cyano-2-[[2-oxo-3-(3-oxo-4H-pyrazino[2,3-b][1,4]oxazin-6-yl)-1-oxa-3,8-diazaspiro[4.5]decan-8-yl]methyl]indan-5-yl]-4-hydroxy-pyrrolidine-2-carboxamide;
(2R)—N-[7-cyano-2-[[2-[2-oxo-3-(3-oxo-4H-pyrazino[2,3-b][1,4]oxazin-6-yl)oxazolidin-5-yl]ethylamino]methyl]indan-5-yl]-1-methyl-pyrrolidine-2-carboxamide;
(2S,4R)—N-[7-fluoro-2-[[2-oxo-3-(3-oxo-4H-pyrazino[2,3-b][1,4]oxazin-6-yl)-1-oxa-3,8-diazaspiro[4.5]decan-8-yl]methyl]indan-5-yl]-4-hydroxy-1-methyl-pyrrolidine-2-carboxamide;
(2R)-2-(dimethylamino)-N-[7-fluoro-2-[[2-[(5S)-2-oxo-3-(3-oxo-4H-pyrazino[2,3-b][1,4]oxazin-6-yl)oxazolidin-5-yl]ethylamino]methyl]indan-5-yl]propanamide;
(2R)-2-(dimethylamino)-N-[7-fluoro-2-[[2-[(5R)-2-oxo-3-(3-oxo-4H-pyrazino[2,3-b][1,4]oxazin-6-yl)oxazolidin-5-yl]ethylamino]methyl]indan-5-yl]propanamide;

or pharmaceutically acceptable salts thereof.

Most particular examples of formula (I) as described herein are selected from
(2R)-2-(dimethylamino)-N-[7-fluoro-2-[[2-[2-oxo-3-(3-oxo-4H-pyrazino[2,3-b][1,4]oxazin-6-yl)oxazolidin-5-yl]ethylamino]methyl]indan-5-yl]propanamide;
(2R)-2-(dimethylamino)-N-[7-fluoro-2-[[2-[(5S)-2-oxo-3-(3-oxo-4H-pyrazino[2,3-b][1,4]oxazin-6-yl)oxazolidin-5-yl]ethylamino]methyl]indan-5-yl]propanamide;
(2R)-2-(dimethylamino)-N-[7-fluoro-2-[[2-[(5R)-2-oxo-3-(3-oxo-4H-pyrazino[2,3-b][1,4]oxazin-6-yl)oxazolidin-5-yl]ethylamino]methyl]indan-5-yl]propanamide;

or pharmaceutically acceptable salts thereof.

Also an object of the present invention is a compound according to formula (I) as described herein for use as a therapeutically active substance.

Likewise an object of the present invention is a pharmaceutical composition comprising a compound according to formula (I) as described herein and a therapeutically inert carrier.

As described above, the compounds of formula (I) and their pharmaceutically acceptable salts possess valuable pharmacological properties for the treatment or prevention of infections and resulting diseases, particularly bacteremia, pneumonia, meningitis, urinary tract infection, and wound infection, caused by pathogens, particularly by bacteria, more particularly by Pseudomonas aeruginosa, Klebsiella pneumoniae, Escherichia coli, Acinetobacter baumannii or Staphylococcus aureus, more particularly by Gram negative bacterias, furthermore particularly by Escherichia coli.

The compounds of formula (I) and their pharmaceutically acceptable salts exhibit activity as antibiotics, particularly as antibiotics against Pseudomonas aeruginosa, Klebsiella pneumoniae, Escherichia coli, Acinetobacter baumannii or Staphylococcus aureus, more particularly against Gram negative bacterias, furthermore particularly against Escherichia coli.

The compounds of formula (I) and their pharmaceutically acceptable salts can be used as antibiotics, i.e. as antibacterial pharmaceutical ingredients suitable in the treatment and prevention of bacterial infections, particularly in the treatment and prevention of bacterial infections caused by pathogens, particularly by bacteria, more particularly by Pseudomonas aeruginosa, Klebsiella pneumoniae, Escherichia coli, Acinetobacter baumannii or Staphylococcus aureus, more particularly by Gram negative bacterias, furthermore particularly by Escherichia coli.

The compounds of the present invention can be used, either alone or in combination with other drugs, for the treatment or prevention of infections and resulting diseases, particularly bacteremia, pneumonia, meningitis, urinary tract infection, and wound infection, caused by pathogens, particularly by bacteria, more particularly by Pseudomonas aeruginosa, Klebsiella pneumoniae, Escherichia coli, Acinetobacter baumannii or Staphylococcus aureus, more particularly by Gram negative bacterias, furthermore particularly by Escherichia coli.

A particular embodiment of the present invention relates to pharmaceutical compositions comprising compounds of formula (I) as defined above or their pharmaceutically acceptable salts as defined above and one or more pharmaceutically acceptable excipients.

A particular embodiment of the present invention relates to pharmaceutical compositions comprising compounds of formula (I) or their pharmaceutically acceptable salts as defined above and one or more pharmaceutically acceptable excipients for the treatment or prevention of infections and resulting diseases, particularly bacteremia, pneumonia, meningitis, urinary tract infection, and wound infection, caused by pathogens, particularly by bacteria, more particularly by Pseudomonas aeruginosa, Klebsiella pneumoniae, Escherichia coli, Acinetobacter baumannii or Staphylococcus aureus, more particularly by Gram negative bacterias, furthermore particularly by Escherichia coli.

A particular embodiment of the present invention relates to compounds of formula (I) or their pharmaceutically acceptable salts as defined above for use as therapeutically active substances, especially for use as therapeutically active substances for the treatment or prevention of infections and resulting diseases, particularly bacteremia, pneumonia, meningitis, urinary tract infection, and wound infection, caused by pathogens, particularly by bacteria, more particularly by Pseudomonas aeruginosa, Klebsiella pneumoniae, Escherichia coli, Acinetobacter baumannii or Staphylococcus aureus, more particularly by Gram negative bacterias, furthermore particularly by Escherichia coli.

A particular embodiment of the present invention relates to compounds of formula (I) or their pharmaceutically acceptable salts as defined above for the use in the treatment or prevention of infections and resulting diseases, particularly bacteremia, pneumonia, meningitis, urinary tract infection, and wound infection, caused by pathogens, particularly by bacteria, more particularly by Pseudomonas aeruginosa, Klebsiella pneumoniae, Escherichia coli, Acinetobacter baumannii or Staphylococcus aureus, more particularly by Gram negative bacterias, furthermore particularly by Escherichia coli.

A particular embodiment of the present invention relates to a method for the treatment or prevention of infections and resulting diseases, particularly bacteremia, pneumonia, meningitis, urinary tract infection, and wound infection, caused by pathogens, particularly by bacteria, more particularly by Pseudomonas aeruginosa, Klebsiella pneumoniae, Escherichia col, Acinetobacter baumannii or Staphylococcus aureus, more particularly by Gram negative bacterias, furthermore particularly by Escherichia coli, which method comprises administering compounds of formula (I) or their pharmaceutically acceptable salts as defined above to a subject.

A particular embodiment of the present invention relates to the use of compounds of formula (I) or their pharmaceutically acceptable salts as defined above for the treatment or prevention of infections and resulting diseases, particularly bacteremia, pneumonia, meningitis, urinary tract infection, and wound infection, caused by pathogens, particularly by bacteria, more particularly by Pseudomonas aeruginosa, Klebsiella pneumoniae, Escherichia coli, Acinetobacter baumannii or Staphylococcus aureus, more particularly by Gram negative bacterias, furthermore particularly by Escherichia coli.

A particular embodiment of the present invention relates to the use of compounds of formula (I) or their pharmaceutically acceptable salts as defined above for the preparation of medicaments for the treatment or prevention of infections and resulting diseases, particularly bacteremia, pneumonia, meningitis, urinary tract infection, and wound infection, caused by pathogens, particularly by bacteria, more particularly caused by pathogens, particularly by bacteria, more particularly by Pseudomonas aeruginosa, Klebsiella pneumoniae, Escherichia coli, Acinetobacter baumannii or Staphylococcus aureus, more particularly by Gram negative bacterias, furthermore particularly by Escherichia coli. Such medicaments comprise compounds of formula (I) or their pharmaceutically acceptable salts as defined above.

Also an embodiment of the present invention are compounds of formula (I) as described herein, when manufactured according to any one of the described processes.

### Assay Procedures

### Minimal Inhibitory Concentration (MIC) Determination:

The MIC of antibacterial compounds against multiple species and strains was determined using the broth microdilution method according to M07-A10 Clinical and Laboratory Standards Institute (CLSI) guidelines [1], using the appropriate broth medium. In short, the compound plates were prepared by dispensing 100 μL of each stock solution in the first well of a 96 well microtiter plate (MTP) at a concentration 100-fold higher than the final concentration desired in broth. Eleven serial 2-fold dilutions of the highest concentration were made in DMSO for new compounds and in water (or appropriate solution) for reference compounds [2]. 1 μL of each well was transferred in a new MTP, which served as the test plate by subsequent inoculation. This bacterial suspension was prepared from strains that were first sub-cultured on agar plates and incubated for 18-24 hours as appropriate. Following incubation, the inoculum was prepared from isolated colonies and adjusted to 0.5 McFarland turbidity standard (1 to 2×108 Colony Forming Units (CFU)/mL) in 0.9% saline. The bacterial suspension was then diluted 1:200 in appropriate sterile medium and within 15 minutes 100 μL/well were dispensed. Negative controls (lack of bacterial cells) and growth control wells (lack of compound) were included in all plates. MTPs were incubated for 18-24 hours at 35±2° C. in ambient air. The MIC value of each compound, expressed as μg/mL, was determined as the lowest concentration required for complete growth inhibition (no visible growth).
[1] CLSI (2015) Methods for dilution antimicrobial susceptibility tests for bacteria that grow aerobically; Approved Standard, Tenth Edition. CLSI Document M7-A10 (ISBN 1-56238-987-4). Wayne, PA: Clinical and Laboratory Standards Institute, 19087, USA.
[2] CLSI (2017) Performance Standards for Antimicrobial Susceptibility Testing; Twenty-Fifth Informational Supplement. CLSI Document M100-S27, CLSI, Wayne, Pennsylvania 19087, USA.

Table 1 provides the Minimal Inhibitory Concentrations (MIC) in micrograms per millilitre of the compounds of present invention obtained against the strain Escherichia coli ATCC 25922.

Particular compounds of the present invention exhibit an MIC (Escherichia coli ATCC 25922)≤4 μg/mL.

More particular compounds of the present invention exhibit an MIC (Escherichia coli ATCC 25922)≤1 μg/mL.

Most particular compounds of the present invention exhibit an MIC (Escherichia coli ATCC 25922)≤0.25 μg/mL.

TABLE 1

| | Escherichia |
|---|---|
| | coli growth |
| | inhibition |
| Example | MIC (μg/mL) |
| 1 | 0.125 |
| 2 | 1 |
| 3 | 0.25 |
| 4 | 0.125 |
| 5 | 1 |
| 6 | 0.25 |
| 7 | 0.25 |
| 8 | 0.125 |
| 9 | 0.125 |
| 10 | 0.125 |
| 11 | 0.5 |
| 12 | 0.125 |
| 13 | 1 |
| 14 | 0.125 |
| 15 | 0.25 |
| 16 | 0.125 |
| 17 | 1 |
| 18 | 0.25 |
| 19 | 2 |
| 20 | 0.5 |
| 21 | 0.5 |
| 22 | 0.125 |
| 23 | 0.5 |
| 24 | 0.5 |
| 25 | 1 |
| 26 | 0.5 |
| 27 | 1 |
| 28 | 0.5 |
| 29 | 0.5 |
| 30 | 1 |
| 31 | 4 |
| 32 | 0.5 |
| 33 | 0.5 |
| 34 | 4 |
| 35 | 1 |
| 36 | 0.25 |
| 37 | 0.5 |
| 38 | 0.5 |
| 39 | 0.5 |
| 40 | 0.25 |
| 41 | 1 |
| 42 | 0.125 |
| 43 | 0.25 |
| 44 | 1 |
| 45 | 1 |
| 46 | 0.5 |
| 47 | 0.25 |
| 48 | 4 |
| 49 | 0.25 |
| 50 | 0.5 |
| 51 | 0.125 |
| 52 | 0.25 |
| 53 | 0.25 |
| 54 | 0.25 |
| 55 | 0.125 |
| 56 | ≤0.06 |
| 57 | ≤0.06 |
| 58 | 0.25 |
| 59 | ≤0.06 |
| 60 | 0.5 |
| 61 | 0.5 |
| 62 | ≤0.06 |
| 63 | 0.25 |
| 64 | 0.25 |
| 65 | 0.5 |
| 66 | 4 |
| 67 | 0.5 |
| 68 | 0.125 |
| 69 | 0.125 |
| 70 | 0.125 |
| 71 | 0.125 |
| 72 | 0.5 |
| 73 | 0.5 |

### Case 33955

### New Isoxazolyl Ether Derivatives as GABA A ALPHA5 PAM

The compounds of formula (I) and their pharmaceutically acceptable salts can be used as medicaments (e.g. in the form of pharmaceutical preparations). The pharmaceutical preparations can be administered internally, such as orally (e.g. in the form of tablets, coated tablets, dragées, hard and soft gelatin capsules, solutions, emulsions or suspensions), nasally (e.g. in the form of nasal sprays), rectally (e.g. in the form of suppositories) or topical ocularly (e.g. in the form of solutions, ointments, gels or water soluble polymeric inserts). However, the administration can also be effected parenterally, such as intramuscularly, intravenously, or intraocularly (e.g. in the form of sterile injection solutions).

The compounds of formula (I) and their pharmaceutically acceptable salts can be processed with pharmaceutically inert, inorganic or organic adjuvants for the production of tablets, coated tablets, dragées, hard gelatin capsules, injection solutions or topical formulations Lactose, corn starch or derivatives thereof, talc, stearic acid or its salts etc. can be used, for example, as such adjuvants for tablets, dragées and hard gelatin capsules.

Suitable adjuvants for soft gelatin capsules, are, for example, vegetable oils, waxes, fats, semi-solid substances and liquid polyols, etc.

Suitable adjuvants for the production of solutions and syrups are, for example, water, polyols, saccharose, invert sugar, glucose, etc.

Suitable adjuvants for injection solutions are, for example, water, alcohols, polyols, glycerol, vegetable oils, etc.

Suitable adjuvants for suppositories are, for example, natural or hardened oils, waxes, fats, semi-solid or liquid polyols, etc.

Suitable adjuvants for topical ocular formulations are, for example, cyclodextrins, mannitol or many other carriers and excipients known in the art.

Moreover, the pharmaceutical preparations can contain preservatives, solubilizers, viscosity-increasing substances, stabilizers, wetting agents, emulsifiers, sweeteners, colorants, flavorants, salts for varying the osmotic pressure, buffers, masking agents or antioxidants. They can also contain still other therapeutically valuable substances.

The dosage can vary in wide limits and will, of course, be fitted to the individual requirements in each particular case. In general, in the case of oral administration a daily dosage of about 0.1 mg to 20 mg per kg body weight, preferably about 0.5 mg to 4 mg per kg body weight (e.g. about 300 mg per person), divided into preferably 1-3 individual doses, which can consist, for example, of the same amounts, should it be appropriate. In the case of topical administration, the formulation can contain 0.001% to 15% by weight of medicament and the required dose, which can be between 0.1 and 25 mg in can be administered either by single dose per day or per week, or by multiple doses (2 to 4) per day, or by multiple doses per week It will, however, be clear that the upper or lower limit given herein can be exceeded when this is shown to be indicated.

### Preparation of Pharmaceutical Compositions Comprising Compounds of the Invention:

Tablets of the following composition are manufactured in the usual manner:

mg/tablet

| Ingredient | 5 | 25 | 100 | 500 |
|---|---|---|---|---|
| Compound of formula I | 5 | 25 | 100 | 500 |
| Lactose Anhydrous DTG | 125 | 105 | 30 | 150 |
| Sta-Rx 1500 | 6 | 6 | 6 | 60 |
| Microcrystalline Cellulose | 30 | 30 | 30 | 450 |
| Magnesium Stearate | 1 | 1 | 1 | 1 |
| Total | 167 | 167 | 167 | 1161 |

### Manufacturing Procedure

1. Mix ingredients 1, 2, 3 and 4 and granulate with purified water.
2. Dry the granules at 50° C.
3. Pass the granules through suitable milling equipment.
4. Add ingredient 5 and mix for three minutes; compress on a suitable press.

Capsules of the following composition are manufactured:

mg/capsule

| ingredient | 5 | 25 | 100 | 500 |
|---|---|---|---|---|
| Compound of formula I | 5 | 25 | 100 | 500 |
| Hydrous Lactose | 159 | 123 | 148 | — |
| Corn Starch | 25 | 35 | 40 | 70 |
| Talk | 10 | 15 | 10 | 25 |
| Magnesium Stearate | 1 | 2 | 2 | 5 |
| Total | 200 | 200 | 300 | 600 |

### Manufacturing Procedure

1. Mix ingredients 1, 2 and 3 in a suitable mixer for 30 minutes.
2. Add ingredients 4 and 5 and mix for 3 minutes.
3. Fill into a suitable capsule.

A compound of formula I lactose and corn starch are firstly mixed in a mixer and then in a comminuting machine. The mixture is returned to the mixer; the talc is added thereto and mixed thoapproximatively. The mixture is filled by machine into suitable capsules, e.g. hard gelatin capsules.

Injection solutions of the following composition are manufactured:

| ingredient | mg/injection solution. |
|---|---|
| Compound of formula I | 3 |
| Polyethylene Glycol 400 | 150 |
| acetic acid | q.s. ad pH 5.0 |
| water for injection solutions | ad 1.0 ml |

The invention is illustrated hereinafter by Examples, which have no limiting character.

In case the preparative examples are obtained as a mixture of enantiomers, the pure enantiomers can be obtained by methods described herein or by methods known to those skilled in the art, such as e.g. chiral chromatography or crystallization.

### EXAMPLES

### Abbreviations

MeOH: methanol, AcOH: acetic acid, prep-HPLC: preparative reversed phase high-performance liquid chromatography, MS: mass spectrum, M: molecular mass, ESP: electrospray ionisation (positive charge detection), ESN: electrospray ionisation (negative charge detection), DCM: dichloromethane, EtOAc: ethyl acetate, DMF: N,N-dimethylformamide, RT, r.t. or rt: room temperature, DIPEA: N,N-diisopropylethylamine, i-PrOH: 2-propanol, h: hours, min.: minutes, THF: tetrahydrofuran, xantphos: 4,5-bis(diphenylphosphino)-9,9-dimethylxanthene, TFA: trifluoroacetic acid, dioxane: 1,4-dioxane, sat.: saturated, quant.: quantitative, EI: electron ionization, dppf: 1,1′-ferrocenediyl-bis(diphenylphosphine), HATU: 1-[bis(dimethylamino)methylene]-1H-1,2,3-triazolo[4,5-b]pyridinium 3-oxide hexafluorophosphate, RM: reaction mixture, t-BuOH: 2-methylpropan-2-ol, prep-TLC: preparative thin layer chromatography, SFC: supercritical fluid chromatography, ether: diethyl ether, BOC: tert-butyloxycarbonyl, HV: high vacuum, Pd/C: palladium on carbon, TMSCl: trimethylsilyl chloride, MeCN: acetonitrile, NMR: nuclear magnetic resonance spectroscopy, s: singlet, d (NMR): doublet, t: triplet, q: quartet, m: multiplet, dd: doublet of doublets, dt: doublet of triplets, br: broad, J: coupling constant, δ, chemical shift in parts per million, PE: petroleum ether, NMP: N-methyl-2-pyrrolidone, DMA: N,N-dimethylacetamide, MTBE: methyl tert-butyl ether, Dess-Martin periodinane or DMP: 1,1,1-triacetoxy-1,1-dihydro-1,2-benziodoxol-3(1H)-one, LDA: lithium diisopropylamide, MOMCl: methylchloromethyl ether, DMAP: 4-dimethylaminopyridine, tBu: tert-butyl, LiHMDS: lithium bis(trimethylsilyl)amide, TsCl: tosyl chloride, HPLC: preparative reversed phase high-performance liquid chromatography, TBSOTf: tert-butyldimethylsilyl trifluoromethanesulfonate, Grubbs Catalyst 2nd Generation: 1,3-Bis(2,4,6-trimethylphenyl)-2-imidazolidinylidene)dichloro(phenylmethylene) (tricyclohexylphosphine)ruthenium, DMSO: dimethyl sulfoxide, AcOEt: ethyl acetate, d (time): days, TEMPO: (2,2,6,6-tetramethyl-piperidin-1-yl)oxyl, TBSCl: tert-butyldimethylsilyl chloride, dba: dibenzylideneacetone, XPhos: dicyclohexyl[2′,4′,6′-tris(propan-2-yl)[1,1′-biphenyl]-2-yl]phosphane, t-BuXPhos: 2-di-tert-butylphosphino-2′,4′,6′-triisopropylbiphenyl, t-BuXPhos-Pd-G3: [(2-di-tert-butylphosphino-2′,4′,6′-triisopropyl-1,1′-biphenyl)-2-(2′-amino-1,1′-biphenyl)] palladium(II) methanesulfonate, XPhos-G3-Pd: (2-dicyclohexylphosphino-2′,4′,6′-triisopropyl-1,1′-biphenyl)[2-(2′-amino-1,1′-biphenyl)]palladium(II) methanesulfonate, T3P: 2,4,6-tripropyl-1,3,5,2,4,6-trioxatriphosphorinane-2,4,6-trioxide, TBAF: tetra-n-butylammonium fluoride, TCFH: chloro-N,N,N′N′-tetramethylformamidinium hexafluorophosphate, SCX: strong cation exchange.

### Example 1

### 2-(Dimethylamino)-N-[7-fluoro-2-[[2-[2-oxo-3-(3-oxo-4H-pyrazino[2,3-b][1,4]oxazin-6-yl)oxazolidin-5-yl]ethylamino]methyl]indan-5-yl]acetamide

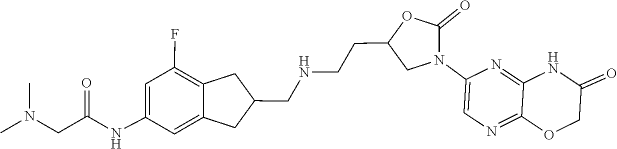 [SMILES: CN(C)CC(=O)Nc1cc(F)c2c(c1)CC(CNCCC1CN(c3cnc4c(n3)NC(=O)CO4)C(=O)O1)C2]

A mixture of 6-[5-(2-aminoethyl)-2-oxo-oxazolidin-3-yl]-4H-pyrazino[2,3-b][1,4]oxazin-3-one; 2,2,2-trifluoroacetic acid (Intermediate 1, 51.13 mg, 0.130 mmol, 1 eq), 2-(dimethylamino)-N-(7-fluoro-2-formyl-indan-5-yl)acetamide (Intermediate 7, 85.9 mg, 0.130 mmol, 1 eq), DIPEA (33.96 uL, 0.190 mmol, 1.5 eq) in THF (3 mL) and DMA (2 mL) was stirred at RT for 15 min. Acetic acid (18.61 uL, 0.330 mmol, 2.5 eq) and sodium triacetoxyborohydride (41.33 mg, 0.190 mmol, 1.5 eq) were added and the mixture was stirred at RT for 1 h. EtOAc and NaHCO₃ (sat. aq.) were added, then the phases were separated. The organic phase was dried over Na₂SO₄, filtered and concentrated. The crude product was purified by flash column chromatography (0 to 10% MeOH/DCM) to give the title compound (12 mg, 0.020 mmol, 17.3% yield). MS (ESP) m/z=528.2 [M+H]⁺.

The following examples were prepared by analogy to Example 1.

| Ex. | Name, Structure, MS | Starting Materials |
|---|---|---|
| 2 | 1-(dimethylamino)-N-[7-fluoro-2-[[2-[2-oxo-3-(3-oxo-4H- | Intermediate 1 and |
| | pyrazino[2,3-b][1,4]oxazin-6-yl)oxazolidin-5- | Intermediate 8 |
| | yl]ethylaminomethyl]indan-5-yl]cyclopropanecarboxamide | |
| | 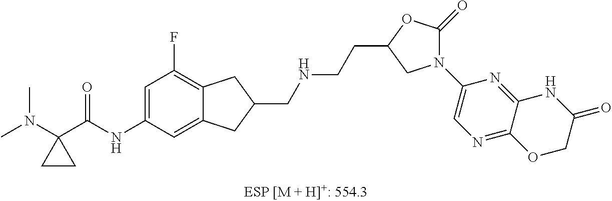 [SMILES: CN(C)C1(C(=O)Nc2cc(F)c3c(c2)CC(CNCCC2CN(c4cnc5c(n4)NC(=O)CO5)C(=O)O2)C3)CC1] | |
| 3 | 2-(dimethylamino)-N-[7-fluoro-2-[[2-[2-oxo-3-(3-oxo-4H- | Intermediate 1 and |
| | pyrazino[2,3-b][1,4]oxazin-6-yl)oxazolidin-5- | Intermediate 9 |
| | ylethylamino methyl]indan-5-yl]-2-methyl-propanamide | |
| | 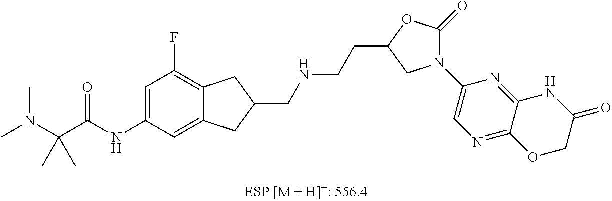 [SMILES: CN(C)C(C)(C)C(=O)Nc1cc(F)c2c(c1)CC(CNCCC1CN(c3cnc4c(n3)NC(=O)CO4)C(=O)O1)C2] | |
| 4 | (2R)-2-(dimethylamino)-N-[7-fluoro-2-[[2-[2-oxo-3-(3-oxo-4H- | Intermediate 1 and |
| | pyrazino[2,3-b][1,4]oxazin-6-yl)oxazolidin-5- | Intermediate 10 |
| | yl]ethylamino methyl]indan-5-yl]propanamide | |
| | 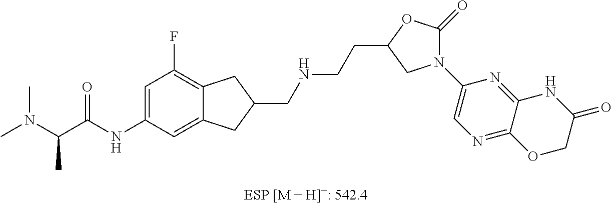 [SMILES: C[C@H](C(=O)Nc1cc(F)c2c(c1)CC(CNCCC1CN(c3cnc4c(n3)NC(=O)CO4)C(=O)O1)C2)N(C)C] | |
| 5 | N-[7-fluoro-2-[[2-[2-oxo-3-(3-oxo-4H-pyrazino[2,3-b][1,4]oxazin-6- | Intermediate 1 and |
| | yl)oxazolidin-5-yl]ethylamino]methyl]indan-5-yl]-2-morpholino- | Intermediate 11 |
| | acetamide | |
| | 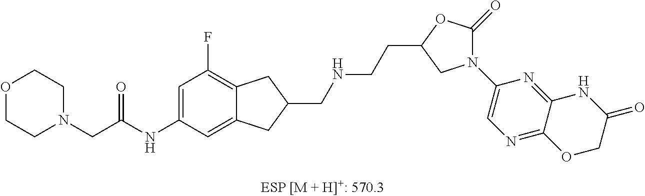 [SMILES: O=C(CN1CCOCC1)Nc1cc(F)c2c(c1)CC(CNCCC1CN(c3cnc4c(n3)NC(=O)CO4)C(=O)O1)C2] | |
| 6 | 2-(dimethylamino)-N-[7-fluoro-2-[[2-oxo-3-(3-oxo-4H]-pyrazino[2,3- | Intermediate 4 and |
| | b][1,4]oxazin-6-yl)-1-oxa-3,8-diazaspiro[4.5]decan-8- | Intermediate 9 |
| | yl]methyl]indan-5-yl]-2-methyl-propanamide | |
| | 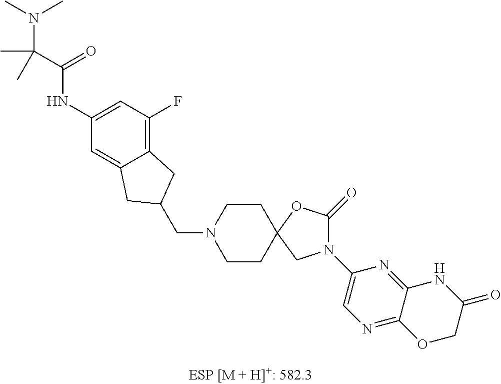 [SMILES: CN(C)C(C)(C)C(=O)Nc1cc(F)c2c(c1)CC(CN1CCC3(CC1)CN(c1cnc4c(n1)NC(=O)CO4)C(=O)O3)C2] | |
| 7 | (2R)-2-(dimethylamino)-N-[7-fluoro-2-[[2-oxo-3-(3-oxo-4H- | Intermediate 4 and |
| | pyrazino[2,3-b][1,4]oxazin-6-yl)-1-oxa-3,8-diazaspiro[4.5]decan-8- | Intermediate 10 |
| | yl]methyl]indan-5-yl]propanamide | |
| | 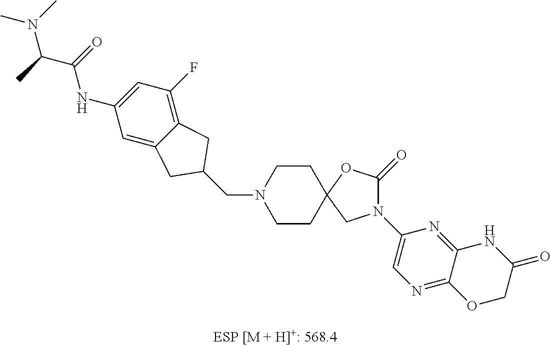 [SMILES: C[C@H](C(=O)Nc1cc(F)c2c(c1)CC(CN1CCC3(CC1)CN(c1cnc4c(n1)NC(=O)CO4)C(=O)O3)C2)N(C)C] | |
| 8 | 2-(azetidin-1-yl)-N-[7-fluoro-2-[2-[2-oxo-3-(3-oxo-4H- | Intermediate 1 and |
| | pyrazino[2,3-b][1,4]oxazin-6-yl)oxazolidin-5- | Intermediate 12 |
| | yl]ethylamino methyl]indan-5-yl]acetamide | |
| | 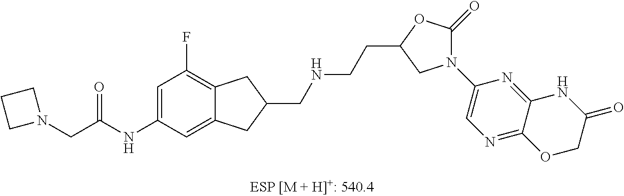 [SMILES: O=C(CN1CCC1)Nc1cc(F)c2c(c1)CC(CNCCC1CN(c3cnc4c(n3)NC(=O)CO4)C(=O)O1)C2] | |
| 9 | (2S)-2-(dimethylamino)-N-[7-fluoro-2-[[2-[2-oxo-3-(3-oxo-4H- | Intermediate 1 and |
| | pyrazino[2,3-b][1,4]oxazin-6-yl)oxazolidin-5- | Intermediate 13 |
| | yl]ethylamino methyl]indan-5-yl]propanamide | |
| | 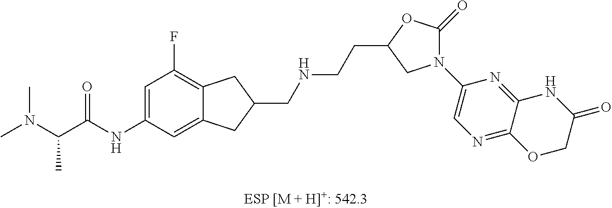 [SMILES: C[C@@H](C(=O)Nc1cc(F)c2c(c1)CC(CNCCC1CN(c3cnc4c(n3)NC(=O)CO4)C(=O)O1)C2)N(C)C] | |
| 10 | (2S)-N-[7-fluoro-2-[[2-[2-oxo-3-(3-oxo-4H-pyrazino[2,3- | Intermediate 1 and |
| | b][1,4]oxazin-6-yl)oxazolidin-5-yl]ethylamino]methyl]indan-5-yl]-1- | Intermediate 14 |
| | methyl-pyrrolidine-2-carboxamide | |
| | 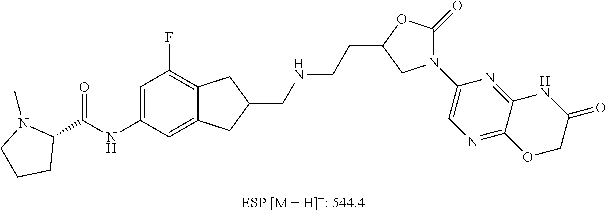 [SMILES: CN1CCC[C@H]1C(=O)Nc1cc(F)c2c(c1)CC(CNCCC1CN(c3cnc4c(n3)NC(=O)CO4)C(=O)O1)C2] | |
| 11 | N-[7-fluoro-2-[[2-[2-oxo-3-(3-oxo-4H-pyrazino[2,3-b][1,4]oxazin-6- | Intermediate 1 and |
| | yl)oxazolidin-5-yl]ethylamino methyl]indan-5-yl]-2-[methyl(oxetan- | Intermediate 15 |
| | 3-yl)amino]acetamide | |
| | 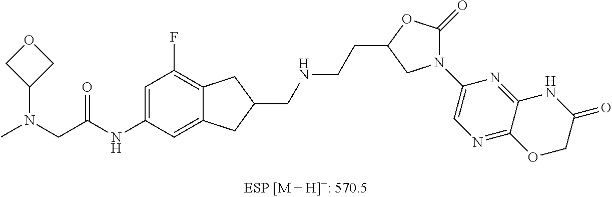 [SMILES: CN(CC(=O)Nc1cc(F)c2c(c1)CC(CNCCC1CN(c3cnc4c(n3)NC(=O)CO4)C(=O)O1)C2)C1COC1] | |
| 12 | 2-(3-azabicyclo[3.1.0]hexan-3-yl)-N-[7-fluoro-2-[2-[2-oxo-3-(3- | Intermediate 1 and |
| | oxo-4H-pyrazino[2,3-b][1,4]oxazin-6-yl)oxazolidin-5- | Intermediate 16 |
| | yl]ethylamino methyl]indan-5-yl]acetamide | |
| | 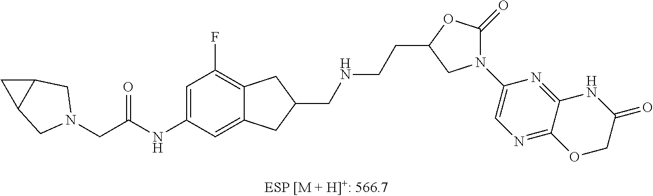 [SMILES: O=C(CN1CC2CC2C1)Nc1cc(F)c2c(c1)CC(CNCCC1CN(c3cnc4c(n3)NC(=O)CO4)C(=O)O1)C2] | |
| 13 | 2-[cyclopropyl(methyl)amino]-N-[7-fluoro-2-[[2-[2-oxo-3-(3-oxo- | Intermediate 1 and |
| | 4H-pyrazino[2,3-b][1,4]oxazin-6-yl)oxazolidin-5- | Intermediate 17 |
| | ylethylamino methyl]indan-5-yl]acetamide | |
| | 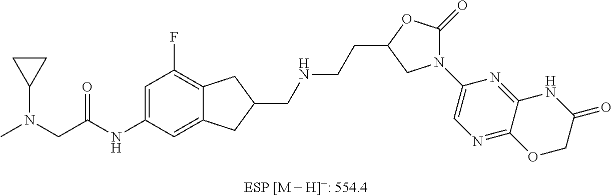 [SMILES: CN(CC(=O)Nc1cc(F)c2c(c1)CC(CNCCC1CN(c3cnc4c(n3)NC(=O)CO4)C(=O)O1)C2)C1CC1] | |
| 14 | (2R)-N-[7-fluoro-2-[[2-[2-oxo-3-(3-oxo-4H-pyrazino[2,3- | Intermediate 1 and |
| | b][1,4]oxazin-6-yl)oxazolidin-5-yl]ethylamino]methyl]indan-5-yl]-1- | Intermediate 18 |
| | methyl-pyrrolidine-2-carboxamide | |
| | 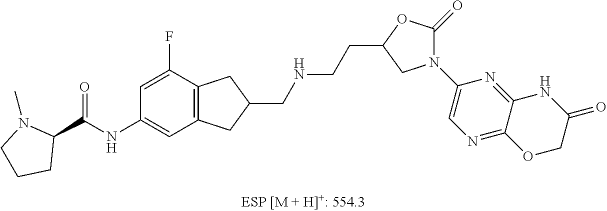 [SMILES: CN1CCC[C@@H]1C(=O)Nc1cc(F)c2c(c1)CC(CNCCC1CN(c3cnc4c(n3)NC(=O)CO4)C(=O)O1)C2] | |
| 15 | N-[7-fluoro-2-[[2-[2-oxo-3-(3-oxo-4H-pyrazino[2,3-b][1,4]oxazin-6- | Intermediate 1 and |
| | yl)oxazolidin-5-yl]ethylamino]methyl]indan-5-yl]-2-methyl-oxazole- | Intermediate 19 |
| | 5-carboxamide | |
| | 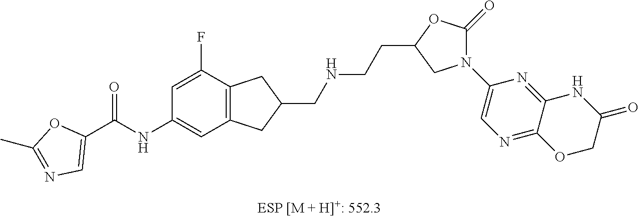 [SMILES: Cc1ncc(C(=O)Nc2cc(F)c3c(c2)CC(CNCCC2CN(c4cnc5c(n4)NC(=O)CO5)C(=O)O2)C3)o1] | |
| 16 | 2-(azetidin-1-yl)-N-[7-fluoro-2-[[2-[(5R)-2-oxo-3-(3-oxo-4H- | Intermediate 2 and |
| | pyrazino[2,3-b][1,4]oxazin-6-yl)oxazolidin-5- | Intermediate 20 |
| | yl]ethylamino methyl]indan-5-yl]propanamide | |
| | 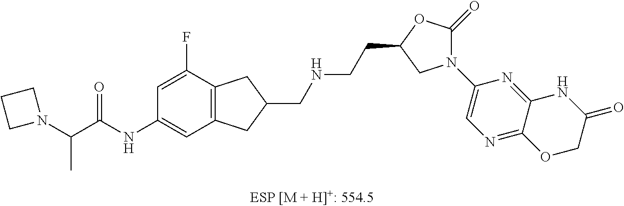 [SMILES: CC(C(=O)Nc1cc(F)c2c(c1)CC(CNCC[C@@H]1CN(c3cnc4c(n3)NC(=O)CO4)C(=O)O1)C2)N1CCC1] | |
| 17 | (2S,4R)-N-[7-fluoro-2-[[2-[2-oxo-3-(3-oxo-4H-pyrazino[2,3- | Intermediate 1 and |
| | b][1,4]oxazin-6-yl)oxazolidin-5-yl]ethylamino]methyl]indan-5-yl]-4- | Intermediate 21 |
| | methoxy-1-methyl-pyrrolidine-2-carboxamide | |
| | 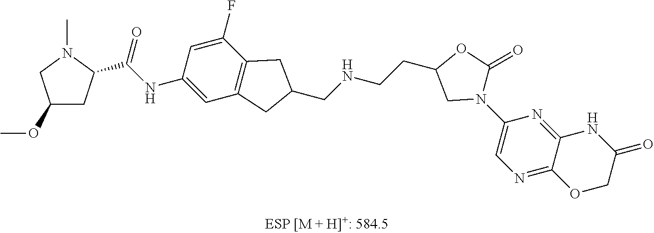 [SMILES: CO[C@@H]1C[C@@H](C(=O)Nc2cc(F)c3c(c2)CC(CNCCC2CN(c4cnc5c(n4)NC(=O)CO5)C(=O)O2)C3)N(C)C1] | |
| 18 | N-[7-fluoro-2-[[2-[2-oxo-3-(3-oxo-4H-pyrazino[2,3-b][1,4]oxazin-6- | Intermediate 1 and |
| | yl)oxazolidin-5-yl]ethylamino]methyl]indan-5-yl]-2-hydroxy-2- | Intermediate 22 |
| | methyl-propanamide | |
| | 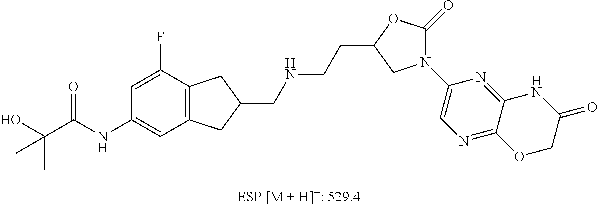 [SMILES: CC(C)(O)C(=O)Nc1cc(F)c2c(c1)CC(CNCCC1CN(c3cnc4c(n3)NC(=O)CO4)C(=O)O1)C2] | |
| 19 | (3R)-3-(dimethylamino)-N-[7-fluoro-2-[[2-[2-oxo-3-(3-oxo-4H- | Intermediate 1 and |
| | pyrazino[2,3-b][1,4]oxazin-6-yl)oxazolidin-5- | Intermediate 23 |
| | yl]ethylamino methyl]indan-5-yl]tetrahydrofuran-3-carboxamide | |
| | 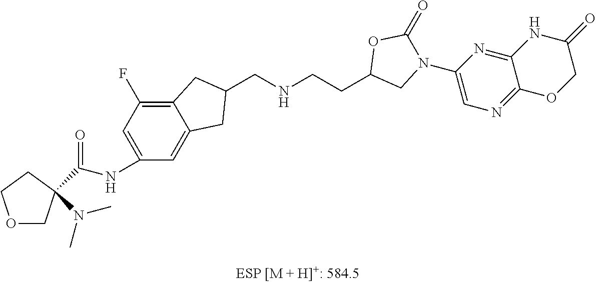 [SMILES: CN(C)[C@]1(C(=O)Nc2cc(F)c3c(c2)CC(CNCCC2CN(c4cnc5c(n4)NC(=O)CO5)C(=O)O2)C3)CCOC1] | |
| 20 | N-[7-cyano-2-[[2-[2-oxo-3-(3-oxo-4H-pyrazino[2,3-b][1,4]oxazin-6- | Intermediate 1 and |
| | yl)oxazolidin-5-ylethylamino]methyl]indan-5-yl]-2-hydroxy-2- | Intermediate 24 |
| | methyl-propanamide | |
| | 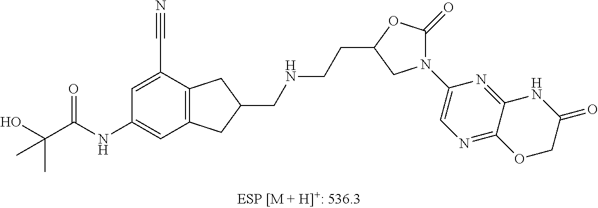 [SMILES: CC(C)(O)C(=O)Nc1cc(C#N)c2c(c1)CC(CNCCC1CN(c3cnc4c(n3)NC(=O)CO4)C(=O)O1)C2] | |
| 21 | N-[7-cyano-2-[[2-[2-oxo-3-(3-oxo-4H-pyrido[3,2-b][1,4]oxazin-6- | CAS# 2156619-15-5 |
| | yl)oxazolidin-5-yl]ethylamino]methyl]indan-5-yl]-2-hydroxy-2- | and Intermediate 24 |
| | methyl-propanamide | |
| | 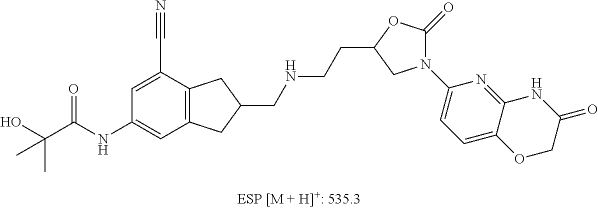 [SMILES: CC(C)(O)C(=O)Nc1cc(C#N)c2c(c1)CC(CNCCC1CN(c3ccc4c(n3)NC(=O)CO4)C(=O)O1)C2] | |
| 22 | (2R)-N-[7-cyano-2-[[2-[(5R)-2-oxo-3-(3-oxo-4H-pyrazino[2,3- | Intermediate 2 and |
| | b][1,4]oxazin-6-yl)oxazolidin-5-yl]ethylamino]methyl]indan-5-yl]-2- | Intermediate 25 |
| | (dimethylamino)propanamide | |
| | 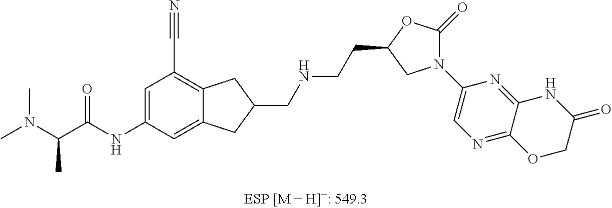 [SMILES: C[C@H](C(=O)Nc1cc(C#N)c2c(c1)CC(CNCC[C@@H]1CN(c3cnc4c(n3)NC(=O)CO4)C(=O)O1)C2)N(C)C] | |
| 23 | (2R)-2-(dimethylamino)-N-[4-fluoro-2-[[2-[2-oxo-3-(3-oxo-4H- | Intermediate 1 and |
| | pyrazino[2,3-b][1,4]oxazin-6-yl)oxazolidin-5- | Intermediate 26 |
| | yl]ethylamino methyl]indan-5-yl]propanamide | |
| | 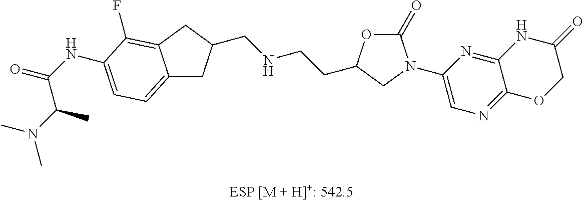 [SMILES: C[C@H](C(=O)Nc1ccc2c(c1F)CC(CNCCC1CN(c3cnc4c(n3)NC(=O)CO4)C(=O)O1)C2)N(C)C] | |
| 24 | 2-(dimethylamino)-N-[4-methyl-6-[2-[2-oxo-3-(3-oxo-4H- | Intermediate 1 and |
| | pyrazino[2,3-b][1,4]oxazin-6-yl)oxazolidin-5-yl]ethylamino]methyl]- | Intermediate 27 |
| | 6,7-dihydro-5H-cyclopenta[b]pyridin-2-yl]acetamide | |
| | 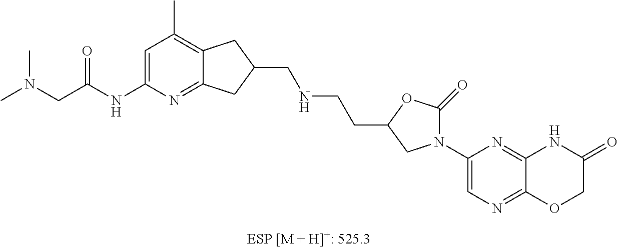 [SMILES: Cc1cc(NC(=O)CN(C)C)nc2c1CC(CNCCC1CN(c3cnc4c(n3)NC(=O)CO4)C(=O)O1)C2] | |
| 25 | (2R)-2-(dimethylamino)-N-[4-methyl-6-[[2-[2-oxo-3-(3-oxo-4H- | Intermediate 1 and |
| | pyrazino[2,3-b][1,4]oxazin-6-yl)oxazolidin-5-yl]ethylamino]methyl]- | Intermediate 28 |
| | 6,7-dihydro-5H-cyclopenta[b]pyridin-2-yl]propanamide | |
| | 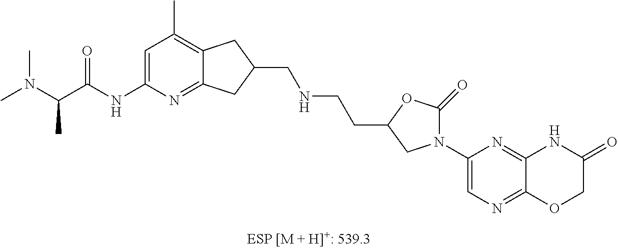 [SMILES: Cc1cc(NC(=O)[C@@H](C)N(C)C)nc2c1CC(CNCCC1CN(c3cnc4c(n3)NC(=O)CO4)C(=O)O1)C2] | |
| 26 | 2-(azetidin-1-yl)-N-[4-methyl-6-[[2-[2-oxo-3-(3-oxo-4H- | Intermediate 1 and |
| | pyrazino[2,3-b][1,4]oxazin-6-yl)oxazolidin-5-yl]ethylamino]methyl]- | Intermediate 29 |
| | 6,7-dihydro-5H-cyclopenta[b]pyridin-2-yl]acetamide | |
| | 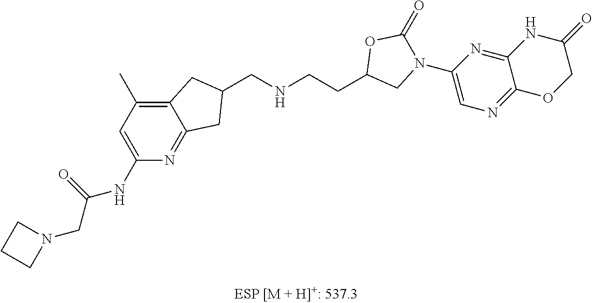 [SMILES: Cc1cc(NC(=O)CN2CCC2)nc2c1CC(CNCCC1CN(c3cnc4c(n3)NC(=O)CO4)C(=O)O1)C2] | |
| 27 | (2S)-1-methyl-N-[4-methyl-6-[[2-[2-oxo-3-(3-oxo-4H-pyrazino[2,3- | Intermediate 1 and |
| | b][1,4]oxazin-6-yl)oxazolidin-5-yl]ethylamino]methyl]-6,7-dihydro- | Intermediate 30 |
| | 5H-cyclopenta[b]pyridin-2-yl]pyrrolidine-2-carboxamide | |
| | 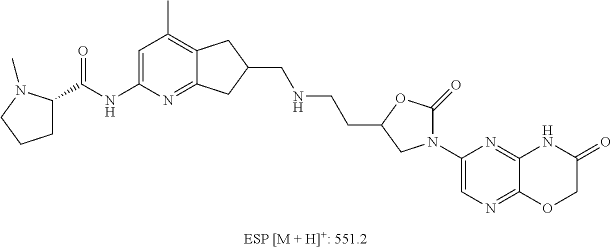 [SMILES: Cc1cc(NC(=O)[C@@H]2CCCN2C)nc2c1CC(CNCCC1CN(c3cnc4c(n3)NC(=O)CO4)C(=O)O1)C2] | |
| 28 | (2R)-2-(dimethylamino)-N-[1-methyl-6-[[2-[2-oxo-3-(3-oxo-4H- | Intermediate 1 and |
| | pyrazino[2,3-b][1,4]oxazin-6-yl)oxazolidin-5-yl]ethylamino]methyl]- | Intermediate 31 |
| | 6,7-dihydro-5H-cyclopenta[c]pyridin-3-yl]propanamide | |
| | 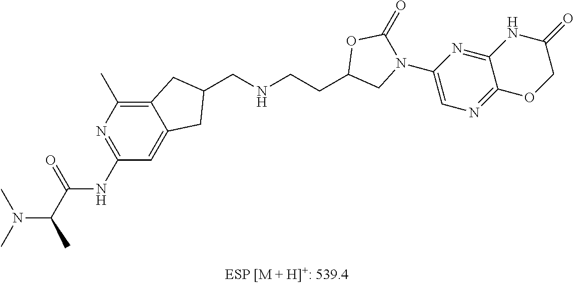 [SMILES: Cc1nc(NC(=O)[C@@H](C)N(C)C)cc2c1CC(CNCCC1CN(c3cnc4c(n3)NC(=O)CO4)C(=O)O1)C2] | |
| 29 | (2R)-2-(dimethylamino)-N-[1-methyl-6-[[2-oxo-3-(3-oxo-4H- | Intermediate 4 and |
| | pyrazino[2,3-b][1,4]oxazin-6-yl)-1-oxa-3,8-diazaspiro[4.5]decan-8- | Intermediate 31 |
| | yl]methyl]-6,7-dihydro-5H-cyclopenta[c]pyridin-3-yl]propanamide | |
| | 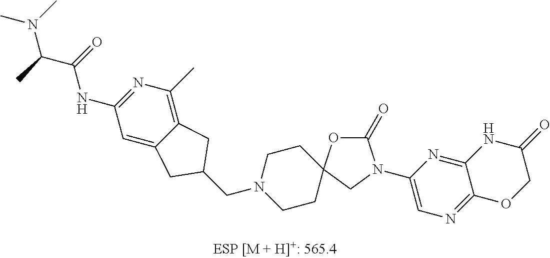 [SMILES: Cc1nc(NC(=O)[C@@H](C)N(C)C)cc2c1CC(CN1CCC3(CC1)CN(c1cnc4c(n1)NC(=O)CO4)C(=O)O3)C2] | |
| 30 | (2R)-N-[4,7-difluoro-2-hydroxy-2-[[2-[2-oxo-3-(3-oxo-4H- | Intermediate 1 and |
| | pyrazino[2,3-b][1,4]oxazin-6-yl)oxazolidin-5- | Intermediate 32 |
| | ylethylamino]methyl]indan-5-yl]-2-(dimethylamino)propanamide | |
| | 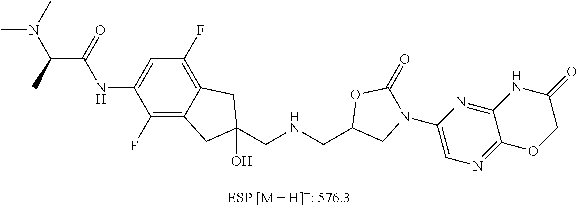 [SMILES: C[C@H](C(=O)Nc1cc(F)c2c(c1F)CC(O)(CNCC1CN(c3cnc4c(n3)NC(=O)CO4)C(=O)O1)C2)N(C)C] | |
| 31 | (2R)-N-[4,7-difluoro-2-methoxy-2-[[2-[2-oxo-3-(3-oxo-4H- | Intermediate 1 and |
| | pyrazino[2,3-b][1,4]oxazin-6-yl)oxazolidin-5- | Intermediate 33 |
| | ylethylamino]methyl]indan-5-yl]-2-(dimethylamino)propanamide | |
| | 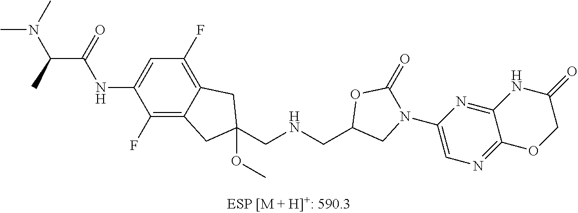 [SMILES: COC1(CNCC2CN(c3cnc4c(n3)NC(=O)CO4)C(=O)O2)Cc2c(F)cc(NC(=O)[C@@H](C)N(C)C)c(F)c2C1] | |
| 32 | (2R)-2-(dimethylamino)-N-[2,4,7-trifluoro-2-[[2-[2-oxo-3-(3-oxo- | Intermediate 1 and |
| | 4H-pyrazino[2,3-b][1,4]oxazin-6-yl)oxazolidin-5- | Intermediate 34 |
| | yl]ethylaminomethyl]indan-5-yl]propanamide | |
| | 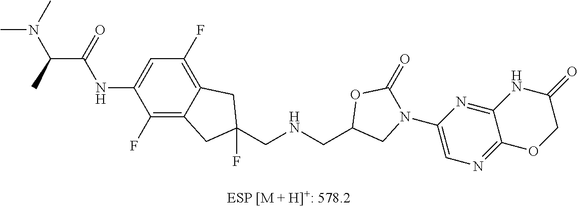 [SMILES: C[C@H](C(=O)Nc1cc(F)c2c(c1F)CC(F)(CNCC1CN(c3cnc4c(n3)NC(=O)CO4)C(=O)O1)C2)N(C)C] | |
| 33 | (2R)-N-[2,7-difluoro-2-[[2-[2-oxo-3-(3-oxo-4H-pyrazino[2,3- | Intermediate 1 and |
| | b][1,4]oxazin-6-yl)oxazolidin-5-yl]ethylamino]methyl]indan-5-yl]-2- | Intermediate 35 |
| | (dimethylamino)propanamide | |
| | 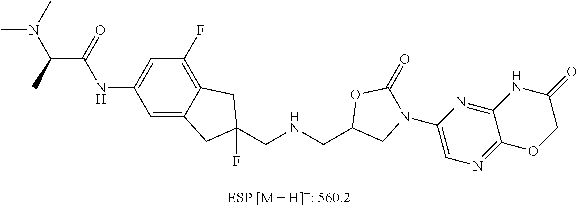 [SMILES: C[C@H](C(=O)Nc1cc(F)c2c(c1)CC(F)(CNCC1CN(c3cnc4c(n3)NC(=O)CO4)C(=O)O1)C2)N(C)C] | |
| 34 | (2R)-N-[7-cyano-2-hydroxy-2-[[2-[2-oxo-3-(3-oxo-4H-pyrazino[2,3- | Intermediate 1 and |
| | b][1,4]oxazin-6-yl)oxazolidin-5-yl]ethylamino]methyl]indan-5-yl]-2- | Intermediate 36 |
| | (dimethylamino)propanamide | |
| | 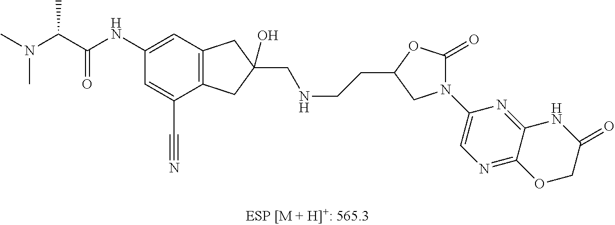 [SMILES: C[C@H](C(=O)Nc1cc(C#N)c2c(c1)CC(O)(CNCCC1CN(c3cnc4c(n3)NC(=O)CO4)C(=O)O1)C2)N(C)C] | |
| 35 | (2R)-N-[7-cyano-2-hydroxy-2-[[2-oxo-3-(3-oxo-4H-pyrazino[2,3- | Intermediate 4 and |
| | b][1,4]oxazin-6-yl)-1-oxa-3,8-diazaspiro[4.5]decan-8- | Intermediate 36 |
| | yl]methyl]indan-5-yl]-2-(dimethylamino)propanamide | |
| | 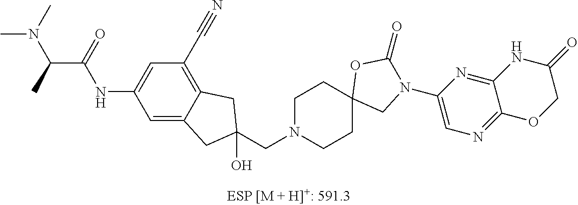 [SMILES: C[C@H](C(=O)Nc1cc(C#N)c2c(c1)CC(O)(CN1CCC3(CC1)CN(c1cnc4c(n1)NC(=O)CO4)C(=O)O3)C2)N(C)C] | |

### Example 36

### (2S)—N-[7-Fluoro-2-[[2-[2-oxo-3-(3-oxo-4H-pyrazino[2,3-b][1,4]oxazin-6-yl)oxazolidin-5-yl]ethylamino]methyl]indan-5-yl]pyrrolidine-2-carboxamide

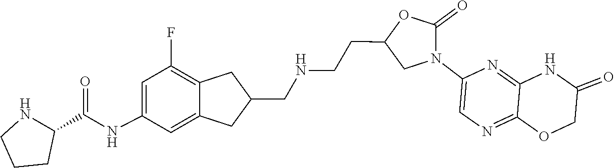 [SMILES: O=C1COc2ncc(N3CC(CCNCC4Cc5cc(NC(=O)[C@@H]6CCCN6)cc(F)c5C4)OC3=O)nc2N1]

A mixture of 6-[5-(2-aminoethyl)-2-oxo-oxazolidin-3-yl]-4H-pyrazino[2,3-b][1,4]oxazin-3-one; 2,2,2-trifluoroacetic acid (Intermediate 1, 62.69 mg, 0.160 mmol, 1 eq), tert-butyl (2S)-2-[(7-fluoro-2-formyl-indan-5-yl)carbamoyl]pyrrolidine-1-carboxylate (Intermediate 37, 60.0 mg, 0.160 mmol, 1 eq) and DIPEA (41.64 uL, 0.240 mmol, 1.5 eq) in THE (1.5 mL) and DMA (0.5 mL) was stirred at room temperature for 15 min. Acetic acid (22.81 uL, 0.400 mmol, 2.5 eq) and sodium triacetoxyborohydride (50.67 mg, 0.240 mmol, 1.5 eq) were added and the mixture was stirred at r.t. for 1 h. EtOAc and NaHCO₃ (sat. aq.) were added and the phases were separated. The organic phase was dried over Na₂SO₄, filtered and concentrated. The obtained crude was purified by flash column chromatography (0-5% MeOH in DCM, then 0-10% MeOH in DCM) to give tert-butyl (2S)-2-[[7-fluoro-2-[[2-[2-oxo-3-(3-oxo-4H-pyrazino[2,3-b][1,4]oxazin-6-yl)oxazolidin-5-yl]ethylamino]methyl]indan-5-yl]carbamoyl]pyrrolidine-1-carboxylate (47.0 mg, 0.070 mmol, 55.32% yield) as a white solid. To a solution of this product in DCM (2 mL) was added trifluoroacetic acid (0.11 mL, 1.47 mmol, 20 eq). The mixture was stirred at r.t. overnight. EtOAc and NaHCO₃ solution (sat. aq.) were added. The phases were separated, the aqueous phase was extracted with EtOAc (×2). The combined organic phases were dried over Na₂SO₄, filtered and concentrated. The obtained crude was purified by flash column chromatography (0-10% MeOH in DCM, then 10-30% MeOH in DCM) to give the title compound (13 mg, 0.020 mmol, 31.15% yield) as a white solid. MS (ESP) m/z=540.3 [M+H]⁺.

The following examples were prepared by analogy to Example 36.

| Ex. | Name, Structure, MS | Starting Materials |
|---|---|---|
| 37 | (2S,4R)-N-[7-fluoro-2-[[2-[2-oxo-3-(3-oxo-4H-pyrazino[2,3- | Intermediate 1 and |
| | b][1,4]oxazin-6-yl)oxazolidin-5-yl]ethylamino]methyl]indan-5-yl]-4- | Intermediate 38 |
| | hydroxy-pyrrolidine-2-carboxamide | |
| | 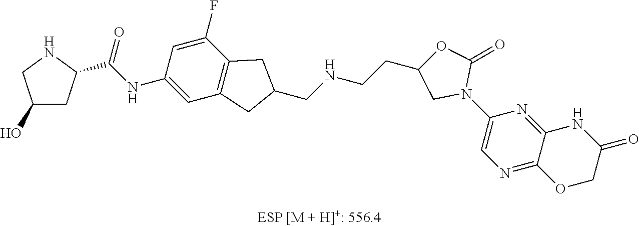 [SMILES: O=C1COc2ncc(N3CC(CCNCC4Cc5cc(NC(=O)[C@@H]6C[C@@H](O)CN6)cc(F)c5C4)OC3=O)nc2N1] | |
| 38 | (2R)-2-amino-N-[7-fluoro-2-[[2-[2-oxo-3-(3-oxo-4H-pyrazino[2,3- | Intermediate 1 and |
| | b][1,4]oxazin-6-yl)oxazolidin-5-yl]ethylamino]methyl]indan-5-yl]-3- | Intermediate 39 |
| | hydroxy-propanamide | |
| | 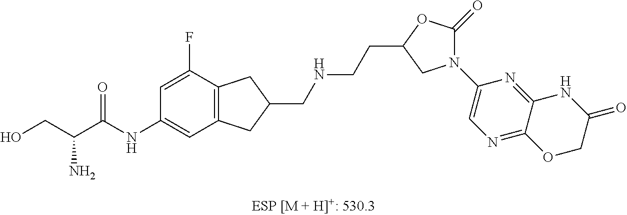 [SMILES: N[C@H](CO)C(=O)Nc1cc(F)c2c(c1)CC(CNCCC1CN(c3cnc4c(n3)NC(=O)CO4)C(=O)O1)C2] | |
| 39 | (2R)-2-amino-N-[7-fluoro-2-[[2-[2-oxo-3-(3-oxo-4H-pyrazino[2,3- | Intermediate 1 and |
| | b][1,4]oxazin-6-yl)oxazolidin-5-yl]ethylamino]methyl]indan-5-yl]-3- | Intermediate 40 |
| | methoxy-propanamide | |
| | 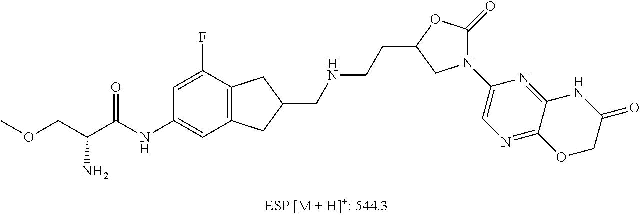 [SMILES: COC[C@@H](N)C(=O)Nc1cc(F)c2c(c1)CC(CNCCC1CN(c3cnc4c(n3)NC(=O)CO4)C(=O)O1)C2] | |
| 40 | (3R)-3-amino-N-[7-fluoro-2-[[2-[2-oxo-3-(3-oxo-4H-pyrazino[2,3- | Intermediate 1 and |
| | b][1,4]oxazin-6-yl)oxazolidin-5-yl]ethylamino]methyl]indan-5- | Intermediate 41 |
| | yl]tetrahydrofuran-3-carboxamide; 2,2,2-trifluoroacetic acid | |
| | 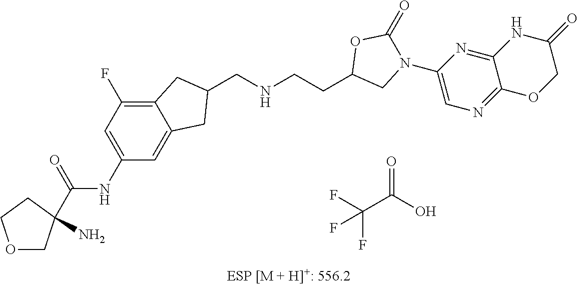 [SMILES: N[C@]1(C(=O)Nc2cc(F)c3c(c2)CC(CNCCC2CN(c4cnc5c(n4)NC(=O)CO5)C(=O)O2)C3)CCOC1 ; O=C(O)C(F)(F)F] | |
| 41 | (3S)-3-amino-N-[7-fluoro-2-[[2-[2-oxo-3-(3-oxo-4H-pyrazino[2,3- | Intermediate 1 and |
| | b][1,4]oxazin-6-yl)oxazolidin-5-yl]ethylamino]methyl]indan-5- | Intermediate 42 |
| | yl]tetrahydrofuran-3-carboxamide; hydrochloride | |
| | 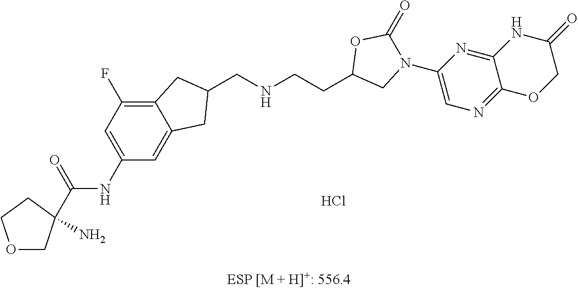 [SMILES: N[C@@]1(C(=O)Nc2cc(F)c3c(c2)CC(CNCCC2CN(c4cnc5c(n4)NC(=O)CO5)C(=O)O2)C3)CCOC1 ; Cl] | |
| 42 | (2S,4R)-N-[7-fluoro-2-[[2-oxo-3-(3-oxo-4H-pyrazino[2,3- | Intermediate 4 and |
| | b][1,4]oxazin-6-yl)-1-oxa-3,8-diazaspiro[4.5]decan-8- | Intermediate 38 |
| | yl]methyl]indan-5-yl]-4-hydroxy-pyrrolidine-2-carboxamide | |
| | 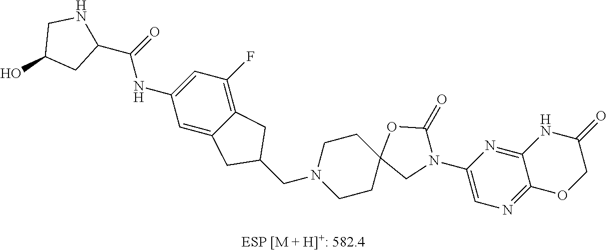 [SMILES: O=C1COc2ncc(N3CC4(CCN(CC5Cc6cc(NC(=O)C7C[C@@H](O)CN7)cc(F)c6C5)CC4)OC3=O)nc2N1] | |
| 43 | 2-amino-N-[7-fluoro-2-[[2-[2-oxo-3-(3-oxo-4H-pyrazino[2,3- | Intermediate 1 and |
| | b][1,4]oxazin-6-yl)oxazolidin-5-yl]ethylamino]methyl]indan-5-yl]-2- | Intermediate 43 |
| | methyl-propanamide | |
| | 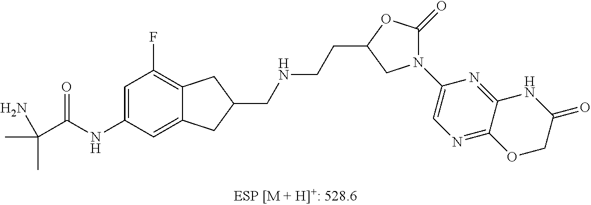 [SMILES: CC(C)(N)C(=O)Nc1cc(F)c2c(c1)CC(CNCCC1CN(c3cnc4c(n3)NC(=O)CO4)C(=O)O1)C2] | |
| 44 | (2S,4R)-N-[7-fluoro-2-[[2-[2-oxo-3-(3-oxo-4H-pyrazino[2,3- | Intermediate 1 and |
| | b][1,4]oxazin-6-yl)oxazolidin-5-yl]ethylamino]methyl]indan-5-yl]-4- | Intermediate 44 |
| | methoxy-pyrrolidine-2-carboxamide | |
| | 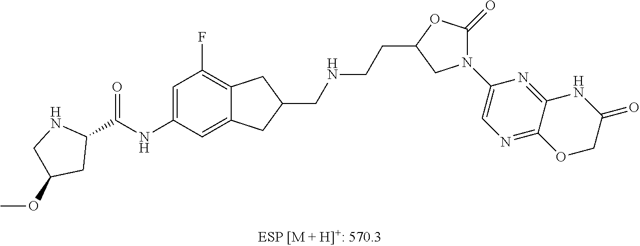 [SMILES: CO[C@H]1CN[C@H](C(=O)Nc2cc(F)c3c(c2)CC(CNCCC2CN(c4cnc5c(n4)NC(=O)CO5)C(=O)O2)C3)C1] | |
| 45 | (2R,4R)-N-[7-fluoro-2-[2-[2-oxo-3-(3-oxo-4H-pyrazino[2,3- | Intermediate 1 and |
| | b][1,4]oxazin-6-yl)oxazolidin-5-yl]ethylamino]methyl]indan-5-yl]-4- | Intermediate 45 |
| | hydroxy-pyrrolidine-2-carboxamide | |
| | 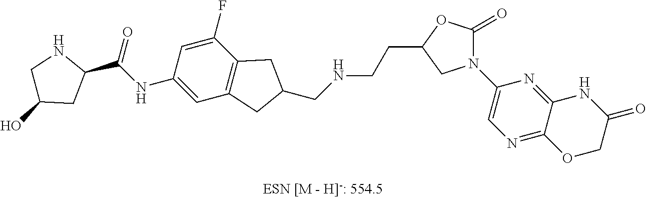 [SMILES: O=C1COc2ncc(N3CC(CCNCC4Cc5cc(NC(=O)[C@H]6C[C@@H](O)CN6)cc(F)c5C4)OC3=O)nc2N1] | |
| 46 | (2S,4S)-N-[7-fluoro-2-[[2-[2-oxo-3-(3-oxo-4H-pyrazino[2,3- | Intermediate 1 and |
| | b][1,4]oxazin-6-yl)oxazolidin-5-yl]ethylamino]methyl]indan-5-yl]-4- | Intermediate 46 |
| | hydroxy-pyrrolidine-2-carboxamide | |
| | 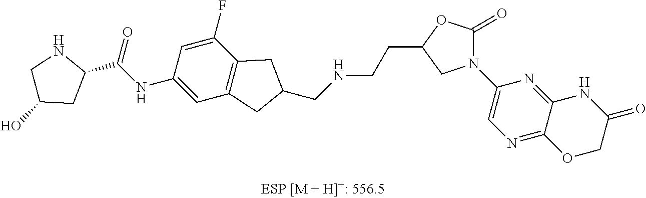 [SMILES: O=C1COc2ncc(N3CC(CCNCC4Cc5cc(NC(=O)[C@@H]6C[C@H](O)CN6)cc(F)c5C4)OC3=O)nc2N1] | |
| 47 | (2R,4S)-N-[7-fluoro-2-[[2-[2-oxo-3-(3-oxo-4H-pyrazino[2,3- | Intermediate 1 and |
| | b][1,4]oxazin-6-yl)oxazolidin-5-yl]ethylamino]methyl]indan-5-yl]-4- | Intermediate 47 |
| | hydroxy-pyrrolidine-2-carboxamide | |
| | 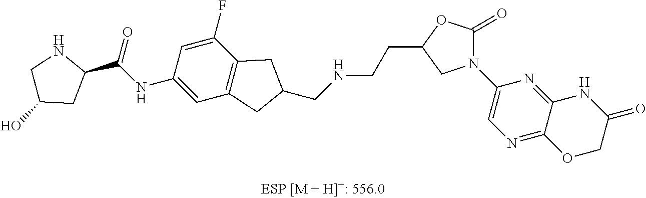 [SMILES: O=C1COc2ncc(N3CC(CCNCC4Cc5cc(NC(=O)[C@H]6C[C@H](O)CN6)cc(F)c5C4)OC3=O)nc2N1] | |
| 48 | (3S)-3-amino-4-[[7-fluoro-2-[[2-[2-oxo-3-(3-oxo-4H-pyrazino[2,3- | Intermediate 1 and |
| | b][1,4]oxazin-6-yl)oxazolidin-5-yl]ethylamino]methyl]indan-5- | Intermediate 48 |
| | yl]amino]-4-oxo-butanoic acid; 2,2,2-trifluoroacetic acid | |
| | 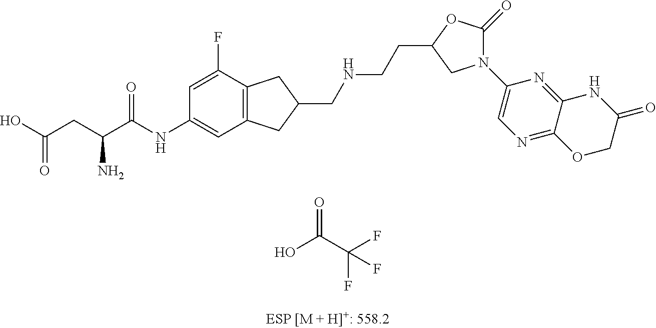 [SMILES: N[C@@H](CC(=O)O)C(=O)Nc1cc(F)c2c(c1)CC(CNCCC1CN(c3cnc4c(n3)NC(=O)CO4)C(=O)O1)C2 ; O=C(O)C(F)(F)F] | |

### Example 49

### (2S,4R)—N-[7-Fluoro-2-[[2-[2-oxo-3-(3-oxo-4H-pyrazino[2,3-b][1,4]oxazin-6-yl)oxazolidin-5-yl]ethylamino]methyl]indan-5-yl]-4-hydroxy-1-methyl-pyrrolidine-2-carboxamide

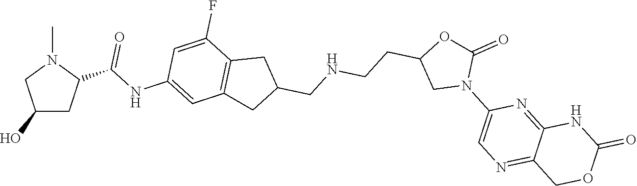

A mixture of 6-[5-(2-aminoethyl)-2-oxo-oxazolidin-3-yl]-4H-pyrazino[2,3-b][1,4]oxazin-3-one; 2,2,2-trifluoroacetic acid (Intermediate 1, 35.35 mg, 0.090 mmol, 1 eq), (2S,4R)-4-[tert-butyl(dimethyl)silyl]oxy-N-(7-fluoro-2-formyl-indan-5-yl)-1-methyl-pyrrolidine-2-carboxamide (Intermediate 49, 37.8 mg, 0.090 mmol, 1 eq) and N,N-diisopropylethylamine (0.02 mL, 0.130 mmol, 1.5 eq) in THF (1.5 mL) and DMA (0.500 mL) was stirred at RT for 15 min. then sodium triacetoxyborohydride (28.57 mg, 0.130 mmol, 1.5 eq) and acetic acid (0.01 mL, 0.220 mmol, 2.5 eq) were added. The mixture was stirred at 20° C. for 48 h. NaHCO₃ (sat. aq.) and EtOAc were added. The mixture was stirred for 15 min. then the phases were separated. The organic phase was dried over Na₂SO₄, filtered and concentrated. The obtained crude was purified by flash column chromatography (0-10% MeOH in DCM) to give (2S,4R)-4-[tert-butyl(dimethyl)silyl]oxy-N-[7-fluoro-2-[[2-[2-oxo-3-(3-oxo-4H-pyrazino[2,3-b][1,4]oxazin-6-yl)oxazolidin-5-yl]ethylamino]methyl]indan-5-yl]-1-methyl-pyrrolidine-2-carboxamide (28.5 mg, 0.040 mmol, 46.37% yield) as a yellow solid. To a solution of this material in tetrahydrofuran (2 mL), tetrabutylammonium fluoride (83.35 uL, 0.080 mmol, 2 eq) was added at RT. The mixture was stirred for 24 h at RT then it was concentrated. The residue was purified by flash column chromatography (1-20% MeOH in DCM) to give the title compound (17 mg, 0.030 mmol, 70.18% yield) as a light yellow solid. MS (ESP) m/z=570.4 [M+H]⁺.

### Example 50

### N-[7-Fluoro-2-[[2-[2-oxo-3-(3-oxo-4H-pyrazino[2,3-b][1,4]oxazin-6-yl)oxazolidin-5-yl]ethylamino]methyl]indan-5-yl]-2-[(3R)-3-hydroxypyrrolidin-1-yl]acetamide

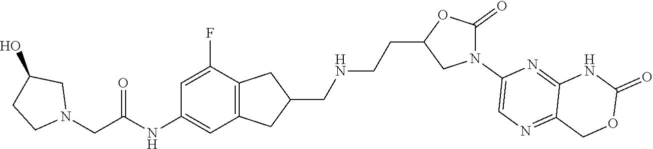 [SMILES: O=C(CN1CC[C@@H](O)C1)Nc1cc(F)c2c(c1)CC(CNCCC1CN(c3cnc4c(n3)NC(=O)OC4)C(=O)O1)C2]

The title compound was prepared by analogy to Example 49 using 2-[(3R)-3-[tert-butyl(diphenyl)silyl]oxypyrrolidin-1-yl]-N-(7-fluoro-2-formyl-indan-5-yl)acetamide (Intermediate 50) in place of (2S,4R)-4-[tert-butyl(dimethyl)silyl]oxy-N-(7-fluoro-2-formyl-indan-5-yl)-1-methyl-pyrrolidine-2-carboxamide and was obtained as a white solid. MS (ESP) m/z=570.3 [M+H]⁺.

### Example 51

### (2S)-2-(Dimethylamino)-N-[7-fluoro-2-[[2-oxo-3-(3-oxo-4H-pyrazino[2,3-b][1,4]oxazin-6-yl)-1-oxa-3,8-diazaspiro[4.5]decan-8-yl]methyl]indan-5-yl]propanamide

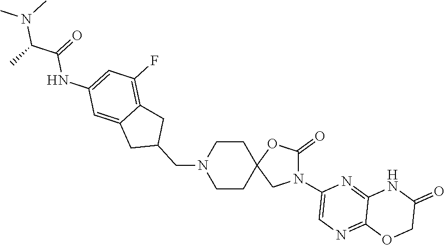

A suspension of 6-[8-[(6-amino-4-fluoro-indan-2-yl)methyl]-2-oxo-1-oxa-3,8-diazaspiro[4.5]decan-3-yl]-4H-pyrazino[2,3-b][1,4]oxazin-3-one (Intermediate 51, 30.0 mg, 0.060 mmol, 1 eq), (2S)-2-(dimethylamino)propanoic acid (8.25 mg, 0.070 mmol, 1.1 eq), TCFH (21.56 mg, 0.080 mmol, 1.2 eq) and 1-methylimidazole (17.87 uL, 0.220 mmol, 3.5 eq) in MeCN (0.637 mL) was stirred at r.t. for 16 h. Volatiles were removed under reduced pressure to give a residue which was purified by reverse phase chromatography (from water+0.1% ammonium hydroxide/acetonitrile 95:5 to water+0.1% ammonium hydroxide/acetonitrile 65:35) and then by silica cartridge chromatography (5 g silica-NH, from DCM 100% to DCM:[EtOAc/MeOH 9:1] 7:3) to give the title compound (15 mg, 0.030 mmol, 41.27% yield) as a white solid. MS (ESP) m/z=568.3 [M+H]⁺.

### Example 52

### (2S,4R)—N-[4,7-Difluoro-2-[[2-oxo-3-(3-oxo-4H-pyrazino[2,3-b][1,4]oxazin-6-yl)-1-oxa-3,8-diazaspiro[4.5]decan-8-yl]methyl]indan-5-yl]-4-hydroxy-pyrrolidine-2-carboxamide

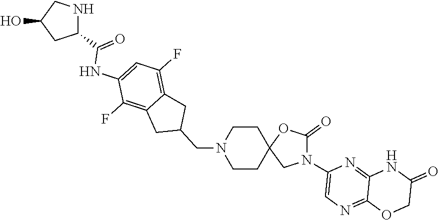

A suspension of 6-[8-[(5-amino-4,7-difluoro-indan-2-yl)methyl]-2-oxo-1-oxa-3,8-diazaspiro[4.5]decan-3-yl]-4H-pyrazino[2,3-b][1,4]oxazin-3-one (Intermediate 52, 17.5 mg, 0.040 mmol, 1 eq), (2S,4R)-1-tert-butoxycarbonyl-4-[tert-butyl(dimethyl)silyl]oxy-pyrrolidine-2-carboxylic acid (Intermediate 38, Step 1, 16.16 mg, 0.050 mmol, 1.3 eq), TCFH (16.15 mg, 0.060 mmol, 1.6 eq) and 1-methylimidazole (11.47 uL, 0.140 mmol, 4 eq) in MeCN (2 mL) was stirred at RT for 6 h. Then TCFH (7.07 mg, 0.030 mmol, 0.700 eq), (2S,4R)-1-tert-butoxycarbonyl-4-[tert-butyl(dimethyl)silyl]oxy-pyrrolidine-2-carboxylic acid (16.16 mg, 0.050 mmol, 1.3 eq) and DMF (0.800 mL) were added and the mixture stirred overnight. The reaction mixture was diluted with EtOAc and NaHCO₃ (sat. aq.). The phases were separated and the aqueous layer was extracted with EtOAc (×2). The combined organic phases were filtered through a phase separator and volatiles were removed under reduced pressure to afford a residue which was purified by silica cartridge chromatography (5 g, from DCM 100% to DCM/MeOH 95:5) to give tert-butyl (2S,4R)-4-[tert-butyl(dimethyl)silyl]oxy-2-[[4,7-difluoro-2-[[2-oxo-3-(3-oxo-4H-pyrazino[2,3-b][1,4]oxazin-6-yl)-1-oxa-3,8-diazaspiro[4.5]decan-8-yl]methyl]indan-5-yl]carbamoyl]pyrrolidine-1-carboxylate (22.5 mg, 0.030 mmol, 76.84% yield) as a white solid. To a solution of this material in DCM (2 mL), trifluoroacetic acid (0.8 mL, 10.38 mmol, 393.12 eq) was added and the mixture was stirred at RT overnight. Then DCM was evaporated, trifluoroacetic acid (0.8 mL, 10.38 mmol, 393.12 eq) was added and the mixture stirred for a further 18 h. The mixture was concentrated under vacuum then NaHCO₃ (sat. aq.) was added and the mixture stirred for 15 min. The mixture was extracted with EtOAc (×2) and EtOAc/MeOH (9:1). The combined organic phases were dried through a phase separator and concentrated under vacuum. The resulting crude was purified by flash column chromatography (10 g, from 100% DCM to 90:10 DCM/MeOH) to give the title compound (11 mg, 0.020 mmol, 67.37% yield). MS (ESP) m/z=600.5 [M+H]⁺.

### Example 53

### (2R)—N-[7-Fluoro-2-[[2-oxo-3-(3-oxo-4H-pyrazino[2,3-b][1,4]oxazin-6-yl)-1-oxa-3,8-diazaspiro[4.5]decan-8-yl]methyl]indan-5-yl]-2-(hydroxymethyl)pyrrolidine-2-carboxamide; 2,2,2-trifluoroacetic Acid

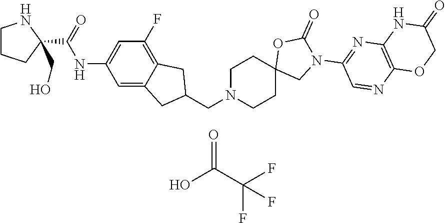

To a solution of tert-butyl (2R)-2-[[tert-butyl(dimethyl)silyl]oxymethyl]-2-[[7-fluoro-2-[[2-oxo-3-(3-oxo-4H-pyrazino[2,3-b][1,4]oxazin-6-yl)-1-oxa-3,8-diazaspiro[4.5]decan-8-yl]methyl]indan-5-yl]carbamoyl]pyrrolidine-1-carboxylate (Intermediate 53, 125.0 mg, 0.150 mmol, 1 eq) in THF (1.54 mL) at 25° C., 1 M tetrabutylammonium fluoride in THF (0.43 mL, 0.430 mmol, 2.77 eq) was added. The reaction mixture was stirred at 25° C. for 5 min. then at RT for 4 h. The reaction mixture was diluted with EtOAc and NH₄Cl (sat. aq.). The phases were separated and the organic phase was washed with NH₄Cl (sat. aq.) (×2). The organic phase was filtered through a phase separator and volatiles were removed under reduced pressure to give a residue which was purified by silica cartridge chromatography (10 g, from DCM 100% to DCM/MeOH 9:1+0.1% NH₄OH) to give tert-butyl (2R)-2-[[7-fluoro-2-[[2-oxo-3-(3-oxo-4H-pyrazino[2,3-b][1,4]oxazin-6-yl)-1-oxa-3,8-diazaspiro[4.5]decan-8-yl]methyl]indan-5-yl]carbamoyl]-2-(hydroxymethyl)pyrrolidine-1-carboxylate (63 mg, 0.090 mmol, 58.68% yield) as an off-white solid. A solution of this material in DCM (0.906 mL) and trifluoroacetic acid (0.3 mL, 3.91 mmol, 43.18 eq) was stirred at r.t. for 0.75 h. Volatiles were removed under reduced pressure and the residue evaporated with MeCN several times until all TFA was removed. The residue was suspended in DCM and pentane and stirred, the supernatant was removed and this operation was repeated 3 times. The remaining solid was dried under reduced pressure to give the title compound (39.1 mg, 0.050 mmol, 65.25% yield) as an off-white solid. MS (ESP) m/z=596.3 [M+H]⁺.

### Example 54

### (2S,3S)—N-[7-Fluoro-2-[[2-oxo-3-(3-oxo-4H-pyrazino[2,3-b][1,4]oxazin-6-yl)-1-oxa-3,8-diazaspiro[4.5]decan-8-yl]methyl]indan-5-yl]-3-hydroxy-pyrrolidine-2-carboxamide; 2,2,2-trifluoroacetic Acid

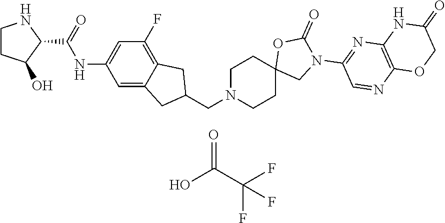

The title compound was prepared from tert-butyl (2S,3S)-3-[tert-butyl(dimethyl)silyl]oxy-2-[[7-fluoro-2-[[2-oxo-3-(3-oxo-4H-pyrazino[2,3-b][1,4]oxazin-6-yl)-1-oxa-3,8-diazaspiro[4.5]decan-8-yl]methyl]indan-5-yl]carbamoyl]pyrrolidine-1-carboxylate (Intermediate 54) by analogy to Example 53 and was obtained as an off-white solid. MS (ESP) m/z=582.3 [M+H]⁺.

### Example 55

### (2R,4R)—N-[7-Fluoro-2-[[2-oxo-3-(3-oxo-4H-pyrazino[2,3-b][1,4]oxazin-6-yl)-1-oxa-3,8-diazaspiro[4.5]decan-8-yl]methyl]indan-5-yl]-4-hydroxy-pyrrolidine-2-carboxamide

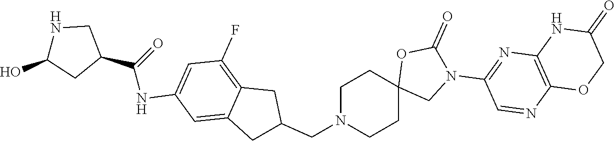 [SMILES: O=C1COc2ncc(N3CC4(CCN(CC5Cc6cc(NC(=O)[C@@H]7CN[C@H](O)C7)cc(F)c6C5)CC4)OC3=O)nc2N1]

The title compound was prepared by analogy to Example 52 using 6-[8-[(6-amino-4-fluoro-indan-2-yl)methyl]-2-oxo-1-oxa-3,8-diazaspiro[4.5]decan-3-yl]-4H-pyrazino[2,3-b][1,4]oxazin-3-one (Intermediate 51) in place of 6-[8-[(5-amino-4,7-difluoro-indan-2-yl)methyl]-2-oxo-1-oxa-3,8-diazaspiro[4.5]decan-3-yl]-4H-pyrazino[2,3-b][1,4]oxazin-3-one, and (2R,4R)-1-tert-butoxycarbonyl-4-[tert-butyl(dimethyl)silyl]oxy-pyrrolidine-2-carboxylic acid (Intermediate 45, Step 1) in place of (2S,4R)-1-tert-butoxycarbonyl-4-[tert-butyl(dimethyl)silyl]oxy-pyrrolidine-2-carboxylic acid, and was obtained as a white solid. MS (ESP) m/z=582.4 [M+H]⁺.

### Example 56

### (2R,4S)—N-[7-Fluoro-2-[[2-oxo-3-(3-oxo-4H-pyrazino[2,3-b][1,4]oxazin-6-yl)-1-oxa-3,8-diazaspiro[4.5]decan-8-yl]methyl]indan-5-yl]-4-hydroxy-pyrrolidine-2-carboxamide

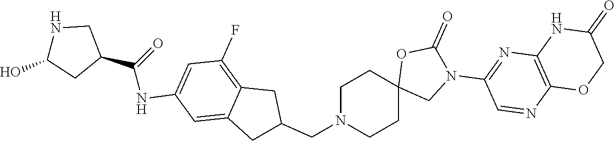 [SMILES: O=C1COc2ncc(N3CC4(CCN(CC5Cc6cc(NC(=O)[C@@H]7CN[C@@H](O)C7)cc(F)c6C5)CC4)OC3=O)nc2N1]

The title compound was prepared by analogy to Example 55 using (2R,4S)-1-tert-butoxycarbonyl-4-[tert-butyl(dimethyl)silyl]oxy-pyrrolidine-2-carboxylic acid (Intermediate 47, Step 1) in place of (2R,4R)-1-tert-butoxycarbonyl-4-[tert-butyl(dimethyl)silyl]oxy-pyrrolidine-2-carboxylic acid and was obtained as a white solid. MS (ESP) m/z=582.4 [M+H]⁺.

### Example 57

### (2S,4S)—N-[7-Fluoro-2-[[2-oxo-3-(3-oxo-4H-pyrazino[2,3-b][1,4]oxazin-6-yl)-1-oxa-3,8-diazaspiro[4.5]decan-8-yl]methyl]indan-5-yl]-4-hydroxy-pyrrolidine-2-carboxamide

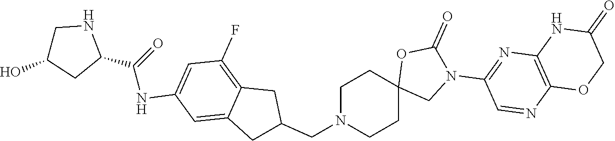 [SMILES: O=C1COc2ncc(N3CC4(CCN(CC5Cc6cc(NC(=O)[C@@H]7C[C@H](O)CN7)cc(F)c6C5)CC4)OC3=O)nc2N1]

The title compound was prepared by analogy to Example 55 using (2S,4S)-1-tert-butoxycarbonyl-4-[tert-butyl(dimethyl)silyl]oxy-pyrrolidine-2-carboxylic acid (Intermediate 46, Step 1) in place of (2R,4R)-1-tert-butoxycarbonyl-4-[tert-butyl(dimethyl)silyl]oxy-pyrrolidine-2-carboxylic acid and was obtained as a white solid. MS (ESP) m/z=582.4 [M+H]⁺.

### Example 58

### (2R)-2-(Dimethylamino)-N-[7-fluoro-2-[[methyl-[2-[2-oxo-3-(3-oxo-4H-pyrazino[2,3-b][1,4]oxazin-6-yl)oxazolidin-5-yl]ethyl]amino]methyl]indan-5-yl]propanamide

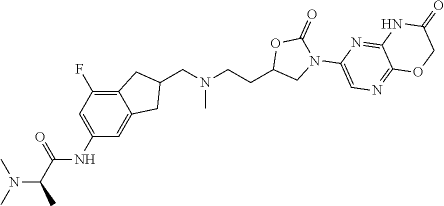 [SMILES: C[C@H](C(=O)Nc1cc(F)c2c(c1)CC(CN(C)CCC1CN(c3cnc4c(n3)NC(=O)CO4)C(=O)O1)C2)N(C)C]

To a stirred solution of (2R)-2-(dimethylamino)-N-[7-fluoro-2-[[2-[2-oxo-3-(3-oxo-4H-pyrazino[2,3-b][1,4]oxazin-6-yl)oxazolidin-5-yl]ethylamino]methyl]indan-5-yl]propanamide (Example 4, 74.0 mg, 0.140 mmol, 1 eq) in THF (2.28 mL), formaldehyde 37 wt % solution in water (10.17 uL, 0.140 mmol, 1 eq) and sodium triacetoxyborohydride (57.92 mg, 0.270 mmol, 2 eq) were added and the mixture was stirred overnight. Additional formaldehyde 37 wt % solution in water (10.17 uL, 0.140 mmol, 1 eq) and sodium triacetoxyborohydride (28.% mg, 0.140 mmol, 1 eq) were added and stirring was continued for 2 h. Then the reaction was quenched with a diluted solution of NaHCO₃ (aq.) and extracted with DCM (3×5 mL). The combined organic layers were dried through a phase separator and concentrated under reduced pressure. The residue was purified by silica cartridge chromatography (10 g, methanol/DCM 0 to 100%) then by a reverse phase silica cartridge (5 g, acetonitrile/(water+0.1% NH₃) 1% to 53%) followed by lyophilization to afford the title compound (35 mg, 0.060 mmol, 45.64% yield) as a white solid. MS (ESP) m/z=556.5 [M+H]⁺.

### Example 59

### (2S,4R)—N-[7-cyano-2-[[2-oxo-3-(3-oxo-4H-pyrazino[2,3-b][1,4]oxazin-6-yl)-1-oxa-3,8-diazaspiro[4.5]decan-8-yl]methyl]indan-5-yl]-4-hydroxy-pyrrolidine-2-carboxamide

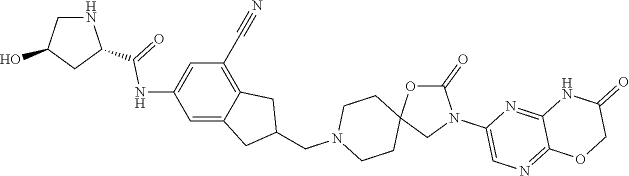 [SMILES: N#Cc1cc(NC(=O)[C@@H]2C[C@@H](O)CN2)cc2c1CC(CN1CCC3(CC1)CN(c1cnc4c(n1)NC(=O)CO4)C(=O)O3)C2]

The title compound was prepared by analogy to Example 52 using 6-amino-2-[[2-oxo-3-(3-oxo-4H-pyrazino[2,3-b][1,4]oxazin-6-yl)-1-oxa-3,8-diazaspiro[4.5]decan-8-yl]methyl]indane-4-carbonitrile (Intermediate 55) in place of 6-[8-[(5-amino-4,7-difluoro-indan-2-yl)methyl]-2-oxo-1-oxa-3,8-diazaspiro[4.5]decan-3-yl]-4H-pyrazino[2,3-b][1,4]oxazin-3-one and was obtained as a white solid. MS (ESP) m/z=589.4 [M+H]⁺.

### Example 60

### (2S,4R)-4-Hydroxy-N-[6-[[2-oxo-3-(3-oxo-4H-pyrazino[2,3-b][1,4]oxazin-6-yl)-1-oxa-3,8-diazaspiro[4.5]decan-8-yl]methyl]-6,7-dihydro-5H-cyclopenta[b]pyridin-3-yl]pyrrolidine-2-carboxamide

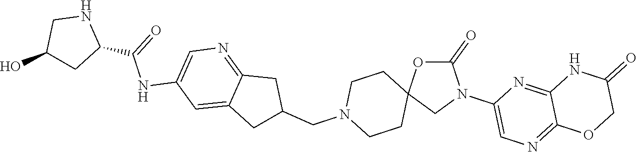 [SMILES: O=C1COc2ncc(N3CC4(CCN(CC5Cc6cc(NC(=O)[C@@H]7C[C@@H](O)CN7)cnc6C5)CC4)OC3=O)nc2N1]

A solution of tert-butyl (2S,4R)-4-[tert-butyl(dimethyl)silyl]oxy-2-[[6-[[2-oxo-3-(3-oxo-4H-pyrazino[2,3-b][1,4]oxazin-6-yl)-1-oxa-3,8-diazaspiro[4.5]decan-8-yl]methyl]-6,7-dihydro-5H-cyclopenta[b]pyridin-3-yl]carbamoyl]pyrrolidine-1-carboxylate (Intermediate 56, 30.0 mg, 0.040 mmol, 1 eq) in trifluoroacetic acid (1.0 mL, 12.98 mmol, 337.03 eq) was stirred at r.t. for 19 h. Volatiles were removed under reduced pressure to afford a residue which was purified by silica cartridge chromatography (5 g silica-NH, from DCM 100% to DCM/[DCM/MeOH 9:1] 45:55) to give the title compound (12 mg, 0.020 mmol, 52.43% yield) as a white solid. MS (ESP) m/z=565.4 [M+H]⁺.

### Example 61

### (2S,4R)—N-[7-Cyano-2-[[2-[2-oxo-3-(3-oxo-4H-pyrazino[2,3-b][1,4]oxazin-6-yl)oxazolidin-5-yl]ethylamino]methyl]indan-5-yl]-4-hydroxy-pyrrolidine-2-carboxamide

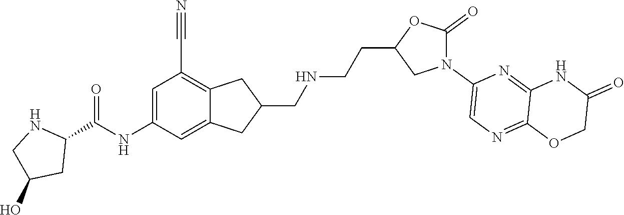 [SMILES: N#Cc1cc(NC(=O)[C@@H]2C[C@@H](O)CN2)cc2c1CC(CNCCC1CN(c3cnc4c(n3)NC(=O)CO4)C(=O)O1)C2]

The title compound was prepared from tert-butyl (2S,4R)-2-[[2-[[tert-butoxycarbonyl-[2-[2-oxo-3-(3-oxo-4H-pyrazino[2,3-b][1,4]oxazin-6-yl)oxazolidin-5-yl]ethyl]amino]methyl]-7-cyano-indan-5-yl]carbamoyl]-4-[tert-butyl(dimethyl)silyl]oxy-pyrrolidine-1-carboxylate (Intermediate 57) by analogy to Example 53 and was obtained as a white solid. MS (ESP) m/z=563.3 [M+H]⁺.

### Example 62

### (2R)—N-[7-Cyano-2-[[2-[2-oxo-3-(3-oxo-4H-pyrazino[2,3-b][1,4]oxazin-6-yl)oxazolidin-5-yl]ethylamino]methyl]indan-5-yl]-1-methyl-pyrrolidine-2-carboxamide

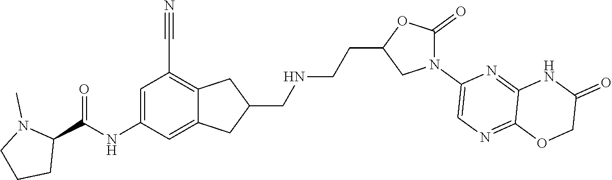 [SMILES: CN1CCC[C@@H]1C(=O)Nc1cc(C#N)c2c(c1)CC(CNCCC1CN(c3cnc4c(n3)NC(=O)CO4)C(=O)O1)C2]

The title compound was prepared from tert-butyl N-[(6-amino-4-cyano-indan-2-yl)methyl]-N-[2-[2-oxo-3-(3-oxo-4H-pyrazino[2,3-b][1,4]oxazin-6-yl)oxazolidin-5-yl]ethyl]carbamate (Intermediate 57, Step 5) and (2R)-1-methylpyrrolidine-2-carboxylic acid [CAS #58123-62-9] by analogy to Example 52 and was obtained as a white solid. MS (ESP) m/z=561.4 [M+H]⁺.

### Example 63

### (2S,4R)—N-[7-Fluoro-2-[[methyl-[2-[2-oxo-3-(3-oxo-4H-pyrazino[2,3-b][1,4]oxazin-6-yl)oxazolidin-5-yl]ethyl]amino]methyl]indan-5-yl]-4-hydroxy-pyrrolidine-2-carboxamide

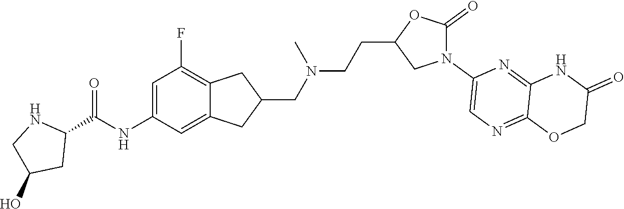 [SMILES: CN(CCC1CN(c2cnc3c(n2)NC(=O)CO3)C(=O)O1)CC1Cc2cc(NC(=O)[C@@H]3C[C@@H](O)CN3)cc(F)c2C1]

The title compound was prepared from tert-butyl (2S,4R)-4-[tert-butyl(dimethyl)silyl]oxy-2-[[7-fluoro-2-[[methyl-[2-[2-oxo-3-(3-oxo-4H-pyrazino[2,3-b][1,4]oxazin-6-yl)oxazolidin-5-yl]ethyl]amino]methyl]indan-5-yl]carbamoyl]pyrrolidine-1-carboxylate (Intermediate 58) by analogy to Example 60. MS (ESP) m/z=570.4 [M+H]⁺.

### Example 64

### (2S,4R)—N-[7-Fluoro-2-[[2-oxo-3-(3-oxo-4H-pyrazino[2,3-b][1,4]oxazin-6-yl)-1-oxa-3,8-diazaspiro[4.5]decan-8-yl]methyl]indan-5-yl]-4-hydroxy-1-methyl-pyrrolidine-2-carboxamide

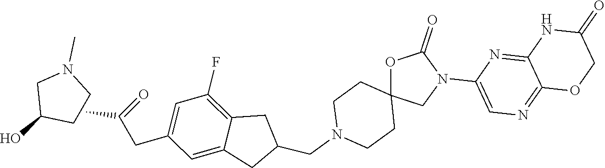

The title compound was prepared by analogy to Example 58 using (2S,4R)—N-[7-fluoro-2-[[2-oxo-3-(3-oxo-4H-pyrazino[2,3-b][1,4]oxazin-6-yl)-1-oxa-3,8-diazaspiro[4.5]decan-8-yl]methyl]indan-5-yl]-4-hydroxy-pyrrolidine-2-carboxamide (Example 42) in place of (2R)-2-(dimethylamino)-N-[7-fluoro-2-[[2-[2-oxo-3-(3-oxo-4H-pyrazino[2,3-b][1,4]oxazin-6-yl)oxazolidin-5-yl]ethylamino]methyl]indan-5-yl]propanamide and was obtained as a white solid. MS (ESP) m/z=596.4 [M+H]⁺.

### Example 65

### (2R)-2-(Dimethylamino)-N-[7-fluoro-3-hydroxy-2-[[2-oxo-3-(3-oxo-4H-pyrazino[2,3-b][1,4]oxazin-6-yl)-1-oxa-3,8-diazaspiro[4.5]decan-8-yl]methyl]indan-5-yl]propanamide

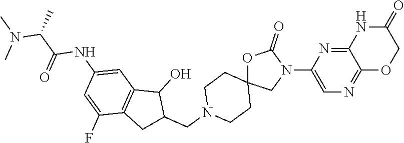 [SMILES: C[C@H](C(=O)Nc1cc(F)c2c(c1)C(O)C(CN1CCC3(CC1)CN(c1cnc4c(n1)NC(=O)CO4)C(=O)O3)C2)N(C)C]

The title compound was prepared by analogy to Example 49 using (2R)—N-[3-[tert-butyl(dimethyl)silyl]oxy-7-fluoro-2-formyl-indan-5-yl]-2-(dimethylamino)propanamide (Intermediate 59) in place of (2S,4R)-4-[tert-butyl(dimethyl)silyl]oxy-N-(7-fluoro-2-formyl-indan-5-yl)-1-methyl-pyrrolidine-2-carboxamide, and 6-(2-oxo-1-oxa-3,8-diazaspiro[4.5]decan-3-yl)-4H-pyrazino[2,3-b][1,4]oxazin-3-one; 2,2,2-trifluoroacetic acid (Intermediate 4) in place of 6-[5-(2-aminoethyl)-2-oxo-oxazolidin-3-yl]-4H-pyrazino[2,3-b][1,4]oxazin-3-one; 2,2,2-trifluoroacetic acid, and was obtained as a light yellow solid. MS (ESP) m/z=584.3 [M+H]⁺.

### Example 66

### (2R)-2-(Dimethylamino)-N-[7-fluoro-3-hydroxy-2-[[2-[(5R)-2-oxo-3-(3-oxo-4H-pyrazino[2,3-b][1,4]oxazin-6-yl)oxazolidin-5-yl]ethylamino]methyl]indan-5-yl]propanamide

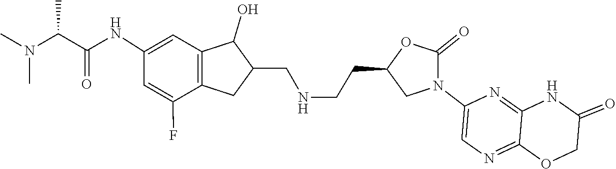 [SMILES: C[C@H](C(=O)Nc1cc(F)c2c(c1)C(O)C(CNCC[C@@H]1CN(c3cnc4c(n3)NC(=O)CO4)C(=O)O1)C2)N(C)C]

The title compound was prepared by analogy to Example 65 using 6-[(5R)-5-(2-aminoethyl)-2-oxo-oxazolidin-3-yl]-4H-pyrazino[2,3-b][1,4]oxazin-3-one; 2,2,2-trifluoroacetic acid (Intermediate 62) in place of 6-(2-oxo-1-oxa-3,8-diazaspiro[4.5]decan-3-yl)-4H-pyrazino[2,3-b][1,4]oxazin-3-one; 2,2,2-trifluoroacetic acid and was obtained as a brown solid. MS (ESP) m/z=558.2 [M+H]⁺.

### Example 67

### (2R)—N-[3,7-Difluoro-2-[[2-[(5R)-2-oxo-3-(3-oxo-4H-pyrazino[2,3-b][1,4]oxazin-6-yl)oxazolidin-5-yl]ethylamino]methyl]indan-5-yl]-2-(dimethylamino)propanamide

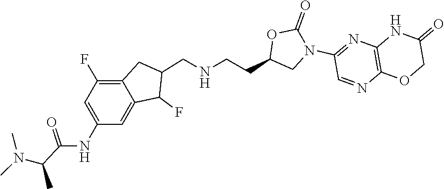 [SMILES: C[C@H](C(=O)Nc1cc(F)c2c(c1)C(F)C(CNCC[C@@H]1CN(c3cnc4c(n3)NC(=O)CO4)C(=O)O1)C2)N(C)C]

A solution of (2R)—N-[3,7-difluoro-2-[[2-[(5R)-2-oxo-3-[3-oxo-4-(2-trimethylsilylethoxymethyl)pyrazino[2,3-b][1,4]oxazin-6-yl]oxazolidin-5-yl]ethylamino]methyl]indan-5-yl]-2-(dimethylamino)propanamide (Intermediate 60, 18.9 mg, 0.030 mmol, 1 eq) in DCM (0.274 mL) and trifluoroacetic acid (0.15 mL, 1.95 mmol, 71.06 eq) was stirred at 25° C. for 1 h. Volatiles were removed under reduced pressure and the residue was purified by silica cartridge chromatography (5 g, DCM/MeOH from 99:1 to 8:2) to afford the title compound (4.4 mg, 0.010 mmol, 24.39% yield) as an off-white solid. MS (ESP) m/z=560.3 [M+H]⁺.

### Example 68

### (2R)-2-(Dimethylamino)-N-[7-fluoro-2-[[2-[(5S)-2-oxo-3-(3-oxo-4H-pyrazino[2,3-b][1,4]oxazin-6-yl)oxazolidin-5-yl]ethylamino]methyl]indan-5-yl]propanamide Epimer A

### And

### Example 69

### (2R)-2-(Dimethylamino)-N-[7-fluoro-2-[[2-[(5S)-2-oxo-3-(3-oxo-4H-pyrazino[2,3-b][1,4]oxazin-6-yl)oxazolidin-5-yl]ethylamino]methyl]indan-5-yl]propanamide Epimer B

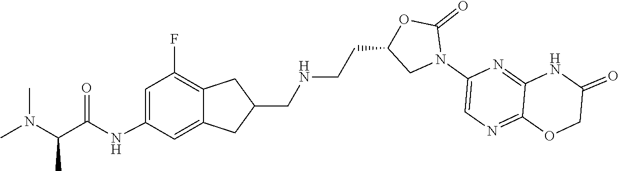 [SMILES: C[C@H](C(=O)Nc1cc(F)c2c(c1)CC(CNCC[C@H]1CN(c3cnc4c(n3)NC(=O)CO4)C(=O)O1)C2)N(C)C]

A mixture of (2R)-2-(dimethylamino)-N-(7-fluoro-2-formyl-indan-5-yl)propanamide (Intermediate 10, 516.0 mg, 1.85 mmol, 1 eq), 6-[(5S)-5-(2-aminoethyl)-2-oxo-oxazolidin-3-yl]-4H-pyrazino[2,3-b][1,4]oxazin-3-one; 2,2,2-trifluoroacetic acid (Intermediate 63, 780.17 mg, 1.98 mmol, 1.07 eq) and N,N-diisopropylethylamine (0.65 mL, 3.71 mmol, 2 eq) in DMA (9.26 mL) and THF (27.81 mL) was stirred at r.t. for 45 min. Acetic acid (0.32 mL, 5.56 mmol, 3 eq) was added followed by sodium triacetoxyborohydride (589.4 mg, 2.78 mmol, 1.5 eq) and then the mixture was stirred at r.t. for 1.5 h. EtOAc and NaHCO₃ (sat. aq.) were added, the mixture was left stirring for 20 min. then the phases were separated. The aqueous phase was extracted with EtOAc (×3). The combined organic phases were dried through a phase separator and concentrated under reduced pressure. The obtained crude was purified by silica cartridge chromatography (25 g, 0-20% MeOH in DCM) to give (2R)-2-(dimethylamino)-N-[7-fluoro-2-[[2-[(5S)-2-oxo-3-(3-oxo-4H-pyrazino[2,3-b][1,4]oxazin-6-yl)oxazolidin-5-yl]ethylamino]methyl]indan-5-yl]propanamide (579 mg, 1.07 mmol, 57.67% yield) as a light brown foam.

The epimers of (2R)-2-(dimethylamino)-N-[7-fluoro-2-[[2-[(5R)-2-oxo-3-(3-oxo-4H-pyrazino[2,3-b][1,4]oxazin-6-yl)oxazolidin-5-yl]ethylamino]methyl]indan-5-yl]propanamide (547.0 mg, 1.01 mmol, 1 eq) were separated by preparative chiral SFC to give peak 1, (2R)-2-(dimethylamino)-N-[7-fluoro-2-[[2-[(5R)-2-oxo-3-(3-oxo-4H-pyrazino[2,3-b][1,4]oxazin-6-yl)oxazolidin-5-yl]ethylamino]methyl]indan-5-yl]propanamide Epimer A (207 mg, 0.380 mmol, 37.84% yield) as an off-white solid, and peak 2, (2R)-2-(dimethylamino)-N-[7-fluoro-2-[[2-[(5R)-2-oxo-3-(3-oxo-4H-pyrazino[2,3-b][1,4]oxazin-6-yl)oxazolidin-5-yl]ethylamino]methyl]indan-5-yl]propanamide Epimer B (207 mg, 0.380 mmol, 37.84% yield) as a white solid. MS (ESP) m/z=542.3 [M+H]⁺.

### Example 70

### (2R)-2-(Dimethylamino)-N-[7-fluoro-2-[[2-[(5R)-2-oxo-3-(3-oxo-4H-pyrazino[2,3-b][1,4]oxazin-6-yl)oxazolidin-5-yl]ethylamino]methyl]indan-5-yl]propanamide Epimer A

### And

### Example 71

### (2R)-2-(Dimethylamino)-N-[7-fluoro-2-[[2-[(5R)-2-oxo-3-(3-oxo-4H-pyrazino[2,3-b][1,4]oxazin-6-yl)oxazolidin-5-yl]ethylamino]methyl]indan-5-yl]propanamide Epimer B

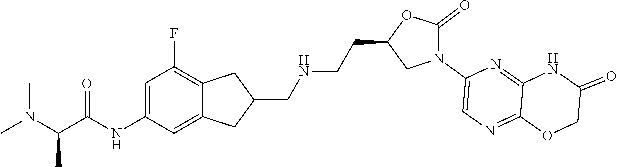 [SMILES: C[C@H](C(=O)Nc1cc(F)c2c(c1)CC(CNCC[C@@H]1CN(c3cnc4c(n3)NC(=O)CO4)C(=O)O1)C2)N(C)C]

The title compounds were prepared by analogy to Examples 68 and 69 using 6-[(5R)-5-(2-aminoethyl)-2-oxo-oxazolidin-3-yl]-4H-pyrazino[2,3-b][1,4]oxazin-3-one; 2,2,2-trifluoroacetic acid (Intermediate 62) in place of 6-[(5S)-5-(2-aminoethyl)-2-oxo-oxazolidin-3-yl]-4H-pyrazino[2,3-b][1,4]oxazin-3-one; 2,2,2-trifluoroacetic acid and were obtained as white solids. MS (ESP) m/z=542.2 [M+H]⁺.

### Example 72

### (2S,4R)—N-[7-Fluoro-2-[[2-oxo-3-(3-oxo-4H-pyrazino[2,3-b][1,4]oxazin-6-yl)-1-oxa-3,8-diazaspiro[4.5]decan-8-yl]methyl]indan-5-yl]-4-hydroxy-2-methyl-pyrrolidine-2-carboxamide Epimer A

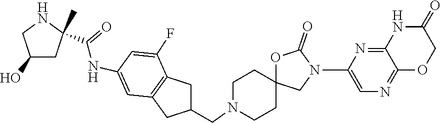 [SMILES: C[C@@]1(C(=O)Nc2cc(F)c3c(c2)CC(CN2CCC4(CC2)CN(c2cnc5c(n2)NC(=O)CO5)C(=O)O4)C3)C[C@@H](O)CN1]

### And

### Example 73

### (2S,4R)—N-[7-Fluoro-2-[[2-oxo-3-(3-oxo-4H-pyrazino[2,3-b][1,4]oxazin-6-yl)-1-oxa-3,8-diazaspiro[4.5]decan-8-yl]methyl]indan-5-yl]-4-hydroxy-2-methyl-pyrrolidine-2-carboxamide Epimer B

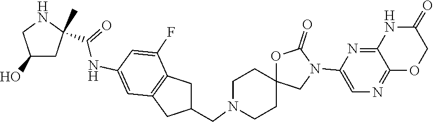 [SMILES: C[C@@]1(C(=O)Nc2cc(F)c3c(c2)CC(CN2CCC4(CC2)CN(c2cnc5c(n2)NC(=O)CO5)C(=O)O4)C3)C[C@@H](O)CN1]

To a solution of 6-[8-[(6-amino-4-fluoro-indan-2-yl)methyl]-2-oxo-1-oxa-3,8-diazaspiro[4.5]decan-3-yl]-4H-pyrazino[2,3-b][1,4]oxazin-3-one; hydrochloride (Intermediate 51, 120.0 mg, 0.24 mmol, 1.0 eq) and (2S,4R)-1-tert-butoxycarbonyl-4-[tert-butyl(dimethyl)silyl]oxy-2-methyl-pyrrolidine-2-carboxylic acid (Intermediate 63, 111.08 mg, 0.31 mmol, 1.3 eq) in MeCN (3 mL) was added TCFH (106.69 mg, 0.38 mmol, 1.6 eq) and 1-methylimidazole (58.54 mg, 0.71 mmol, 3.0 eq) to give a white suspension. The resulting suspension was stirred at 25° C. for 2 h. The mixture was concentrated under reduced pressure to give a residue. The residue was purified by preparative TLC to give tert-butyl (2S,4R)-4-[tert-butyl(dimethyl)silyl]oxy-2-[[7-fluoro-2-[[2-oxo-3-(3-oxo-4H-pyrazino[2,3-b][1,4]oxazin-6-yl)-1-oxa-3,8-diazaspiro[4.5]decan-8-yl]methyl]indan-5-yl]carbamoyl]-2-methyl-pyrrolidine-1-carboxylate (130.0 mg, 0.16 mmol, 62.13% yield) as a light yellow solid.

To a solution of tert-butyl (2S,4R)-4-[tert-butyl(dimethyl)silyl]oxy-2-[[7-fluoro-2-[[2-oxo-3-(3-oxo-4H-pyrazino[2,3-b][1,4]oxazin-6-yl)-1-oxa-3,8-diazaspiro[4.5]decan-8-yl]methyl]indan-5-yl]carbamoyl]-2-methyl-pyrrolidine-1-carboxylate (125.0 mg, 0.15 mmol, 1.0 eq) in methanol (6 mL) cooled to 0° C. was added MeOH/HCl (6.0 mL) to give a white suspension. The resulting mixture was stirred at 20° C. for 2 h. The suspension was dried under reduced pressure to give a residue. The residue was purified by preparative HPLC and freeze-dried to give (2S,4R)—N-[7-fluoro-2-[[2-oxo-3-(3-oxo-4H-pyrazino[2,3-b][1,4]oxazin-6-yl)-1-oxa-3,8-diazaspiro[4.5]decan-8-yl]methyl]indan-5-yl]-4-hydroxy-2-methyl-pyrrolidine-2-carboxamide; formic acid (45.0 mg, 0.08 mmol, 53% yield) as a white solid.

The epimers of (2S,4R)—N-[7-fluoro-2-[[2-oxo-3-(3-oxo-4H-pyrazino[2,3-b][1,4]oxazin-6-yl)-1-oxa-3,8-diazaspiro[4.5]decan-8-yl]methyl]indan-5-yl]-4-hydroxy-2-methyl-pyrrolidine-2-carboxamide; formic acid (45.0 mg, 0.08 mmol, 1.0 eq) were separated by chiral SFC to give Peak 1, (2S,4R)—N-[7-fluoro-2-[[2-oxo-3-(3-oxo-4H-pyrazino[2,3-b][1,4]oxazin-6-yl)-1-oxa-3,8-diazaspiro[4.5]decan-8-yl]methyl]indan-5-yl]-4-hydroxy-2-methyl-pyrrolidine-2-carboxamide Epimer A (14.2 mg, 0.02 mmol, 30.7% yield) as a white solid and Peak 2, (2S,4R)—N-[7-Fluoro-2-[[2-oxo-3-(3-oxo-4H-pyrazino[2,3-b][1,4]oxazin-6-yl)-1-oxa-3,8-diazaspiro[4.5]decan-8-yl]methyl]indan-5-yl]-4-hydroxy-2-methyl-pyrrolidine-2-carboxamide Epimer B (19.4 mg, 0.03 mmol, 41.17% yield) as a white solid. MS (ESP) m/z=596.3 [M+H]⁺.

### INTERMEDIATES

### Intermediate 1

### 6-[5-(2-Aminoethyl)-2-oxo-oxazolidin-3-yl]-4H-pyrazino[2,3-b][1,4]oxazin-3-one; 2,2,2-trifluoroacetic Acid

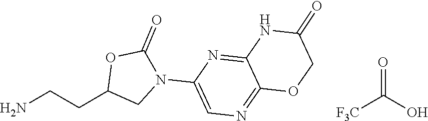 [SMILES: NCCC1CN(c2cnc3c(n2)NC(=O)CO3)C(=O)O1 ; O=C(O)C(F)(F)F]

### Step 1: 6-bromo-4H-pyrazino[2,3-b][1,4]oxazin-3-one

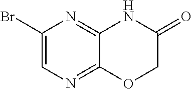 [SMILES: O=C1COc2ncc(Br)nc2N1]

To a solution of methyl glycolate (60.0 mL, 777.31 mmol, 7.86 eq) in 3,6-dibromopyrazin-2-amine [CAS #2376926-19-9](25.0 g, 98.86 mmol, 1 eq) was added potassium tert-butoxide (34.0 g, 303 mmol, 3.07 eq) at 60° C. under N₂. Methyl glycolate (10.0 mL, 98.86 mmol, 1 eq) was added and the mixture was stirred at 60° C. for 3 h. The reaction mixture was poured into aqueous HCl (1 N, 350 mL) and filtered. The filter cake was washed with H₂O (50 mL×3) and MTBE (50 mL×3), then dried under reduced pressure. The aqueous washings were extracted with EtOAc (150 mL×3) and combined with the organic filtrate (MTBE), and the mixture was concentrated to give a residue. The filter cake and the residue were combined to give the crude title compound (23 g, 100 mmol, quant. yield).

### Step 2: 5: 6-bromo-4-(2-trimethylsilylethoxymethyl)pyrazino[2,3-b][1,4]oxazin-3-one

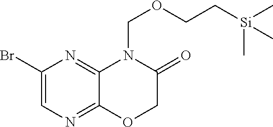 [SMILES: C[Si](C)(C)CCOCN1C(=O)COc2ncc(Br)nc21]

To a mixture of 6-bromo-4H-pyrazino[2,3-b][1,4]oxazin-3-one (15.0 g, 65.21 mmol, 1 eq) and potassium carbonate (27.0 g, 195.36 mmol, 3 eq) in DMF (100 mL) was added 2-(trimethylsilyl)ethoxymethyl chloride (18.0 mL, 101.7 mmol, 1.56 eq) at 0° C. Then the reaction was stirred at 15° C. for 0.5 h. The reaction mixture was diluted with H₂O (600 mL) and the resulting solid was collected by filtration. The solid was washed with water (50 mL×3) and dried under reduced pressure to give the title compound (23 g, 63.84 mmol, 97.9% yield) as a yellow solid.

### Step 3: tert-butyl N-[2-[2-oxo-3-[3-oxo-4-(2-trimethylsilylethoxymethyl)pyrazino[2,3-b][1,4]oxazin-6-yl]oxazolidin-5-yl]ethyl]carbamate

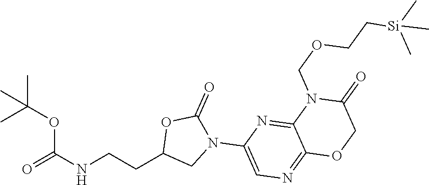 [SMILES: CC(C)(C)OC(=O)NCCC1CN(c2cnc3c(n2)N(COCC[Si](C)(C)C)C(=O)CO3)C(=O)O1]

To a solution of 6-bromo-4-(2-trimethylsilylethoxymethyl)pyrazino[2,3-b][1,4]oxazin-3-one (23.47 g, 65.14 mmol, 1 eq) and tert-butyl N-[2-(2-oxooxazolidin-5-yl)ethyl]carbamate [CAS #2353503-50-9](15.0 g, 65.14 mmol, 1 eq) in 1,4-dioxane (150 mL) was added copper(I) iodide (3722 mg, 19.54 mmol, 0.300 eq), trans-N,N′-dimethylcyclohexane-1,2-diamine (5560.5 mg, 39.09 mmol, 0.600 eq) and potassium carbonate (27009.8 mg, 195.43 mmol, 3 eq) The mixture was stirred at 100° C. for 3 h. The mixture was concentrated to give a residue. The residue was diluted with EtOAc (200 mL) and washed with brine (100 mL×2). The organic phase was dried over Na₂SO₄, then concentrated to give the crude product. The crude was purified by flash column chromatography (PE/EtOAc=3:1 to 1:1) and concentrated to give the title compound (26.25 g, 51.51 mmol, 79.1% yield).

### Step 4: 6-[5-(2-aminoethyl)-2-oxo-oxazolidin-3-yl]-4H-pyrazino[2,3-b][1,4]oxazin-3-one; 2,2,2-trifluoroacetic Acid

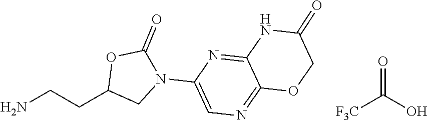 [SMILES: NCCC1CN(c2cnc3c(n2)NC(=O)CO3)C(=O)O1 ; O=C(O)C(F)(F)F]

To a mixture of tert-butyl N-[2-[2-oxo-3-[3-oxo-4-(2-trimethylsilylethoxymethyl)pyrazino[2,3-b][1,4]oxazin-6-yl]oxazolidin-5-yl]ethyl]carbamate (47.2 g, 92.62 mmol, 1 eq) in DCM (100 mL) was added trifluoroacetic acid (472.0 mL, 6126 mmol, 66.15 eq), and then the solution was stirred at 40° C. for 16 h. About 100 mL solvent was removed by air distillation, and then another 100 mL trifluoroacetic acid was added and the mixture was stirred at 70° C. for 48 h. The solvent was removed by concentration and then the residue was taken up in water (100 mL), and washed with EtOAc (100 mL×2), the organics were discarded and a solid formed which was collected by filtration and dried under reduced pressure to give the title compound (14.0 g, 35.6 mmol, 35.36% yield) as a white solid. The filtrate was concentrated to give a crude, which was azeotroped with toluene (100 mL), and the resulting solid was triturated in MeCN (100 mL), and the resulting solid was collected by filtration and dried under reduced pressure to give the title compound (15.3 g, 38.9 mmol, 38.6% yield) as an off-white solid. MS (ESP) m/z=280.1 [M+H]⁺.

### Intermediate 2

### 6-[(5R)-5-(2-Aminoethyl)-2-oxo-oxazolidin-3-yl]-4H-pyrazino[2,3-b][1,4]oxazin-3-one; Formic Acid

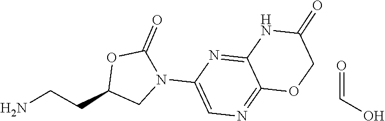

### Step 1: tert-butyl N-[2-[2-oxo-3-(3-oxo-4H-pyrazino[2,3-b][1,4]oxazin-6-yl)oxazolidin-5-yl]ethyl]carbamate

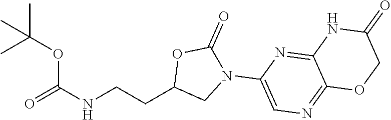 [SMILES: CC(C)(C)OC(=O)NCCC1CN(c2cnc3c(n2)NC(=O)CO3)C(=O)O1]

To a solution of 6-[5-(2-aminoethyl)-2-oxo-oxazolidin-3-yl]-4H-pyrazino[2,3-b][1,4]oxazin-3-one; 2,2,2-trifluoroacetic acid (Intermediate 1, 5.0 g, 12.71 mmol, 1 eq) in THF (50 mL) and methanol (100 mL) under ice-water cooling was added di-tert-butyldicarbonate (4162.11 mg, 19.07 mmol, 1.5 eq), and then the mixture was stirred at 25° C. for 2 h. The mixture was concentrated under vacuum to give a residue. The residue was triturated with EtOAc (20 mL×2) to give the title compound (3.8 g, 10.02 mmol, 78.79% yield) as an off-white solid. MS (ESP) m/z=402.0 [M+Na]⁺.

### Step 2: tert-butyl N-[2-[(5S)-2-oxo-3-(3-oxo-4H-pyrazino[2,3-b][1,4]oxazin-6-yl)oxazolidin-5-yl]ethyl]carbamate and tert-butyl N-[2-[(5R)-2-oxo-3-(3-oxo-4H-pyrazino[2,3-b][1,4]oxazin-6-yl)oxazolidin-5-yl]ethyl]carbamate

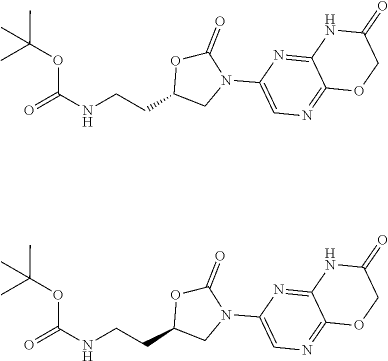 [SMILES: CC(C)(C)OC(=O)NCC[C@H]1CN(c2cnc3c(n2)NC(=O)CO3)C(=O)O1 ; CC(C)(C)OC(=O)NCC[C@@H]1CN(c2cnc3c(n2)NC(=O)CO3)C(=O)O1]

The enantiomers of tert-butyl N-[2-[2-oxo-3-(3-oxo-4H-pyrazino[2,3-b][1,4]oxazin-6-yl)oxazolidin-5-yl]ethyl]carbamate (3.7 g, 9.8 mmol) were separated by preparative chiral SFC to give peak 1, tert-butyl N-[2-[(5S)-2-oxo-3-(3-oxo-4H-pyrazino[2,3-b][1,4]oxazin-6-yl)oxazolidin-5-yl]ethyl]carbamate (1.8 g, 4.74 mmol, 48.65% yield) as a yellow solid and peak 2, tert-butyl N-[2-[(5R)-2-oxo-3-(3-oxo-4H-pyrazino[2,3-b][1,4]oxazin-6-yl)oxazolidin-5-yl]ethyl]carbamate (1.7 g, 4.48 mmol, 45.95% yield) as a yellow solid.

### Step 3: 6-[(5R)-5-(2-Aminoethyl)-2-oxo-oxazolidin-3-yl]-4H-pyrazino[2,3-b][1,4]oxazin-3-one; Formic Acid

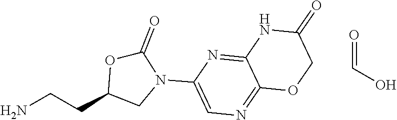

A solution of tert-butyl N-[2-[(5R)-2-oxo-3-(3-oxo-4H-pyrazino[2,3-b][1,4]oxazin-6-yl)oxazolidin-5-yl]ethyl]carbamate (1.7 g, 4.48 mmol, 1 eq) in HCOOH (10.0 mL, 4.48 mmol, 1 eq) was stirred at 25° C. for 2 h. Then the solvent was removed to give a crude and the residue was taken up in MeCN (10 mL), then the solid was collected by filtration and dried under reduced pressure to give the title compound (1400 mg, 4.3 mmol, 96.05% yield) as an off-white solid.

### Intermediate 3

### 6-[(5S)-5-(2-Aminoethyl)-2-oxo-oxazolidin-3-yl]-4H-pyrazino[2,3-b][1,4]oxazin-3-one; Formic Acid

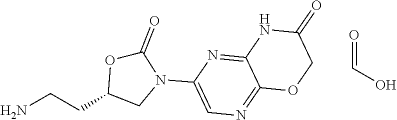

The title compound was prepared from tert-butyl N-[2-[(5S)-2-oxo-3-(3-oxo-4H-pyrazino[2,3-b][1,4]oxazin-6-yl)oxazolidin-5-yl]ethyl]carbamate (Intermediate 2, Step 2) by analogy to Intermediate 2, Step 3 and was obtained as an off-white solid.

### Intermediate 4

### 6-(2-Oxo-1-oxa-3,8-diazaspiro[4.5]decan-3-yl)-4H-pyrazino[2,3-b][1,4]oxazin-3-one; 2,2,2-formic Acid

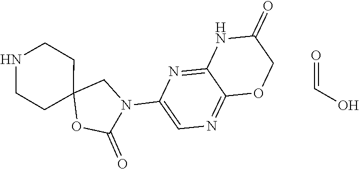

The title compound was prepared by analogy to Intermediate 1 using tert-butyl 2-oxo-1-oxa-3,8-diazaspiro[4.5]decane-8-carboxylate [CAS #169206-55-7] in place of tert-butyl N-[2-(2-oxooxazolidin-5-yl)ethyl]carbamate and was obtained as a light yellow solid. MS (ESP) m/z=306.1 [M+H]⁺.

### Intermediate 5

### (6-Amino-4-fluoro-indan-2-yl)methanol

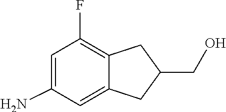 [SMILES: Nc1cc(F)c2c(c1)CC(CO)C2]

### Step 1: ethyl 4-fluoro-1-oxo-indane-2-carboxylate

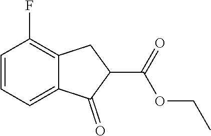 [SMILES: CCOC(=O)C1Cc2c(F)cccc2C1=O]

A mixture of sodium hydride, 60% in oil (7.99 g, 199.8 mmol, 3 eq) in toluene (120 mL) was cooled to 0° C. then diethyl carbonate (18.56 mL, 153.18 mmol, 2.3 eq) was added dropwise. The mixture was heated to 120° C. and a solution of 4-fluoro-indan-1-one [CAS #699-99-0] (10.0 g, 66.6 mmol, 1 eq) in toluene (120 mL) was added dropwise in 1.5 h. The mixture was left stirring at 120° C. for 1 h then cooled to RT. Then the reaction mixture was slowly poured into cooled NH₄Cl (sat. aq.) and acetic acid was added to adjust pH to 5. The product was extracted with EtOAc, the phases were separated, the organic phase was dried over Na₂SO₄, filtered and concentrated. The obtained crude (18.1 g) was purified by silica cartridge chromatography (200 g, 0-12% EtOAc/cyclohexane) to give title compound (13.86 g, 62.37 mmol, 91.78% yield) as a yellow oil. MS (ESP) m/z=223.1 [M+H]⁺.

### Step 2: ethyl 4-fluoroindane-2-carboxylate

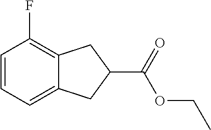

A solution of ethyl 4-fluoro-1-oxo-indane-2-carboxylate (13.2 g, 59.4 mmol, 1 eq) in ethanol (480 mL) and HCl (4.5 mL) was stirred for 4 h under a hydrogen atmosphere in presence of Pd/C (12.64 g, 11.88 mmol, 0.200 eq). The mixture was filtered and washed with EtOAc then it was concentrated in vacuo. It was diluted with EtOAc, washed with water (2×) and brine (1×) and concentrated in vacuo to afford the title compound (12.7 g, 60.99 mmol, quant. yield). MS (ESP) m/z=209.1 [M+H]⁺.

### Step 3: ethyl 4-fluoro-5-(4,4,5,5-tetramethyl-1,3,2-dioxaborolan-2-yl)indane-2-carboxylate; ethyl 4-fluoro-6-(4,4,5,5-tetramethyl-1,3,2-dioxaborolan-2-yl)indane-2-carboxylate; ethyl 4-fluoro-7-(4,4,5,5-tetramethyl-1,3,2-dioxaborolan-2-yl)indane-2-carboxylate

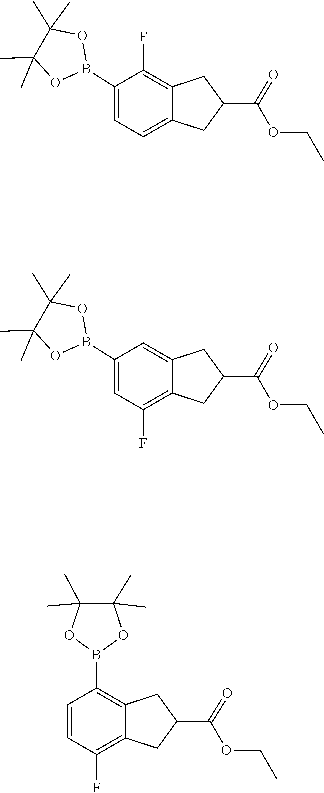

A stirred solution of 4,4,5,5-tetramethyl-1,3,2-dioxaborolane (27.09 mL, 186.72 mmol, 2 eq) and 3,4,7,8-tetramethyl-1,10-phenanthroline (882.46 mg, 3.73 mmol, 0.040 eq) was degassed 3 times and (1,5-cyclooctadiene)(methoxy)iridium(I) dimer (1237.67 mg, 1.87 mmol, 0.020 eq) was added under N₂ atmosphere. N₂ was bubbled into the reaction mixture for 15 min. then ethyl 4-fluoroindane-2-carboxylate (19.44 g, 93.36 mmol, 1 eq) was added over 20 min. The mixture was stirred at 65° C. for 4 h and then cooled to RT. EtOAc (200 mL) was added, the mixture was stirred for 30 min., the catalyst was filtered off and solvent was evaporated under reduced pressure to obtain the crude title compound mixture (35 g, 104.73 mmol, quant. yield) which was used directly in the next step. MS (ESP) m/z=335.3 [M+H]⁺.

### Step 4: ethyl 4-fluoro-5-hydroxy-indane-2-carboxylate; ethyl 4-fluoro-6-hydroxy-indane-2-carboxylate; ethyl 4-fluoro-7-hydroxy-indane-2-carboxylate

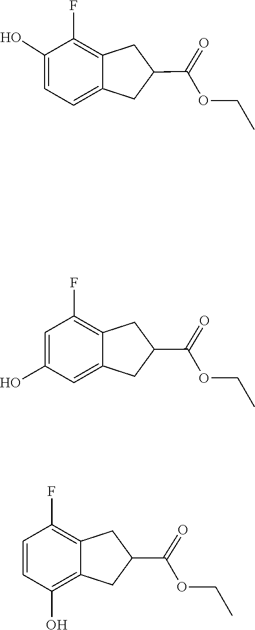 [SMILES: CCOC(=O)C1Cc2ccc(O)c(F)c2C1 ; CCOC(=O)C1Cc2cc(O)cc(F)c2C1 ; CCOC(=O)C1Cc2c(O)ccc(F)c2C1]

To a stirred solution of ethyl 4-fluoro-5-(4,4,5,5-tetramethyl-1,3,2-dioxaborolan-2-yl)indane-2-carboxylate; ethyl 4-fluoro-6-(4,4,5,5-tetramethyl-1,3,2-dioxaborolan-2-yl)indane-2-carboxylate; ethyl 4-fluoro-7-(4,4,5,5-tetramethyl-1,3,2-dioxaborolan-2-yl)indane-2-carboxylate (24.07 g, 72.02 mmol, 1 eq) in THF (50 mL), sodium perborate monohydrate (21.78 g, 216.07 mmol, 3 eq) in water (50 mL) was added at 0° C. The mixture was stirred for 1.5 h at RT. The solid was filtered and washed with EtOAc. Water was added to wash the organic layer which was separated, dried over Na₂SO₄, filtered and concentrated. The crude material was purified by flash column chromatography (silica 350 g, from 100% cyclohexane to 80:20 cyclohexane:EtOAc) to give the title compound (9.5 g, 42.37 mmol, 58.85% yield) as a mixture of the three isomers (25:72:3). MS (ESP) m/z=225.1 [M+H]⁺.

### Step 5: ethyl 4-fluoro-6-(trifluoromethylsulfonyloxy)indane-2-carboxylate

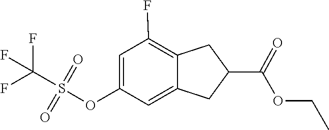 [SMILES: CCOC(=O)C1Cc2cc(OS(=O)(=O)C(F)(F)F)cc(F)c2C1]

To a solution of ethyl 4-fluoro-5-hydroxy-indane-2-carboxylate; ethyl 4-fluoro-6-hydroxy-indane-2-carboxylate; ethyl 4-fluoro-7-hydroxy-indane-2-carboxylate (9.5 g, 42.37 mmol, 1 eq) in DCM (30 mL) was added 2,6-lutidine (19.74 mL, 169.47 mmol, 4 eq) and the solution was cooled to 0° C. A solution of trifluoromethanesulfonic anhydride (14.32 mL, 84.73 mmol, 2 eq) in DCM (30 mL) was added slowly and then the mixture was left stirring at 0° C. for 1 h. Further DCM (200 mL) was added and the organic phase was washed with NH₄Cl (sat/aq.) (3×200 mL), separated, dried with Na₂SO₄, filtered and concentrated under reduced pressure. The crude product mixture was loaded on a silica column (200 g) for a coarse purification (0 to 5% ethyl acetate in cyclohexane) to obtain a mixture containing the title compound. This crude product was purified by flash chromatography (silica 350 g, from 0 to 8% ethyl acetate in cyclohexane) to give the pure title compound (9.24 g, 25.93 mmol, 61.21% yield) as a light yellow oil. MS (ESP) m/z=357.1 [M+H]⁺.

### Step 6: ethyl 6-(benzhydrylideneamino)-4-fluoro-indane-2-carboxylate

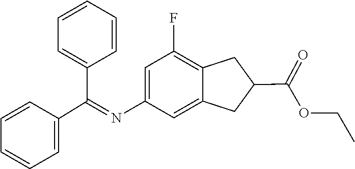 [SMILES: CCOC(=O)C1Cc2cc(N=C(c3ccccc3)c3ccccc3)cc(F)c2C1]

A mixture of ethyl 4-fluoro-6-(trifluoromethylsulfonyloxy)indane-2-carboxylate (9.24 g, 25.93 mmol, 1 eq), cesium carbonate (25.35 g, 77.8 mmol, 3 eq), XPhos (3.71 g, 7.78 mmol, 0.300 eq), tris(dibenzylideneacetone)dipalladium (0) (3.56 g, 3.89 mmol, 0.150 eq) and alpha-phenyl-benzenemethanamine (8.7 mL, 51.87 mmol, 2 eq) in 1,4-dioxane (31.51 mL) was stirred at 90° C. for 20 h. EtOAc (200 mL) was added and organics were washed with water (2×200 mL) and brine (1×200 mL). The organic phase was separated, dried over Na₂SO₄, filtered and concentrated to obtain a crude material which was loaded on a silica column (200 g) for a coarse purification (0 to 10% EtOAc in cyclohexane) to obtain a mixture containing the desired product. This crude product was then purified by flash column chromatography (200 g, from 0 to 10% EtOAc in cyclohexane) to give the title compound (8.46 g, 22.6 mmol, 87.2% yield) as a yellow oil. MS (ESP) m/z=388.2 [M+H]⁺.

### Step 7: ethyl 6-amino-4-fluoro-indane-2-carboxylate

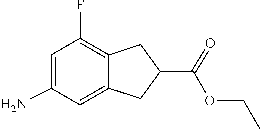 [SMILES: CCOC(=O)C1Cc2cc(N)cc(F)c2C1]

A mixture of ethyl 6-(benzhydrylideneamino)-4-fluoro-indane-2-carboxylate (8.76 g, 22.61 mmol, 1 eq) and 6M HCl (7.54 mL, 45.22 mmol, 2 eq) in THF (82.52 mL) was stirred at RT for 1 h. EtOAc was added and organic phase was washed with water, and brine. Then NaHCO₃ was added to the aqueous layers until reaching pH 8 and the product was extracted with EtOAc. The organic phase was separated, dried over Na₂SO₄, filtered and concentrated under reduced pressure to obtain a crude material which was purified by flash column chromatography (50 g, 0 to 50% EtOAc in cyclohexane) to obtain the title compound (3.05 g, 13.66 mmol, 54.4% yield) as an off-white solid. MS (ESP) m/z=224.1 [M+H]⁺.

### Step 8: (6-amino-4-fluoro-indan-2-yl)methanol

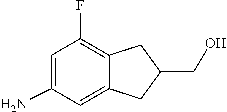 [SMILES: Nc1cc(F)c2c(c1)CC(CO)C2]

To a solution of ethyl 6-amino-4-fluoro-indane-2-carboxylate (2.8 g, 12.54 mmol, 1 eq) in dry THF (100.34 mL) at 0° C., lithium aluminum hydride 2.3 M in 2-MeTHF (12.54 mL, 28.85 mmol, 2.3 eq) was carefully added dropwise, keeping the temperature below 5° C. (over 30 min.). The reaction mixture was left stirring for 30 min. at the same temperature then it was quenched with sodium sulfate decahydrate and diluted with EtOAc. The resulting suspension was stirred at RT for 30 min. then the solid was filtered-off through a Celite® pad. The filtrate was collected, volatiles were removed under reduced pressure and the obtained crude was purified by flash column chromatography (EtOAc in cyclohexane from 0 to 100%) to give the title compound (2.14 g, 11.81 mmol, 94.16% yield) as a light yellow solid. MS (ESP) m/z=182.0 [M+H]⁺.

### Intermediate 6

### 6-[[tert-Butyl(dimethyl)silyl]oxymethyl]-4-methyl-6,7-dihydro-5H-cyclopenta[b]pyridin-2-amine

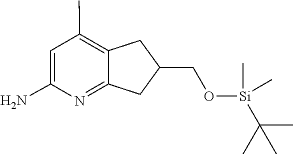 [SMILES: Cc1cc(N)nc2c1CC(CO[Si](C)(C)C(C)(C)C)C2]

### Step 1: dimethyl 4-methylpyridine-2,3-dicarboxylate

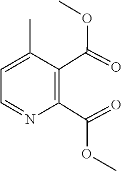

To a solution of 4-methylpyridine-2,3-dicarboxylic acid (CAS #51740-8, 1000.0 mg, 5.52 mmol, 1 eq) in DMF (20 mL) was added (trimethylsilyl)diazomethane (8.28 mL, 16.56 mmol, 3 eq) at 0° C., and then the temperature was allowed to warm to 25° C. and the mixture stirred for 16 h. The solvent was removed to give the crude product, which was purified by chromatography on silica gel (PE/EtOAc=3:1) to give the title compound (800 mg, 3.82 mmol, 69.3% yield) as a brown oil. MS (ESP): m/z=210.1 [M+H]⁺.

### Step 2: [2-(hydroxymethyl)-4-methyl-3-pyridyl]methanol

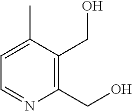

To a solution of dimethyl 4-methylpyridine-2,3-dicarboxylate (800.0 mg, 3.82 mmol, 1 eq) in ethanol (20 mL) was added sodium borohydride (868.0 mg, 22.94 mmol, 6 eq), and then the solution was stirred at 25° C. for 16 h. The pH of the solution was adjusted to around 6 by progressively adding formic acid, and then the solid was filtered off and the filtrate was concentrated to give the title compound (600 mg, 3.92 mmol, quant. yield) as a white solid. MS (ESP): m/z=154.1 [M+H]⁺.

### Step 3: 2,3-bis(chloromethyl)-4-methyl-pyridine

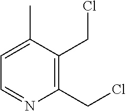

To a solution of [2-(hydroxymethyl)-4-methyl-3-pyridyl]methanol (0.6 g, 3.92 mmol, 1 eq) in DCM (10 mL) was added thionyl chloride (4.66 g, 39.17 mmol, 10 eq), and then the solution was stirred at 25° C. for 16 h. The solvent was removed by concentration and the residue was taken up in 50 mL DCM, and then the solution was poured into ice-water (20 mL), and the pH of the solution was adjusted to around 8 by progressively adding aq. NaHCO₃, and then the solution was extracted with DCM (20 mL×2), combined with the organics and dried over Na₂SO₄. The solvent was removed to give the title compound (400 mg, 2.1 mmol, 53.7% yield) as a brown oil.

### Step 4: diethyl 4-methyl-5,7-dihydrocyclopenta[b]pyridine-6,6-dicarboxylate

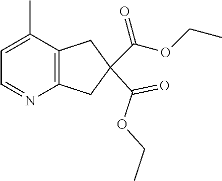 [SMILES: CCOC(=O)C1(C(=O)OCC)Cc2nccc(C)c2C1]

To a solution of diethyl malonate (0.337 g, 2.1 mmol, 1 eq) in 200 mL THF was added potassium tert-butoxide (0.5 g, 4.42 mmol, 2.1 eq) at 0° C., and the solution was stirred at 0° C. for 1 h. Then the solution was added to a stirred cooled (0° C.) solution of 2,3-bis(chloromethyl)-4-methyl-pyridine (0.4 g, 2.1 mmol, 1 eq) in THF (10.0 mL), and the resulting mixture was stirred at 70° C. for 16 h. The mixture was poured into 50 mL ice-water and extracted with EtOAc (30 mL×2), combined with the organics and dried over Na₂SO₄. The solvent was removed to give the crude product, which was purified by silica gel chromatography (PE/EtOAc=10:1) to give the title compound (350 mg, 1.26 mmol, 60% yield) as a colorless oil. MS (ESP): m/z=278.1 [M+H]⁺.

### Step 5: 4-methyl-6,7-dihydro-5H-cyclopenta[b]pyridine-6-carboxylic Acid Hydrochloride

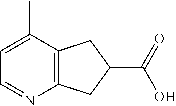

A solution of diethyl 4-methyl-5,7-dihydrocyclopenta[b]pyridine-6,6-dicarboxylate (350.0 mg, 1.26 mmol, 1 eq) in hydrochloric acid (0.13 mL, 1.26 mmol, 1 eq) was stirred at 100° C. for 16 h. The solvent was removed by concentration and to the residue was added toluene (20 mL), then the mixture was azeotroped to give the title compound (200 mg, 0.940 mmol, 74.2% yield) as a yellow solid. MS (ESP): m/z=178.1 [M+H]⁺.

### Step 6: ethyl 4-methyl-6,7-dihydro-5H-cyclopenta[b]pyridine-6-carboxylate

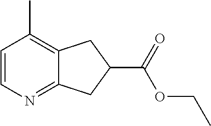

4-Methyl-6,7-dihydro-5H-cyclopenta[b]pyridine-6-carboxylic acid hydrochloride (758 mg, 3.55 mmol, 1 eq) was dissolved in ethanol (40 mL). At 0° C. conc. sulfuric acid (990 μL) was added then the solution was stirred at RT for 5 min., then at reflux for 2.5 h. The volatiles were removed, the residue was taken up carefully in Na₂CO₃ (sat. aq.) and extracted with EtOAc (3×). The organic layers were washed with brine (basic with Na₂CO₃), dried over Na₂SO₄ and evaporated to give a residue which was purified by flash column chromatography (50 g silica, heptane/EtOAc 90:10-50:50) to give the title compound (656 mg, 3.20 mmol, 90.1% yield) as a colorless oil. MS (ESP) m/z=206.1 [M+H]⁺.

### Step 7: (4-methyl-6,7-dihydro-5H-cyclopenta[b]pyridin-6-yl)methanol

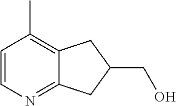

Lithium aluminum hydride (181 mg, 4.78 mmol, 1.5 eq) was added to a solution of ethyl 4-methyl-6,7-dihydro-5H-cyclopenta[b]pyridine-6-carboxylate (654 mg, 3.19 mmol, 1 eq) in THF (22 mL). The resulting grey suspension was stirred at RT for 3 h. Et₂O (30 mL) was added and the mixture was cooled to 0° C. Na₂SO₄ (sat. aq.) was added dropwise until gas evolution ceased. Solid Na₂SO₄ was added. The solid was filtered off and washed well with Et₂O. The filtrate was concentrated to give a residue which was purified by flash column chromatography (50 g silica, DCM/MeOH 98:2-95:5) to give the title compound (444 mg, 2.72 mmol, 85.38% yield) as a white solid. MS (ESP) m/z=164.1 [M+H]⁺.

### Step 8: tert-butyl-dimethyl-[(4-methyl-6,7-dihydro-5H-cyclopenta[b]pyridin-6-yl)methoxy]silane

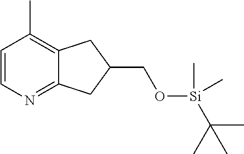 [SMILES: Cc1ccnc2c1CC(CO[Si](C)(C)C(C)(C)C)C2]

Imidazole (321.17 mg, 4.72 mmol, 2.2 eq) was dissolved in THF (65 mL) and (4-methyl-6,7-dihydro-5H-cyclopenta[b]pyridin-6-yl)methanol (350.0 mg, 2.14 mmol, 1 eq) was added. The reaction was cooled to 0° C. and tert-butyldimethylchlorosilane (646.39 mg, 4.29 mmol, 2 eq) in THF (20 mL) was added dropwise. The reaction was stirred at RT for 16 h. Then water was added and the mixture extracted with ethyl acetate (3×30 mL). The organic layers were collected, dried over Na₂SO₄, filtered and concentrated under vacuum to give a crude compound that was purified by silica cartridge chromatography (50 g, cyclohexane/EtOAc=80/20) to give the title compound (584 mg, 2.1 mmol, 98.15% yield) as a yellow oil. MS (ESP) m/z=278.2 [M+H]⁺.

### Step 9: tert-butyl-dimethyl-[(4-methyl-1-oxido-6,7-dihydro-5H-cyclopenta[b]pyridin-1-ium-6-yl)methoxy]silane

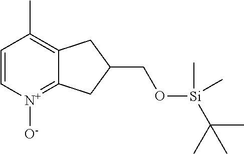 [SMILES: Cc1cc[n+]([O-])c2c1CC(CO[Si](C)(C)C(C)(C)C)C2]

To a stirred solution of tert-butyl-dimethyl-[(4-methyl-6,7-dihydro-5H-cyclopenta[b]pyridin-6-yl)methoxy]silane (584.0 mg, 2.1 mmol, 1 eq) in DCM (4.21 mL) at 0° C. was added 3-chloroperbenzoic acid (467.91 mg, 2.71 mmol, 1.29 eq). The reaction mixture was stirred at RT for 2 h. Then a saturated aqueous solution of Na₂CO₃ was added (pH=7 to 8) and the mixture extracted with DCM (3×10 mL). The organic layers were collected, dried over Na₂SO₄, filtered and concentrated under vacuum to give the title compound (622 mg, 2.12 mmol, quant. yield) as a colorless oil. MS (ESP) m/z=294.2 [M+H]⁺.

### Step 10: 6-[[tert-butyl(dimethyl)silyl]oxymethyl]-N-[(2,4-dimethoxyphenyl)methyl]-4-methyl-6,7-dihydro-5H-cyclopenta[b]pyridin-2-amine

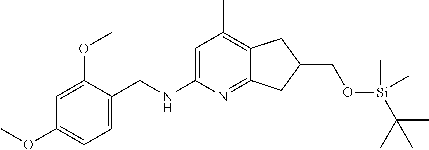 [SMILES: COc1ccc(CNc2cc(C)c3c(n2)CC(CO[Si](C)(C)C(C)(C)C)C3)c(OC)c1]

To a solution of tert-butyl-dimethyl-[(4-methyl-1-oxido-6,7-dihydro-5H-cyclopenta[b]pyridin-1-ium-6-yl)methoxy]silane (491.0 mg, 1.67 mmol, 1 eq) in DCM (16.73 mL) at 0° C. were added bromotripyrrolidinophosphonium hexafluorophosphate (1403.88 mg, 3.01 mmol, 1.8 eq), 2,4-dimethoxybenzylamine (0.33 mL, 2.17 mmol, 1.3 eq) and N,N-diisopropylethylamine (1.02 mL, 5.86 mmol, 3.5 eq). The resulting mixture was stirred at 25° C. for 16 h. Then water was added and the mixture extracted with DCM (3×50 mL). Organic layers were collected, dried over Na₂SO₄, filtered and concentrated under vacuum to give a crude product that was purified by silica cartridge chromatography (50 g, cyclohexane/ethyl acetate from 100:0 to 80:20) to give the title compound (184 mg, 0.420 mmol, 24.84% yield) as a yellow oil. MS (ESP) m/z=443.3 [M+H]⁺.

### Step 11: (2-amino-4-methyl-6,7-dihydro-5H-cyclopenta[b]pyridin-6-yl)methanol

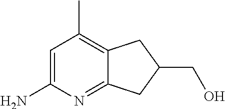

A solution of 6-[[tert-butyl(dimethyl)silyl]oxymethyl]-N-[(2,4-dimethoxyphenyl)methyl]-4-methyl-6,7-dihydro-5H-cyclopenta[b]pyridin-2-amine (230.0 mg, 0.520 mmol, 1 eq) in HCl (0.67 mL, 4.02 mmol, 7.75 eq) was stirred at 70° C. for 1 h. The mixture was concentrated under vacuum and purified by SCX chromatography to give the title compound (72 mg, 0.400 mmol, 77.75% yield) as a light yellow solid. MS (ESP) m/z=179.1 [M+H]⁺.

### Step 12: 6-[[tert-butyl(dimethyl)silyl]oxymethyl]-4-methyl-6,7-dihydro-5H-cyclopenta[b]pyridin-2-amine

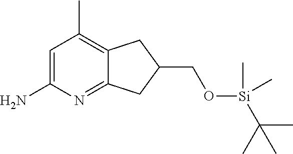 [SMILES: Cc1cc(N)nc2c1CC(CO[Si](C)(C)C(C)(C)C)C2]

To a stirred solution of (2-amino-4-methyl-6,7-dihydro-5H-cyclopenta[b]pyridin-6-yl)methanol (376.0 mg, 2.11 mmol, 1 eq) in THF (14 mL) was added imidazole (315.97 mg, 4.64 mmol, 2.2 eq). The mixture was cooled to 0° C. and a solution of tert-butyldimethylchlorosilane (635.44 mg, 4.22 mmol, 2 eq) in THF (12 mL) was added dropwise. The reaction mixture was stirred at 25° C. for 16 h. Then a saturated aqueous solution of NH₄Cl was added and the mixture extracted with ethyl acetate (3×50 mL). The combined organic extracts were dried over Na₂SO₄, filtered and concentrated under vacuum to give the crude product that was purified by silica cartridge chromatography (50 g, cyclohexane/ethyl acetate from 0:100 to 90:10) to give the title compound (442 mg, 1.51 mmol, 71.63% yield) as a light yellow oil. MS (ESP) m/z=293.3 [M+H]⁺.

### Intermediate 7

### 2-(Dimethylamino)-N-(7-fluoro-2-formyl-indan-5-yl)acetamide

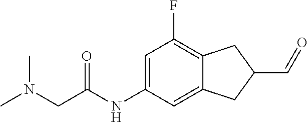 [SMILES: CN(C)CC(=O)Nc1cc(F)c2c(c1)CC(C=O)C2]

### Step 1: 2-(dimethylamino)-N-[7-fluoro-2-(hydroxymethyl)indan-5-yl]acetamide

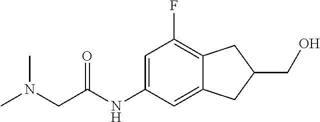 [SMILES: CN(C)CC(=O)Nc1cc(F)c2c(c1)CC(CO)C2]

To a mixture of (6-amino-4-fluoro-indan-2-yl)methanol (Intermediate 5, 85.0 mg, 0.470 mmol, 1 eq) and N,N-dimethylglycine [CAS #1118-68-9] (53.21 mg, 0.520 mmol, 1.1 eq) in MeCN (5 mL), 1-methylimidazole (0.13 mL, 1.64 mmol, 3.5 eq) and TCFH (157.93 mg, 0.560 mmol, 1.2 eq) were added. The mixture was stirred at RT overnight then concentrated and purified by flash column chromatography (20-100% MeCN in DCM) to give the title compound (35 mg, 0.130 mmol, 28.02% yield). MS (ESP) m/z=267.0 [M+H]⁺.

### Step 2: 2-(dimethylamino)-N-(7-fluoro-2-formyl-indan-5-yl)acetamide

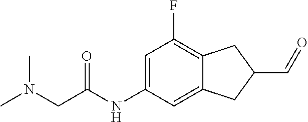 [SMILES: CN(C)CC(=O)Nc1cc(F)c2c(c1)CC(C=O)C2]

A mixture of 2-(dimethylamino)-N-[7-fluoro-2-(hydroxymethyl)indan-5-yl]acetamide (35.0 mg, 0.130 mmol, 1 eq) in DCM (2 mL) was cooled to 0° C. and Dess-Martin periodinane (83.61 mg, 0.200 mmol, 1.5 eq) was added. The mixture was warmed to RT over 30 min., then it was stirred at RT for 3 h. Further DCM and NaHCO₃ (diluted sat. aq.) were added. The phases were separated and the organic phase was dried over Na₂SO₄, filtered and concentrated to give the crude title compound (77 mg) which was used in the next step without further purification. MS (ESP) m/z=265.0 [M+H]⁺.

### Intermediate 8

### 1-(Dimethylamino)-N-(7-fluoro-2-formyl-indan-5-yl)cyclopropanecarboxamide

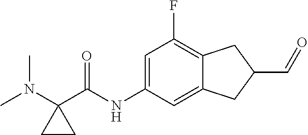 [SMILES: CN(C)C1(C(=O)Nc2cc(F)c3c(c2)CC(C=O)C3)CC1]

The title compound was prepared by analogy to Intermediate 7 using 1-(dimethylamino)cyclopropanecarboxylic acid [CAS #119111-65-8] in place of N,N-dimethylglycine and was obtained as a colourless viscous oil. MS (ESP) m/z=291.1 [M+H]⁺.

### Intermediate 9

### 2-(dimethylamino)-N-(7-fluoro-2-formyl-indan-5-yl)-2-methyl-propanamide

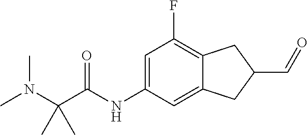 [SMILES: CN(C)C(C)(C)C(=O)Nc1cc(F)c2c(c1)CC(C=O)C2]

### Step 1: 2-(dimethylamino)-N-[7-fluoro-2-(hydroxymethyl)indan-5-yl]-2-methyl-propanamide

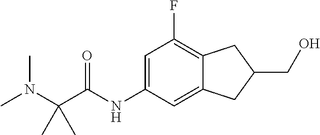 [SMILES: CN(C)C(C)(C)C(=O)Nc1cc(F)c2c(c1)CC(CO)C2]

A mixture of 2-(dimethylamino)-2-methyl-propanoic acid; hydrochloride [CAS #97874-26-5] (233.33 mg, 1.4 mmol, 1.01 eq) in DCM (7 mL) was cooled to −15° C. 4-Dimethylaminopyridine (505.63 mg, 4.14 mmol, 3 eq) was added followed by T3P (1053.52 mg, 1.66 mmol, 1.2 eq) dropwise. The mixture was stirred at −15° C. for 1 h then (6-amino-4-fluoro-indan-2-yl)methanol (Intermediate 5, 250.0 mg, 1.38 mmol, 1 eq) was added. The mixture was stirred at −15° C. for 1 h then warmed to RT over 2 h. DCM and NaHCO₃ (sat. aq.) were added, the phases were separated, the aqueous phase was extracted with DCM (×3). The combined organic phases were dried over Na₂SO₄, filtered and concentrated under reduced pressure. The obtained crude was purified by flash column chromatography (0-10% MeOH in DCM) to give the title compound (122 mg, 0.410 mmol, 30.04% yield) as a colorless oil. MS (ESP) m/z=295.1 [M+H]⁺.

### Step 2: 2-(dimethylamino)-N-(7-fluoro-2-formyl-indan-5-yl)-2-methyl-propanamide

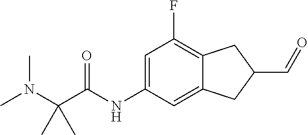 [SMILES: CN(C)C(C)(C)C(=O)Nc1cc(F)c2c(c1)CC(C=O)C2]

The title compound was prepared by analogy to Intermediate 7, Step 2 and was obtained as a brown oil. MS (ESP) m/z=293.1 [M+H]⁺.

### Intermediate 10

### (2R)-2-(Dimethylamino)-N-(7-fluoro-2-formyl-indan-5-yl)propanamide

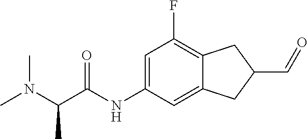 [SMILES: C[C@H](C(=O)Nc1cc(F)c2c(c1)CC(C=O)C2)N(C)C]

The title compound was prepared by analogy to Intermediate 7 using (2R)-2-(dimethylamino)propanoic acid [CAS #157431-09-9] in place of N,N-dimethylglycine and was obtained as a brown oil. MS (ESP) m/z=279.1 [M+H]⁺.

### Intermediate 11

### N-(7-Fluoro-2-formyl-indan-5-yl)-2-morpholino-acetamide

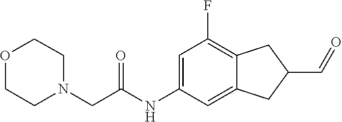 [SMILES: O=CC1Cc2cc(NC(=O)CN3CCOCC3)cc(F)c2C1]

### Step 1:

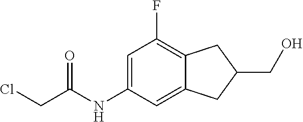 [SMILES: O=C(CCl)Nc1cc(F)c2c(c1)CC(CO)C2]

A mixture of (6-amino-4-fluoro-indan-2-yl)methanol (Intermediate 5, 100.0 mg, 0.550 mmol, 1 eq) in DCM (2.5 mL) was cooled to 0° C. then triethylamine (0.15 mL, 1.1 mmol, 2 eq) was added. A solution of chloroacetyl chloride (0.04 mL, 0.550 mmol, 1 eq) in DCM (2.5 mL) was added dropwise. The mixture was left stirring at 0° C. for 2 h. Water and further DCM were added, the phases were separated, the aqueous phase was extracted with DCM. The combined organic phases were dried over Na₂SO₄, filtered and concentrated under reduced pressure to give the title compound (174 mg, 0.680 mmol, quant. yield) as a brown oil. MS (ESP) m/z=258.1 [M+H]⁺.

### Step 2: N-[7-fluoro-2-(hydroxymethyl)indan-5-yl]-2-morpholino-acetamide

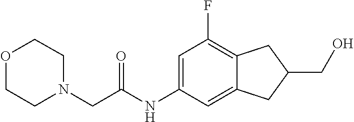 [SMILES: O=C(CN1CCOCC1)Nc1cc(F)c2c(c1)CC(CO)C2]

A mixture of 2-chloro-N-[7-fluoro-2-(hydroxymethyl)indan-5-yl]acetamide (177.16 mg, 0.550 mmol, 1 eq), N,N-diisopropylethylamine (0.19 mL, 1.1 mmol, 2 eq) and morpholine (0.14 mL, 1.65 mmol, 3 eq) in THF (5 mL) was stirred at 60° C. for 5 h. The mixture was warmed to RT, then EtOAc and H₂O were added. The phases were separated, the aqueous phase was extracted with EtOAc (×2). The combined organic phases were dried over Na₂SO₄, filtered and concentrated. The obtained crude was purified by flash column chromatography (0-100% MeCN in EtOAc) to give the title compound (27 mg, 0.090 mmol, 15.9% yield). MS (ESP) m/z=309.2 [M+H]⁺.

### Step 3: N-(7-fluoro-2-formyl-indan-5-yl)-2-morpholino-acetamide

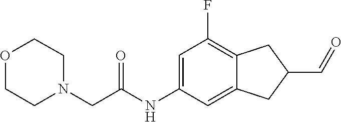 [SMILES: O=CC1Cc2cc(NC(=O)CN3CCOCC3)cc(F)c2C1]

The title compound was prepared by analogy to Intermediate 7, Step 2. MS (ESP) m/z=307.2 [M+H]⁺.

### Intermediate 12

### 2-(Azetidin-1-yl)-N-(7-fluoro-2-formyl-indan-5-yl)acetamide

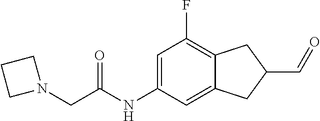 [SMILES: O=CC1Cc2cc(NC(=O)CN3CCC3)cc(F)c2C1]

### Step 1: 2-(azetidin-1-yl)-N-[7-fluoro-2-(hydroxymethyl)indan-5-yl]acetamide

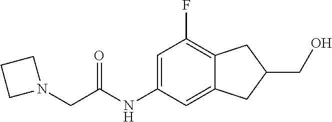 [SMILES: O=C(CN1CCC1)Nc1cc(F)c2c(c1)CC(CO)C2]

A mixture of (6-amino-4-fluoro-indan-2-yl)methanol (Intermediate 5, 80.0 mg, 0.440 mmol, 1 eq) in THF (4 mL) was cooled to 0° C. then N,N-diisopropylethylamine (0.15 mL, 0.880 mmol, 2 eq) and chloroacetyl chloride (0.04 mL, 0.440 mmol, 1 eq) in solution in THF (1 mL) was added dropwise. The mixture was stirred at 0° C. for 30 min. Further N,N-diisopropylethylamine (0.15 mL, 0.880 mmol, 2 eq) was added followed by azetidine (0.15 mL, 2.21 mmol, 5 eq). The mixture was heated to 50° C. and stirred at this temperature for 4 h. Water and EtOAc were added, the phases were separated. The organic phase was dried over Na₂SO₄, filtered and concentrated under reduced pressure. The obtained crude was purified by flash column chromatography (0-10% MeOH in DCM) to give the title compound (63 mg, 0.230 mmol, 51.27% yield) as a yellow oil. MS (ESP) m/z=279.1 [M+H]⁺.

### Step 2: 2-(azetidin-1-yl)-N-(7-fluoro-2-formyl-indan-5-yl)acetamide

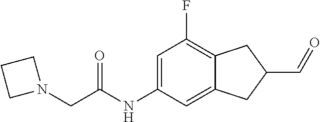 [SMILES: O=CC1Cc2cc(NC(=O)CN3CCC3)cc(F)c2C1]

The title compound was prepared by analogy to Intermediate 7, Step 2 and was obtained as a yellow foam. MS (ESP) m/z=277.1 [M+H]⁺.

### Intermediate 13

### (2S)-2-(Dimethylamino)-N-(7-fluoro-2-formyl-indan-5-yl)propanamide

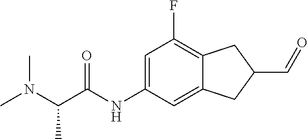 [SMILES: C[C@@H](C(=O)Nc1cc(F)c2c(c1)CC(C=O)C2)N(C)C]

The title compound was prepared by analogy to Intermediate 7 using (2S)-2-(dimethylamino)propanoic acid [CAS #2812-31-9] in place of N,N-dimethylglycine. MS (ESP) m/z=279.2 [M+H]⁺.

### Intermediate 14

### (2S)—N-(7-Fluoro-2-formyl-indan-5-yl)-1-methyl-pyrrolidine-2-carboxamide

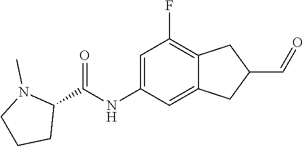 [SMILES: CN1CCC[C@H]1C(=O)Nc1cc(F)c2c(c1)CC(C=O)C2]

The title compound was prepared by analogy to Intermediate 7 using (2S)-1-methylpyrrolidine-2-carboxylic acid; hydrate [CAS #199917-42-5] in place of N,N-dimethylglycine and was obtained as a colorless oil. MS (ESP) m/z=291.2 [M+H]⁺.

### Intermediate 15

### N-(7-Fluoro-2-formyl-indan-5-yl)-2-[methyl(oxetan-3-yl)amino]acetamide

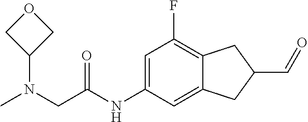 [SMILES: CN(CC(=O)Nc1cc(F)c2c(c1)CC(C=O)C2)C1COC1]

The title compound was prepared by analogy to Intermediate 12 using N-methyl-3-aminooxetane [CAS #952182-03-5] in place of azetidine and was obtained as a white foam. MS (ESP) m/z=307.5 [M+H]⁺.

### Intermediate 16

### 2-(3-Azabicyclo[3.1.0]hexan-3-yl)-N-(7-fluoro-2-formyl-indan-5-yl)acetamide

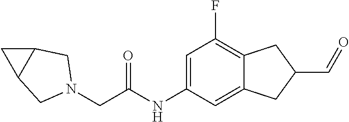 [SMILES: O=CC1Cc2cc(NC(=O)CN3CC4CC4C3)cc(F)c2C1]

The title compound was prepared by analogy to Intermediate 12 using 3-azabicyclo[3.1.0]hexane, hydrochloride [CAS #73799-64-1] in place of azetidine and was obtained as a yellow oil. MS (ESP) m/z=303.3 [M+H]⁺.

### Intermediate 17

### 2-[Cyclopropyl(methyl)amino]-N-(7-fluoro-2-formyl-indan-5-yl)acetamide

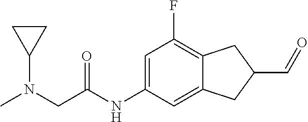 [SMILES: CN(CC(=O)Nc1cc(F)c2c(c1)CC(C=O)C2)C1CC1]

The title compound was prepared by analogy to Intermediate 12 using N-methylcyclopropanamine [CAS #5163-20-2] in place of azetidine and was obtained as a white solid. MS (ESP) m/z=291.4 [M+H]⁺.

### Intermediate 18

### (2R)—N-(7-Fluoro-2-formyl-indan-5-yl)-1-methyl-pyrrolidine-2-carboxamide

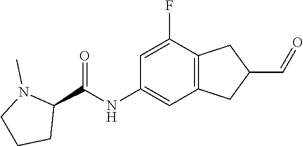 [SMILES: CN1CCC[C@@H]1C(=O)Nc1cc(F)c2c(c1)CC(C=O)C2]

The title compound was prepared by analogy to Intermediate 7 using (2R)-1-methylpyrrolidine-2-carboxylic acid [CAS #58123-62-9] in place of N,N-dimethylglycine and was obtained as a pale yellow semisolid. MS (ESP) m/z=291.3 [M+H]⁺.

### Intermediate 19

### N-(7-Fluoro-2-formyl-indan-5-yl)-2-methyl-oxazole-5-carboxamide

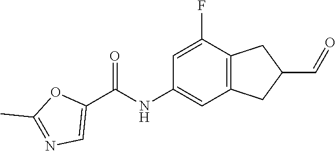 [SMILES: Cc1ncc(C(=O)Nc2cc(F)c3c(c2)CC(C=O)C3)o1]

The title compound was prepared by analogy to Intermediate 7 using 2-methyl-oxazole-5-carboxylic acid [CAS #1216012-87-1] in place of N,N-dimethylglycine and was obtained as a yellow oil. MS (ESP) m/z=289.1 [M+H]⁺.

### Intermediate 20

### 2-(Azetidin-1-yl)-N-(7-fluoro-2-formyl-indan-5-yl)propanamide

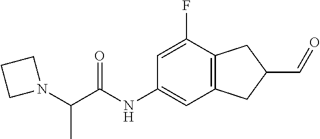 [SMILES: CC(C(=O)Nc1cc(F)c2c(c1)CC(C=O)C2)N1CCC1]

The title compound was prepared by analogy to Intermediate 7 using 2-(azetidin-1-yl)propanoic acid; hydrochloride [CAS #2219370-85-9] in place of N,N-dimethylglycine and was obtained as a yellow oil. MS (ESP) m/z=291.3 [M+H]⁺.

### Intermediate 21

### (2S,4R)—N-(7-Fluoro-2-formyl-indan-5-yl)-4-methoxy-1-methyl-pyrrolidine-2-carboxamide

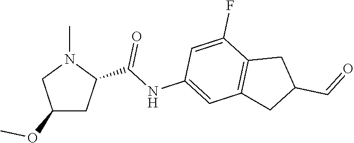 [SMILES: CO[C@@H]1C[C@@H](C(=O)Nc2cc(F)c3c(c2)CC(C=O)C3)N(C)C1]

### Step 1: tert-butyl (2S,4R)-2-[[7-fluoro-2-(hydroxymethyl)indan-5-yl]carbamoyl]-4-methoxy-pyrrolidine-1-carboxylate

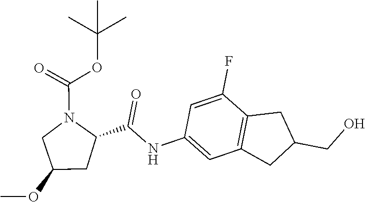 [SMILES: CO[C@@H]1C[C@@H](C(=O)Nc2cc(F)c3c(c2)CC(CO)C3)N(C(=O)OC(C)(C)C)C1]

The title compound was prepared by analogy to Intermediate 7, Step 1 using (2S,4R)-1-tert-butoxycarbonyl-4-methoxy-pyrrolidine-2-carboxylic acid [CAS #83624-01-5] in place of N,N-dimethylglycine and was obtained as a colorless oil. (ESP) m/z=409.3 [M+H]⁺.

### Step 2: (2S,4R)—N-[7-fluoro-2-(hydroxymethyl)indan-5-yl]-4-methoxy-pyrrolidine-2-carboxamide

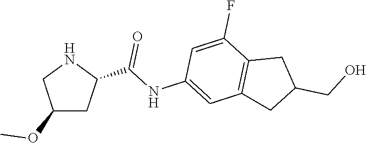 [SMILES: CO[C@H]1CN[C@H](C(=O)Nc2cc(F)c3c(c2)CC(CO)C3)C1]

A mixture of tert-butyl (2S,4R)-2-[[7-fluoro-2-(hydroxymethyl)indan-5-yl]carbamoyl]-4-methoxy-pyrrolidine-1-carboxylate (198.0 mg, 0.480 mmol, 1 eq) and trifluoroacetic acid (0.37 mL, 4.85 mmol, 10 eq) in DCM (5 mL) was stirred at RT overnight. The mixture was concentrated and purified by flash column chromatography (1-50% MeCN in H₂O+0.1% NH₃) to give the title compound (81 mg, 0.260 mmol, 54.19% yield) as a viscous oil. MS (ESP) m/z=309.2 [M+H]⁺.

### Step 3: (2S,4R)—N-[7-fluoro-2-(hydroxymethyl)indan-5-yl]-4-methoxy-1-methyl-pyrrolidine-2-carboxamide

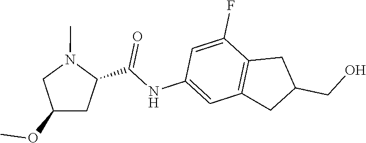 [SMILES: CO[C@@H]1C[C@@H](C(=O)Nc2cc(F)c3c(c2)CC(CO)C3)N(C)C1]

A mixture of (2S,4R)—N-[7-fluoro-2-(hydroxymethyl)indan-5-yl]-4-methoxy-pyrrolidine-2-carboxamide (81.0 mg, 0.260 mmol, 1 eq) and formaldehyde (25.43 uL, 0.340 mmol, 1.3 eq) in tetrahydrofuran (5 mL) was stirred at RT for 5 min. then sodium triacetoxyborohydride (16.7 mg, 0.080 mmol, 0.300 eq) was added. The mixture was left stirring overnight at 20° C. It was diluted with EtOAc and washed with NaHCO₃ (sat.aq.). The aqueous layer was extracted with EtOAc (×3). The combined organic phases were dried over Na₂SO₄, filtered and concentrated. The obtained crude was purified by flash column chromatography (1-10% MeOH in DCM) to give the title compound (69 mg, 0.210 mmol, 81.48% yield) as a colorless oil. MS (ESP) m/z=323.4 [M+H]⁺.

### Step 4: (2S,4R)—N-(7-fluoro-2-formyl-indan-5-yl)-4-methoxy-1-methyl-pyrrolidine-2-carboxamide

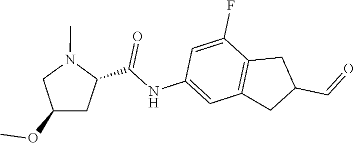 [SMILES: CO[C@@H]1C[C@@H](C(=O)Nc2cc(F)c3c(c2)CC(C=O)C3)N(C)C1]

The title compound was prepared by analogy to Intermediate 7, Step 2 and was obtained as a pink viscous oil. MS (ESP) m/z=321.4 [M+H]⁺.

### Intermediate 22

### N-(7-Fluoro-2-formyl-indan-5-yl)-2-hydroxy-2-methyl-propanamide

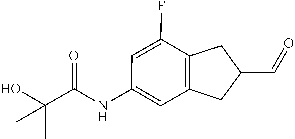 [SMILES: CC(C)(O)C(=O)Nc1cc(F)c2c(c1)CC(C=O)C2]

### Step 1: ethyl 4-fluoro-6-[(2-hydroxy-2-methyl-propanoyl)amino]indane-2-carboxylate

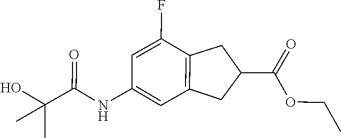 [SMILES: CCOC(=O)C1Cc2cc(NC(=O)C(C)(C)O)cc(F)c2C1]

Thionyl chloride (45.74 uL, 0.630 mmol, 1.4 eq) was added to a solution of 2-hydroxyisobutyric acid (65.28 mg, 0.630 mmol, 1.4 eq) in DMA (1 mL) at −10° C. After stirring for 30 min., ethyl 6-amino-4-fluoro-indane-2-carboxylate (Intermediate 5, Step 7, 100.0 mg, 0.450 mmol, 1 eq) in DMA was added and the mixture was stirred at RT for 2 h. The mixture was taken up in EtOAc and the organics were washed with water, 1M HCl and NaHCO₃ solution (sat. aq.). The organics were dried (Na₂SO₄) and concentrated to dryness to afford the title compound (134 mg, 0.430 mmol, 96.71% yield) as a light yellow oil. MS (ESP) m/z=268.2 [M+H]⁺.

### Step 2: N-[7-fluoro-2-(hydroxymethyl)indan-5-yl]-2-hydroxy-2-methyl-propanamide

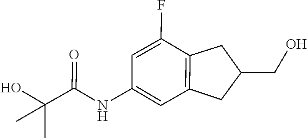 [SMILES: CC(C)(O)C(=O)Nc1cc(F)c2c(c1)CC(CO)C2]

To a stirred solution of ethyl 4-fluoro-6-[(2-hydroxy-2-methyl-propanoyl)amino]indane-2-carboxylate (135.0 mg, 0.440 mmol, 1 eq) in dry THF (3.64 mL) and ethanol (1.82 mL), calcium chloride (96.87 mg, 0.870 mmol, 2 eq) and sodium borohydride (49.53 mg, 1.31 mmol, 3 eq) were added and the mixture was stirred at RT for 18 h. The mixture was cooled to 0° C., carefully quenched with NH₄Cl (sat. aq.) and extracted with EtOAc (×3). The organic phase was dried over Na₂SO₄ and concentrated in vacuo. The crude was purified by silica cartridge chromatography (5 g, 50-80% EtOAc in cyclohexane) to afford the title compound (90 mg, 0.340 mmol, 77.15% yield) as a yellow oil. MS (ESP) m/z=310.2 [M+H]⁺.

### Step 3: N-(7-fluoro-2-formyl-indan-5-yl)-2-hydroxy-2-methyl-propanamide

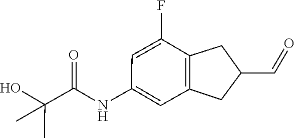 [SMILES: CC(C)(O)C(=O)Nc1cc(F)c2c(c1)CC(C=O)C2]

The title compound was prepared by analogy to Intermediate 7, Step 2 and was obtained as a brown oil. MS (ESP) m/z=266.3 [M+H]⁺.

### Intermediate 23

### (3R)-3-(Dimethylamino)-N-(7-fluoro-2-formyl-indan-5-yl)tetrahydrofuran-3-carboxamide

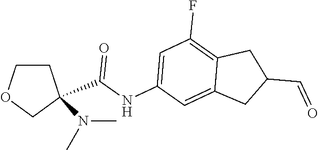 [SMILES: CN(C)[C@]1(C(=O)Nc2cc(F)c3c(c2)CC(C=O)C3)CCOC1]

### Step 1: (3R)-3-(tert-butoxycarbonylamino)tetrahydrofuran-3-carboxylic Acid

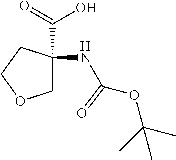 [SMILES: CC(C)(C)OC(=O)N[C@]1(C(=O)O)CCOC1]

To a solution of (3R)-3-aminotetrahydrofuran-3-carboxylic acid [CAS #1315053-78-1] (200.0 mg, 1.53 mmol, 1 eq) in water (3 mL) and 1,4-dioxane (3 mL), sodium hydroxide (1.6 mL, 1.6 mmol, 1.05 eq) (1M solution) was added. The mixture was cooled to 0° C. and di-tert-butyldicarbonate (366.16 mg, 1.68 mmol, 1.1 eq) was added and the mixture was allowed to warm to RT and stirred overnight. The reaction mixture was acidified to pH 2.0 with 0.1 M HCl, extracted with EtOAc, dried (Na₂SO₄), filtered and concentrated under reduced pressure to obtain the title compound (195 mg, 0.840 mmol, 55.29% yield). MS (ESP) m/z=232.1 [M+H]⁺.

### Steps 2 and 3: ethyl 6-[[(3R)-3-aminotetrahydrofuran-3-carbonyl]amino]-4-fluoro-indane-2-carboxylate

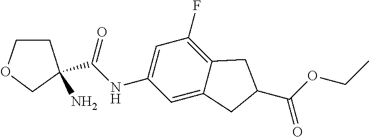 [SMILES: CCOC(=O)C1Cc2cc(NC(=O)[C@@]3(N)CCOC3)cc(F)c2C1]

The title compound was prepared by analogy to Intermediate 21, Steps 1 and 2 using (3R)-3-(tert-butoxycarbonylamino)tetrahydrofuran-3-carboxylic acid in place of (2S,4R)-1-tert-butoxycarbonyl-4-methoxy-pyrrolidine-2-carboxylic acid and was obtained as a light yellow oil. MS (ESP) m/z=337.7 [M+H]⁺.

### Step 4: ethyl 6-[[(3R)-3-(dimethylamino)tetrahydrofuran-3-carbonyl]amino]-4-fluoro-indane-2-carboxylate

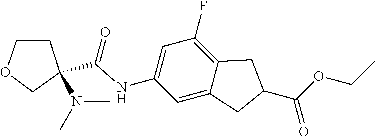 [SMILES: CCOC(=O)C1Cc2cc(NC(=O)[C@@]3(N(C)C)CCOC3)cc(F)c2C1]

To a solution of ethyl 6-[[(3R)-3-aminotetrahydrofuran-3-carbonyl]amino]-4-fluoro-indane-2-carboxylate (77.0 mg, 0.230 mmol, 1 eq) in methanol (3.3 mL) formaldehyde, 37% wt in water (14.51 uL, 0.530 mmol, 2.3 eq) was added followed by acetic acid (0.05 mL, 0.920 mmol, 4 eq) and sodium cyanoborohydride (28.77 mg, 0.460 mmol, 2 eq). The mixture was stirred at RT for 5 h. The mixture was concentrated under reduced pressure, then quenched with water and extracted with EtOAc (2×). The organic phase was dried, filtered and evaporated. The crude was purified by silica cartridge chromatography (5 g, 0-40% EtOAc in cyclohexane) to afford the title compound (77 mg, 0.210 mmol, 92.3% yield) as a colorless oil. MS (ESP) m/z=365.2 [M+H]⁺.

### Steps 5 and 6: (3R)-3-(dimethylamino)-N-(7-fluoro-2-formyl-indan-5-yl)tetrahydrofuran-3-carboxamide

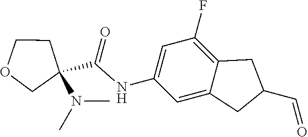 [SMILES: CN(C)[C@]1(C(=O)Nc2cc(F)c3c(c2)CC(C=O)C3)CCOC1]

The title compound was prepared by analogy to Intermediate 22, Steps 2 and 3 and was obtained as a light pink oil. MS (ESP) m/z=321.4 [M+H]⁺.

### Intermediate 24

### N-(7-Cyano-2-formyl-indan-5-yl)-2-hydroxy-2-methyl-propanamide

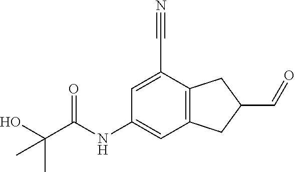 [SMILES: CC(C)(O)C(=O)Nc1cc(C#N)c2c(c1)CC(C=O)C2]

### Step 1: ethyl 4-cyanoindane-2-carboxylate

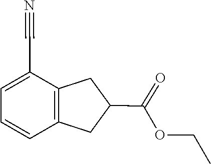 [SMILES: CCOC(=O)C1Cc2cccc(C#N)c2C1]

Ethyl 4-bromoindane-2-carboxylate [CAS #2091562-14-8] (8.7 g, 32.3 mmol, 1 eq) was dissolved in N,N-dimethylacetamide (12.5 mL). Zinc cyanide (5.31 g, 45.3 mmol, 1.4 eq) and 2-di-tert-butylphosphino-2′,4′,6′-triisopropylbiphenyl (2.75 g, 6.47 mmol, 0.2 eq) were added. Argon was bubbled through the solution, then tris(dibenzylideneacetone)dipalladium (0) (5.92 g, 6.47 mmol, 0.2 eq) was added and the mixture stirred at 80° C. for 2 h. The mixture was poured onto water and extracted with EtOAc (3×), dried over Na₂SO₄ and evaporated totally. The residue was purified by silica cartridge chromatography (70 g, heptane to 5% EtOAc/heptane then 100 g, heptane to 10% MTBE/heptane) to give the title compound (5.99 g, 27.8 mmol, 86.1% yield) as a light yellow oil. MS (ESP) m/z=216.2 [M+H]⁺.

### Step 2: ethyl 4-cyano-6-nitro-indane-2-carboxylate

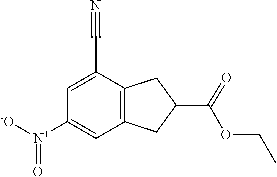

To a stirred solution of ethyl 4-cyanoindane-2-carboxylate (1700.0 mg, 7.9 mmol, 1 eq) in sulfuric acid (24.42 mL, 458.07 mmol, 58 eq) at −7° C., nitric acid (0.5 mL, 7.9 mmol, 1 eq) was added dropwise over 30 min. The mixture was stirred at −7° C. for 1 h. The reaction mixture was quenched by pouring it carefully dropwise onto an ice-cold brine and water mixture (50 mL) and stirred for 15 min. The product was extracted with EtOAc (3×30 mL). The combined organic phase was dried through a phase separator and evaporated under reduced pressure to afford a crude material which was purified by silica cartridge chromatography (200 g, 0-60% EtOAc in cyclohexane) to give the title compound (1216 mg, 4.67 mmol, 54.43% yield) as a yellow waxy solid. MS (ESP) m/z=261.1 [M+H]⁺.

### Step 3: ethyl 6-amino-4-cyano-indane-2-carboxylate

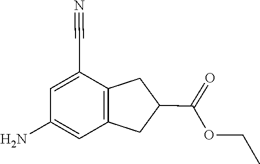 [SMILES: CCOC(=O)C1Cc2cc(N)cc(C#N)c2C1]

A solution of ethyl 4-cyano-6-nitro-indane-2-carboxylate (1060.0 mg, 3.75 mmol, 1 eq) in ethanol (67.88 mL) was stirred under H₂ atmosphere for 75 min. in the presence of Pd/C (174.14 mg, 0.160 mmol, 0.040 eq). The solution was filtered, the palladium was washed with EtOH and the filtrate concentrated under reduced pressure to give the title compound (921 mg, 4 mmol, 97.14% yield) as a yellow oil. MS (ESP) m/z=231.2 [M+H]⁺.

### Steps 4 to 6: N-(7-cyano-2-formyl-indan-5-yl)-2-hydroxy-2-methyl-propanamide

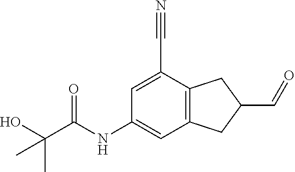 [SMILES: CC(C)(O)C(=O)Nc1cc(C#N)c2c(c1)CC(C=O)C2]

The title compound was prepared by analogy to Intermediate 22 and was obtained as a brown oil. MS (ESP) m/z=273.1 [M+H]⁺.

### Intermediate 25

### (2R)—N-(7-Cyano-2-formyl-indan-5-yl)-2-(dimethylamino)propanamide

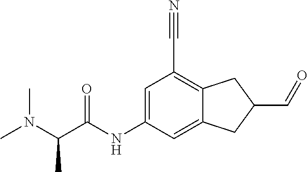 [SMILES: C[C@H](C(=O)Nc1cc(C#N)c2c(c1)CC(C=O)C2)N(C)C]

### Step 1: ethyl 4-cyano-6-[[(2R)-2-(dimethylamino)propanoyl]amino]indane-2-carboxylate

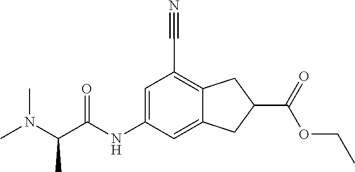 [SMILES: CCOC(=O)C1Cc2cc(NC(=O)[C@@H](C)N(C)C)cc(C#N)c2C1]

The title compound was prepared from ethyl 6-amino-4-cyano-indane-2-carboxylate (Intermediate 24, Step 3) and (2R)-2-(dimethylamino)propanoic acid [CAS #157431-09-9] by analogy to Intermediate 7, Step 1 and was obtained as a pale yellow oil. MS (ESP) m/z=330.3 [M+H]⁺.

### Steps 2 and 3: (2R)—N-(7-cyano-2-formyl-indan-5-yl)-2-(dimethylamino)propanamide

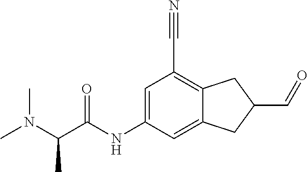 [SMILES: C[C@H](C(=O)Nc1cc(C#N)c2c(c1)CC(C=O)C2)N(C)C]

The title compound was prepared by analogy to Intermediate 22, Steps 2 and 3 and was obtained as a light yellow oil. MS (ESP) m/z=286.2 [M+H]⁺.

### Intermediate 26

### (2R)-2-(Dimethylamino)-N-(4-fluoro-2-formyl-indan-5-yl)propanamide

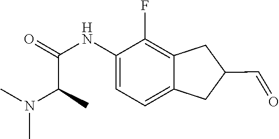 [SMILES: C[C@H](C(=O)Nc1ccc2c(c1F)CC(C=O)C2)N(C)C]

The title compound was prepared by analogy to Intermediate 7 using (5-amino-4-fluoro-indan-2-yl)methanol in place of (6-amino-4-fluoro-indan-2-yl)methanol and (2R)-2-(dimethylamino)propanoic acid [CAS #157431-09-9] in place of N,N-dimethylglycine. MS (ESP) m/z=279.5 [M+H]⁺.

The (5-amino-4-fluoro-indan-2-yl)methanol used as starting material was prepared from ethyl 4-fluoro-5-hydroxy-indane-2-carboxylate (Intermediate 5, Step 4) by analogy to Intermediate 5, Steps 5 to 8 and was obtained as a light yellow solid. MS (ESP) m/z=182.1 [M+H]⁺.

### Intermediate 27

### 2-(Dimethylamino)-N-(6-formyl-4-methyl-6,7-dihydro-5H-cyclopenta[b]pyridin-2-yl)acetamide

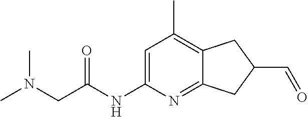 [SMILES: Cc1cc(NC(=O)CN(C)C)nc2c1CC(C=O)C2]

### Step 1: N-[6-[[tert-butyl(dimethyl)silyl]oxymethyl]-4-methyl-6,7-dihydro-5H-cyclopenta[b]pyridin-2-yl]-2-(dimethylamino)acetamide

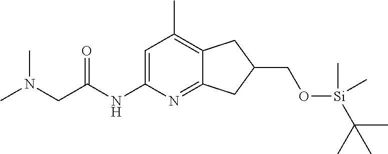 [SMILES: Cc1cc(NC(=O)CN(C)C)nc2c1CC(CO[Si](C)(C)C(C)(C)C)C2]

To a mixture of 6-[[tert-butyl(dimethyl)silyl]oxymethyl]-4-methyl-6,7-dihydro-5H-cyclopenta[b]pyridin-2-amine (Intermediate 6, 22.0 mg, 0.080 mmol, 1 eq) and N,N-dimethylglycine (19.39 mg, 0.190 mmol, 2.5 eq) in MeCN (0.752 mL) was added 1-methylimidazole (0.04 mL, 0.530 mmol, 7 eq) and TCFH (52.76 mg, 0.190 mmol, 2.5 eq) at 0° C. The resulting mixture was stirred at 20° C. for 1 h. Then a saturated aqueous solution of NH₄Cl was added and the mixture extracted with EtOAc (3×10 mL). Organic layers were collected, dried over Na₂SO₄, filtered and concentrated under vacuum to give the crude product that was purified by flash column chromatography (25 g, DCM/MeOH from 100:0 to 90:10) to give the title compound (23 mg, 0.060 mmol, 80.98% yield) as a colorless oil. MS (ESP) m/z=378.4 [M+H]⁺.

### Step 2: 2-(dimethylamino)-N-[6-(hydroxymethyl)-4-methyl-6,7-dihydro-5H-cyclopenta[b]pyridin-2-yl]acetamide

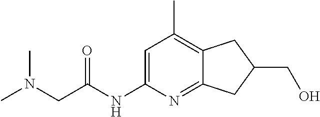 [SMILES: Cc1cc(NC(=O)CN(C)C)nc2c1CC(CO)C2]

To a stirred solution of N-[6-[[tert-butyl(dimethyl)silyl]oxymethyl]-4-methyl-6,7-dihydro-5H-cyclopenta[b]pyridin-2-yl]-2-(dimethylamino)acetamide (37.0 mg, 0.100 mmol, 1 eq) in methanol (0.980 mL) was added HCl (0.13 mL, 0.760 mmol, 7.75 eq). The mixture was stirred at 25° C. for 3 h. Then the mixture was concentrated under vacuum and purified by SCX chromatography to give the title compound (15.5 mg, 0.060 mmol, 60.07% yield) as a light yellow solid. MS (ESP) m/z=264.4 [M+H]⁺.

### Step 3: 2-(dimethylamino)-N-(6-formyl-4-methyl-6,7-dihydro-5H-cyclopenta[b]pyridin-2-yl)acetamide

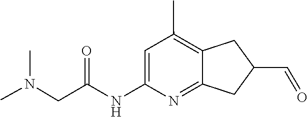 [SMILES: Cc1cc(NC(=O)CN(C)C)nc2c1CC(C=O)C2]

The title compound was prepared by analogy to Intermediate 7, Step 2 and was obtained as a light brown oil. It was used for the next step without purification. MS (ESP) m/z=262.4 [M+H]⁺.

### Intermediate 28

### (2R)-2-(Dimethylamino)-N-(6-formyl-4-methyl-6,7-dihydro-5H-cyclopenta[b]pyridin-2-yl)propanamide

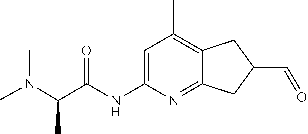 [SMILES: Cc1cc(NC(=O)[C@@H](C)N(C)C)nc2c1CC(C=O)C2]

The title compound was prepared by analogy to Intermediate 27 using (2R)-2-(dimethylamino)propanoic acid in place of N,N-dimethylglycine and was obtained as a brown oil. It was used for the next step without purification. MS (ESP) m/z=276.2 [M+H]⁺.

### Intermediate 29

### 2-(Azetidin-1-yl)-N-(6-formyl-4-methyl-6,7-dihydro-5H-cyclopenta[b]pyridin-2-yl)acetamide

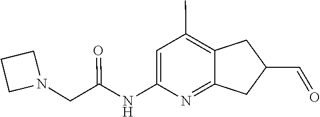 [SMILES: Cc1cc(NC(=O)CN2CCC2)nc2c1CC(C=O)C2]

### Step 1: 2-(azetidin-1-yl)-N-[6-[[tert-butyl(dimethyl)silyl]oxymethyl]-4-methyl-6,7-dihydro-5H-cyclopenta[b]pyridin-2-yl]acetamide

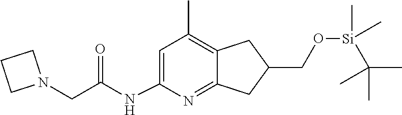 [SMILES: Cc1cc(NC(=O)CN2CCC2)nc2c1CC(CO[Si](C)(C)C(C)(C)C)C2]

The title compound was prepared from 6-[[tert-butyl(dimethyl)silyl]oxymethyl]-4-methyl-6,7-dihydro-5H-cyclopenta[b]pyridin-2-amine (Intermediate 6) by analogy to Intermediate 12, Step 1 and was obtained as colorless oil. MS (ESP) m/z=390.4 [M+H]⁺.

### Steps 2 and 3: 2-(azetidin-1-yl)-N-(6-formyl-4-methyl-6,7-dihydro-5H-cyclopenta[b]pyridin-2-yl)acetamide

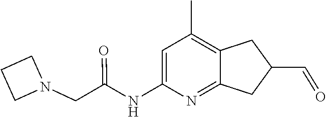 [SMILES: Cc1cc(NC(=O)CN2CCC2)nc2c1CC(C=O)C2]

The title compound was prepared by analogy to Intermediate 27, Steps 2 and 3 and was obtained as a dark brown oil. It was used for the next step without purification. MS (ESP) m/z=274.4 [M+H]⁺.

### Intermediate 30

### (2S)—N-(6-Formyl-4-methyl-6,7-dihydro-5H-cyclopenta[b]pyridin-2-yl)-1-methyl-pyrrolidine-2-carboxamide

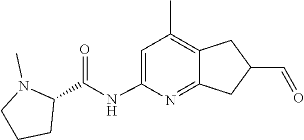 [SMILES: Cc1cc(NC(=O)[C@@H]2CCCN2C)nc2c1CC(C=O)C2]

The title compound was prepared by analogy to Intermediate 27 using (2S)-1-methylpyrrolidine-2-carboxylic acid; hydrate [CAS #199917-42-5] in place of N,N-dimethylglycine and was obtained as a dark brown foam. It was used for the next step without purification. MS (ESP) m/z=288.4 [M+H]⁺.

### Intermediate 31

### (2R)-2-(Dimethylamino)-N-(6-formyl-1-methyl-6,7-dihydro-5H-cyclopenta[c]pyridin-3-yl)propanamide

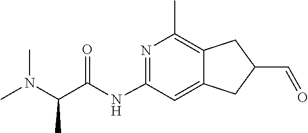 [SMILES: Cc1nc(NC(=O)[C@@H](C)N(C)C)cc2c1CC(C=O)C2]

### Steps 1 to 6: tert-butyl-[(1-chloro-6,7-dihydro-5H-cyclopenta[c]pyridin-6-yl)methoxy]-dimethyl-silane

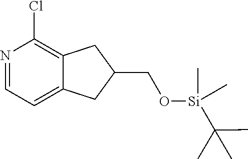 [SMILES: CC(C)(C)[Si](C)(C)OCC1Cc2ccnc(Cl)c2C1]

The title compound was prepared by analogy to Intermediate 6, Steps 3 to 8 using [2-chloro-3-(hydroxymethyl)-4-pyridyl]methanol [CAS #1454285-52-9] in place of [2-(hydroxymethyl)-4-methyl-3-pyridyl]methanol and was obtained as a colorless oil.

### Step 7: tert-butyl-dimethyl-[(1-methyl-6,7-dihydro-5H-cyclopenta[c]pyridin-6-yl)methoxy]silane

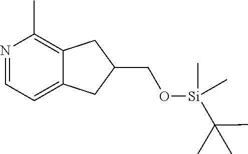 [SMILES: Cc1nccc2c1CC(CO[Si](C)(C)C(C)(C)C)C2]

In a sealed tube, a suspension of tert-butyl-[(1-chloro-6,7-dihydro-5H-cyclopenta[c]pyridin-6-yl)methoxy]-dimethyl-silane (431.0 mg, 1.45 mmol, 1 eq), trimethylboroxine (0.3 mL, 2.17 mmol, 1.5 eq), [1,1′-bis(diphenylphosphino)ferrocene]dichloropalladium(II) (105.86 mg, 0.140 mmol, 0.100 eq) and potassium carbonate (599.87 mg, 4.34 mmol, 3 eq) in 1,4-dioxane (7.23 mL) was degassed by purging the system with three vacuum/nitrogen atmosphere then the mixture was stirred at 100° C. for 1.5 h. The reaction mixture was cooled to r.t. and partitioned between EtOAc and water. The phases were separated and the aqueous layer was extracted with EtOAc (×3). The combined organic phases were filtered through a phase separator and volatiles were removed under reduced pressure to afford the crude compound which was purified by silica cartridge chromatography (25 g, from cyclohexane 100% to cyclohexane/EtOAc 7:3) to give the title compound (337 mg, 1.21 mmol, 83.94% yield) as a light yellow oil. MS (ESP) m/z=278.2 [M+H]⁺.

### Steps 8 to 12: 6-[[tert-butyl(dimethyl)silyl]oxymethyl]-1-methyl-6,7-dihydro-5H-cyclopenta[c]pyridin-3-amine

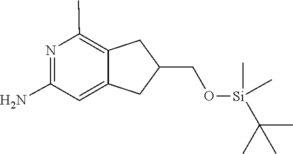 [SMILES: Cc1nc(N)cc2c1CC(CO[Si](C)(C)C(C)(C)C)C2]

The title compound was prepared by analogy to Intermediate 6, Steps 8 to 12 and was obtained as a light yellow solid. MS (ESP) m/z=293.4 [M+H]⁺.

### Steps 12 to 14: (2R)-2-(dimethylamino)-N-(6-formyl-1-methyl-6,7-dihydro-5H-cyclopenta[c]pyridin-3-yl)propanamide

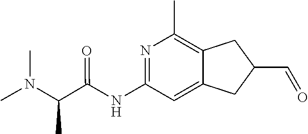 [SMILES: Cc1nc(NC(=O)[C@@H](C)N(C)C)cc2c1CC(C=O)C2]

The title compound was prepared by analogy to Intermediate 28 and was obtained as a light brown viscous oil that was used in the next step without further purification. MS (ESP) m/z=276.2 [M+H]⁺.

### Intermediate 32

### (2R)—N-(4,7-Difluoro-2-formyl-2-hydroxy-indan-5-yl)-2-(dimethylamino)propanamide

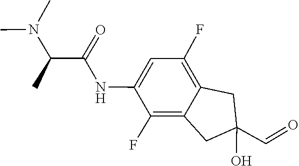 [SMILES: C[C@H](C(=O)Nc1cc(F)c2c(c1F)CC(O)(C=O)C2)N(C)C]

### Step 1: methyl 4,7-difluoro-1-oxo-indane-2-carboxylate

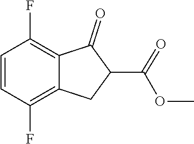 [SMILES: COC(=O)C1Cc2c(F)ccc(F)c2C1=O]

The title compound was prepared by analogy to Intermediate 5, Step 1 using 4,7-difluoroindan-1-one [CAS #130408-16-1] in place of 4-fluoroindan-1-one and dimethyl carbonate in place of diethyl carbonate and was obtained as an orange solid. MS (ESP) m/z=227.0 [M+H]⁺.

### Step 2: methyl 4,7-difluoro-2-hydroxy-1-oxo-indane-2-carboxylate

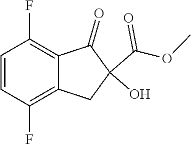 [SMILES: COC(=O)C1(O)Cc2c(F)ccc(F)c2C1=O]

To a solution of 3-chloroperoxybenzoic acid (8545.34 mg, 49.52 mmol, 1.6 eq) in DCM (140.22 mL), cooled to 0° C., a solution of methyl 4,7-difluoro-1-oxo-indane-2-carboxylate (7.0 g, 30.95 mmol, 1 eq) in DCM (109.37 mL) was added. The mixture was stirred for 30 min. After completion of the reaction, aqueous sodium thiosulfate solution and aqueous sodium bicarbonatesulfate were added and the mixture extracted twice with DCM. The organic layer was washed with sodium chloride solution (sat. aq.), dried over anhydrous sodium sulfate, and concentrated under reduced pressure. The residue was purified by silica cartridge chromatography (100 g, cyclohexane/ethyl acetate=98:2 to 70:30) to give the title compound (5300 mg, 21.88 mmol, 70.71% yield) as a yellow solid. MS (ESP) m/z=243.0 [M+H]⁺.

### Step 3: methyl 4,7-difluoro-1,2-dihydroxy-indane-2-carboxylate

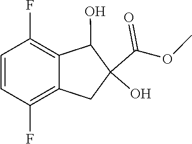 [SMILES: COC(=O)C1(O)Cc2c(F)ccc(F)c2C1O]

A solution of methyl 4,7-difluoro-2-hydroxy-1-oxo-indane-2-carboxylate (5.83 g, 24.07 mmol, 1 eq) in EtOAc (130 mL) was stirred at 25° C. under hydrogen atmosphere (3 atm.) in the presence of 10% palladium on carbon (5.0 mg, 4.81 mmol, 0.200 eq) for 16 h. The resulting filtrate was concentrated under reduced pressure to afford the title compound (5570 mg, 22.81 mmol, 94.75% yield) as a yellow gum.

### Step 4: methyl 1-chloro-4,7-difluoro-2-hydroxy-indane-2-carboxylate

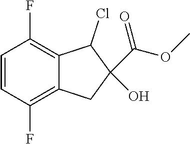 [SMILES: COC(=O)C1(O)Cc2c(F)ccc(F)c2C1Cl]

To a stirred solution of methyl 4,7-difluoro-1,2-dihydroxy-indane-2-carboxylate (1.34 g, 5.49 mmol, 1 eq) in DCM (26 mL), cooled to 0° C., triethylamine (0.92 mL, 6.59 mmol, 1.2 eq) was added followed by dropwise addition of a solution of thionyl chloride (0.4 mL, 5.49 mmol, 1 eq) in DCM (3 mL). The mixture was left stirring at 0° C. for 30 min. The homogeneous mixture was stirred at 5° C. overnight then it was poured into ice-water, extracted with DCM (1×), filtered over a phase separator and concentrated in vacuo. The crude product was purified by silica cartridge chromatography (10 g, 100% cyclohexane to 70:30 cyclohexane/EtOAc) to afford the title compound (652 mg, 2.48 mmol, 45.24% yield) as a colorless oil.

### Step 5: methyl 4,7-difluoro-2-hydroxy-indane-2-carboxylate

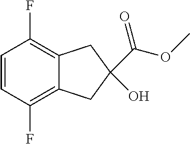 [SMILES: COC(=O)C1(O)Cc2c(F)ccc(F)c2C1]

A solution of methyl 1-chloro-4,7-difluoro-2-hydroxy-indane-2-carboxylate (652.0 mg, 2.48 mmol, 1 eq) in EtOAc (30 mL) was stirred at 25° C. under a hydrogen atmosphere (3 atm.) in the presence of palladium catalyst (1.03 mL, 0.500 mmol, 0.200 eq) (5% palladium on carbon, 50% in water). The resulting filtrate was concentrated under reduced pressure to afford the title compound (460 mg, 2.02 mmol, 81.2% yield) as a colorless oil. MS (ESP) m/z=229.0 [M+H]⁺.

### Step 6: methyl 4,7-difluoro-2-hydroxy-5-nitro-indane-2-carboxylate

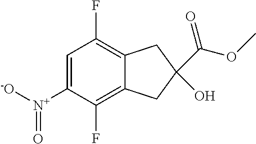

To a solution of ethyl 4,7-difluoro-2-hydroxy-indane-2-carboxylate (392.0 mg, 1.62 mmol, 1 eq) in sulfuric acid (5.0 mL, 93.87 mmol, 58 eq) at 0° C., nitric acid (0.1 mL, 1.62 mmol, 1 eq) was added and the mixture was stirred for 30 minutes at 0° C. The mixture was poured onto water and ice at 0° C. then extracted with EtOAc. The organic phase was washed with brine, dried over Na₂SO₄, filtered and concentrated under vacuum. The resulting crude material was purified by silica cartridge chromatography (10 g, cyclohexane to 70:30 cyclohexane/EtOAc) to obtain the title compound (323 mg, 1.12 mmol, 69.49% yield) as a light yellow liquid. MS (ESP) m/z=274.0 [M+H]⁺.

### Step 7: methyl 5-amino-4,7-difluoro-2-hydroxy-indane-2-carboxylate

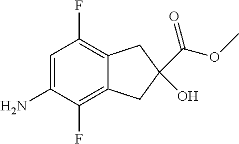 [SMILES: COC(=O)C1(O)Cc2c(F)cc(N)c(F)c2C1]

A mixture of methyl 4,7-difluoro-2-hydroxy-5-nitro-indane-2-carboxylate (323.0 mg, 1.18 mmol, 1 eq) and Pd/C (32.07 mg, 0.030 mmol, 0.030 eq) in methanol (10 mL) was stirred under H₂ atmosphere for 1 h. The solution was filtered, the palladium was washed with EtOH and the filtrate was concentrated under reduced pressure to give the title compound (252 mg, 1.04 mmol, 87.64% yield) as a red oil. MS (ESP) m/z=244.1 [M+H]⁺.

### Step 8: methyl 2-[2-(dimethylamino)propanoyloxy]-4,7-difluoro-5-[[(2R)-2-(dimethylamino)propanoyl]amino]indane-2-carboxylate

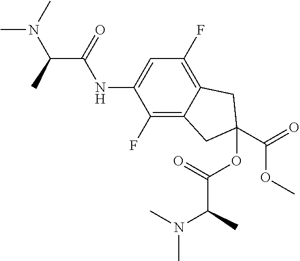

The title compound was prepared by analogy to Intermediate 10, Step 1 and was obtained as a red oil which was employed in the next step without further purification. MS (ESN) m/z=440.3 [M−H]⁻.

### Step 9: (2R)—N-[4,7-difluoro-2-hydroxy-2-(hydroxymethyl)indan-5-yl]-2-(dimethylamino)propanamide

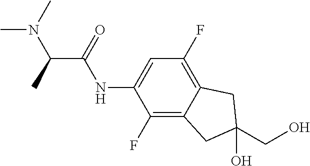 [SMILES: C[C@H](C(=O)Nc1cc(F)c2c(c1F)CC(O)(CO)C2)N(C)C]

Lithium aluminum hydride 2.3 M in Me-THF (1.5 mL, 3.45 mmol, 4.2 eq) was added to a solution of methyl 2-[2-(dimethylamino)propanoyloxy]-4,7-difluoro-5-[[(2R)-2-(dimethylamino)propanoyl]amino]indane-2-carboxylate (363.0 mg, 0.820 mmol, 1 eq) in tetrahydrofuran (16.45 mL), cooled to 0° C. The mixture was stirred for 0.5 h then was quenched at 0° C. with sodium sulfate decahydrate, stirred for 10 min. and then filtered off and concentrated in vacuo. The crude product was purified by reverse phase cartridge chromatography (97:3 to 70:30 aq. NH₄OH 0.1%/MeCN) to afford the title compound (131 mg, 0.420 mmol, 50.69% yield) as an off-white gum. MS (ESP) m/z=316.0 [M+H]⁺.

### Step 10: (2R)—N-(4,7-difluoro-2-formyl-2-hydroxy-indan-5-yl)-2-(dimethylamino)propanamide

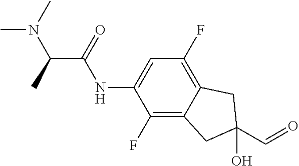 [SMILES: C[C@H](C(=O)Nc1cc(F)c2c(c1F)CC(O)(C=O)C2)N(C)C]

Sulfur trioxide pyridine complex (134.75 mg, 0.850 mmol, 5.12 eq) was added to a solution of (2R)—N-[4,7-difluoro-2-hydroxy-2-(hydroxymethyl)indan-5-yl]-2-(dimethylamino)propanamide (51.97 mg, 0.170 mmol, 1 eq), triethylamine (0.43 mL, 3.08 mmol, 18.6 eq) and dry dimethylsulfoxide (1.23 mL, 17.26 mmol, 104.4 eq) in dry dichloromethane (1 mL), cooled to 0° C., under a nitrogen atmosphere. The resulting mixture was warmed to 25° C. and stirred for 1 h. The reaction was quenched with NaHCO₃ solution (sat. aq.) and extracted with DCM (2×). The collected organic phase was dried with anhydrous Na₂SO₄ and concentrated in vacuo to afford the crude title compound as a colorless liquid. MS (ESP) m/z=331.1 [M+H+H₂O]⁺.

### Intermediate 33

### (2R)—N-(4,7-Difluoro-2-formyl-2-methoxy-indan-5-yl)-2-(dimethylamino)propanamide

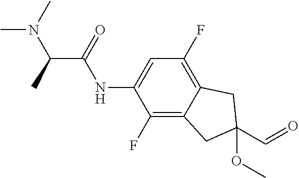 [SMILES: COC1(C=O)Cc2c(F)cc(NC(=O)[C@@H](C)N(C)C)c(F)c2C1]

### Step 1: methyl 4,7-difluoro-2-methoxy-indane-2-carboxylate

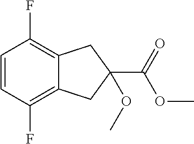 [SMILES: COC(=O)C1(OC)Cc2c(F)ccc(F)c2C1]

To a solution of methyl 4,7-difluoro-2-hydroxy-indane-2-carboxylate (Intermediate 32, Step 5, 200.0 mg, 0.880 mmol, 1 eq) in tetrahydrofuran (3 mL) at 0° C. was added sodium hydride, 60% in oil (42.07 mg, 1.05 mmol, 1.2 eq) followed by iodomethane (0.08 mL, 1.31 mmol, 1.5 eq). The reaction mixture was warmed to 45° C. under N₂ and stirred for 1 h. The mixture was cooled to room temperature and NH₄Cl solution (sat. aq.) was added. EtOAc was added and the organic layer was separated, washed with water and brine then dried over Na₂SO₄. Volatiles were removed to give a crude that was purified by silica-gel chromatography (from 100% cyclohexane to 50:50 cyclohexane/EtOAc) to afford the title compound (106 mg, 0.440 mmol, 49.93% yield). MS (ESP) m/z=243.1 [M+H]⁺.

### Steps 2 to 6: (2R)—N-(4,7-difluoro-2-formyl-2-methoxy-indan-5-yl)-2-(dimethylamino)propanamide

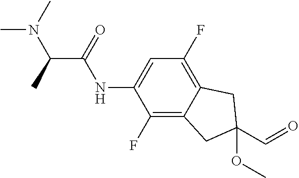 [SMILES: COC1(C=O)Cc2c(F)cc(NC(=O)[C@@H](C)N(C)C)c(F)c2C1]

The title compound was prepared by analogy to Intermediate 32, Steps 6 to 10 and was obtained as a brown oil. MS (ESP) m/z=345.1 [M+H+H₂O]⁺.

### Intermediate 34

### (2R)-2-(Dimethylamino)-N-(2,4,7-trifluoro-2-formyl-indan-5-yl)propanamide

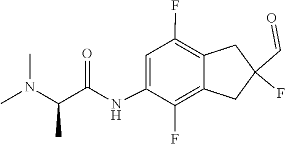 [SMILES: C[C@H](C(=O)Nc1cc(F)c2c(c1F)CC(F)(C=O)C2)N(C)C]

### Step 1: methyl 2,4,7-trifluoroindane-2-carboxylate

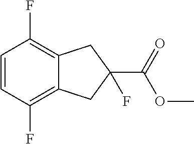 [SMILES: COC(=O)C1(F)Cc2c(F)ccc(F)c2C1]

To methyl 4,7-difluoro-2-hydroxy-indane-2-carboxylate (Intermediate 32, Step 5, 424.0 mg, 1.86 mmol, 1 eq) in DCM (8.01 mL) at −78° C., was added a solution of diethylaminosulfur trifluoride (0.37 mL, 2.79 mmol, 1.5 eq) in DCM (800.79 uL) dropwise. The reaction mixture was gradually warmed up to between 0° C. and 5° C. and stirred for 16 h. It was then diluted with dichloromethane, washed with NaHCO₃ (sat. aq.), filtered through a phase separator and concentrated in vacuo to afford the title compound (402 mg, 1.75 mmol, 93.99% yield) as a colorless oil.

### Steps 2 to 6: (2R)-2-(dimethylamino)-N-(2,4,7-trifluoro-2-formyl-indan-5-yl)propanamide

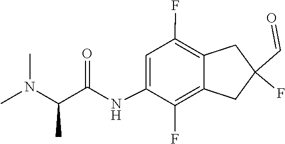 [SMILES: C[C@H](C(=O)Nc1cc(F)c2c(c1F)CC(F)(C=O)C2)N(C)C]

The title compound was prepared by analogy to Intermediate 32, Steps 6 to 10 and was obtained as a light brown solid. MS (ESP) m/z=315.1 [M+H]⁺.

### Intermediate 35

### (2R)—N-(2,7-Difluoro-2-formyl-indan-5-yl)-2-(dimethylamino)propanamide

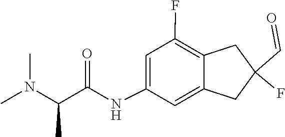 [SMILES: C[C@H](C(=O)Nc1cc(F)c2c(c1)CC(F)(C=O)C2)N(C)C]

### Step 1: ethyl 6-(tert-butoxycarbonylamino)-4-fluoro-indane-2-carboxylate

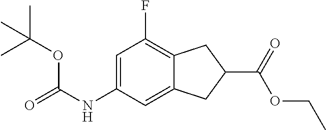 [SMILES: CCOC(=O)C1Cc2cc(NC(=O)OC(C)(C)C)cc(F)c2C1]

To a stirred solution of ethyl 6-amino-4-fluoro-indane-2-carboxylate (Intermediate 5, Step 7, 718.0 mg, 3.22 mmol, 1 eq) in dry THF (20 mL), di-tert-butyldicarbonate (842.34 mg, 3.86 mmol, 1.2 eq) was added and the reaction mixture was heated to 60° C. After 17 h the reaction was cooled down and the mixture was concentrated under reduced pressure. The obtained crude was purified by flash column chromatography (0-50% EtOAc in cyclohexane) to afford the title compound (979 mg, 3.03 mmol, 94.13% yield) as a light yellow oil. MS (ESP) m/z=324.2 [M+H]⁺.

### Step 2: ethyl 6-[bis(tert-butoxycarbonyl)amino]-4-fluoro-indane-2-carboxylate

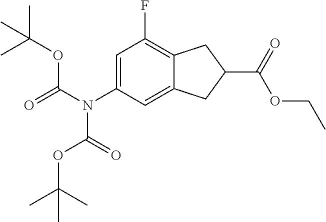 [SMILES: CCOC(=O)C1Cc2cc(N(C(=O)OC(C)(C)C)C(=O)OC(C)(C)C)cc(F)c2C1]

To a stirring solution of ethyl 6-(tert-butoxycarbonylamino)-4-fluoro-indane-2-carboxylate (879.0 mg, 2.72 mmol, 1 eq) in dry MeCN (25 mL), di-tert-butyldicarbonate (652.6 mg, 2.99 mmol, 1.1 eq) and DMAP (332.1 mg, 2.72 mmol, 1 eq) were added and the reaction mixture was stirred for 17 h at room temperature. Then the reaction volatiles were evaporated and the crude product was purified by flash column chromatography (0-10% EtOAc in cyclohexane) to give the title compound (1.24 g, 2.93 mmol, 107.72% yield) as a colorless oil. MS (ESP) m/z=446.3 [M+Na]⁺.

### Step 3: ethyl 6-[bis(tert-butoxycarbonyl)amino]-2,4-difluoro-indane-2-carboxylate

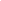

A solution of ethyl 6-[bis(tert-butoxycarbonyl)amino]-4-fluoro-indane-2-carboxylate (300.0 mg, 0.710 mmol, 1 eq) in THF (5 mL) was stirred at −78° C. for 20 minutes. After this time, lithium diisopropylamide solution (0.35 mL, 0.710 mmol, 1 eq) in THE (5 mL) was added dropwise. The mixture was left stirring at −78° C. for 15 minutes then N-fluorobenzenesulfonimide (268.07 mg, 0.850 mmol, 1.2 eq) in THF (2 mL) was added. The mixture was stirred at −78° C. for 1 h then it was quenched with NH₄Cl solution (sat. aq.). EtOAc was added, the mixture was left to reach room temperature. The phases were separated and the organic phase was dried over Na₂SO₄, filtered and concentrated under reduced pressure. The obtained crude was purified by flash column chromatography (0-30% EtOAc in cyclohexane) to give the title compound (416 mg, 0.940 mmol, quant. yield) as a colorless oil. MS (ESP) m/z=442.3 [M+H]⁺.

### Step 4: tert-butyl N-[2,7-difluoro-2-(hydroxymethyl)indan-5-yl]carbamate

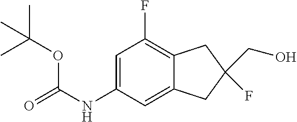 [SMILES: CC(C)(C)OC(=O)Nc1cc(F)c2c(c1)CC(F)(CO)C2]

A mixture of ethyl 6-[bis(tert-butoxycarbonyl)amino]-2,4-difluoro-indane-2-carboxylate (416.0 mg, 0.940 mmol, 1 eq) in THF (2 mL) was cooled to −15° C. then lithium aluminum hydride (0.45 mL, 1.04 mmol, 1.1 eq) was added. The mixture was left stirring at −15° C. for 0.25 h then it was quenched with Na₂SO₄·10H₂O. The resulting mixture was left stirring for 30 min., diluted with EtOAc and filtered. The filtrate was concentrated under reduced pressure and the obtained crude was purified by flash column chromatography (5-60% EtOAc in cyclohexane) to give the title compound (157.8 mg, 0.530 mmol, 55.95% yield) as a colorless oil. MS (ESN) m/z=298.3 [M−H]⁻.

### Step 5: (6-amino-2,4-difluoro-indan-2-yl)methanol

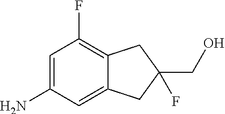 [SMILES: Nc1cc(F)c2c(c1)CC(F)(CO)C2]

A mixture of tert-butyl N-[2,7-difluoro-2-(hydroxymethyl)indan-5-yl]carbamate (157.8 mg, 0.530 mmol, 1 eq) and trifluoroacetic acid (0.41 mL, 5.27 mmol, 10 eq) in DCM (5 mL) was stirred at 20° C. for 6 h. It was partitioned between DCM and NaHCO₃ solution (sat. aq.). The phases were separated, the aqueous phase was extracted with DCM (×2). The combined organic phases were dried over Na₂SO₄, filtered and concentrated. The obtained crude was purified by flash column chromatography (10-100% EtOAc in cyclohexane) to give the title compound (41.6 mg, 0.210 mmol, 39.61% yield) a colorless oil. MS (ESP) m/z=200.1 [M+H]⁺.

### Step 6: (2R)—N-[2,7-difluoro-2-(hydroxymethyl)indan-5-yl]-2-(dimethylamino)propanamide

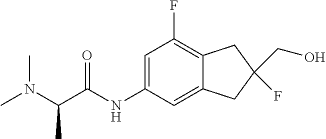 [SMILES: C[C@H](C(=O)Nc1cc(F)c2c(c1)CC(F)(CO)C2)N(C)C]

To a mixture of (6-amino-2,4-difluoro-indan-2-yl)methanol (41.5 mg, 0.210 mmol, 1 eq), (2R)-2-(dimethylamino)propanoic acid (26.85 mg, 0.230 mmol, 1.1 eq) in MeCN (2 mL), 1-methylimidazole (58.12 uL, 0.730 mmol, 3.5 eq) and TCFH (64.3 mg, 0.230 mmol, 1.1 eq) were added at 0° C. The mixture was warmed to RT over 20 min. and stirred at RT for 1 h. It was concentrated under reduced pressure and the residue purified by flash column chromatography (0-10% MeOH/DCM) to the title compound (38.8 mg, 0.130 mmol, 62.43% yield) as a colorless oil. MS (ESP) m/z=299.1 [M+H]⁺.

### Step 7: (2R)—N-(2,7-difluoro-2-formyl-indan-5-yl)-2-(dimethylamino)propanamide

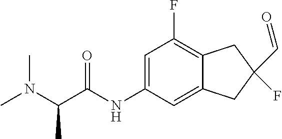 [SMILES: C[C@H](C(=O)Nc1cc(F)c2c(c1)CC(F)(C=O)C2)N(C)C]

The title compound was prepared by analogy to Intermediate 7, Step 2 and was obtained as a yellow oil. MS (ESP) m/z=297.1 [M+H]⁺.

### Intermediate 36

### (2R)—N-(7-Cyano-2-formyl-2-hydroxy-indan-5-yl)-2-(dimethylamino)propanamide

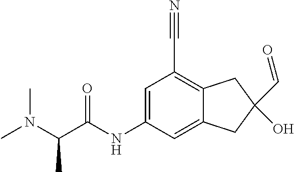 [SMILES: C[C@H](C(=O)Nc1cc(C#N)c2c(c1)CC(O)(C=O)C2)N(C)C]

### Step 1: ethyl 4-bromo-2-hydroxy-1-oxo-indane-2-carboxylate

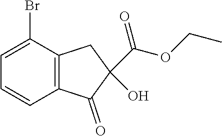 [SMILES: CCOC(=O)C1(O)Cc2c(Br)cccc2C1=O]

The title compound was prepared from ethyl 4-bromo-1-oxo-indane-2-carboxylate [CAS #1693851-34-1] by analogy to Intermediate 32, Step 2 and was obtained as a white solid. MS (ESP) m/z=299 [M+H]⁺.

### Step 2: ethyl 4-cyano-2-hydroxy-1-oxo-indane-2-carboxylate

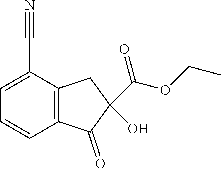 [SMILES: CCOC(=O)C1(O)Cc2c(C#N)cccc2C1=O]

A mixture of ethyl 4-bromo-2-hydroxy-1-oxo-indane-2-carboxylate (1.6 g, 5.35 mmol, 1 eq), tetrapotassium; hexacyanoiron; trihydrate (564.84 mg, 1.34 mmol, 0.250 eq), XPhos-G3-Pd (362.64 mg, 0.430 mmol, 0.080 eq) and tBuXPhos (427.92 mg, 1.01 mmol, 0.190 eq) in a sealed vial was evacuated and backfilled with N₂. 1,4-Dioxane (10 mL) and a solution of potassium acetate (78.74 mg, 0.800 mmol, 0.150 eq) in degassed water (10 mL) were added. The mixture was degassed for 2 min. then it was stirred at 90° C. for 5 h. The mixture was left to reach room temperature. The preceding procedure was carried out a further two times and the three reaction mixtures were then combined. EtOAc and brine were added, the phases were separated and the organic phase was extracted with EtOAc (×2). The combined organic phases were dried over Na₂SO₄, filtered and concentrated under reduced pressure. The obtained crude was purified by flash column chromatography (5-50% EtOAc/cyclohexane) to give the title compound (2.9 g, 11.83 mmol, 73.69% yield) as a yellow solid. MS (ESP) m/z=246.1 [M+H]⁺.

### Step 3: ethyl 4-cyano-1,2-dihydroxy-indane-2-carboxylate

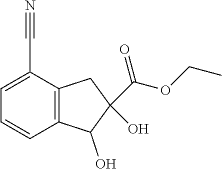 [SMILES: CCOC(=O)C1(O)Cc2c(C#N)cccc2C1O]

To a stirred solution of ethyl 4-cyano-2-hydroxy-1-oxo-indane-2-carboxylate (2.9 g, 11.83 mmol, 1 eq) in ethanol (80 mL) at 0° C., sodium borohydride (536.84 mg, 14.19 mmol, 1.2 eq) was added portionwise and the reaction mixture was stirred for 0.25 h. The reaction was quenched with ammonium chloride solution (sat. aq.) and extracted with DCM. The phases were separated and the water phase was extracted with DCM twice. The organic layers were combined and dried over sodium sulfate, filtered and concentrated under reduced pressure to obtain a crude which was purified by flash column chromatography (5-50% EtOAc in cyclohexane) to afford the title compound (1.37 g, 5.54 mmol, 46.86% yield) as a yellow oil. MS (ESP) m/z=248.0 [M+H]⁺.

### Steps 4 to 6: ethyl 4-cyano-2-hydroxy-6-nitro-indane-2-carboxylate

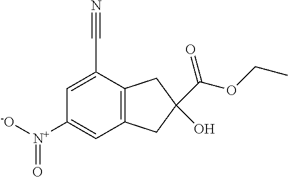

The title compound was prepared by analogy to Intermediate 32, Steps 4 to 6 and was obtained as a colourless oil. MS (ESP) m/z=277.1 [M+H]⁺.

### Step 7: ethyl 6-amino-4-cyano-2-hydroxy-indane-2-carboxylate

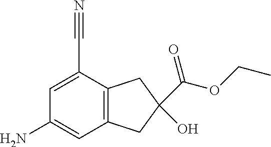 [SMILES: CCOC(=O)C1(O)Cc2cc(N)cc(C#N)c2C1]

To a mixture of ethyl 4-cyano-2-hydroxy-6-nitro-indane-2-carboxylate (289.0 mg, 1.05 mmol, 1 eq) in ethanol (15 mL) and water (5 mL), NH₄Cl (223.84 mg, 4.18 mmol, 4 eq) and iron (292.12 mg, 5.23 mmol, 5 eq) were added. The mixture was stirred at 75° C. for 0.5 h then was allowed to cool to room temperature. The mixture was filtered and partitioned between H₂O and EtOAc. The combined organic phases were dried over Na₂SO₄, filtered and concentrated. The obtained crude was purified by flash column chromatography (5-60% EtOAc in cyclohexane) to give the title compound (157 mg, 0.640 mmol, 60.94% yield) as a yellow oil. MS (ESP) m/z=247.1 [M+H]⁺.

### Step 8: ethyl 4-cyano-6-[[(2R)-2-(dimethylamino)propanoyl]amino]-2-hydroxy-indane-2-carboxylate

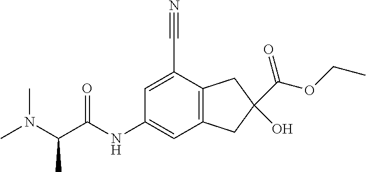 [SMILES: CCOC(=O)C1(O)Cc2cc(NC(=O)[C@@H](C)N(C)C)cc(C#N)c2C1]

The title compound was prepared by analogy to Intermediate 10, Step 1 and was obtained as an off-white solid. MS (ESP) m/z=346.3 [M+H]⁺.

### Step 9: (2R)—N-[7-cyano-2-hydroxy-2-(hydroxymethyl)indan-5-yl]-2-(dimethylamino)propanamide

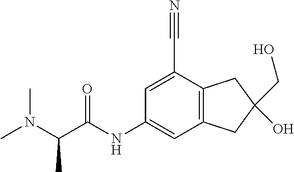 [SMILES: C[C@H](C(=O)Nc1cc(C#N)c2c(c1)CC(O)(CO)C2)N(C)C]

The title compound was prepared by analogy to Intermediate 22, Step 2 and was obtained as a colourless oil. MS (ESP) m/z=304.2 [M+H]⁺.

### Step 10: (2R)—N-(7-cyano-2-formyl-2-hydroxy-indan-5-yl)-2-(dimethylamino)propanamide

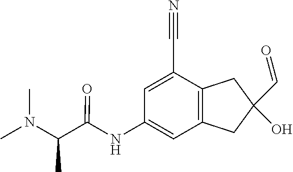 [SMILES: C[C@H](C(=O)Nc1cc(C#N)c2c(c1)CC(O)(C=O)C2)N(C)C]

The title compound was prepared by analogy to Intermediate 32, Step 10 and was obtained as a yellow oil. MS (ESP) m/z=302.2 [M+H]⁺.

### Intermediate 37

### tert-Butyl (2S)-2-[(7-fluoro-2-formyl-indan-5-yl)carbamoyl]pyrrolidine-1-carboxylate

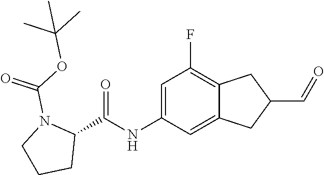 [SMILES: CC(C)(C)OC(=O)N1CCC[C@H]1C(=O)Nc1cc(F)c2c(c1)CC(C=O)C2]

The title compound was prepared by analogy to Intermediate 7 using BOC-L-proline [CAS #15761-39-4] in place of N,N-dimethylglycine and was obtained as a yellow oil. MS (ESP) m/z=377.2 [M+H]⁺.

### Intermediate 38

### tert-Butyl (2S,4R)-4-[tert-butyl(dimethyl)silyl]oxy-2-[(7-fluoro-2-formyl-indan-5-yl)carbamoyl]pyrrolidine-1-carboxylate

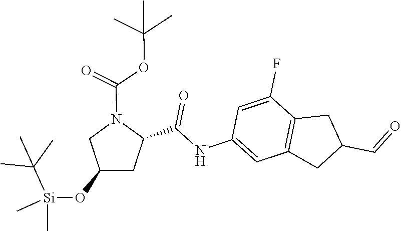 [SMILES: CC(C)(C)OC(=O)N1C[C@H](O[Si](C)(C)C(C)(C)C)C[C@H]1C(=O)Nc1cc(F)c2c(c1)CC(C=O)C2]

### Step 1: (2S,4R)-1-tert-butoxycarbonyl-4-[tert-butyl(dimethyl)silyl]oxy-pyrrolidine-2-carboxylic Acid

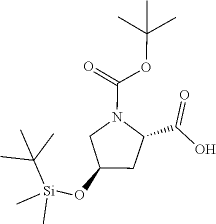 [SMILES: CC(C)(C)OC(=O)N1C[C@H](O[Si](C)(C)C(C)(C)C)C[C@H]1C(=O)O]

A mixture of tert-butyl (2S,4R)-4-hydroxy-1,2-pyrrolidinedicarboxylate [CAS #13726-69-7] (300.0 mg, 1.3 mmol, 1 eq) and imidazole (441.58 mg, 6.49 mmol, 5 eq) in DCM (4 mL) and DMF (1 mL) was cooled to 0° C. then tert-butyldimethylchlorosilane (430.16 mg, 2.85 mmol, 2.2 eq) was added. The mixture was left to reach room temperature and stirred for 36 h. EtOAc was added, the mixture was washed with water (×3), then dried over Na₂SO₄ and concentrated under reduced pressure to give the title compound (455 mg, 1.32 mmol, quant. yield) as a colorless oil. MS (ESN) m/z=344.5 [M−H]⁻.

### Steps 2 and 3: tert-butyl (2S,4R)-4-[tert-butyl(dimethyl)silyl]oxy-2-[(7-fluoro-2-formyl-indan-5-yl)carbamoyl]pyrrolidine-1-carboxylate

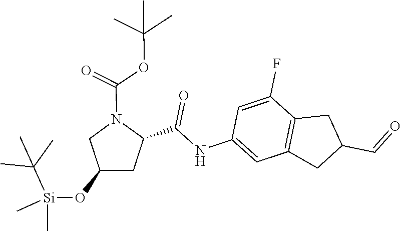 [SMILES: CC(C)(C)OC(=O)N1C[C@H](O[Si](C)(C)C(C)(C)C)C[C@H]1C(=O)Nc1cc(F)c2c(c1)CC(C=O)C2]

The title compound was prepared by analogy to Intermediate 7 using (2S,4R)-1-tert-butoxycarbonyl-4-[tert-butyl(dimethyl)silyl]oxy-pyrrolidine-2-carboxylic acid in place of N,N-dimethylglycine. MS (ESP) m/z=507.3 [M+H]⁺.

### Intermediate 39

### tert-Butyl N-[(1R)-1-(tert-butoxymethyl)-2-[(7-fluoro-2-formyl-indan-5-yl)amino]-2-oxo-ethyl]carbamate

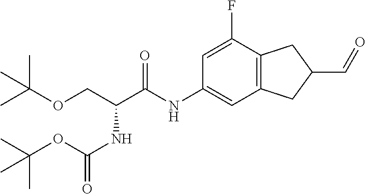 [SMILES: CC(C)(C)OC[C@@H](NC(=O)OC(C)(C)C)C(=O)Nc1cc(F)c2c(c1)CC(C=O)C2]

The title compound was prepared by analogy to Intermediate 7 using (2R)-3-tert-butoxy-2-(tert-butoxycarbonylamino)propanoic acid [CAS #248921-66-6] in place of N,N-dimethylglycine. MS (ESP) m/z=423.2 [M+H]⁺.

### Intermediate 40

### tert-Butyl N-[(1R)-2-[(7-fluoro-2-formyl-indan-5-yl)amino]-1-(methoxymethyl)-2-oxo-ethyl]carbamate

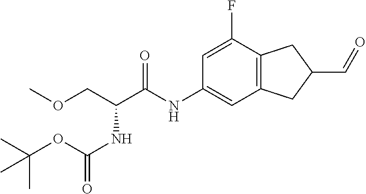 [SMILES: COC[C@@H](NC(=O)OC(C)(C)C)C(=O)Nc1cc(F)c2c(c1)CC(C=O)C2]

The title compound was prepared by analogy to Intermediate 7 using (2R)-2-(tert-butoxycarbonylamino)-3-methoxy-propanoic acid [CAS #86123-95-7] in place of N,N-dimethylglycine. MS (ESP) m/z=381.2 [M+H]⁺.

### Intermediate 41

### tert-Butyl N-[(3R)-3-[(7-fluoro-2-formyl-indan-5-yl)carbamoyl]tetrahydrofuran-3-yl]carbamate

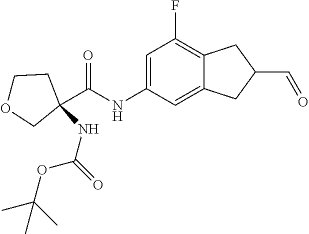 [SMILES: CC(C)(C)OC(=O)N[C@]1(C(=O)Nc2cc(F)c3c(c2)CC(C=O)C3)CCOC1]

### Step 1: (3R)-3-(tert-butoxycarbonylamino)tetrahydrofuran-3-carboxylic Acid

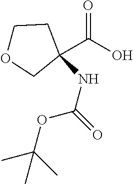 [SMILES: CC(C)(C)OC(=O)N[C@]1(C(=O)O)CCOC1]

To a solution of (3R)-3-aminotetrahydrofuran-3-carboxylic acid [CAS #1315053-78-1] (200.0 mg, 1.53 mmol, 1 eq) in water (3 mL) and 1,4-dioxane (3 mL), sodium hydroxide (1.6 mL, 1.6 mmol, 1.05 eq) (1M solution) was added. The mixture was cooled to 0° C. and di-tert-butyldicarbonate (366.16 mg, 1.68 mmol, 1.1 eq) was added and the mixture was allowed to warm to r.t. and stirred overnight. The reaction mixture was acidified to pH 2.0 with 0.1 M HCl, extracted with EtOAc, dried (Na₂SO₄), filtered and concentrated under reduced pressure to obtain the title compound (195 mg, 0.840 mmol, 55.29% yield). MS (ESP) m/z=232.1 [M+H]⁺.

### Steps 2 and 3: tert-butyl N-[(3R)-3-[(7-fluoro-2-formyl-indan-5-yl)carbamoyl]tetrahydrofuran-3-yl]carbamate

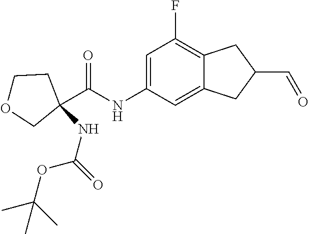 [SMILES: CC(C)(C)OC(=O)N[C@]1(C(=O)Nc2cc(F)c3c(c2)CC(C=O)C3)CCOC1]

The title compound was prepared by analogy to Intermediate 7 using (3R)-3-(tert-butoxycarbonylamino)tetrahydrofuran-3-carboxylic acid in place of N,N-dimethylglycine. MS (ESP) m/z=393.2 [M+H]⁺.

### Intermediate 42

### tert-Butyl N-[(3S)-3-[(7-fluoro-2-formyl-indan-5-yl)carbamoyl]tetrahydrofuran-3-yl]carbamate

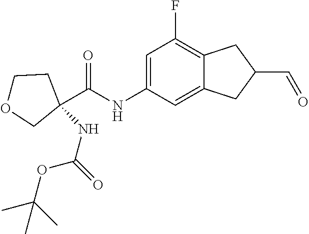 [SMILES: CC(C)(C)OC(=O)N[C@@]1(C(=O)Nc2cc(F)c3c(c2)CC(C=O)C3)CCOC1]

The title compound was prepared by analogy to Intermediate 41 using (3S)-3-aminotetrahydrofuran-3-carboxylic acid [CAS #1315052-80-2] in place of (3R)-3-aminotetrahydrofuran-3-carboxylic acid.

### Intermediate 43

### tert-Butyl N-[2-[(7-fluoro-2-formyl-indan-5-yl)amino]-1,1-dimethyl-2-oxo-ethyl]carbamate

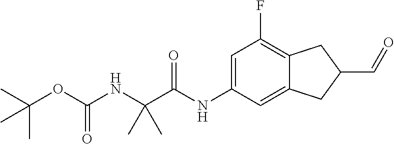 [SMILES: CC(C)(C)OC(=O)NC(C)(C)C(=O)Nc1cc(F)c2c(c1)CC(C=O)C2]

The title compound was prepared by analogy to Intermediate 9 using N—BOC-2-methylalanine [CAS #30992-29-1] in place of 2-(dimethylamino)-2-methyl-propanoic acid; hydrochloride and was obtained as a light yellow solid. (ESP) m/z=365.2 [M+H]⁺.

### Intermediate 44

### tert-Butyl (2S,4R)-2-[(7-fluoro-2-formyl-indan-5-yl)carbamoyl]-4-methoxy-pyrrolidine-1-carboxylate

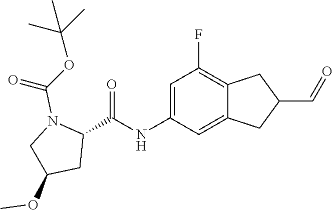 [SMILES: CO[C@@H]1C[C@@H](C(=O)Nc2cc(F)c3c(c2)CC(C=O)C3)N(C(=O)OC(C)(C)C)C1]

The title compound was prepared by analogy to Intermediate 7 using (2S,4R)-1-tert-butoxycarbonyl-4-methoxy-pyrrolidine-2-carboxylic acid [CAS #83624-01-5] in place of N,N-dimethylglycine and was obtained as a yellow semi-solid. MS (ESP) m/z=407.4 [M+H]⁺.

### Intermediate 45

### tert-Butyl (2R,4R)-4-[tert-butyl(dimethyl)silyl]oxy-2-[(7-fluoro-2-formyl-indan-5-yl)carbamoyl]pyrrolidine-1-carboxylate

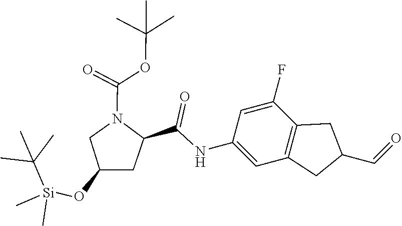 [SMILES: CC(C)(C)OC(=O)N1C[C@H](O[Si](C)(C)C(C)(C)C)C[C@@H]1C(=O)Nc1cc(F)c2c(c1)CC(C=O)C2]

The title compound was prepared by analogy to Intermediate 38 using tert-butyl (2R,4R)-4-hydroxy-1,2-pyrrolidinedicarboxylate [CAS #135042-12-5] in place of tert-butyl (2S,4R)-4-hydroxy-1,2-pyrrolidinedicarboxylate. MS (ESP) m/z=507.4 [M+H]⁺.

### Intermediate 46

### tert-Butyl (2S,4S)-4-[tert-butyl(dimethyl)silyl]oxy-2-[(7-fluoro-2-formyl-indan-5-yl)carbamoyl]pyrrolidine-1-carboxylate

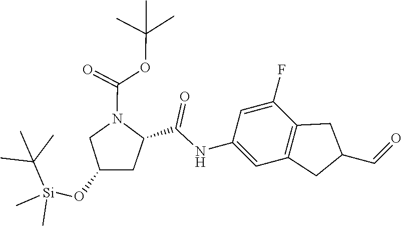 [SMILES: CC(C)(C)OC(=O)N1C[C@@H](O[Si](C)(C)C(C)(C)C)C[C@H]1C(=O)Nc1cc(F)c2c(c1)CC(C=O)C2]

The title compound was prepared by analogy to Intermediate 38 using tert-butyl (2S,4S)-4-hydroxy-1,2-pyrrolidinedicarboxylate [CAS #87691-27-8] in place of tert-butyl (2S,4R)-4-hydroxy-1,2-pyrrolidinedicarboxylate. MS (ESP) m/z=507.4 [M+H]⁺.

### Intermediate 47

### tert-Butyl (2R,4S)-4-[tert-butyl(dimethyl)silyl]oxy-2-[(7-fluoro-2-formyl-indan-5-yl)carbamoyl]pyrrolidine-1-carboxylate

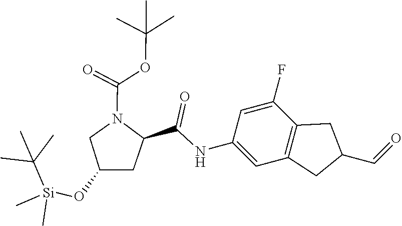 [SMILES: CC(C)(C)OC(=O)N1C[C@@H](O[Si](C)(C)C(C)(C)C)C[C@@H]1C(=O)Nc1cc(F)c2c(c1)CC(C=O)C2]

The title compound was prepared by analogy to Intermediate 38 using tert-butyl (2R,4S)-4-hydroxy-1,2-pyrrolidinedicarboxylate [CAS #147266-92-0] in place of tert-butyl (2S,4R)-4-hydroxy-1,2-pyrrolidinedicarboxylate and was obtained as a light yellow oil. MS (ESP) m/z=507.4 [M+H]⁺.

### Intermediate 48

### tert-Butyl (3S)-3-(tert-butoxycarbonylamino)-4-[(7-fluoro-2-formyl-indan-5-yl)amino]-4-oxo-butanoate

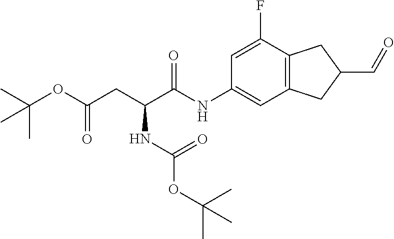 [SMILES: CC(C)(C)OC(=O)C[C@H](NC(=O)OC(C)(C)C)C(=O)Nc1cc(F)c2c(c1)CC(C=O)C2]

The title compound was prepared by analogy to Intermediate 7 using 4-tert-butyl N-(tert-butoxycarbonyl)-L-aspartate [CAS #1676-90-0] in place of N,N-dimethylglycine and was obtained as a yellow oil. MS (ESP) m/z=451.5 [M+H]⁺.

### Intermediate 49

### (2S,4R)-4-[tert-Butyl(dimethyl)silyl]oxy-N-(7-fluoro-2-formyl-indan-5-yl)-1-methyl-pyrrolidine-2-carboxamide

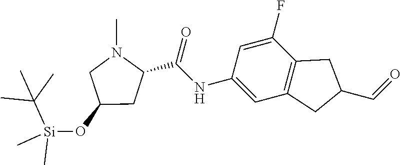 [SMILES: CN1C[C@H](O[Si](C)(C)C(C)(C)C)C[C@H]1C(=O)Nc1cc(F)c2c(c1)CC(C=O)C2]

The title compound was prepared by analogy to Intermediate 38 using (2S,4R)-4-hydroxy-1-methyl-pyrrolidine-2-carboxylic acid [CAS #4252-82-8] in place of tert-butyl (2S,4R)-4-hydroxy-1,2-pyrrolidinedicarboxylate and was obtained as an orange viscous oil. MS (ESP) m/z=421.5 [M+H]⁺.

### Intermediate 50

### 2-[(3R)-3-[tert-Butyl(diphenyl)silyl]oxypyrrolidin-1-yl]-N-(7-fluoro-2-formyl-indan-5-yl)acetamide

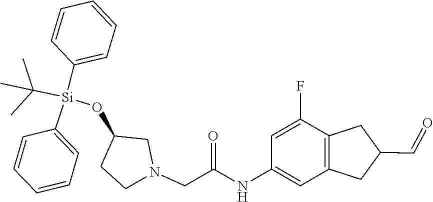 [SMILES: CC(C)(C)[Si](O[C@@H]1CCN(CC(=O)Nc2cc(F)c3c(c2)CC(C=O)C3)C1)(c1ccccc1)c1ccccc1]

### Step 1: tert-butyl-diphenyl-[(3R)-pyrrolidin-3-yl]oxy-silane

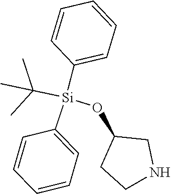 [SMILES: CC(C)(C)[Si](O[C@@H]1CCNC1)(c1ccccc1)c1ccccc1]

A mixture of (R)-3-hydroxypyrrolidine [CAS #2799-21-5] (0.19 mL, 2.3 mmol, 1 eq) and imidazole (390.71 mg, 5.74 mmol, 2.5 eq) in dichloromethane (15 mL) was cooled to 0° C. then tert-butylchlorodiphenylsilane (757.19 mg, 2.75 mmol, 1.2 eq) was added portionwise. The mixture was left to reach room temperature and stirred overnight. The mixture was concentrated and purified by flash column chromatography (0-10% MeOH in DCM) to give the title compound (253 mg, 0.780 mmol, 33.86% yield). MS (ESP) m/z=326.3 [M+H]⁺.

### Step 2: 2-[(3R)-3-[tert-butyl(diphenyl)silyl]oxypyrrolidin-1-yl]-N-[7-fluoro-2-(hydroxymethyl)indan-5-yl]acetamide

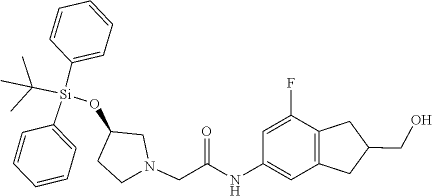 [SMILES: CC(C)(C)[Si](O[C@@H]1CCN(CC(=O)Nc2cc(F)c3c(c2)CC(CO)C3)C1)(c1ccccc1)c1ccccc1]

The title compound was prepared by analogy to Intermediate 12, Step 1 using tert-butyl-diphenyl-[(3R)-pyrrolidin-3-yl]oxy-silane in place of azetidine and was obtained as a yellow oil. MS (ESN) m/z=545.4 [M−H]⁻.

### Step 3: 2-[(3R)-3-[tert-butyl(diphenyl)silyl]oxypyrrolidin-1-yl]-N-(7-fluoro-2-formyl-indan-5-yl)acetamide

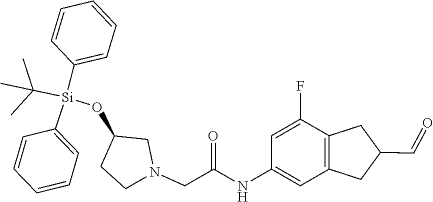 [SMILES: CC(C)(C)[Si](O[C@@H]1CCN(CC(=O)Nc2cc(F)c3c(c2)CC(C=O)C3)C1)(c1ccccc1)c1ccccc1]

The title compound was prepared by analogy to Intermediate 7, Step 3 and was obtained as a brown foam. MS (ESN) m/z=543.4 [M−H]⁻.

### Intermediate 51

### 6-[8-[(6-Amino-4-fluoro-indan-2-yl)methyl]-2-oxo-1-oxa-3,8-diazaspiro[4.5]decan-3-yl]-4H-pyrazino[2,3-b][1,4]oxazin-3-one

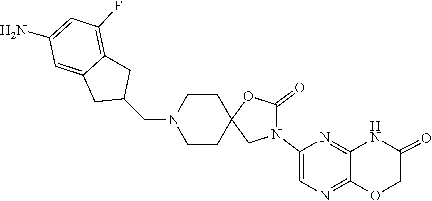 [SMILES: Nc1cc(F)c2c(c1)CC(CN1CCC3(CC1)CN(c1cnc4c(n1)NC(=O)CO4)C(=O)O3)C2]

### Step 1: tert-butyl N-[7-fluoro-2-(hydroxymethyl)indan-5-yl]carbamate

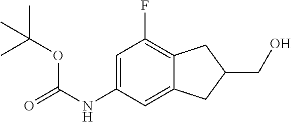 [SMILES: CC(C)(C)OC(=O)Nc1cc(F)c2c(c1)CC(CO)C2]

To a solution of (6-amino-4-fluoro-indan-2-yl)methanol (Intermediate 5, 244.0 mg, 1.35 mmol, 1 eq) in THF (9 mL) at RT, di-tert-butyldicarbonate (295.76 mg, 1.36 mmol, 1.01 eq) was added and the reaction mixture was shaken at RT for 17 h. The reaction mixture was diluted with EtOAc and water, the phases were separated and the aqueous layer was extracted with EtOAc (×3). The combined organic phases were filtered through a phase separator and volatiles were removed under reduced pressure to afford the a residue which was purified by silica cartridge chromatography (25 g silica, from cyclohexane 100% to cyclohexane/EtOAc 7:3) to give the title compound (290 mg, 1.03 mmol, 76.56% yield) as a white foam. MS (ESP) m/z=282.2 [M+H]⁺.

### Step 2: tert-butyl N-(7-fluoro-2-formyl-indan-5-yl)carbamate

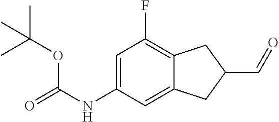 [SMILES: CC(C)(C)OC(=O)Nc1cc(F)c2c(c1)CC(C=O)C2]

The title compound was prepared by analogy to Intermediate 7, Step 2 and was obtained as a yellow viscous oil. MS (ESP) m/z=224.02 [M+H-tBu]⁺.

### Step 3: tert-butyl N-[7-fluoro-2-[[2-oxo-3-(3-oxo-4H-pyrazino[2,3-b][1,4]oxazin-6-yl)-1-oxa-3,8-diazaspiro[4.5]decan-8-yl]methyl]indan-5-yl]carbamate

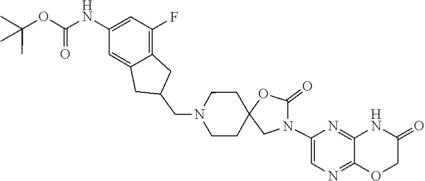 [SMILES: CC(C)(C)OC(=O)Nc1cc(F)c2c(c1)CC(CN1CCC3(CC1)CN(c1cnc4c(n1)NC(=O)CO4)C(=O)O3)C2]

To a suspension of 6-(2-oxo-1-oxa-3,8-diazaspiro[4.5]decan-3-yl)-4H-pyrazino[2,3-b][1,4]oxazin-3-one; hydrochloride (Intermediate 4, 264.29 mg, 0.770 mmol, 1 eq) and tert-butyl N-(7-fluoro-2-formyl-indan-5-yl)carbamate (216.0 mg, 0.770 mmol, 1 eq) in DMA (2 mL) and tetrahydrofuran (6 mL) at RT, N-ethyldiisopropylamine (0.34 mL, 1.93 mmol, 2.5 eq) was added. The reaction mixture was stirred at RT for 30 min. Then acetic acid (0.2 mL, 3.48 mmol, 4.5 eq) and sodium triacetoxyborohydride (409.75 mg, 1.93 mmol, 2.5 eq) were added and the mixture stirred at RT for 15.5 h. The reaction mixture was diluted with EtOAc and NaHCO₃ (sat. aq.). The phases were separated and the organic layer was washed with NaHCO₃ (sat. aq.) (×2) and brine. The combined organic phases were filtered through a phase separator and volatiles were removed under reduced pressure to afford a residue which was purified by silica cartridge chromatography (28 g silica-NH, from DCM 100% to DCM/[EtOAc/MeOH 9:1] 95:5) to give the title compound (315 mg, 0.550 mmol, 71.64% yield) as a white solid. MS (ESP) m/z=569.4 [M+H]⁺.

### Step 4: 6-[8-[(6-amino-4-fluoro-indan-2-yl)methyl]-2-oxo-1-oxa-3,8-diazaspiro[4.5]decan-3-yl]-4H-pyrazino[2,3-b][1,4]oxazin-3-one

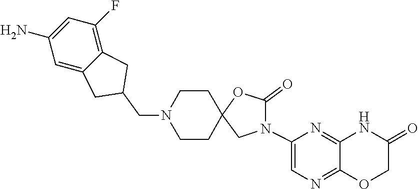 [SMILES: Nc1cc(F)c2c(c1)CC(CN1CCC3(CC1)CN(c1cnc4c(n1)NC(=O)CO4)C(=O)O3)C2]

A solution of tert-butyl N-[7-fluoro-2-[[2-oxo-3-(3-oxo-4H-pyrazino[2,3-b][1,4]oxazin-6-yl)-1-oxa-3,8-diazaspiro[4.5]decan-8-yl]methyl]indan-5-yl]carbamate (315.0 mg, 0.550 mmol, 1 eq) in DCM (4 mL) and trifluoroacetic acid (2.0 mL, 25.96 mmol, 46.86 eq) was stirred at RT for 2 h. Volatiles were removed under reduced pressure, the residue was partitioned between EtOAc/MeOH 9:1 and NaHCO₃ (sat. aq.). The phases were separated and the aqueous layer was extracted with EtOAc/MeOH 9:1 (×3). The combined organic phases were filtered through a phase separator and volatiles were removed under reduced pressure to afford the crude title compound (260 mg, 0.550 mmol, 100.18% yield) as an off-white solid. MS (ESP) m/z=469.4 [M+H]⁺.

### Intermediate 52

### 6-[8-[(5-Amino-4,7-difluoro-indan-2-yl)methyl]-2-oxo-1-oxa-3,8-diazaspiro[4.5]decan-3-yl]-4H-pyrazino[2,3-b][1,4]oxazin-3-one

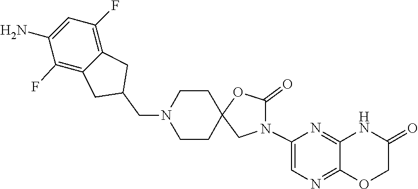 [SMILES: Nc1cc(F)c2c(c1F)CC(CN1CCC3(CC1)CN(c1cnc4c(n1)NC(=O)CO4)C(=O)O3)C2]

### Step 1: diethyl 4,7-difluoroindane-2,2-dicarboxylate

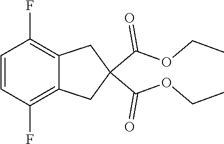 [SMILES: CCOC(=O)C1(C(=O)OCC)Cc2c(F)ccc(F)c2C1]

To a solution of diethyl malonate (3.39 mL, 22.34 mmol, 1 eq) in THF (709.14 mL), potassium tert-butoxide (5.26 g, 46.91 mmol, 2.1 eq) was added at 0° C. The mixture was stirred at the same temperature for 1 h, then 2,3-bis(bromomethyl)-1,4-difluoro-benzene [CAS #912999-77-0] (6.7 g, 22.34 mmol, 1 eq) dissolved in THF (354.57 mL) was added and the mixture was stirred at 60° C. for 24 h. The reaction was concentrated in vacuo. The mixture was partioned between water and EtOAC. The phase were separated and the aqueous phase was extracted with EtOAc (×1). The combined organic phase was dried over Na₂SO₄, filtered and concentrated under vacuum to afford the crude title compound (6.7 g, 22.46 mmol, quant. yield) which was used in the following reaction without further purification.

### Step 2: ethyl 4,7-difluoroindane-2-carboxylate

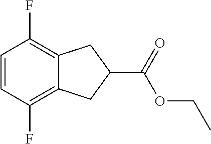 [SMILES: CCOC(=O)C1Cc2c(F)ccc(F)c2C1]

A mixture of diethyl 4,7-difluoroindane-2,2-dicarboxylate (1250.0 mg, 4.19 mmol, 1 eq), water (75.5 uL, 4.19 mmol, 1 eq) and lithium chloride (461.92 mg, 10.9 mmol, 2.6 eq) in DMSO (14.97 mL) was stirred at 160° C. for 3.5 h. Additional lithium chloride (177.66 mg, 4.19 mmol, 1 eq) was added and stirring prolonged for 1 h. The temperature was increased to 175° C. and stirring prolonged for 1 h. The reaction was cooled to room temperature and diluted with EtOAc and washed with water (×1) and then with brine (×1). The combined organic phase was dried over Na₂SO₄ and concentrated in vacuo to afford the crude title compound (330 mg, 1.46 mmol, 34.8% crude yield) which was purified by silica cartridge chromatography (cyclohexane:EtOAc from 99:1 to 90:10 then to 20:80) to afford the title compound. MS (ESP) m/z=227.0 [M+H]⁺.

### Steps 3 and 4: ethyl 5-amino-4,7-difluoro-indane-2-carboxylate

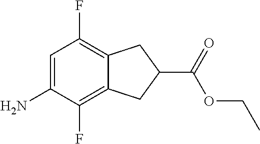 [SMILES: CCOC(=O)C1Cc2c(F)cc(N)c(F)c2C1]

The title compound was prepared by analogy Intermediate 24, Steps 2 and 3 and was obtained as a red oil. MS (ESP) m/z=242.1 [M+H]⁺.

### Step 5: ethyl 5-(tert-butoxycarbonylamino)-4,7-difluoro-indane-2-carboxylate

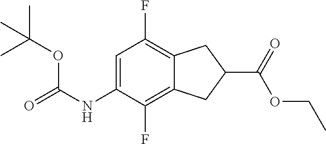 [SMILES: CCOC(=O)C1Cc2c(F)cc(NC(=O)OC(C)(C)C)c(F)c2C1]

The title compound was prepared by analogy to Intermediate 51, Step 1. MS (ESP) m/z=286.1 [M+H-tBu]⁺.

### Step 6: tert-butyl N-[4,7-difluoro-2-(hydroxymethyl)indan-5-yl]carbamate

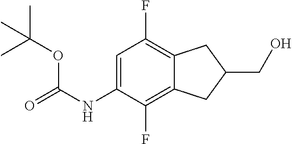 [SMILES: CC(C)(C)OC(=O)Nc1cc(F)c2c(c1F)CC(CO)C2]

To a solution of ethyl 5-(tert-butoxycarbonylamino)-4,7-difluoro-indane-2-carboxylate (149.0 mg, 0.440 mmol, 1 eq) in THF (5 mL) at −15° C., lithium aluminum hydride (0.19 mL, 0.440 mmol, 1 eq) was added and the mixture was stirred for 15 min. The reaction mixture was quenched with sodium sulfate decahydrate and stirred for 30 min. The resulting suspension was diluted with EtOAc and filtered. The filtrate was collected and volatiles were removed under reduced pressure to obtain the title compound (130 mg, 0.430 mmol, 99.5% yield). MS (ESP) m/z=244.2 [M+H-tBu]⁺.

### Steps 7 to 9: 6-[8-[(5-amino-4,7-difluoro-indan-2-yl)methyl]-2-oxo-1-oxa-3,8-diazaspiro[4.5]decan-3-yl]-4H-pyrazino[2,3-b][1,4]oxazin-3-one

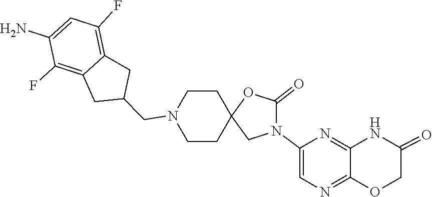 [SMILES: Nc1cc(F)c2c(c1F)CC(CN1CCC3(CC1)CN(c1cnc4c(n1)NC(=O)CO4)C(=O)O3)C2]

The title compound was prepared by analogy to Intermediate 51, Steps 2 to 4. MS (ESP) m/z=487.4 [M+H]⁺.

### Intermediate 53

### tert-Butyl (2R)-2-[[tert-butyl(dimethyl)silyl]oxymethyl]-2-[[7-fluoro-2-[[2-oxo-3-(3-oxo-4H-pyrazino[2,3-b][1,4]oxazin-6-yl)-1-oxa-3,8-diazaspiro[4.5]decan-8-yl]methyl]indan-5-yl]carbamoyl]pyrrolidine-1-carboxylate

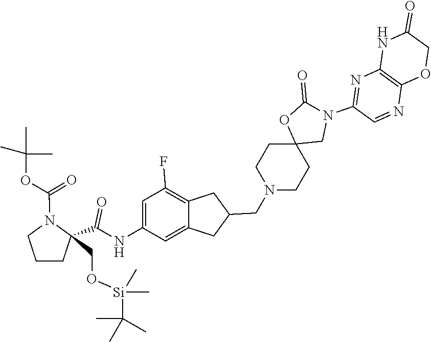 [SMILES: CC(C)(C)OC(=O)N1CCC[C@@]1(CO[Si](C)(C)C(C)(C)C)C(=O)Nc1cc(F)c2c(c1)CC(CN1CCC3(CC1)CN(c1cnc4c(n1)NC(=O)CO4)C(=O)O3)C2]

### Step 1: (2R)-1-tert-butoxycarbonyl-2-[[tert-butyl(dimethyl)silyl]oxymethyl]pyrrolidine-2-carboxylic acid

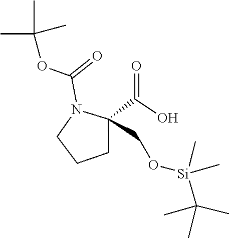 [SMILES: CC(C)(C)OC(=O)N1CCC[C@@]1(CO[Si](C)(C)C(C)(C)C)C(=O)O]

The title compound was prepared by analogy to Intermediate 38, Step 1 using (2R)-1-tert-butoxycarbonyl-2-(hydroxymethyl)pyrrolidine-2-carboxylic acid [CAS #287401-41-6] in place of tert-butyl (2S,4R)-4-hydroxy-1,2-pyrrolidinedicarboxylate and was obtained as a colorless oil.

### Step 2: tert-butyl (2R)-2-[[tert-butyl(dimethyl)silyl]oxymethyl]-2-[(2-ethoxycarbonyl-7-fluoro-indan-5-yl)carbamoyl]pyrrolidine-1-carboxylate

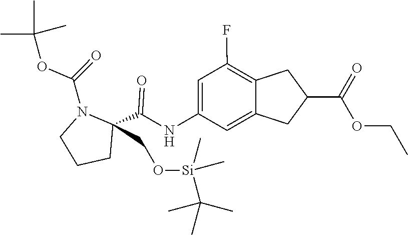 [SMILES: CCOC(=O)C1Cc2cc(NC(=O)[C@]3(CO[Si](C)(C)C(C)(C)C)CCCN3C(=O)OC(C)(C)C)cc(F)c2C1]

The title compound was prepared from ethyl 6-amino-4-fluoro-indane-2-carboxylate (Intermediate 5, Step 7) and (2R)-1-tert-butoxycarbonyl-2-[[tert-butyl(dimethyl)silyl]oxymethyl]pyrrolidine-2-carboxylic acid by analogy to Intermediate 7, Step 1 and was obtained as an off-white solid. MS (ESN) m/z=563.3 [M−H]⁻.

### Step 3: tert-butyl (2R)-2-[[tert-butyl(dimethyl)silyl]oxymethyl]-2-[[7-fluoro-2-(hydroxymethyl)indan-5-yl]carbamoyl]pyrrolidine-1-carboxylate

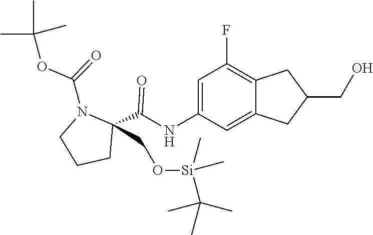 [SMILES: CC(C)(C)OC(=O)N1CCC[C@@]1(CO[Si](C)(C)C(C)(C)C)C(=O)Nc1cc(F)c2c(c1)CC(CO)C2]

The title compound was prepared by analogy to Intermediate 22, Step 2 and was obtained as an off-white solid. MS (ESP) m/z=523.3 [M+H]⁺.

### Step 4 tert-butyl (2R)-2-[[tert-butyl(dimethyl)silyl]oxymethyl]-2-[(7-fluoro-2-formyl-indan-5-yl)carbamoyl]pyrrolidine-1-carboxylate

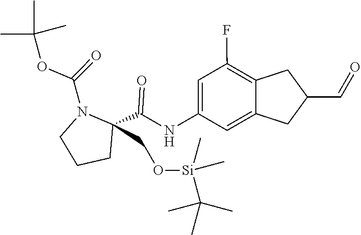 [SMILES: CC(C)(C)OC(=O)N1CCC[C@@]1(CO[Si](C)(C)C(C)(C)C)C(=O)Nc1cc(F)c2c(c1)CC(C=O)C2]

The title compound was prepared by analogy to Intermediate 7, Step 2 and was obtained as a colorless oil. MS (ESP) m/z=521.3 [M+H]⁺.

### Step 5: tert-butyl (2R)-2-[[tert-butyl(dimethyl)silyl]oxymethyl]-2-[[7-fluoro-2-[[2-oxo-3-(3-oxo-4H-pyrazino[2,3-b][1,4]oxazin-6-yl)-1-oxa-3,8-diazaspiro[4.5]decan-8-yl]methyl]indan-5-yl]carbamoyl]pyrrolidine-1-carboxylate

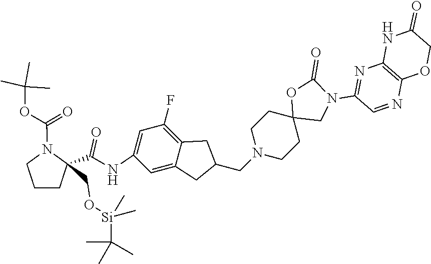 [SMILES: CC(C)(C)OC(=O)N1CCC[C@@]1(CO[Si](C)(C)C(C)(C)C)C(=O)Nc1cc(F)c2c(c1)CC(CN1CCC3(CC1)CN(c1cnc4c(n1)NC(=O)CO4)C(=O)O3)C2]

The title compound was prepared by analogy to Intermediate 51, Step 3 using tert-butyl (2R)-2-[[tert-butyl(dimethyl)silyl]oxymethyl]-2-[(7-fluoro-2-formyl-indan-5-yl)carbamoyl]pyrrolidine-1-carboxylate in place of and tert-butyl N-(7-fluoro-2-formyl-indan-5-yl)carbamate and was obtained as an off-white solid. MS (ESP) m/z=810.5 [M+H]⁺.

### Intermediate 54

### tert-Butyl (2S,3S)-3-[tert-butyl(dimethyl)silyl]oxy-2-[[7-fluoro-2-[[2-oxo-3-(3-oxo-4H-pyrazino[2,3-b][1,4]oxazin-6-yl)-1-oxa-3,8-diazaspiro[4.5]decan-8-yl]methyl]indan-5-yl]carbamoyl]pyrrolidine-1-carboxylate

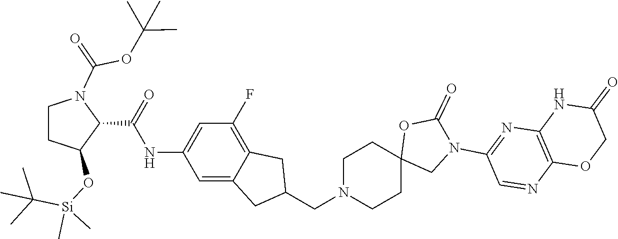 [SMILES: CC(C)(C)OC(=O)N1CC[C@H](O[Si](C)(C)C(C)(C)C)[C@H]1C(=O)Nc1cc(F)c2c(c1)CC(CN1CCC3(CC1)CN(c1cnc4c(n1)NC(=O)CO4)C(=O)O3)C2]

The title compound was prepared by analogy to Intermediate 41, Steps 1 and 2 using (2S,3S)-1-tert-butoxycarbonyl-3-hydroxy-pyrrolidine-2-carboxylic acid [CAS #187039-57-2] in place of (3R)-3-aminotetrahydrofuran-3-carboxylic acid in Step 1 and using 6-[8-[(6-amino-4-fluoro-indan-2-yl)methyl]-2-oxo-1-oxa-3,8-diazaspiro[4.5]decan-3-yl]-4H-pyrazino[2,3-b][1,4]oxazin-3-one (Intermediate 51) in place of (6-amino-4-fluoro-indan-2-yl)methanol in Step 2 and was obtained as a light yellow oil. MS (ESP) m/z=796.2 [M+H]⁺.

### Intermediate 55

### 6-Amino-2-[[2-oxo-3-(3-oxo-4H-pyrazino[2,3-b][1,4]oxazin-6-yl)-1-oxa-3,8-diazaspiro[4.5]decan-8-yl]methyl]indane-4-carbonitrile

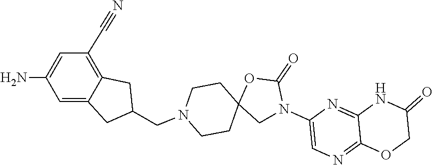 [SMILES: N#Cc1cc(N)cc2c1CC(CN1CCC3(CC1)CN(c1cnc4c(n1)NC(=O)CO4)C(=O)O3)C2]

### Step 1: ethyl 6-(tert-butoxycarbonylamino)-4-cyano-indane-2-carboxylate

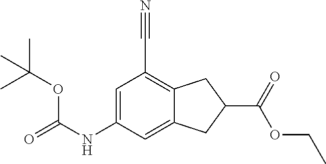 [SMILES: CCOC(=O)C1Cc2cc(NC(=O)OC(C)(C)C)cc(C#N)c2C1]

The title compound was prepared from ethyl 6-amino-4-cyano-indane-2-carboxylate (Intermediate 24, Step 3) by analogy to Intermediate 51, Step 1 and was obtained as a pale yellow oil. MS (ESP) m/z=331.2 [M+H]⁺.

### Step 2: tert-butyl N-[7-cyano-2-(hydroxymethyl)indan-5-yl]carbamate

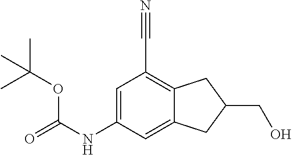 [SMILES: CC(C)(C)OC(=O)Nc1cc(C#N)c2c(c1)CC(CO)C2]

The title compound was prepared by analogy to Intermediate 22, Step 2 and was obtained as a colourless oil. MS (ESP) m/z=289.3 [M+H]⁺.

### Steps 3 to 5: 6-amino-2-[[2-oxo-3-(3-oxo-4H-pyrazino[2,3-b][1,4]oxazin-6-yl)-1-oxa-3,8-diazaspiro[4.5]decan-8-yl]methyl]indane-4-carbonitrile

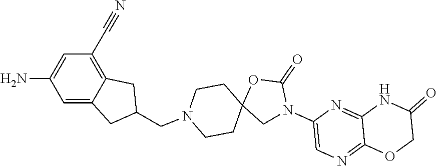 [SMILES: N#Cc1cc(N)cc2c1CC(CN1CCC3(CC1)CN(c1cnc4c(n1)NC(=O)CO4)C(=O)O3)C2]

The title compound was prepared by analogy to Intermediate 51, Steps 2 to 4 and was obtained as a light yellow solid. MS (ESP) m/z=476.4 [M+H]⁺.

### Intermediate 56

### tert-Butyl (2S,4R)-4-[tert-butyl(dimethyl)silyl]oxy-2-[[6-[[2-oxo-3-(3-oxo-4H-pyrazino[2,3-b][1,4]oxazin-6-yl)-1-oxa-3,8-diazaspiro[4.5]decan-8-yl]methyl]-6,7-dihydro-5H-cyclopenta[b]pyridin-3-yl]carbamoyl]pyrrolidine-1-carboxylate

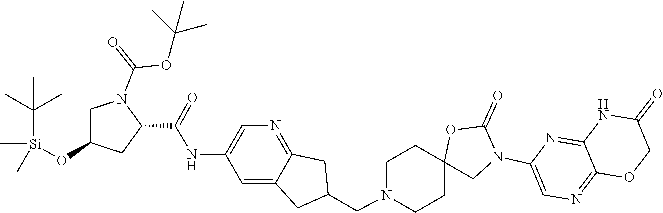 [SMILES: CC(C)(C)OC(=O)N1C[C@H](O[Si](C)(C)C(C)(C)C)C[C@H]1C(=O)Nc1cnc2c(c1)CC(CN1CCC3(CC1)CN(c1cnc4c(n1)NC(=O)CO4)C(=O)O3)C2]

### Steps 1 to 3: diethyl 3-bromo-5,7-dihydrocyclopenta[b]pyridine-6,6-dicarboxylate

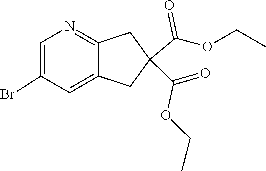 [SMILES: CCOC(=O)C1(C(=O)OCC)Cc2cc(Br)cnc2C1]

The title compound was prepared by analogy to Intermediate 6, Steps 2 to 4 using dimethyl 5-bromopyridine-2,3-dicarboxylate [CAS #521980-82-5] in place of dimethyl 4-methylpyridine-2,3-dicarboxylate and was obtained as a dark brown viscous oil. MS (ESP) m/z=342.1 [M+H]⁺.

### Step 4: 3-bromo-6,7-dihydro-5H-cyclopenta[b]pyridine-6-carboxylic Acid; Sulfuric Acid

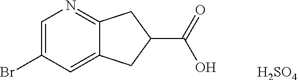

A suspension of diethyl 3-bromo-5,7-dihydrocyclopenta[b]pyridine-6,6-dicarboxylate (245.0 mg, 0.720 mmol, 1 eq) in water (3.6 mL) and sulfuric acid (0.46 mL, 8.63 mmol, 12.05 eq) was stirred at 100° C. for 16 h. The reaction mixture was cooled to r.t. and volatiles were removed under reduced pressure to give the crude title compound (2150 mg) as an orange oil. MS (ESP) m/z=241.2 [M+H]⁺.

### Step 5: ethyl 3-bromo-6,7-dihydro-5H-cyclopenta[b]pyridine-6-carboxylate

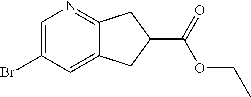

A solution of 3-bromo-6,7-dihydro-5H-cyclopenta[b]pyridine-6-carboxylic acid and sulfuric acid (532.0 mg, 1.56 mmol, 1 eq) in EtOH (20.0 mL, 354.24 mmol, 226.5 eq) was stirred at 85° C. for 21 h. The reaction mixture was cooled to r.t., volatiles were removed under reduced pressure, the residue was poured into NaHCO₃ (sat. aq.) and stirred at r.t. for 15 min. The aqueous layer was extracted with EtOAc (×3). The combined organic phases were filtered through a phase separator and volatiles removed under reduced pressure to give the title compound (390 mg, 1.44 mmol, 92.31% yield) as a brown viscous oil. MS (ESP) m/z=270.0 [M+H]⁺.

### Step 6: ethyl 3-(tert-butoxycarbonylamino)-6,7-dihydro-5H-cyclopenta[b]pyridine-6-carboxylate

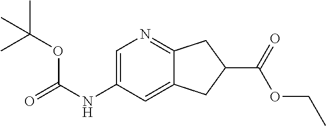 [SMILES: CCOC(=O)C1Cc2cc(NC(=O)OC(C)(C)C)cnc2C1]

In a sealed tube, a suspension of ethyl 3-bromo-6,7-dihydro-5H-cyclopenta[b]pyridine-6-carboxylate (390.0 mg, 1.44 mmol, 1 eq), tert-butyl carbamate (676.57 mg, 5.78 mmol, 4 eq), xantphos (167.09 mg, 0.290 mmol, 0.200 eq), tris(dibenzylideneacetone)dipalladium (0) (132.21 mg, 0.140 mmol, 0.100 eq) and cesium carbonate (1411.26 mg, 4.33 mmol, 3 eq) in 1,4-dioxane (14.1 mL) was purged three times with vacuum/nitrogen atmosphere. The reaction mixture was shaken at 100° C. for 1 h. The reaction mixture was diluted with EtOAc and water, the phases were separated and the aqueous layer was extracted with EtOAc (×3). The combined organic phases were filtered through a phase separator and volatiles were removed under reduced pressure to afford a residue which was purified by silica cartridge chromatography (50 g, from cyclohexane 100% to cyclohexane/EtOAc 6:4) to give the title compound (316 mg, 1.03 mmol, 71.44% yield) as an orange solid. MS (ESP) m/z=307.2 [M+H]⁺.

### Steps 7 to 10: 6-[8-[(3-amino-6,7-dihydro-5H-cyclopenta[b]pyridin-6-yl)methyl]-2-oxo-1-oxa-3,8-diazaspiro[4.5]decan-3-yl]-4H-pyrazino[2,3-b][1,4]oxazin-3-one

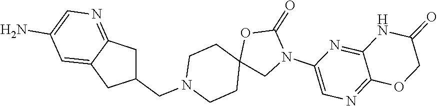 [SMILES: Nc1cnc2c(c1)CC(CN1CCC3(CC1)CN(c1cnc4c(n1)NC(=O)CO4)C(=O)O3)C2]

The title compound was prepared by analogy to Intermediate 52, Steps 6 to 9 and was obtained as an off-white solid. MS (ESP) m/z=452.3 [M+H]⁺.

### Step 11: tert-butyl (2S,4R)-4-[tert-butyl(dimethyl)silyl]oxy-2-[[6-[[2-oxo-3-(3-oxo-4H-pyrazino[2,3-b][1,4]oxazin-6-yl)-1-oxa-3,8-diazaspiro[4.5]decan-8-yl]methyl]-6,7-dihydro-5H-cyclopenta[b]pyridin-3-yl]carbamoyl]pyrrolidine-1-carboxylate

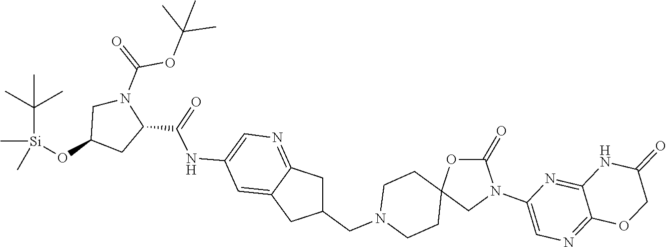 [SMILES: CC(C)(C)OC(=O)N1C[C@H](O[Si](C)(C)C(C)(C)C)C[C@H]1C(=O)Nc1cnc2c(c1)CC(CN1CCC3(CC1)CN(c1cnc4c(n1)NC(=O)CO4)C(=O)O3)C2]

A solution of 6-[8-[(3-amino-6,7-dihydro-5H-cyclopenta[b]pyridin-6-yl)methyl]-2-oxo-1-oxa-3,8-diazaspiro[4.5]decan-3-yl]-4H-pyrazino[2,3-b][1,4]oxazin-3-one (40.0 mg, 0.090 mmol, 1 eq), 1-ethyl-3-(3-dimethylaminopropyl)carbodiimide hydrochloride (30.57 mg, 0.160 mmol, 1.8 eq), (2S,4R)-1-tert-butoxycarbonyl-4-[tert-butyl(dimethyl)silyl]oxy-pyrrolidine-2-carboxylic acid (Intermediate 38, Step 1, 45.92 mg, 0.130 mmol, 1.5 eq), HATU (60.64 mg, 0.160 mmol, 1.8 eq) and N,N-diisopropylethylamine (46.3 uL, 0.270 mmol, 3 eq) in DCM (0.500 mL) and DMF (0.500 mL) was stirred at r.t. for 2 h. The reaction mixture was diluted with EtOAc and NaHCO₃ (sat. aq.). The phases were separated and the organic layer was washed with NaHCO₃ (sat. aq.) (×2) and brine. The organic phase was filtered through a phase separator and volatiles were removed under reduced pressure to afford a residue which was purified by silica cartridge chromatography (5 g silica-NH, from DCM 100% to DCM/[DCM/MeOH 9:1]75:25) to give the title compound (30 mg, 0.040 mmol, 43.47% yield) as a colourless oil. MS (ESP) m/z=779.6 [M+H]⁺.

### Intermediate 57

### tert-Butyl (2S,4R)-2-[[2-[[tert-butoxycarbonyl-[2-[2-oxo-3-(3-oxo-4H-pyrazino[2,3-b][1,4]oxazin-6-yl)oxazolidin-5-yl]ethyl]amino]methyl]-7-cyano-indan-5-yl]carbamoyl]-4-[tert-butyl(dimethyl)silyl]oxy-pyrrolidine-1-carboxylate

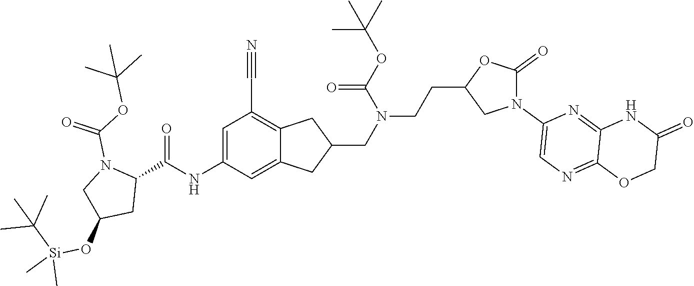 [SMILES: CC(C)(C)OC(=O)N(CCC1CN(c2cnc3c(n2)NC(=O)CO3)C(=O)O1)CC1Cc2cc(NC(=O)[C@@H]3C[C@@H](O[Si](C)(C)C(C)(C)C)CN3C(=O)OC(C)(C)C)cc(C#N)c2C1]

### Steps 1 and 2: 2-formyl-6-nitro-indane-4-carbonitrile

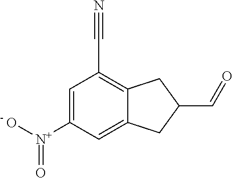

The title compound was prepared from ethyl 4-cyano-6-nitro-indane-2-carboxylate (Intermediate 24, Step 2) by analogy to Intermediate 22, Steps 2 and 3 and was obtained as a light yellow oil. MS (ESN) m/z=215.0 [M−H]⁻.

### Step 3: 6-nitro-2-[[2-[2-oxo-3-(3-oxo-4H-pyrazino[2,3-b][1,4]oxazin-6-yl)oxazolidin-5-yl]ethylamino]methyl]indane-4-carbonitrile

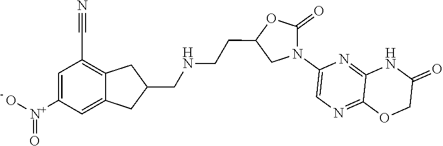

The title compound was prepared from 6-[5-(2-aminoethyl)-2-oxo-oxazolidin-3-yl]-4H-pyrazino[2,3-b][1,4]oxazin-3-one; 2,2,2-trifluoroacetic acid (Intermediate 1) and 2-formyl-6-nitro-indane-4-carbonitrile by analogy to Intermediate 51, Step 3 and was obtained as a light brown foam. MS (ESP) m/z=480.3 [M+H]⁺.

### Step 4: tert-butyl N-[(4-cyano-6-nitro-indan-2-yl)methyl]-N-[2-[2-oxo-3-(3-oxo-4H-pyrazino[2,3-b][1,4]oxazin-6-yl)oxazolidin-5-yl]ethyl]carbamate

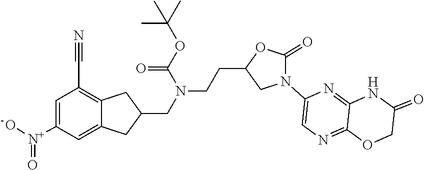

The title compound was prepared by analogy to Intermediate 51, Step 1 and was obtained as a light brown foam. MS (ESP) m/z=580.4 [M+H]⁺.

### Step 5: tert-butyl N-[(6-amino-4-cyano-indan-2-yl)methyl]-N-[2-[2-oxo-3-(3-oxo-4H-pyrazino[2,3-b][1,4]oxazin-6-yl)oxazolidin-5-yl]ethyl]carbamate

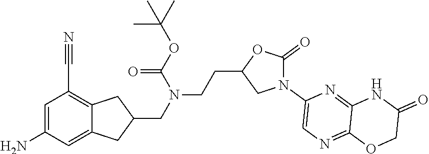 [SMILES: CC(C)(C)OC(=O)N(CCC1CN(c2cnc3c(n2)NC(=O)CO3)C(=O)O1)CC1Cc2cc(N)cc(C#N)c2C1]

The title compound was prepared by analogy to Intermediate 24, Step 3 and was obtained as a light yellow foam. MS (ESN) m/z=548.4 [M−H]⁻.

### Step 6: tert-Butyl (2S,4R)-2-[[2-[[tert-butoxycarbonyl-[2-[2-oxo-3-(3-oxo-4H-pyrazino[2,3-b][1,4]oxazin-6-yl)oxazolidin-5-yl]ethyl]amino]methyl]-7-cyano-indan-5-yl]carbamoyl]-4-[tert-butyl(dimethyl)silyl]oxy-pyrrolidine-1-carboxylate

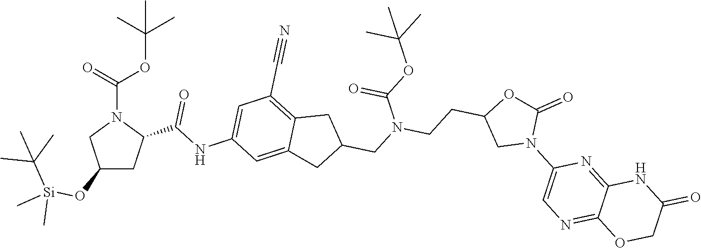 [SMILES: CC(C)(C)OC(=O)N(CCC1CN(c2cnc3c(n2)NC(=O)CO3)C(=O)O1)CC1Cc2cc(NC(=O)[C@@H]3C[C@@H](O[Si](C)(C)C(C)(C)C)CN3C(=O)OC(C)(C)C)cc(C#N)c2C1]

The title compound was prepared from tert-butyl N-[(6-amino-4-cyano-indan-2-yl)methyl]-N-[2-[2-oxo-3-(3-oxo-4H-pyrazino[2,3-b][1,4]oxazin-6-yl)oxazolidin-5-yl]ethyl]carbamate and (2S,4R)-1-tert-butoxycarbonyl-4-[tert-butyl(dimethyl)silyl]oxy-pyrrolidine-2-carboxylic acid (Intermediate 38, Step 1) by analogy to Intermediate 7, Step 1 and was obtained as a white solid. MS (ESN) m/z=875.8 [M−H]⁻.

### Intermediate 58

### tert-Butyl (2S,4R)-4-[tert-butyl(dimethyl)silyl]oxy-2-[[7-fluoro-2-[[methyl-[2-[2-oxo-3-(3-oxo-4H-pyrazino[2,3-b][1,4]oxazin-6-yl)oxazolidin-5-yl]ethyl]amino]methyl]indan-5-yl]carbamoyl]pyrrolidine-1-carboxylate

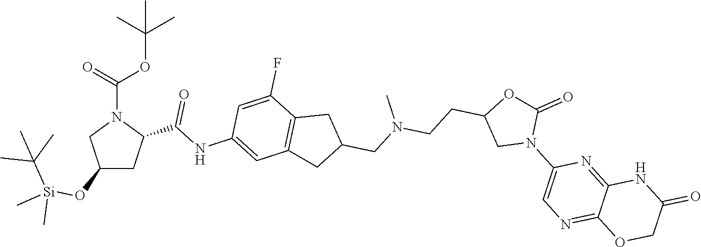 [SMILES: CN(CCC1CN(c2cnc3c(n2)NC(=O)CO3)C(=O)O1)CC1Cc2cc(NC(=O)[C@@H]3C[C@@H](O[Si](C)(C)C(C)(C)C)CN3C(=O)OC(C)(C)C)cc(F)c2C1]

### Step 1: tert-butyl (2S,4R)-4-[tert-butyl(dimethyl)silyl]oxy-2-[[7-fluoro-2-[[2-[2-oxo-3-(3-oxo-4H-pyrazino[2,3-b][1,4]oxazin-6-yl)oxazolidin-5-yl]ethylamino]methyl]indan-5-yl]carbamoyl]pyrrolidine-1-carboxylate

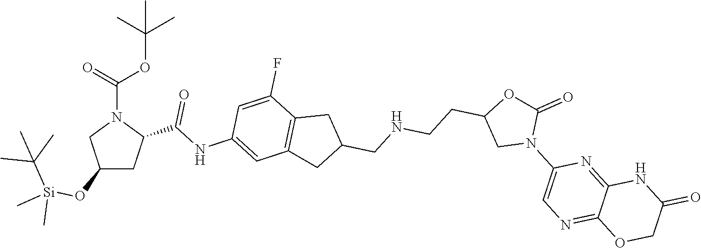 [SMILES: CC(C)(C)OC(=O)N1C[C@H](O[Si](C)(C)C(C)(C)C)C[C@H]1C(=O)Nc1cc(F)c2c(c1)CC(CNCCC1CN(c3cnc4c(n3)NC(=O)CO4)C(=O)O1)C2]

The title compound was prepared by analogy with Example 1 using tert-butyl (2S,4R)-4-[tert-butyl(dimethyl)silyl]oxy-2-[(7-fluoro-2-formyl-indan-5-yl)carbamoyl]pyrrolidine-1-carboxylate (Intermediate 38) in place of 2-(dimethylamino)-N-(7-fluoro-2-formyl-indan-5-yl)acetamide. MS (ESP) m/z=770.6 [M+H]⁺.

### Step 2: tert-butyl (2S,4R)-4-[tert-butyl(dimethyl)silyl]oxy-2-[[7-fluoro-2-[[methyl-[2-[2-oxo-3-(3-oxo-4H-pyrazino[2,3-b][1,4]oxazin-6-yl)oxazolidin-5-yl]ethyl]amino]methyl]indan-5-yl]carbamoyl]pyrrolidine-1-carboxylate

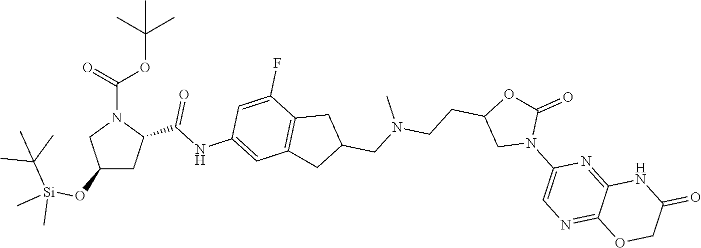 [SMILES: CN(CCC1CN(c2cnc3c(n2)NC(=O)CO3)C(=O)O1)CC1Cc2cc(NC(=O)[C@@H]3C[C@@H](O[Si](C)(C)C(C)(C)C)CN3C(=O)OC(C)(C)C)cc(F)c2C1]

The title compound was prepared by analogy to Intermediate 21, Step 3 and was obtained as a light yellow oil. MS (ESP) m/z=784.6 [M+H]⁺.

### Intermediate 59

### (2R)—N-[3-[tert-Butyl(dimethyl)silyl]oxy-7-fluoro-2-formyl-indan-5-yl]-2-(dimethylamino)propanamide

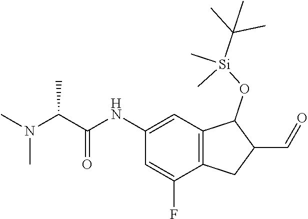 [SMILES: C[C@H](C(=O)Nc1cc(F)c2c(c1)C(O[Si](C)(C)C(C)(C)C)C(C=O)C2)N(C)C]

### Step 1: ethyl 6-bromo-4-fluoro-1-oxo-indane-2-carboxylate

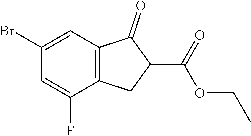 [SMILES: CCOC(=O)C1Cc2c(F)cc(Br)cc2C1=O]

The title compound was prepared by analogy to Intermediate 5, Step 1 using 6-bromo-4-fluoro-indan-1-one [CAS #881189-74-8] in place of 4-fluoro-indan-1-one and was obtained as a pink solid. MS (ESP) m/z=301.0 [M+H]⁺.

### Step 2: ethyl 6-(tert-butoxycarbonylamino)-4-fluoro-1-oxo-indane-2-carboxylate

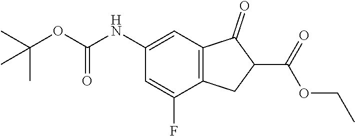 [SMILES: CCOC(=O)C1Cc2c(F)cc(NC(=O)OC(C)(C)C)cc2C1=O]

The title compound was prepared by analogy to Intermediate 56, Step 6 and was obtained as a light yellow solid. MS (ESP) m/z=338.1 [M+H]⁺.

### Step 3: ethyl 6-(tert-butoxycarbonylamino)-4-fluoro-1-hydroxy-indane-2-carboxylate

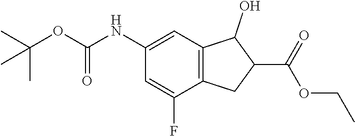 [SMILES: CCOC(=O)C1Cc2c(F)cc(NC(=O)OC(C)(C)C)cc2C1O]

The title compound was prepared by analogy to Intermediate 36, Step 3 and was obtained as a pale yellow oil. MS (ESN) m/z=338.2 [M−H]⁻.

### Step 4: ethyl 6-amino-4-fluoro-1-hydroxy-indane-2-carboxylate

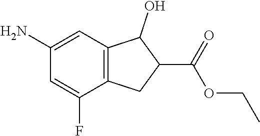 [SMILES: CCOC(=O)C1Cc2c(F)cc(N)cc2C1O]

The title compound was prepared by analogy to Intermediate 35, Step 5 and was obtained as an off-white solid. MS (ESP) m/z=240.1 [M+H]⁺.

### Step 5: ethyl 6-amino-1-[tert-butyl(dimethyl)silyl]oxy-4-fluoro-indane-2-carboxylate

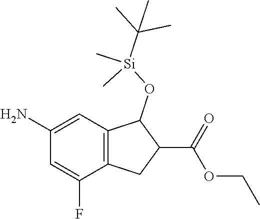 [SMILES: CCOC(=O)C1Cc2c(F)cc(N)cc2C1O[Si](C)(C)C(C)(C)C]

A suspension of ethyl 6-amino-4-fluoro-1-hydroxy-indane-2-carboxylate (275.0 mg, 1.15 mmol, 1 eq), tert-butyldimethylchlorosilane (346.5 mg, 2.3 mmol, 2 eq), imidazole (172.16 mg, 2.53 mmol, 2.2 eq) and 4-dimethylaminopyridine (14.04 mg, 0.110 mmol, 0.100 eq) in DCM (5.5 mL) and DMA (0.800 mL) was stirred at r.t. for 18 h. The reaction mixture was quenched with NHC₁ (sat. aq.) and diluted with DCM. The phases were separated and the aqueous layer was extracted with DCM. The combined organic phases were filtered through a phase separator and volatiles were removed under reduced pressure to give crude material which was purified by silica cartridge chromatography (25 g, from cyclohexane 100% to cyclohexane/EtOAc 7:3) to give the title compound (369 mg, 1.04 mmol, 90.81% yield) as a light yellow oil. MS (ESP) m/z=354.2 [M+H]⁺.

### Steps 6 and 7: (2R)—N-[3-[tert-butyl(dimethyl)silyl]oxy-7-fluoro-2-(hydroxymethyl)indan-5-yl]-2-(dimethylamino)propanamide

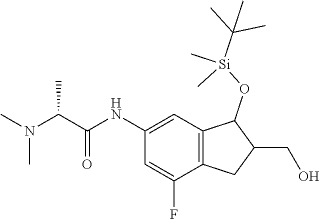 [SMILES: C[C@H](C(=O)Nc1cc(F)c2c(c1)C(O[Si](C)(C)C(C)(C)C)C(CO)C2)N(C)C]

The title compound was prepared by analogy to Intermediate 32, Steps 8 and 9 and was obtained as a light yellow oil. MS (ESP) m/z=411.5 [M+H]⁺.

### Step 8: (2R)—N-[3-[tert-butyl(dimethyl)silyl]oxy-7-fluoro-2-formyl-indan-5-yl]-2-(dimethylamino)propanamide

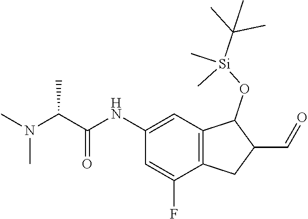 [SMILES: C[C@H](C(=O)Nc1cc(F)c2c(c1)C(O[Si](C)(C)C(C)(C)C)C(C=O)C2)N(C)C]

The title compound was prepared by analogy to Intermediate 7, Step 2 and was obtained as a brown oil. MS (ESP) m/z=409.5 [M+H]⁺.

### Intermediate 60

### (2R)—N-[3,7-Difluoro-2-[[2-[(5R)-2-oxo-3-[3-oxo-4-(2-trimethylsilylethoxymethyl)pyrazino[2,3-b][1,4]oxazin-6-yl]oxazolidin-5-yl]ethylamino]methyl]indan-5-yl]-2-(dimethylamino)propanamide

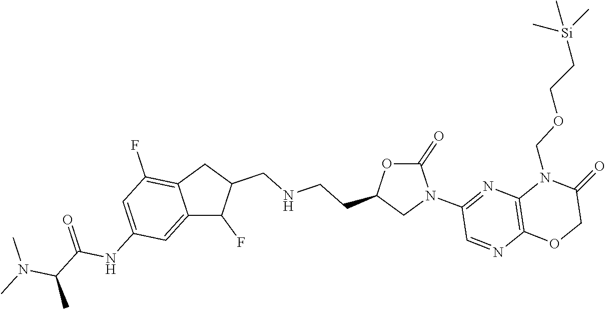 [SMILES: C[C@H](C(=O)Nc1cc(F)c2c(c1)C(F)C(CNCC[C@@H]1CN(c3cnc4c(n3)N(COCC[Si](C)(C)C)C(=O)CO4)C(=O)O1)C2)N(C)C]

### Step 1: ethyl 6-(tert-butoxycarbonylamino)-1,4-difluoro-indane-2-carboxylate

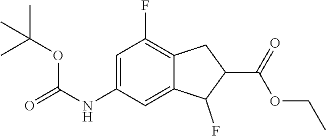 [SMILES: CCOC(=O)C1Cc2c(F)cc(NC(=O)OC(C)(C)C)cc2C1F]

To ethyl 6-(tert-butoxycarbonylamino)-4-fluoro-1-hydroxy-indane-2-carboxylate (Intermediate 59, Step 3, 766.0 mg, 2.03 mmol, 1 eq) in anhydrous DCM (23.93 mL) at −78° C. was added a solution of diethylaminosulfur trifluoride (0.4 mL, 3.05 mmol, 1.5 eq) in anhydrous DCM (8.35 mL) dropwise. The reaction mixture was stirred at −78° C. for 2 h then additional diethylaminosulfur trifluoride (0.13 mL, 0.980 mmol, 0.480 eq) in DCM (8.35 mL) was added dropwise and then the reaction mixture was stirred for 2 h. The mixture was diluted with dichloromethane, washed with cold diluted sodium carbonate solution, filtered through a phase separator and concentrated in vacuo to obtain a crude which was purified by silica cartridge chromatography (EtOAc/cyclohexane 0-20%) to afford the title compound (432 mg, 1.27 mmol, 62.3% yield) as a light yellow foam. MS (ESP) m/z=364.1 [M+Na]⁺.

### Step 2: ethyl 6-amino-1,4-difluoro-indane-2-carboxylate

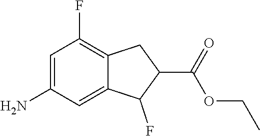 [SMILES: CCOC(=O)C1Cc2c(F)cc(N)cc2C1F]

The title compound was prepared by analogy to Intermediate 35, Step 5 and was obtained as a light yellow oil. MS (ESP) m/z=242.1 [M+H]⁺.

### Steps 3 and 4: (2R)—N-[3,7-difluoro-2-(hydroxymethyl)indan-5-yl]-2-(dimethylamino)propanamide

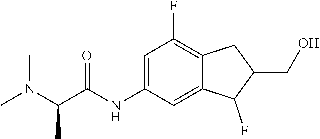 [SMILES: C[C@H](C(=O)Nc1cc(F)c2c(c1)C(F)C(CO)C2)N(C)C]

The title compound was prepared by analogy to Intermediate 32, Steps 8 and 9 and was obtained as a light yellow solid. MS (ESP) m/z=299.1 [M+H]⁺.

### Step 5: [6-[[(2R)-2-(Dimethylamino)propanoyl]amino]-1,4-difluoro-indan-2-yl]methyl Methanesulfonate

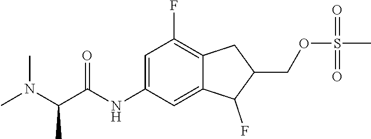

To a solution of (2R)—N-[3,7-difluoro-2-(hydroxymethyl)indan-5-yl]-2-(dimethylamino)propanamide (72.0 mg, 0.240 mmol, 1 eq) in DCM (2.41 mL) at 0° C. was added methanesulfonyl chloride (0.02 mL, 0.250 mmol, 1.05 eq) followed by triethylamine (0.04 mL, 0.310 mmol, 1.3 eq) and the mixture stirred for 3 h allowing the temperature to rise. The reaction mixture was cooled again to 0° C., further methanesulfonyl chloride (0.0 mL, 0.020 mmol, 0.100 eq) was added and the mixture stirred for 30 min. Then the reaction mixture was diluted with DCM and poured into a cold NaHCO₃ solution (sat. aq.). The phases were separated, the aqueous phase was extracted with DCM (×3). The combined organic phases were filtered through a phase separator, concentrated under reduced pressure to obtain a crude which was purified by silica cartridge chromatography (5 g, methanol/DCM from 0 to 20%) to give the title compound (81.5 mg, 0.200 mmol, 89.71% yield) as a light green solid. MS (ESP) m/z=377.3 [M+H]⁺.

### Step 6: (2R)—N-[3,7-Difluoro-2-(iodomethyl)indan-5-yl]-2-(dimethylamino)propanamide

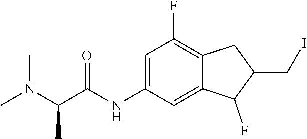 [SMILES: C[C@H](C(=O)Nc1cc(F)c2c(c1)C(F)C(CI)C2)N(C)C]

[6-[[(2R)-2-(dimethylamino)propanoyl]amino]-1,4-difluoro-indan-2-yl]methyl methanesulfonate (71.0 mg, 0.190 mmol, 1 eq) was dissolved in MeCN (0.472 mL), then sodium iodide (282.72 mg, 1.89 mmol, 10 eq) was added and the mixture stirred at 60° C. for 18 h. Then the reaction was cooled to RT and water was added. The mixture was slightly basified with NaHCO₃ (sat. aq.) and was extracted with EtOAc (×3). The combined organic extracts were dried through a phase separator, and concentrated under reduced pressure to afford a residue which was purified by silica cartridge chromatography (5 g, 95:5 DCM/MeOH) to afford the title compound (42.3 mg, 0.100 mmol, 54.94% yield) as a light brown oil. MS (ESP) m/z=409.3 [M+H]⁺.

### Step 7: (2R)—N-[3,7-difluoro-2-[[2-[(5R)-2-oxo-3-[3-oxo-4-(2-trimethylsilylethoxymethyl)pyrazino[2,3-b][1,4]oxazin-6-yl]oxazolidin-5-yl]ethylamino]methyl]indan-5-yl]-2-(dimethylamino)propanamide

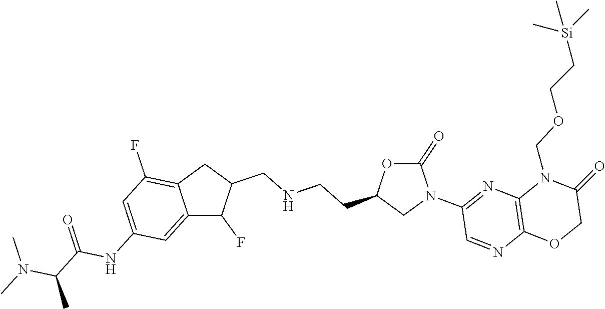 [SMILES: C[C@H](C(=O)Nc1cc(F)c2c(c1)C(F)C(CNCC[C@@H]1CN(c3cnc4c(n3)N(COCC[Si](C)(C)C)C(=O)CO4)C(=O)O1)C2)N(C)C]

6-[(5R)-5-(2-Aminoethyl)-2-oxo-oxazolidin-3-yl]-4-(2-trimethylsilylethoxymethyl)pyrazino[2,3-b][1,4]oxazin-3-one; hydrochloride (Intermediate 61, 45.88 mg, 0.100 mmol, 1 eq) and (2R)—N-[3,7-difluoro-2-(iodomethyl)indan-5-yl]-2-(dimethylamino)propanamide (42.0 mg, 0.100 mmol, 1 eq) were mixed with MeCN (0.935 mL), then potassium carbonate (35.55 mg, 0.260 mmol, 2.5 eq) was added. The mixture was stirred for 3 days at 80° C. Then the reaction mixture was cooled, filtered through a buchner funnel washing with MeCN. The filtrate was concentrated under reduced pressure, the crude product was purified by reverse phase silica cartridge chromatography (MeCN/(H₂O+0.1% NH₄OH), 1 to 75%) followed by lyophilization to give the title compound (18.9 mg, 0.030 mmol, 26.63% yield) as an off-white solid. MS (ESP) m/z=690.4 [M+H]⁺.

### Intermediate 61

### 6-[(5R)-5-(2-Aminoethyl)-2-oxo-oxazolidin-3-yl]-4-(2-trimethylsilylethoxymethyl)pyrazino[2,3-b][1,4]oxazin-3-one; Hydrochloride

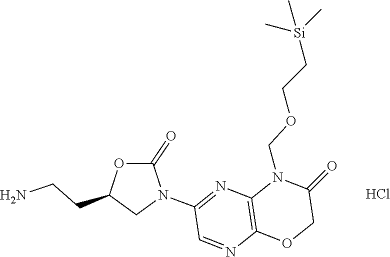 [SMILES: C[Si](C)(C)CCOCN1C(=O)COc2ncc(N3C[C@@H](CCN)OC3=O)nc21 ; Cl]

### Step 1: tert-Butyl (R)-(2-(2-oxo-3-(3-oxo-4-((2-(trimethylsilyl)ethoxy)methyl)-3,4-dihydro-2H-pyrazino[2,3-b][1,4]oxazin-6-yl)oxazolidin-5-yl)ethyl)carbamate

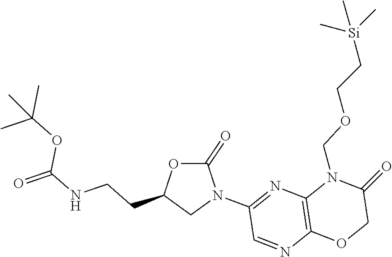 [SMILES: CC(C)(C)OC(=O)NCC[C@@H]1CN(c2cnc3c(n2)N(COCC[Si](C)(C)C)C(=O)CO3)C(=O)O1]

The enantiomers of tert-butyl N-[2-[2-oxo-3-[3-oxo-4-(2-trimethylsilylethoxymethyl)pyrazino[2,3-b][1,4]oxazin-6-yl]oxazolidin-5-yl]ethyl]carbamate (Intermediate 1, Step 3) were separated by preparative chiral SFC to give the title compound as a white solid. MS (ESN) m/z=554.4 [M+HCOO]⁺.

### Step 2: 6-[(5R)-5-(2-aminoethyl)-2-oxo-oxazolidin-3-yl]-4-(2-trimethylsilylethoxymethyl)pyrazino[2,3-b][1,4]oxazin-3-one; hydrochloride

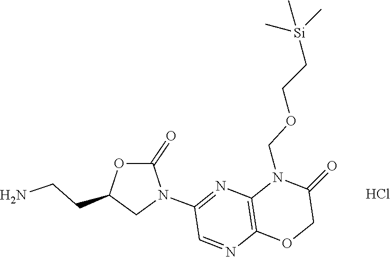 [SMILES: C[Si](C)(C)CCOCN1C(=O)COc2ncc(N3C[C@@H](CCN)OC3=O)nc21 ; Cl]

tert-Butyl (R)-(2-(2-oxo-3-(3-oxo-4-((2-(trimethylsilyl)ethoxy)methyl)-3,4-dihydro-2H-pyrazino[2,3-b][1,4]oxazin-6-yl)oxazolidin-5-yl)ethyl)carbamate (5.40 g, 10.6 mmol, 1 eq) was dissolved in 1,1,1,3,3,3-hexafluoro-2-propanol (75 ml, 10.6 mmol, 1 eq) and the mixture irradiated in a microwave for 90 minutes at 145° C. The mixture was cooled to 0° C. and HCl in water 37% (1.1 g, 929 μl, 11.1 mmol, 1.05 eq) was added. The mixture was evaporated at 25° C. The residue was dissolved in MeCN (5 mL), water (150 mL) was added and the mixture lyophilized to give the title compound (4.52 g, 10.1 mmol, 95.7% yield) as a white solid. MS (ESP) m/z=410.3 [M+H]⁺.

### Intermediate 62

### 6-[(5R)-5-(2-Aminoethyl)-2-oxo-oxazolidin-3-yl]-4H-pyrazino[2,3-b][1,4]oxazin-3-one; 2,2,2-trifluoroacetic Acid

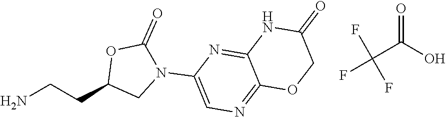

### Step 1: 2-(2-oxooxazolidin-5-yl)ethyl Methanesulfonate

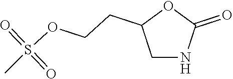

To a mixture of 5-(2-hydroxyethyl)oxazolidin-2-one [CAS #1211594-02-3] (25.0 g, 190.65 mmol, 1 eq) and DCM (250 mL) was added triethylamine (70.0 mL, 502.22 mmol, 2.63 eq). The mixture was cooled below 10° C. with an ice bath. Methanesulfonyl chloride (20.0 mL, 258.4 mmol, 1.36 eq) was added dropwise to the stirred mixture keeping the internal temperature below 20° C. After addition, the mixture was stirred at RT for 30 min. The mixture was filtered on a phase separator and the residue was washed with DCM (100 mL). To the stirred filtrate was added NaHCO₃ (15 g), followed by water (100 mL). The organic phase was separated with a phase separator and concentrated under reduced pressure to afford the title compound (28.3 g, 135.26 mmol, 70.95% yield) as a yellow oil. The product was used in the following step without further purification. ¹H NMR (400 MHz, chloroform-d) δ 6.09 (s, 1H), 4.89-4.76 (m, 1H), 4.46-4.36 (m, 2H), 3.77 (t, J=8.6 Hz, 1H), 3.12 (q, J=7.3 Hz, 1H), 3.05 (s, 3H), 2.17 (dtd, J=7.5, 5.4, 1.9 Hz, 2H).

### Step 2: tert-butyl N-tert-butoxycarbonyl-N-[2-(2-oxooxazolidin-5-yl)ethyl]carbamate

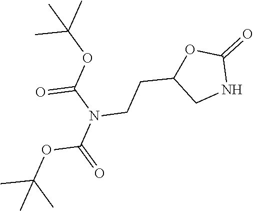 [SMILES: CC(C)(C)OC(=O)N(CCC1CNC(=O)O1)C(=O)OC(C)(C)C]

A 1 L round bottom flask was charged with a solution of di-tert-butyl iminodicarboxylate (31.0 g, 142.7 mmol, 0.890 eq) in DMF (240 mL). The mixture was cooled to 0° C. and potassium tert-butoxide (15.91 g, 141.76 mmol, 0.880 eq) was added. The mixture was stirred at this temperature for 15 min. A solution of 2-(2-oxooxazolidin-5-yl)ethyl methanesulfonate (33.7 g, 161.07 mmol, 1 eq) in DMF (60 mL) was added dropwise while keeping the internal temperature below 5° C., then the mixture was allowed to warm to RT and stirred at this temperature for 16 h. The mixture was cooled with an ice bath and NH₄Cl (sat. aq., 500 mL) was added. The mixture was extracted with EtOAc (3×200 mL), then the combined organic phases were washed with water (300 mL) and brine (300 mL), dried over Na₂SO₄, filtered and concentrated under reduced pressure. The brown oily residue was purified by silica cartridge chromatography (340 g, EtOAc/cyclohexane 25 to 100%) to give the title compound (18 g, 54.48 mmol, 33.82% yield) as a white solid.

### Step 3: tert-butyl N-tert-butoxycarbonyl-N-[2-[(5R)-2-oxo-3-[3-oxo-4-(2-trimethylsilylethoxymethyl)pyrazino[2,3-b][1,4]oxazin-6-yl]oxazolidin-5-yl]ethyl]carbamate and tert-butyl N-tert-butoxycarbonyl-N-[2-[(5S)-2-oxo-3-[3-oxo-4-(2-trimethylsilylethoxymethyl)pyrazino[2,3-b][1,4]oxazin-6-yl]oxazolidin-5-yl]ethyl]carbamate

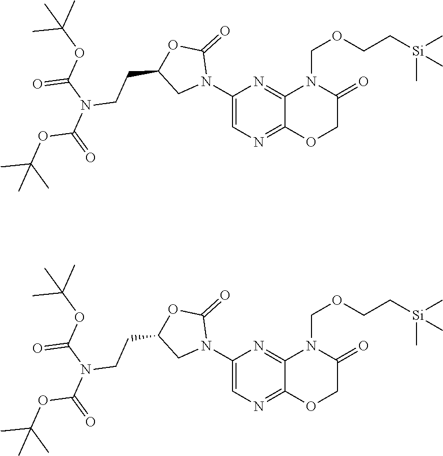

Racemic tert-butyl N-tert-butoxycarbonyl-N-[2-[2-oxo-3-[3-oxo-4-(2-trimethylsilylethoxymethyl)pyrazino[2,3-b][1,4]oxazin-6-yl]oxazolidin-5-yl]ethyl]carbamate was prepared by analogy to Intermediate 1, Step 3 using tert-butyl N-tert-butoxycarbonyl-N-[2-(2-oxooxazolidin-5-yl)ethyl]carbamate in place of tert-butyl N-[2-(2-oxooxazolidin-5-yl)ethyl]carbamate and was obtained as a yellow oil. The enantiomers of tert-butyl N-tert-butoxycarbonyl-N-[2-[2-oxo-3-[3-oxo-4-(2-trimethylsilylethoxymethyl)pyrazino[2,3-b][1,4]oxazin-6-yl]oxazolidin-5-yl]ethyl]carbamate (20 g, 32.8 mmol, 1 eq) were separated by preparative chiral SFC to give peak 1, tert-butyl N-tert-butoxycarbonyl-N-[2-[(5R)-2-oxo-3-[3-oxo-4-(2-trimethylsilylethoxymethyl)pyrazino[2,3-b][1,4]oxazin-6-yl]oxazolidin-5-yl]ethyl]carbamate (7840 mg, 38.02%) as a light brown solid and peak 2, tert-butyl N-tert-butoxycarbonyl-N-[2-[(5S)-2-oxo-3-[3-oxo-4-(2-trimethylsilylethoxymethyl)pyrazino[2,3-b][1,4]oxazin-6-yl]oxazolidin-5-yl]ethyl]carbamate (8480 mg, 39.86%) as a light brown solid.

### Step 4: 6-[(5R)-5-(2-aminoethyl)-2-oxo-oxazolidin-3-yl]-4H-pyrazino[2,3-b][1,4]oxazin-3-one; 2,2,2-trifluoroacetic Acid

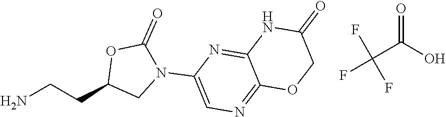

A solution of tert-butyl N-tert-butoxycarbonyl-N-[2-[(5R)-2-oxo-3-[3-oxo-4-(2-trimethylsilylethoxymethyl)pyrazino[2,3-b][1,4]oxazin-6-yl]oxazolidin-5-yl]ethyl]carbamate (2.0 g, 3.28 mmol, 1 eq) in trifluoroacetic acid (18.75 mL, 243.38 mmol, 74.2 eq) was stirred at 60° C. overnight. The mixture was concentrated under vacuum and evaporated with DCM and MeCN. The resulting material was titered in MeCN. The solid was collected and dried under vacuum to give the title compound (827 mg, 2.1 mmol, 64.11% yield) as an off-white solid. MS (ESP) m/z=280.3 [M+H]⁺.

### Intermediate 63

### (2S,4R)-1-tert-Butoxycarbonyl-4-[tert-butyl(dimethyl)silyl]oxy-2-methyl-pyrrolidine-2-carboxylic Acid

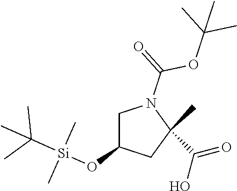 [SMILES: CC(C)(C)OC(=O)N1C[C@H](O[Si](C)(C)C(C)(C)C)C[C@@]1(C)C(=O)O]

### Step 1: O1-tert-butyl O2-methyl (2S,4R)-4-[tert-butyl(dimethyl)silyl]oxypyrrolidine-1,2-dicarboxylate

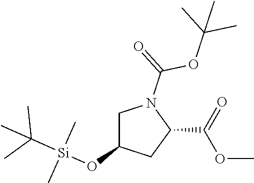 [SMILES: COC(=O)[C@@H]1C[C@@H](O[Si](C)(C)C(C)(C)C)CN1C(=O)OC(C)(C)C]

To a solution of tert-butyldimethylchlorosilane (3687 mg, 24.46 mmol, 1.2 eq) in DMF (20 mL) was added 4-dimethylaminopyridine (249 mg, 2.04 mmol, 0.1 eq), imidazole (3608 mg, 53.0 mmol, 2.6 eq) and N—BOC-trans-4-hydroxy-L-proline methyl ester (5.0 g, 20.39 mmol, 1.0 eq). The resulting mixture was stirred at 25° C. for 16 h. To the mixture was added water (50 mL). The mixture was extracted with EtOAc (50 mL×3). The organic layer was combined, dried over Na₂SO₄ and concentrated under reduced pressure to give a crude product which was purified by flash column chromatography (PE:EtOAc=5:1) to give the title compound (6.9 g, 19.19 mmol, 88.9% yield) as a colorless oil.

### Step 2: O1-tert-butyl O2-methyl (2S,4R)-4-[tert-butyl(dimethyl)silyl]oxy-2-methyl-pyrrolidine-1,2-dicarboxylate

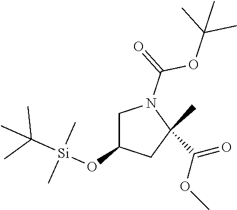 [SMILES: COC(=O)[C@]1(C)C[C@@H](O[Si](C)(C)C(C)(C)C)CN1C(=O)OC(C)(C)C]

To a solution of lithium diisopropylamide (9.6 mL, 19.19 mmol, 1.0 eq) in THF (20 mL) at 0° C. was added hexamethylphosphoramide (3.34 mL, 19.19 mmol, 1.0 eq) at −25° C., then to the solution was added a solution of 01-tert-butyl 02-methyl (2S,4R)-4-[tert-butyl(dimethyl)silyl]oxypyrrolidine-1,2-dicarboxylate (6.9 g, 19.19 mmol, 1.0 eq) in THF (20 mL). After addition, the solution was stirred for 1 h at 0° C. After 1 h, the solution was cooled to −78° C. To the solution was added dropwise iodomethane (4086.11 mg, 28.79 mmol, 1.5 eq), then the solution was stirred at −78° C. for 2 h. The solution was poured into an ice-cold aqueous solution of NH₄Cl. Then the solution was extracted with EtOAc (100 mL×3). The combined organic layers were washed with saturated salt solution and dried over Na₂SO₄. The solution was concentrated under reduced pressure to give a crude. The crude was purified by flash column chromatography (PE:EtOAc=5:1) to give the title compound (2.6 g, 6.96 mmol, 32.6% yield) as a colorless oil.

### Step 3: (2S,4R)-1-tert-butoxycarbonyl-4-[tert-butyl(dimethyl)silyl]oxy-2-methyl-pyrrolidine-2-carboxylic Acid

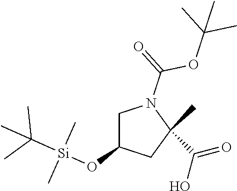 [SMILES: CC(C)(C)OC(=O)N1C[C@H](O[Si](C)(C)C(C)(C)C)C[C@@]1(C)C(=O)O]

To a solution of O1-tert-butyl O2-methyl (2S,4R)-4-[tert-butyl(dimethyl)silyl]oxy-2-methyl-pyrrolidine-1,2-dicarboxylate (600.0 mg, 1.61 mmol, 1.0 eq) in methanol (6 mL) and water (2 mL) was added lithium hydroxide monohydrate (192.34 mg, 4.58 mmol, 2.85 eq) at 20° C. The resulting solution was stirred at 60° C. for 12 h. The reaction mixture pH was adjusted to 3 with 1M aqueous HCl. EtOAc (20 mL) and water (20 mL) were added and layers were separated. The aqueous phase was extracted with EtOAc (20 mL×2). Combined extracts were washed with brine (20 mL×2), dried over Na₂SO₄, filtered, and concentrated under vacuum to give a residue. The residue was purified by preparative TLC (eluent PE/EtOAc=1:1) to give the title compound (150.0 mg, 0.42 mmol, 23.4% yield) as a yellow oil. ¹H NMR (400 MHz, d6-DMSO) δ 12.47 (br s, 1H), 4.41 (m, 1H), 3.52 (m, 1H), 3.25 (m, 1H), 2.26 (m, 1H), 1.83 (m, 1H), 1.52 (s, 3H), 1.37 (s, 9H), 0.86 (s, 9H), 0.06 (s, 6H) ppm.

1. A compound of formula (I) 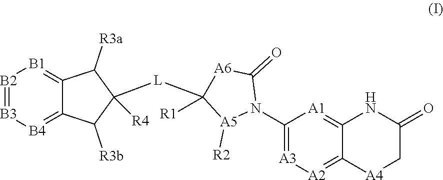
wherein
A¹ is —N—;
A² is selected from
i) —N—, and
ii) —CH—;
A³ is —CH—;
A⁴ is selected from
i) —O—, and
ii) —S—;
A⁵ is —CH—;
A⁶ is —O—;
B¹ is selected from
i) —N—, and
ii) —CR⁵—;
B² is selected from
i) —N—, and
ii) —CR⁶—;
B³ is —CNH—C(═O)R⁷—;
B⁴ is selected from
i) —N—, and
ii) —CR⁸—;
R² is H;
R^{3a} is selected from
i) H,
ii) halogen, and
iii) OH;
R^{3b} is selected from
i) H,
ii) halogen, and
iii) OH;
wherein at least one of R^{3a} and R^{3b} is H;
R⁴ is selected from
i) H,
ii) halogen,
iii) C₁₋₆-alkoxy, and
iii) OH;
R⁵ is selected from
i) H,
ii) halogen,
iii) cyano, and
iv) C₁₋₆-alkyl;
R⁶ is selected from
i) H, and
ii) C₁₋₆-alkyl;
R⁷ is selected from
i) CR^{a}R^{b}NR^{c}R^{d},
ii) CR^{a}R^{b}OH,
iii) CHR^{e}R^{f},
iv) heterocycloalkyl,
v) heterocycloalkylalkyl,
vi) substituted heterocycloalkyl,
vii) substituted heterocycloalkylalkyl, and
vii) substituted heteroaryl,
wherein substituted heterocycloalkyl, substituted heterocycloalkylalkyl or substituted heteroaryl is substituted by 1 to 2 substituents independently selected from C₁₋₆-alkyl, C₁₋₆-alkoxy, OH, hydroxyalkyl, amino, and dialkylamino, or two substituents together form a 3-to-6-membered cycloalkyl ring;
R⁸ is selected from
i) H,
ii) halogen, and
iii) cyano;
L is -L₁-NR⁹-L₂-;
L₁ is (CH₂)ₓ, wherein x represents an integer of 1 to 6;
L₂ is (CH₂)_{y}, wherein y represents an integer of 1 to 6,
R¹ is H and R⁹ is selected from
i) H, and
ii) C₁₋₆-alkyl;
or R¹, R⁹ and L₂ and the atoms to which they are bonded form a 4-6-membered heterocycle ring comprising a single N heteroatom;
or R¹ and L₂ and the atoms to which they are bonded form a 3-6-membered cycloalkyl ring;
R^{a} and R^{b} are independently selected from
iv) H,
v) C₁₋₆-alkyl, and
vi) halogen,
or R^{a} and R^{b} form a 3-membered cycloalkyl ring;
R^{c} and R^{d} are independently selected from
i) H,
ii) C₁₋₆-alkyl,
iii) C₃-C₆-cycloalkyl, and
iv) 4-6-membered heterocycloalkyl comprising a single oxygen heteroatom,
or R^{c} and R^{d} form a 4 membered ring comprising a single N heteroatom;
R^{e} and R^{f} are independently selected from
i) H,
ii) NH₂,
iii) hydroxyalkyl, and
iv) alkyl-COOH;
or pharmaceutically acceptable salts thereof.

2. The compound or a pharmaceutically acceptable salt thereof according to claim 1, wherein A⁴ is —O—.

3. The compound or a pharmaceutically acceptable salt thereof according to claim 1, wherein R⁶ is H.

4. The compound or a pharmaceutically acceptable salt thereof according to claim 1, wherein R⁸ is H or halogen.

5. The compound or a pharmaceutically acceptable salt thereof according to claim 1, wherein R⁸ is H.

6. The compound or a pharmaceutically acceptable salt thereof according to claim 1, wherein A² is —N—.

7. The compound or a pharmaceutically acceptable salt thereof according to claim 1, wherein B¹ is —CR⁵—.

8. The compound or a pharmaceutically acceptable salt thereof according to claim 1, wherein B² is —CR⁶—.

9. The compound or a pharmaceutically acceptable salt thereof according to claim 1, wherein B⁴ is —CR⁸—.

10. The compound or a pharmaceutically acceptable salt thereof according to claim 1, wherein R⁴ is H.

11. The compound or a pharmaceutically acceptable salt thereof according to claim 1, wherein R⁵ is cyano or halogen.

12. The compound or a pharmaceutically acceptable salt thereof according to claim 1, wherein R⁵ is halogen.

13. The compound or a pharmaceutically acceptable salt thereof according to claim 1, wherein R⁷ is selected from
i) CR^{a}R^{b}NR^{c}R^{d},
ii) CR^{a}R^{b}OH,
iii) CHR^{e}R^{f},
iv) a 5-membered heterocycloalkyl comprising a single N heteroatom,
v) a 3-6-membered heterocycloalkylalkyl comprising a single N heteroatom,
vi) a 5-membered substituted heterocycloalkyl comprising a single N or O heteroatom,
vii) a 5-membered substituted heterocycloalkylalkyl comprising a single N heteroatom, and
viii) substituted oxazole,
wherein substituted heterocycloalkyl, substituted heterocycloalkylalkyl or substituted oxazole is substituted by 1 to 2 substituents independently selected from C₁₋₆-alkyl, C₁₋₆-alkoxy, OH, hydroxyalkyl, amino, and dialkylamino, or two substituents together form a 3-to-6-membered cycloalkyl ring.

14. The compound or a pharmaceutically acceptable salt thereof according to claim 1, wherein R⁷ is selected from
i) CR^{a}R^{b}NR^{c}R^{d},
ii) CR^{a}R^{b}OH,
iii) a 4-membered heterocycloalkylalkyl comprising a single N heteroatom,
iv) a 5-membered substituted heterocycloalkyl comprising a single N heteroatom,
wherein substituted heterocycloalkyl is substituted by 1 to 2 substituents independently selected from C₁₋₆-alkyl, OH, or hydroxyalkyl.

15. The compound or a pharmaceutically acceptable salt thereof according to claim 1, wherein R⁷ is CR^{a}R^{b}NR^{c}R^{d}.

16. The compound or a pharmaceutically acceptable salt thereof according to claim 1, wherein R⁹ is H.

17. The compound or a pharmaceutically acceptable salt thereof according to claim 1, wherein R^{a} and R^{b} are independently selected from H, C₁₋₆-alkyl and halogen.

18. The compound or a pharmaceutically acceptable salt thereof according to claim 1, wherein R^{a} is H and R^{b} is C₁₋₆-alkyl.

19. The compound or a pharmaceutically acceptable salt thereof according to claim 1, wherein R^{c} and R^{d} are independently selected from C₁₋₆-alkyl or together form a 4-membered ring comprising a single N heteroatom.

20. The compound or a pharmaceutically acceptable salt thereof according to claim 1, wherein R^{c} and R^{d} are both C₁₋₆-alkyl.

21. The compound or a pharmaceutically acceptable salt thereof according to claim 1, wherein x is 2.

22. The compound or a pharmaceutically acceptable salt thereof according to claim 1, wherein y is 1.

23. The compound or a pharmaceutically acceptable salt thereof according to claim 1, wherein R¹, R⁹ and L₂ and the atoms to which they are bonded form the piperidine ring as below,

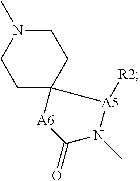

24. The compound or a pharmaceutically acceptable salt thereof according to claim 1, wherein
A¹ is —N—;
A² is —N—;
A³ is —CH—;
A⁴ is —O—;
A⁵ is —CH—;
A⁶ is —O—;
B¹ is —CR⁵—;
B² is —CR⁶—;
B³ is —CNH—C(═O)R⁷—;
B⁴ is —CR⁸—;
R² is H;
R^{3a} H;
R^{3b} is H;
R⁴ is H;
R⁵ is selected from
i) halogen, and
ii) cyano;
R⁶ is H;
R⁷ is selected from
i) CR^{a}R^{b}NR^{c}R^{d},
ii) CR^{a}R^{b}OH,
iii) a 4-membered heterocycloalkylalkyl comprising a single N heteroatom,
iv) a 5-membered substituted heterocycloalkyl comprising a single N heteroatom, wherein substituted heterocycloalkyl is substituted by 1 to 2 substituents independently selected from C₁₋₆-alkyl, OH, and hydroxyalkyl.
R⁸ is selected from
i) H, and
ii) halogen;
L is -L₁-NR⁹-L₂-;
L₁ is (CH₂)ₓ, wherein x is 1;
L₂ is (CH₂)_{y}, wherein y is 2;
R¹ and R⁹ are H,
or R¹, R⁹ and L₂ and the atoms to which they are bonded form the piperidine ring as below, 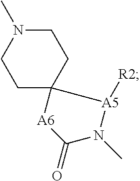
R^{a} and R^{b} are independently selected from
iv) H,
v) C₁₋₆-alkyl, and
vi) halogen,
R^{c} and R^{d} are C₁₋₆-alkyl, or R^{c} and R^{d} form a 4 membered ring comprising a single N heteroatom;
or pharmaceutically acceptable salts thereof.

25. The compound or a pharmaceutically acceptable salt thereof according to claim 1, wherein
A¹ is —N—;
A² is —N—;
A³ is —CH—;
A⁴ is —O—;
A⁵ is —CH—;
A⁶ is —O—;
B¹ is —CR⁵—;
B² is —CR⁶—;
B³ is —CNH—C(═O)R⁷—;
B⁴ is —CR⁸—;
R² is H;
R^{3a} H;
R^{3b} is H;
R⁴ is H;
R⁵ is halogen;
R⁶ is H;
R⁷ is CR^{a}R^{b}NR^{c}R^{d};
R⁸ is H;
L is -L₁-NR⁹-L₂-;
L₁ is (CH₂)ₓ, wherein x is 1;
R¹ and R⁹ are H;
L₂ is (CH₂)_{y}, wherein y is 2;
R^{a} and R^{b} are independently selected from
iii) H, and
iv) C₁₋₆-alkyl;
R^{c} and R^{d} are C₁₋₆-alkyl;
or pharmaceutically acceptable salts thereof.

26. A compound selected from
2-(dimethylamino)-N-[7-fluoro-2-[[2-[2-oxo-3-(3-oxo-4H-pyrazino[2,3-b][1,4]oxazin-6-yl)oxazolidin-5-yl]ethylamino]methyl]indan-5-yl]acetamide;
1-(dimethylamino)-N-[7-fluoro-2-[[2-[2-oxo-3-(3-oxo-4H-pyrazino[2,3-b][1,4]oxazin-6-yl)oxazolidin-5-yl]ethylamino]methyl]indan-5-yl]cyclopropanecarboxamide;
2-(dimethylamino)-N-[7-fluoro-2-[[2-[2-oxo-3-(3-oxo-4H-pyrazino[2,3-b][1,4]oxazin-6-yl)oxazolidin-5-yl]ethylamino]methyl]indan-5-yl]-2-methyl-propanamide;
(2R)-2-(dimethylamino)-N-[7-fluoro-2-[[2-[2-oxo-3-(3-oxo-4H-pyrazino[2,3-b][1,4]oxazin-6-yl)oxazolidin-5-yl]ethylamino]methyl]indan-5-yl]propanamide;
N-[7-fluoro-2-[[2-[2-oxo-3-(3-oxo-4H-pyrazino[2,3-b][1,4]oxazin-6-yl)oxazolidin-5-yl]ethylamino]methyl]indan-5-yl]-2-morpholino-acetamide;
2-(dimethylamino)-N-[7-fluoro-2-[[2-oxo-3-(3-oxo-4H-pyrazino[2,3-b][1,4]oxazin-6-yl)-1-oxa-3,8-diazaspiro[4.5]decan-8-yl]methyl]indan-5-yl]-2-methyl-propanamide;
(2R)-2-(dimethylamino)-N-[7-fluoro-2-[[2-oxo-3-(3-oxo-4H-pyrazino[2,3-b][1,4]oxazin-6-yl)-1-oxa-3,8-diazaspiro[4.5]decan-8-yl]methyl]indan-5-yl]propanamide;
2-(azetidin-1-yl)-N-[7-fluoro-2-[[2-[2-oxo-3-(3-oxo-4H-pyrazino[2,3-b][1,4]oxazin-6-yl)oxazolidin-5-yl]ethylamino]methyl]indan-5-yl]acetamide;
(2S)-2-(dimethylamino)-N-[7-fluoro-2-[[2-[2-oxo-3-(3-oxo-4H-pyrazino[2,3-b][1,4]oxazin-6-yl)oxazolidin-5-yl]ethylamino]methyl]indan-5-yl]propanamide;
(2S)—N-[7-fluoro-2-[[2-[2-oxo-3-(3-oxo-4H-pyrazino[2,3-b][1,4]oxazin-6-yl)oxazolidin-5-yl]ethylamino]methyl]indan-5-yl]-1-methyl-pyrrolidine-2-carboxamide;
N-[7-fluoro-2-[[2-[2-oxo-3-(3-oxo-4H-pyrazino[2,3-b][1,4]oxazin-6-yl)oxazolidin-5-yl]ethylamino]methyl]indan-5-yl]-2-[methyl(oxetan-3-yl)amino]acetamide;
2-(3-azabicyclo[3.1.0]hexan-3-yl)-N-[7-fluoro-2-[[2-[2-oxo-3-(3-oxo-4H-pyrazino[2,3-b][1,4]oxazin-6-yl)oxazolidin-5-yl]ethylamino]methyl]indan-5-yl]acetamide;
2-[cyclopropyl(methyl)amino]-N-[7-fluoro-2-[[2-[2-oxo-3-(3-oxo-4H-pyrazino[2,3-b][1,4]oxazin-6-yl)oxazolidin-5-yl]ethylamino]methyl]indan-5-yl]acetamide;
(2R)—N-[7-fluoro-2-[[2-[2-oxo-3-(3-oxo-4H-pyrazino[2,3-b][1,4]oxazin-6-yl)oxazolidin-5-yl]ethylamino]methyl]indan-5-yl]-1-methyl-pyrrolidine-2-carboxamide;
N-[7-fluoro-2-[[2-[2-oxo-3-(3-oxo-4H-pyrazino[2,3-b][1,4]oxazin-6-yl)oxazolidin-5-yl]ethylamino]methyl]indan-5-yl]-2-methyl-oxazole-5-carboxamide;
2-(azetidin-1-yl)-N-[7-fluoro-2-[[2-[(5R)-2-oxo-3-(3-oxo-4H-pyrazino[2,3-b][1,4]oxazin-6-yl)oxazolidin-5-yl]ethylamino]methyl]indan-5-yl]propanamide;
(2S,4R)—N-[7-fluoro-2-[[2-[2-oxo-3-(3-oxo-4H-pyrazino[2,3-b][1,4]oxazin-6-yl)oxazolidin-5-yl]ethylamino]methyl]indan-5-yl]-4-methoxy-1-methyl-pyrrolidine-2-carboxamide;
N-[7-fluoro-2-[[2-[2-oxo-3-(3-oxo-4H-pyrazino[2,3-b][1,4]oxazin-6-yl)oxazolidin-5-yl]ethylamino]methyl]indan-5-yl]-2-hydroxy-2-methyl-propanamide;
(3R)-3-(dimethylamino)-N-[7-fluoro-2-[[2-[2-oxo-3-(3-oxo-4H-pyrazino[2,3-b][1,4]oxazin-6-yl)oxazolidin-5-yl]ethylamino]methyl]indan-5-yl]tetrahydrofuran-3-carboxamide;
N-[7-cyano-2-[[2-[2-oxo-3-(3-oxo-4H-pyrazino[2,3-b][1,4]oxazin-6-yl)oxazolidin-5-yl]ethylamino]methyl]indan-5-yl]-2-hydroxy-2-methyl-propanamide;
N-[7-cyano-2-[[2-[2-oxo-3-(3-oxo-4H-pyrido[3,2-b][1,4]oxazin-6-yl)oxazolidin-5-yl]ethylamino]methyl]indan-5-yl]-2-hydroxy-2-methyl-propanamide;
(2R)—N-[7-cyano-2-[[2-[(5R)-2-oxo-3-(3-oxo-4H-pyrazino[2,3-b][1,4]oxazin-6-yl)oxazolidin-5-yl]ethylamino]methyl]indan-5-yl]-2-(dimethylamino)propanamide;
(2R)-2-(dimethylamino)-N-[4-fluoro-2-[[2-[2-oxo-3-(3-oxo-4H-pyrazino[2,3-b][1,4]oxazin-6-yl)oxazolidin-5-yl]ethylamino]methyl]indan-5-yl]propanamide;
2-(dimethylamino)-N-[4-methyl-6-[[2-[2-xo-3-(3-oxo-4H-pyrazino[2,3-b][1,4]oxazin-6-yl)oxazolidin-5-yl]ethylamino]methyl]-6,7-dihydro-5H-cyclopenta[b]pyridin-2-yl]acetamide;
(2R)-2-(dimethylamino)-N-[4-methyl-6-[[2-[2-oxo-3-(3-oxo-4H-pyrazino[2,3-b][1,4]oxazin-6-yl)oxazolidin-5-yl]ethylamino]methyl]-6,7-dihydro-5H-cyclopenta[b]pyridin-2-yl]propanamide;
2-(azetidin-1-yl)-N-[4-methyl-6-[[2-[2-oxo-3-(3-oxo-4H-pyrazino[2,3-b][1,4]oxazin-6-yl)oxazolidin-5-yl]ethylamino]methyl]-6,7-dihydro-5H-cyclopenta[b]pyridin-2-yl]acetamide;
(2S)-1-methyl-N-[4-methyl-6-[[2-[2-oxo-3-(3-oxo-4H-pyrazino[2,3-b][1,4]oxazin-6-yl)oxazolidin-5-yl]ethylamino]methyl]-6,7-dihydro-5H-cyclopenta[b]pyridin-2-yl]pyrrolidine-2-carboxamide;
(2R)-2-(dimethylamino)-N-[1-methyl-6-[[2-[2-oxo-3-(3-oxo-4H-pyrazino[2,3-b][1,4]oxazin-6-yl)oxazolidin-5-yl]ethylamino]methyl]-6,7-dihydro-5H-cyclopenta[c]pyridin-3-yl]propanamide;
(2R)-2-(dimethylamino)-N-[1-methyl-6-[[2-oxo-3-(3-oxo-4H-pyrazino[2,3-b][1,4]oxazin-6-yl)-1-oxa-3,8-diazaspiro[4.5]decan-8-yl]methyl]-6,7-dihydro-5H-cyclopenta[c]pyridin-3-yl]propanamide;
(2R)—N-[4,7-difluoro-2-hydroxy-2-[[2-[2-oxo-3-(3-oxo-4H-pyrazino[2,3-b][1,4]oxazin-6-yl)oxazolidin-5-yl]ethylamino]methyl]indan-5-yl]-2-(dimethylamino)propanamide;
(2R)—N-[4,7-difluoro-2-methoxy-2-[[2-[2-oxo-3-(3-oxo-4H-pyrazino[2,3-b][1,4]oxazin-6-yl)oxazolidin-5-yl]ethylamino]methyl]indan-5-yl]-2-(dimethylamino)propanamide;
(2R)-2-(dimethylamino)-N-[2,4,7-trifluoro-2-[[2-[2-oxo-3-(3-oxo-4H-pyrazino[2,3-b][1,4]oxazin-6-yl)oxazolidin-5-yl]ethylamino]methyl]indan-5-yl]propanamide;
(2R)—N-[2,7-difluoro-2-[[2-[2-oxo-3-(3-oxo-4H-pyrazino[2,3-b][1,4]oxazin-6-yl)oxazolidin-5-yl]ethylamino]methyl]indan-5-yl]-2-(dimethylamino)propanamide;
(2R)—N-[7-cyano-2-hydroxy-2-[[2-[2-oxo-3-(3-oxo-4H-pyrazino[2,3-b][1,4]oxazin-6-yl)oxazolidin-5-yl]ethylamino]methyl]indan-5-yl]-2-(dimethylamino)propanamide;
(2R)—N-[7-cyano-2-hydroxy-2-[[2-oxo-3-(3-oxo-4H-pyrazino[2,3-b][1,4]oxazin-6-yl)-1-oxa-3,8-diazaspiro[4.5]decan-8-yl]methyl]indan-5-yl]-2-(dimethylamino)propanamide;
(2S)—N-[7-fluoro-2-[[2-[2-oxo-3-(3-oxo-4H-pyrazino[2,3-b][1,4]oxazin-6-yl)oxazolidin-5-yl]ethylamino]methyl]indan-5-yl]pyrrolidine-2-carboxamide;
(2S,4R)—N-[7-fluoro-2-[[2-[2-oxo-3-(3-oxo-4H-pyrazino[2,3-b][1,4]oxazin-6-yl)oxazolidin-5-yl]ethylamino]methyl]indan-5-yl]-4-hydroxy-pyrrolidine-2-carboxamide;
(2R)-2-amino-N-[7-fluoro-2-[[2-[2-oxo-3-(3-oxo-4H-pyrazino[2,3-b][1,4]oxazin-6-yl)oxazolidin-5-yl]ethylamino]methyl]indan-5-yl]-3-hydroxy-propanamide;
(2R)-2-amino-N-[7-fluoro-2-[[2-[2-oxo-3-(3-oxo-4H-pyrazino[2,3-b][1,4]oxazin-6-yl)oxazolidin-5-yl]ethylamino]methyl]indan-5-yl]-3-methoxy-propanamide;
(3R)-3-amino-N-[7-fluoro-2-[[2-[2-oxo-3-(3-oxo-4H-pyrazino[2,3-b][1,4]oxazin-6-yl)oxazolidin-5-yl]ethylamino]methyl]indan-5-yl]tetrahydrofuran-3-carboxamide;
(3R)-3-amino-N-[7-fluoro-2-[[2-[2-oxo-3-(3-oxo-4H-pyrazino[2,3-b][1,4]oxazin-6-yl)oxazolidin-5-yl]ethylamino]methyl]indan-5-yl]tetrahydrofuran-3-carboxamide; 2,2,2-trifluoroacetic acid;
(3S)-3-amino-N-[7-fluoro-2-[[2-[2-oxo-3-(3-oxo-4H-pyrazino[2,3-b][1,4]oxazin-6-yl)oxazolidin-5-yl]ethylamino]methyl]indan-5-yl]tetrahydrofuran-3-carboxamide;
(3S)-3-amino-N-[7-fluoro-2-[[2-[2-oxo-3-(3-oxo-4H-pyrazino[2,3-b][1,4]oxazin-6-yl)oxazolidin-5-yl]ethylamino]methyl]indan-5-yl]tetrahydrofuran-3-carboxamide; hydrochloride;
(2S,4R)—N-[7-fluoro-2-[[2-oxo-3-(3-oxo-4H-pyrazino[2,3-b][1,4]oxazin-6-yl)-1-oxa-3,8-diazaspiro[4.5]decan-8-yl]methyl]indan-5-yl]-4-hydroxy-pyrrolidine-2-carboxamide;
2-amino-N-[7-fluoro-2-[[2-[2-oxo-3-(3-oxo-4H-pyrazino[2,3-b][1,4]oxazin-6-yl)oxazolidin-5-yl]ethylamino]methyl]indan-5-yl]-2-methyl-propanamide;
(2S,4R)—N-[7-fluoro-2-[[2-[2-oxo-3-(3-oxo-4H-pyrazino[2,3-b][1,4]oxazin-6-yl)oxazolidin-5-yl]ethylamino]methyl]indan-5-yl]-4-methoxy-pyrrolidine-2-carboxamide;
(2R,4R)—N-[7-fluoro-2-[[2-[2-oxo-3-(3-oxo-4H-pyrazino[2,3-b][1,4]oxazin-6-yl)oxazolidin-5-yl]ethylamino]methyl]indan-5-yl]-4-hydroxy-pyrrolidine-2-carboxamide;
(2S,4S)—N-[7-fluoro-2-[[2-[2-oxo-3-(3-oxo-4H-pyrazino[2,3-b][1,4]oxazin-6-yl)oxazolidin-5-yl]ethylamino]methyl]indan-5-yl]-4-hydroxy-pyrrolidine-2-carboxamide;
(2R,4S)—N-[7-fluoro-2-[[2-[2-oxo-3-(3-oxo-4H-pyrazino[2,3-b][1,4]oxazin-6-yl)oxazolidin-5-yl]ethylamino]methyl]indan-5-yl]-4-hydroxy-pyrrolidine-2-carboxamide;
(3S)-3-amino-4-[[7-fluoro-2-[[2-[2-oxo-3-(3-oxo-4H-pyrazino[2,3-b][1,4]oxazin-6-yl)oxazolidin-5-yl]ethylamino]methyl]indan-5-yl]amino]-4-oxo-butanoic acid;
(3S)-3-amino-4-[[7-fluoro-2-[[2-[2-oxo-3-(3-oxo-4H-pyrazino[2,3-b][1,4]oxazin-6-yl)oxazolidin-5-yl]ethylamino]methyl]indan-5-yl]amino]-4-oxo-butanoic acid; 2,2,2-trifluoroacetic acid;
(2S,4R)—N-[7-fluoro-2-[[2-[2-oxo-3-(3-oxo-4H-pyrazino[2,3-b][1,4]oxazin-6-yl)oxazolidin-5-yl]ethylamino]methyl]indan-5-yl]-4-hydroxy-1-methyl-pyrrolidine-2-carboxamide;
N-[7-fluoro-2-[[2-[2-oxo-3-(3-oxo-4H-pyrazino[2,3-b][1,4]oxazin-6-yl)oxazolidin-5-yl]ethylamino]methyl]indan-5-yl]-2-[(3R)-3-hydroxypyrrolidin-1-yl]acetamide;
(2S)-2-(dimethylamino)-N-[7-fluoro-2-[[2-oxo-3-(3-oxo-4H-pyrazino[2,3-b][1,4]oxazin-6-yl)-1-oxa-3,8-diazaspiro[4.5]decan-8-yl]methyl]indan-5-yl]propanamide;
(2S,4R)—N-[4,7-difluoro-2-[[2-oxo-3-(3-oxo-4H-pyrazino[2,3-b][1,4]oxazin-6-yl)-1-oxa-3,8-diazaspiro[4.5]decan-8-yl]methyl]indan-5-yl]-4-hydroxy-pyrrolidine-2-carboxamide;
(2R)—N-[7-fluoro-2-[[2-oxo-3-(3-oxo-4H-pyrazino[2,3-b][1,4]oxazin-6-yl)-1-oxa-3,8-diazaspiro[4.5]decan-8-yl]methyl]indan-5-yl]-2-(hydroxymethyl)pyrrolidine-2-carboxamide;
(2R)—N-[7-fluoro-2-[[2-oxo-3-(3-oxo-4H-pyrazino[2,3-b][1,4]oxazin-6-yl)-1-oxa-3,8-diazaspiro[4.5]decan-8-yl]methyl]indan-5-yl]-2-(hydroxymethyl)pyrrolidine-2-carboxamide; 2,2,2-trifluoroacetic acid;
(2S,3S)—N-[7-fluoro-2-[[2-oxo-3-(3-oxo-4H-pyrazino[2,3-b][1,4]oxazin-6-yl)-1-oxa-3,8-diazaspiro[4.5]decan-8-yl]methyl]indan-5-yl]-3-hydroxy-pyrrolidine-2-carboxamide;
(2S,3S)—N-[7-fluoro-2-[[2-oxo-3-(3-oxo-4H-pyrazino[2,3-b][1,4]oxazin-6-yl)-1-oxa-3,8-diazaspiro[4.5]decan-8-yl]methyl]indan-5-yl]-3-hydroxy-pyrrolidine-2-carboxamide; 2,2,2-trifluoroacetic acid;
(2R,4R)—N-[7-fluoro-2-[[2-oxo-3-(3-oxo-4H-pyrazino[2,3-b][1,4]oxazin-6-yl)-1-oxa-3,8-diazaspiro[4.5]decan-8-yl]methyl]indan-5-yl]-4-hydroxy-pyrrolidine-2-carboxamide;
(2R,4S)—N-[7-fluoro-2-[[2-oxo-3-(3-oxo-4H-pyrazino[2,3-b][1,4]oxazin-6-yl)-1-oxa-3,8-diazaspiro[4.5]decan-8-yl]methyl]indan-5-yl]-4-hydroxy-pyrrolidine-2-carboxamide;
(2S,4S)—N-[7-fluoro-2-[[2-oxo-3-(3-oxo-4H-pyrazino[2,3-b][1,4]oxazin-6-yl)-1-oxa-3,8-diazaspiro[4.5]decan-8-yl]methyl]indan-5-yl]-4-hydroxy-pyrrolidine-2-carboxamide;
(2R)-2-(dimethylamino)-N-[7-fluoro-2-[[methyl-[2-[2-oxo-3-(3-oxo-4H-pyrazino[2,3-b][1,4]oxazin-6-yl)oxazolidin-5-yl]ethyl]amino]methyl]indan-5-yl]propanamide;
(2S,4R)—N-[7-cyano-2-[[2-oxo-3-(3-oxo-4H-pyrazino[2,3-b][1,4]oxazin-6-yl)-1-oxa-3,8-diazaspiro[4.5]decan-8-yl]methyl]indan-5-yl]-4-hydroxy-pyrrolidine-2-carboxamide;
(2S,4R)-4-hydroxy-N-[6-[[2-oxo-3-(3-oxo-4H-pyrazino[2,3-b][1,4]oxazin-6-yl)-1-oxa-3,8-diazaspiro[4.5]decan-8-yl]methyl]-6,7-dihydro-5H-cyclopenta[b]pyridin-3-yl]pyrrolidine-2-carboxamide;
(2S,4R)—N-[7-cyano-2-[[2-[2-oxo-3-(3-oxo-4H-pyrazino[2,3-b][1,4]oxazin-6-yl)oxazolidin-5-yl]ethylamino]methyl]indan-5-yl]-4-hydroxy-pyrrolidine-2-carboxamide;
(2R)—N-[7-cyano-2-[[2-[2-oxo-3-(3-oxo-4H-pyrazino[2,3-b][1,4]oxazin-6-yl)oxazolidin-5-yl]ethylamino]methyl]indan-5-yl]-1-methyl-pyrrolidine-2-carboxamide;
(2S,4R)—N-[7-fluoro-2-[[methyl-[2-[2-oxo-3-(3-oxo-4H-pyrazino[2,3-b][1,4]oxazin-6-yl)oxazolidin-5-yl]ethyl]amino]methyl]indan-5-yl]-4-hydroxy-pyrrolidine-2-carboxamide;
(2S,4R)—N-[7-fluoro-2-[[2-oxo-3-(3-oxo-4H-pyrazino[2,3-b][1,4]oxazin-6-yl)-1-oxa-3,8-diazaspiro[4.5]decan-8-yl]methyl]indan-5-yl]-4-hydroxy-1-methyl-pyrrolidine-2-carboxamide;
(2R)-2-(dimethylamino)-N-[7-fluoro-3-hydroxy-2-[[2-oxo-3-(3-oxo-4H-pyrazino[2,3-b][1,4]oxazin-6-yl)-1-oxa-3,8-diazaspiro[4.5]decan-8-yl]methyl]indan-5-yl]propanamide;
(2R)-2-(dimethylamino)-N-[7-fluoro-3-hydroxy-2-[[2-[(5R)-2-oxo-3-(3-oxo-4H-pyrazino[2,3-b][1,4]oxazin-6-yl)oxazolidin-5-yl]ethylamino]methyl]indan-5-yl]propanamide;
(2R)—N-[3,7-difluoro-2-[[2-[(5R)-2-oxo-3-(3-oxo-4H-pyrazino[2,3-b][1,4]oxazin-6-yl)oxazolidin-5-yl]ethylamino]methyl]indan-5-yl]-2-(dimethylamino)propanamide;
(2R)-2-(dimethylamino)-N-[7-fluoro-2-[[2-[(5S)-2-oxo-3-(3-oxo-4H-pyrazino[2,3-b][1,4]oxazin-6-yl)oxazolidin-5-yl]ethylamino]methyl]indan-5-yl]propanamide;
(2R)-2-(dimethylamino)-N-[7-fluoro-2-[[2-[(5R)-2-oxo-3-(3-oxo-4H-pyrazino[2,3-b][1,4]oxazin-6-yl)oxazolidin-5-yl]ethylamino]methyl]indan-5-yl]propanamide;
or pharmaceutically acceptable salts thereof.

27. A compound according to claim 1, selected from
2-(dimethylamino)-N-[7-fluoro-2-[[2-[2-oxo-3-(3-oxo-4H-pyrazino[2,3-b][1,4]oxazin-6-yl)oxazolidin-5-yl]ethylamino]methyl]indan-5-yl]acetamide;
2-(dimethylamino)-N-[7-fluoro-2-[[2-[2-oxo-3-(3-oxo-4H-pyrazino[2,3-b][1,4]oxazin-6-yl)oxazolidin-5-yl]ethylamino]methyl]indan-5-yl]-2-methyl-propanamide;
(2R)-2-(dimethylamino)-N-[7-fluoro-2-[[2-[2-oxo-3-(3-oxo-4H-pyrazino[2,3-b][1,4]oxazin-6-yl)oxazolidin-5-yl]ethylamino]methyl]indan-5-yl]propanamide;
2-(dimethylamino)-N-[7-fluoro-2-[[2-oxo-3-(3-oxo-4H-pyrazino[2,3-b][1,4]oxazin-6-yl)-1-oxa-3,8-diazaspiro[4.5]decan-8-yl]methyl]indan-5-yl]-2-methyl-propanamide;
(2R)-2-(dimethylamino)-N-[7-fluoro-2-[[2-oxo-3-(3-oxo-4H-pyrazino[2,3-b][1,4]oxazin-6-yl)-1-oxa-3,8-diazaspiro[4.5]decan-8-yl]methyl]indan-5-yl]propanamide;
2-(azetidin-1-yl)-N-[7-fluoro-2-[[2-[2-oxo-3-(3-oxo-4H-pyrazino[2,3-b][1,4]oxazin-6-yl)oxazolidin-5-yl]ethylamino]methyl]indan-5-yl]acetamide;
(2S)-2-(dimethylamino)-N-[7-fluoro-2-[[2-[2-oxo-3-(3-oxo-4H-pyrazino[2,3-b][1,4]oxazin-6-yl)oxazolidin-5-yl]ethylamino]methyl]indan-5-yl]propanamide;
(2S)—N-[7-fluoro-2-[[2-[2-oxo-3-(3-oxo-4H-pyrazino[2,3-b][1,4]oxazin-6-yl)oxazolidin-5-yl]ethylamino]methyl]indan-5-yl]-1-methyl-pyrrolidine-2-carboxamide;
(2R)—N-[7-fluoro-2-[[2-[2-oxo-3-(3-oxo-4H-pyrazino[2,3-b][1,4]oxazin-6-yl)oxazolidin-5-yl]ethylamino]methyl]indan-5-yl]-1-methyl-pyrrolidine-2-carboxamide;
2-(azetidin-1-yl)-N-[7-fluoro-2-[[2-[(5R)-2-oxo-3-(3-oxo-4H-pyrazino[2,3-b][1,4]oxazin-6-yl)oxazolidin-5-yl]ethylamino]methyl]indan-5-yl]propanamide;
N-[7-fluoro-2-[[2-[2-oxo-3-(3-oxo-4H-pyrazino[2,3-b][1,4]oxazin-6-yl)oxazolidin-5-yl]ethylamino]methyl]indan-5-yl]-2-hydroxy-2-methyl-propanamide;
(2R)—N-[7-cyano-2-[[2-[(5R)-2-oxo-3-(3-oxo-4H-pyrazino[2,3-b][1,4]oxazin-6-yl)oxazolidin-5-yl]ethylamino]methyl]indan-5-yl]-2-(dimethylamino)propanamide;
(2S,4R)—N-[7-fluoro-2-[[2-oxo-3-(3-oxo-4H-pyrazino[2,3-b][1,4]oxazin-6-yl)-1-oxa-3,8-diazaspiro[4.5]decan-8-yl]methyl]indan-5-yl]-4-hydroxy-pyrrolidine-2-carboxamide;
(2R,4S)—N-[7-fluoro-2-[[2-[2-oxo-3-(3-oxo-4H-pyrazino[2,3-b][1,4]oxazin-6-yl)oxazolidin-5-yl]ethylamino]methyl]indan-5-yl]-4-hydroxy-pyrrolidine-2-carboxamide;
(2S,4R)—N-[7-fluoro-2-[[2-[2-oxo-3-(3-oxo-4H-pyrazino[2,3-b][1,4]oxazin-6-yl)oxazolidin-5-yl]ethylamino]methyl]indan-5-yl]-4-hydroxy-1-methyl-pyrrolidine-2-carboxamide;
(2S)-2-(dimethylamino)-N-[7-fluoro-2-[[2-oxo-3-(3-oxo-4H-pyrazino[2,3-b][1,4]oxazin-6-yl)-1-oxa-3,8-diazaspiro[4.5]decan-8-yl]methyl]indan-5-yl]propanamide;
(2S,4R)—N-[4,7-difluoro-2-[[2-oxo-3-(3-oxo-4H-pyrazino[2,3-b][1,4]oxazin-6-yl)-1-oxa-3,8-diazaspiro[4.5]decan-8-yl]methyl]indan-5-yl]-4-hydroxy-pyrrolidine-2-carboxamide;
(2R)—N-[7-fluoro-2-[[2-oxo-3-(3-oxo-4H-pyrazino[2,3-b][1,4]oxazin-6-yl)-1-oxa-3,8-diazaspiro[4.5]decan-8-yl]methyl]indan-5-yl]-2-(hydroxymethyl)pyrrolidine-2-carboxamide;
(2R)—N-[7-fluoro-2-[[2-oxo-3-(3-oxo-4H-pyrazino[2,3-b][1,4]oxazin-6-yl)-1-oxa-3,8-diazaspiro[4.5]decan-8-yl]methyl]indan-5-yl]-2-(hydroxymethyl)pyrrolidine-2-carboxamide; 2,2,2-trifluoroacetic acid;
(2S,3S)—N-[7-fluoro-2-[[2-oxo-3-(3-oxo-4H-pyrazino[2,3-b][1,4]oxazin-6-yl)-1-oxa-3,8-diazaspiro[4.5]decan-8-yl]methyl]indan-5-yl]-3-hydroxy-pyrrolidine-2-carboxamide;
(2S,3S)—N-[7-fluoro-2-[[2-oxo-3-(3-oxo-4H-pyrazino[2,3-b][1,4]oxazin-6-yl)-1-oxa-3,8-diazaspiro[4.5]decan-8-yl]methyl]indan-5-yl]-3-hydroxy-pyrrolidine-2-carboxamide; 2,2,2-trifluoroacetic acid;
(2R,4R)—N-[7-fluoro-2-[[2-oxo-3-(3-oxo-4H-pyrazino[2,3-b][1,4]oxazin-6-yl)-1-oxa-3,8-diazaspiro[4.5]decan-8-yl]methyl]indan-5-yl]-4-hydroxy-pyrrolidine-2-carboxamide;
(2R,4S)—N-[7-fluoro-2-[[2-oxo-3-(3-oxo-4H-pyrazino[2,3-b][1,4]oxazin-6-yl)-1-oxa-3,8-diazaspiro[4.5]decan-8-yl]methyl]indan-5-yl]-4-hydroxy-pyrrolidine-2-carboxamide;
(2S,4S)—N-[7-fluoro-2-[[2-oxo-3-(3-oxo-4H-pyrazino[2,3-b][1,4]oxazin-6-yl)-1-oxa-3,8-diazaspiro[4.5]decan-8-yl]methyl]indan-5-yl]-4-hydroxy-pyrrolidine-2-carboxamide;
(2S,4R)—N-[7-cyano-2-[[2-oxo-3-(3-oxo-4H-pyrazino[2,3-b][1,4]oxazin-6-yl)-1-oxa-3,8-diazaspiro[4.5]decan-8-yl]methyl]indan-5-yl]-4-hydroxy-pyrrolidine-2-carboxamide;
(2R)—N-[7-cyano-2-[[2-[2-oxo-3-(3-oxo-4H-pyrazino[2,3-b][1,4]oxazin-6-yl)oxazolidin-5-yl]ethylamino]methyl]indan-5-yl]-1-methyl-pyrrolidine-2-carboxamide;
(2S,4R)—N-[7-fluoro-2-[[2-oxo-3-(3-oxo-4H-pyrazino[2,3-b][1,4]oxazin-6-yl)-1-oxa-3,8-diazaspiro[4.5]decan-8-yl]methyl]indan-5-yl]-4-hydroxy-1-methyl-pyrrolidine-2-carboxamide;
(2R)-2-(dimethylamino)-N-[7-fluoro-2-[[2-[(5S)-2-oxo-3-(3-oxo-4H-pyrazino[2,3-b][1,4]oxazin-6-yl)oxazolidin-5-yl]ethylamino]methyl]indan-5-yl]propanamide;
(2R)-2-(dimethylamino)-N-[7-fluoro-2-[[2-[(5R)-2-oxo-3-(3-oxo-4H-pyrazino[2,3-b][1,4]oxazin-6-yl)oxazolidin-5-yl]ethylamino]methyl]indan-5-yl]propanamide;
(2S,4R)—N-[7-fluoro-2-[[2-oxo-3-(3-oxo-4H-pyrazino[2,3-b][1,4]oxazin-6-yl)-1-oxa-3,8-diazaspiro[4.5]decan-8-yl]methyl]indan-5-yl]-4-hydroxy-2-methyl-pyrrolidine-2-carboxamide
or pharmaceutically acceptable salts thereof.

28. A compound according to claim 1, selected from
(2R)-2-(dimethylamino)-N-[7-fluoro-2-[[2-[2-oxo-3-(3-oxo-4H-pyrazino[2,3-b][1,4]oxazin-6-yl)oxazolidin-5-yl]ethylamino]methyl]indan-5-yl]propanamide;
(2R)-2-(dimethylamino)-N-[7-fluoro-2-[[2-[(5S)-2-oxo-3-(3-oxo-4H-pyrazino[2,3-b][1,4]oxazin-6-yl)oxazolidin-5-yl]ethylamino]methyl]indan-5-yl]propanamide;
(2R)-2-(dimethylamino)-N-[7-fluoro-2-[[2-[(5R)-2-oxo-3-(3-oxo-4H-pyrazino[2,3-b][1,4]oxazin-6-yl)oxazolidin-5-yl]ethylamino]methyl]indan-5-yl]propanamide;
or pharmaceutically acceptable salts thereof.

29. A pharmaceutical composition comprising a compound according to claim 1 and a therapeutically inert carrier.

30. A method for the treatment or prophylaxis of infections and resulting diseases caused by Escherichia coli, which method comprises administering an effective amount of a compound according to claim 1.

31. A method for the treatment or prophylaxis of bacterial infection, which method comprises administering an effective amount of a compound according to claim 1.

32. A process to prepare a compound according to claim 1 comprising the reacting of a compound of formula III with a compound of formula II in reaction conditions that include sodium triacetoxyborohydride or sodium cyanoborohydride, optionally in the presence of additives such as N,N-diisopropylethylamine or triethylamine or acetic acid or powdered molecular sieves in a solvent such as methanol, THF or 1,2-dichloroethane at a temperature such as room temperature 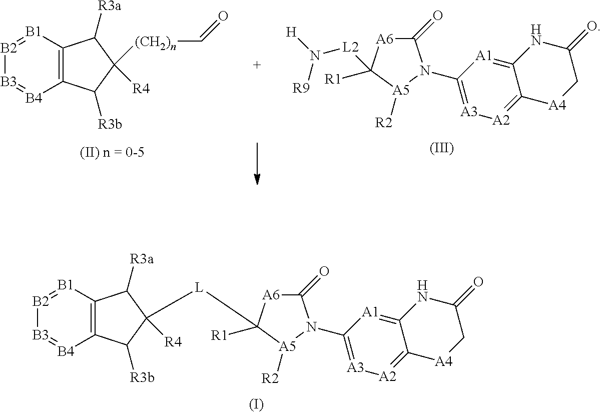
wherein R¹, R², R^{3a}, R^{3b}, R⁴, A¹, A², A³, A⁴, A⁵, A⁶, B¹, B², B³, B⁴, and L are as described above.
